(19) United States
(12) Patent Application Publication
(10) Pub. No.: US 2010/0113502 A1
(43) Pub. Date: May 6, 2010

(54) Novel Tetrahydro-1H-Pyrido[4,3-b] Indole Derivatives as CB1 Receptor Ligands

(75) Inventors: Yun-Xing Cheng, St. Laurent (CA); Miroslaw Tomaszewski, St. Laurent (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 11/909,089

(22) PCT Filed: Mar. 17, 2006
(86) PCT No.: PCT/SE06/00339

§ 371 Date: Sep. 19, 2007

(30) Foreign Application Priority Data
Mar. 22, 2005 (SE) 0500654-9

(51) Int. Cl. A61K 31/437; C07D 471/04; A61P 25/22

(52) U.S. Cl. 514/292; 546/87

(57) ABSTRACT

Compounds of formulae I, or pharmaceutically acceptable salts thereof: wherein R¹, R² and R³ are as defined in the specification as well as salts and pharmaceutical compositions including the compounds are prepared. They are useful in therapy, in particular in the management of pain.

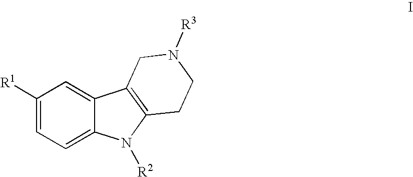

This application claims priority under 35 U.S.C. §119(a)-(d) to Swedish Application No. 0500654-9 filed on Mar. 22, 2005, which is hereby incorporated by reference in its entirety.

### BACKGROUND OF THE INVENTION

1. Field of the Invention

The invention is related to therapeutic compounds, pharmaceutical compositions containing these compounds, manufacturing processes thereof and uses thereof. Particularly, the present invention is related to compounds that may be effective in treating pain, cancer, multiple sclerosis, Parkinson's disease, Huntington's chorea, Alzheimer's disease, anxiety disorders, gastrointestinal disorders and/or cardiovascular disorders.

2. Discussion of Relevant Technology

Pain management has been studied for many years. It is known that cannabinoid receptor (e.g., CB₁ receptor, CB₂ receptor) ligands including agonists, antagonists and inverse agonists produce relief of pain in a variety of animal models by interacting with CB₁ and/or CB₂ receptors. Generally, CB₁ receptors are located predominately in the central nervous system, whereas CB₂ receptors are located primarily in the periphery and are primarily restricted to the cells and tissues derived from the immune system.

While CB₁ receptor agonists, such as Δ⁹-tetrahydrocannabinol (Δ⁹-THC) and anadamide, are useful in anti-nociception models in animals, they tend to exert undesired CNS side-effects, e.g., psychoactive side effects, the abuse potential, drug dependence and tolerance, etc. These undesired side effects are known to be mediated by the CB₁ receptors located in CNS. There are lines of evidence, however, suggesting that CB₁ agonists acting at peripheral sites or with limited CNS exposure can manage pain in humans or animals with much improved overall in vivo profile.

Therefore, there is a need for new CB₁ receptor ligands such as agonists that may be useful in managing pain or treating other related symptoms or diseases with reduced or minimal undesirable CNS side-effects.

### DESCRIPTION OF THE EMBODIMENTS

The present invention provides CB₁ receptor ligands which may be useful in treating pain and/or other related symptoms or diseases.

The term “Cₘ₋ₙ” or “Cₘ₋ₙ group” refers to any group having m to n carbon atoms.

The term “alkyl” refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl; 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkenyl” refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “cycloalkyl” refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “cycloalkenyl” refers to a monovalent ring-containing hydrocarbon radical having at least one carbon-carbon double bond and comprising at least 3 up to about 12 carbon atoms.

The term “aryl” refers to a monovalent hydrocarbon radical having one or more polyunsaturated carbon rings having aromatic character, (e.g., 4n+2 delocalized electrons) and comprising 5 up to about 14 carbon atoms.

The term “heterocycle” refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroaromatic” refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” refers to a radical derived from a heterocycle by removing one or more hydrogens therefrom.

The term “heterocyclyl” refers a monovalent radical derived from a heterocycle by removing one hydrogen therefrom.

The term “heterocyclylene” refers to a divalent radical derived from a heterocycle by removing two hydrogens therefrom, which serves to links two structures together.

The term “heteroaryl” refers to a heterocyclyl having aromatic character.

The term “heterocylcoallyl” refers to a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆ heterocycloalkyl.

The term “heteroarylene” refers to a heterocyclylene having aromatic character.

The term “heterocycloalkylene” refers to a heterocyclylene that does not have aromatic character.

The term “six-membered” refers to a group having a ring that contains six ring atoms.

The term “five-membered” refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

Halogen includes fluorine, chlorine, bromine and iodine.

“RT” or “rt” means room temperature.

In one aspect, an embodiment of the invention provides a compound of Formula I, a pharmaceutically acceptable salt thereof, diastereomers, enantiomers, or mixtures thereof:

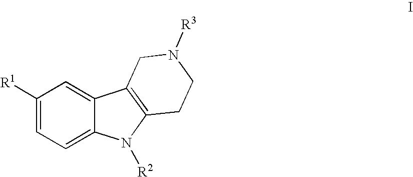

wherein

R¹ is selected from —C(═O)—R⁴, —NH—C(═O)—R⁵, —NH—C(═O)—NH—R⁶, —NR⁷—S(═O)₂—R⁸, —NR⁷—S(═O)₂—NR⁹R¹⁰;

R² is selected from —H, C₁₋₆alkyl, C₂₋₆alkenyl, —C(═O)—NR⁹R¹⁰, —S(═O)₂—NR⁹R¹⁰, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl used in defining R² is optionally substituted with one or more group selected from —OR, R, —CO₂H, —CO₂—R; —SO₂—R; halogen, —NO₂, —OH, —NH₂, —NHR, —C(═O)—NH₂, and —C(═O)—NHR;

R³ is selected from C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₁₋₆alkenyl, C₆₋₁₀aryl-C₁₋₄alkyl, C₃₋₆heteroaryl-C₁₋₄alkyl, —C(═O)—C₁₋₆alkyl, —C(═O)—C₃₋₆cycloalkyl and —C(═NH)—C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₁₋₆alkenyl, C₆₋₁₀aryl-C₁₋₄alkyl, C₃₋₆heteroaryl-C₁₋₄alkyl, —C(═O)—C₁₋₆alkyl, —C(═O)—C₃₋₆cycloalkyl and —C(═NH)—C₁₋₆alkyl used in defining R³ is optionally substituted with one or more groups selected from —OR, R, NO₂, —CO₂H, —CO₂—R; —SO₂—R; halogen; —OH; —NH₂; —NHR, —C(═O)—NH₂, and —C(═O)—NHR;

R is C₁₋₆alkyl;

R⁴ is selected from a nitrogen containing C₃₋₉heterocyclyl and —NR⁹R¹⁰, wherein said nitrogen containing C₃₋₉heterocyclyl may be optionally substituted with one or more groups selected from C₁₋₆alkyl, phenyl, C₁₋₆alkoxy, —NH₂, —OH, halogenated C₁₋₆alkyl, and halogen; and

R⁵, R⁶, R⁷, R⁸, R⁹ and R¹⁰ are independently selected from —H, C₁₋₆alkyl, C₆₋₁₀aryl, C₆₋₁₀aryl-C₁₋₄alkyl, C₃₋₆heterocyclyl, C₃₋₆heterocyclyl-C₁₋₄alkyl, C₂₋₆ alkenyl, C₃₋₆cycloalkyl, and C₃₋₆cycloalkyl-C₁₋₄alkyl; N,N-di(C₁₋₄alkyl)amido-C₁₋₆allyl, hydroxy-C₁₋₆alkyl and C₁₋₆alkoxy-C₁₋₆alkyl.

Another embodiment of the invention provides a compound of formula I, wherein

R¹ is selected from —C(═O)—R⁴, —NH—C(═O)—R⁵, —NH—C(═O)—NH—R⁶, —NR⁷—S(═O)₂—R⁸, —NR⁷—S(═O)₂—NR⁹R¹⁰;

R² is selected from C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—NR⁹R¹⁰, —S(═O)₂—NR⁹R¹⁰, C(═O)—C₁₋₆alkyl; C₆₋₁₀aryl-C₁₋₄alkyl; and C₃₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄allyl used in defining R² is optionally substituted with one or more group selected from methoxy, ethoxy, —S(═O)₂CH₃, methyl, ethyl, —NO₂, —CO₂H, —CO₂CH₃, —CO₂CH₂CH₃ and halogen;

R³ is selected from C₃₋₆heterocycloalkyl, C₃₋₆heterocycloalkyl-C₁₋₄alkyl, C₃₋₆cycloalkyl, C₃₋₆cycloalkyl-C₁₋₄allyl and C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl and C₁₋₆alkyl used in defining R³ is optionally substituted with one or more groups selected from methoxy, ethoxy, —S(═O)₂CH₃, methyl, ethyl, —NO₂, —CO₂H, —CO₂CH₃, —CO₂CH₂CH₃ and halogen;

R⁴ is selected from a nitrogen containing C₃₋₉heterocycloalkyl and —NR⁹R¹⁰, wherein said nitrogen containing C₃₋₉heterocycloalkyl may be optionally substituted with one or more groups selected from methyl, ethyl, phenyl, methoxy, ethoxy, —OH, trifluoromethyl and halogen; and

R⁵, R⁶, R⁷, R⁸, R⁹ and R¹⁰ are independently selected from —H, C₁₋₆alkyl, phenyl, benzyl, C₃₋₆heterocyclyl, C₃₋₆heterocyclyl-methyl, C₂₋₆alkenyl, C₃₋₆cycloalkyl, and C₃₋₆cycloalkyl-methyl; hydroxy-C₁₋₆alkyl, methoxy-C₁₋₆alkyl and ethoxy-C₁₋₆alkyl.

A further embodiment of the invention provides a compound of formula I, wherein

R¹ is selected from —C(═O)—R⁴ and —NH—C(═O)—R⁵;

R² is selected from —H, C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—NR⁹R¹⁰, —S(═O)₂—NR⁹R¹⁰, —C(═O)—C₁₋₆alkyl, benzyl and C₃₋₅heteroaryl-methyl, wherein said C₁₋₆alkyl, C₂₋₆alkenyl, —S(═O)₂—C₁₋₆allyl, —C(═O)—NR⁹R¹⁰, —S(═O)₂—NR⁹R¹⁰, —C(═O)—C₁₋₆alkyl, benzyl and C₃₋₅heteroaryl-methyl used in defining R² is optionally substituted with one or more group selected from methoxy, ethoxy, —NO₂, —S(═O)₂CH₃, methyl, ethyl, —CO₂H, —CO₂CH₃, —CO₂CH₂CH₃ and halogen;

R³ is selected from C₃₋₆heterocycloalkyl, C₃₋₆heterocycloalkyl-methyl, C₃₋₆cycloalkyl, C₃₋₆cycloalkyl-methyl and C₁₋₆alkyl, wherein said C₃₋₆heterocycloalkyl, C₃₋₆heterocycloalkyl-methyl, C₃₋₆cycloalkyl, C₃₋₆cycloalkyl-methyl and C₁₋₆alkyl used in defining R³ is optionally substituted with one or more groups selected from methoxy, ethoxy, methyl, ethyl and halogen;

R⁴ is selected from piperidinyl, piperazinyl and morpholinyl, wherein said piperidinyl, piperazinyl and morpholinyl are optionally substituted with one or more groups selected from methyl, ethyl, methoxy, ethoxy, —OH, hydroxylmethyl, trifluoromethyl and halogen; and

R⁹ and R¹⁰ are independently selected from —H, C₁₋₆allyl and C₂₋₆alkenyl.

An even further embodiment of the invention provides a compound of formula I,

wherein

R¹ is selected from

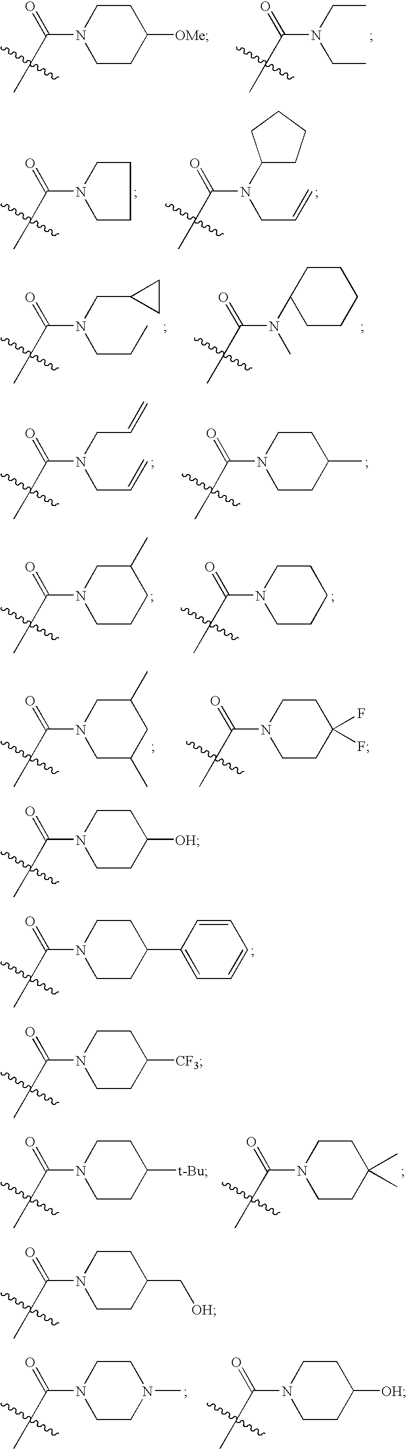

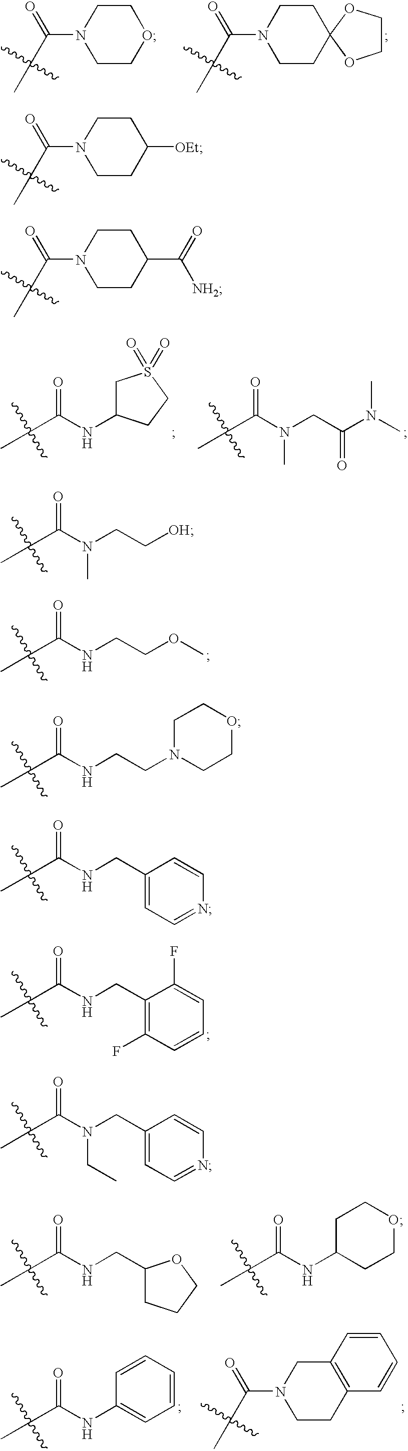

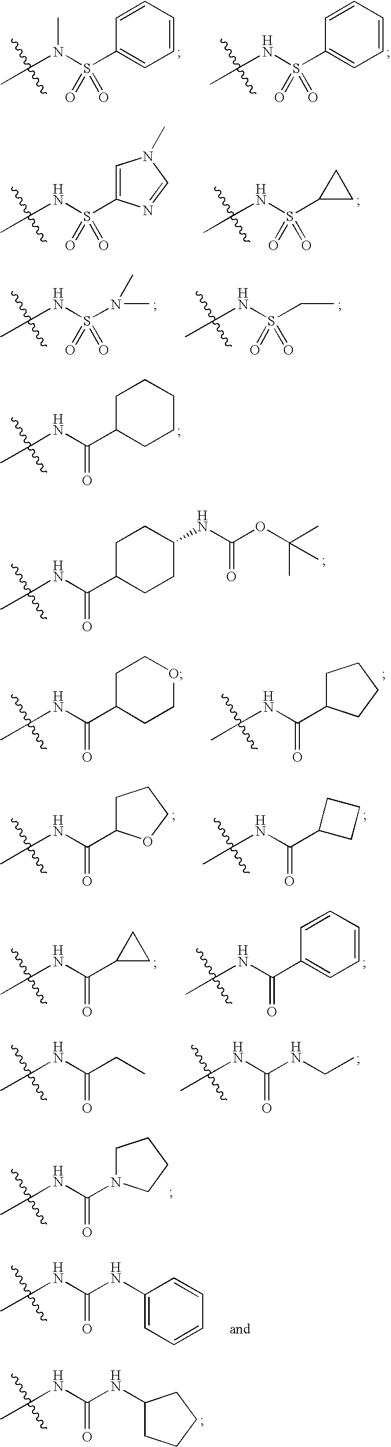

R² is selected from methyl, ethyl, 1-propyl, 2-propyl, 1-butyl, 2-butyl, t-butyl, allyl, —S(═O)₂—CH₃, —S(═O)₂—CH₂CH₃, 2-methoxyethyl, tetrahydropyran-4-yl-methyl, 1-propylsulfonyl, cyclopropylsulfonyl, phenyl, phenylsulfonyl, 2-(methoxycarbonyl)-phenylsulfonyl; 2-(hydroxycarbonyl)-phenylsulfonyl, 1-methyl-1H-imidazol-4-yl-sulfonyl, 1H-imidazol-1-yl-sulfonyl, (5-methylisoxazol-4-yl)sulfonyl, morpholin-4-ylcarbonyl, 4-amino-phenyl, —CH₂—C(═O)—N(CH₃)₂, —C(═O)—N(CH₃)₂, —S(═O)₂—N(CH₃)₂, —S(═O)₂—NHCH₂CH₃, —C(═O)—CH₂CH₂CH₃, —CH₂—C(═O)—OCH₃, —CH₂—C(═O)—OCH₂CH₃, —CH₂—CO₂H, benzyl, 4-aminobenzyl, 4-nitrobenzyl, 4-methylsulfonyl-benzyl, 4-methylthio-benzyl, 4-acetylamino-benzyl, 4-methoxy-benzyl, 4-ethoxy-benzyl, 2,6-difluorobenzyl, (6-chloro-1,3-benzodioxol-5-yl)methyl, (5-ethoxycarbonyl)-fur-2-yl-methyl, (2-methyl-1,3-thiazol-4-yl)-methyl, (5-methylisoxazol-4-yl)-methyl, pyridin-2-ylmethyl, cyclobutylmethyl, and cyclopropylmethyl;

R³ is selected from ethyl, isopropyl, propyl, 2-methyl-propyl, 1-butyl, 1-pentyl, 1-acetyl-piperidin-4-yl, tetrahydrothien-3-yl, cyclopropylmethyl, cyclobutylmethyl, cyclopentylmethyl, cyclobutyl, cyclopentyl, cyclohexyl, 4-tetrahydro-2H-pyranyl, tetrahydro-thiopyran-4-yl, 2-pyrimidinyl, 1-iminoethyl, 2-pyridinyl, 3,4,5,6-tetrahydropyrdin-2-yl, 3,4-dihydro-2H-pyrrol-5-yl, 2-pyridinyl-methyl, 3-pyridinylmethyl, 4-pyridinylmethyl, 1-methyl-4-piperidinyl, 4-piperidinyl, (6-methylpyridin-2-yl)methyl, (2-ethyl-4-methyl-1H-imidazol-5-yl)methyl, tetrahydrofuran-2-yl, tetrahydrofuran-3-ylmethyl, 1-ethyl-1H-pyrazol-4-yl, 1,3-dimethyl-1H-pyrazol-5-yl, (3-methylpyridin-4-yl)methyl, 1,3-oxazol-2-ylmethyl, 1,3-oxazol-5-ylmethyl, 2-(tetrahydro-2H-pyran-4-yl)ethyl, tetrahydro-2H-pyran-4-ylmethyl, 2-phenylethyl, 2-methoxybenzyl, 3,3,3-trifluoropropyl, 2,2-difluoroethyl, 2-hydroxycyclopentyl, (1-ethyl-3-methyl-1H-pyrazol-5-yl)methyl, 2,1,3-benzoxadiazol-5-ylmethyl, 3-thienylmethyl, 2-trifluoromethyl-benzyl, 3-methylbutyl, cyclohex-3-en-1-ylmethyl, 2-fluoro-6-methoxybenzyl, 2-phenyl-propyl, 2-ethyl-butyl, cyclobutylcarbonyl, 2,2-difluoropropanoyl, cyclopentylcarbonyl, tetrahydro-2H-pyran-4-ylcarbonyl, cyclopropylcarbonyl, propylcarbonyl, N-ethylaminocarbonyl, N-isopropylaminocarbonyl, cyclopropylsulfonyl, and ethylsulfonyl.

In another particular embodiment, R³ is selected from ethyl, isopropyl, propyl, 2-methyl-propyl, 1-butyl, 1-pentyl, 1-acetyl-piperidin-4-yl, tetrahydrothien-3-yl, cyclopropylmethyl, cyclobutylmethyl, cyclopentylmethyl, cyclobutyl, cyclopentyl, cyclohexyl, 4-tetrahydro-2H-pyranyl, tetrahydro-thiopyran-4-yl, 2-pyrimidinyl, 1-iminoethyl, 2-pyridinyl, 3,4,5,6-tetrahydropyrdin-2-yl, 3,4-dihydro-2H-pyrrol-5-yl, 2-pyridinyl-methyl, 3-pyridinylmethyl, 4-pyridinylmethyl, 1-methyl-4-piperidinyl, 4-piperidinyl, (6-methyl-pyridin-2-yl)methyl, (2-ethyl-4-methyl-1H-imidazol-5-yl)methyl, tetrahydrofuran-2-yl, tetrahydrofuran-3-yl, tetrahydrofuran-3-ylmethyl, 1-ethyl-1H-pyrazol-4-yl, 1,3-dimethyl-1H-pyrazol-5-yl, (3-methylpyridin-4-yl)methyl, 1,3-oxazol-2-ylmethyl, 1,3-oxazol-5-ylmethyl, 2-(tetrahydro-2H-pyran-4-yl)ethyl, tetrahydro-2H-pyran-4-ylmethyl, 2-phenylethyl, 2-methoxybenzyl, 3,3,3-trifluoropropyl, 2,2-difluoroethyl, 2-hydroxycyclopentyl, (1-ethyl-3-methyl-1H-pyrazol-5-yl)methyl, 2,1,3-benzoxadiazol-5-ylmethyl, 3-thienylmethyl, 2-trifluoromethyl-benzyl, 3-methylbutyl, cyclohex-3-en-1-ylmethyl, 2-fluoro-6-methoxybenzyl, 2-phenyl-propyl, 2-ethyl-butyl, cyclobutylcarbonyl, 2,2-difluoropropanoyl, cyclopentylcarbonyl, tetrahydro-2H-pyran-4-ylcarbonyl, cyclopropylcarbonyl, propylcarbonyl, N-ethylaminocarbonyl, N-isopropylaminocarbonyl, cyclopropylsulfonyl, and ethylsulfonyl.

A yet even further embodiment of the invention provides a compound of formula I,

wherein

R¹ is selected from

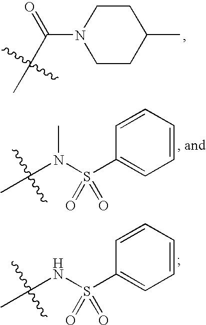

R² is selected from —H, methyl, ethyl, 1-propyl, 2-propyl, 1-butyl, 2-butyl, t-butyl, allyl, —S(═O)₂—CH₃, —S(═O)₂—CH₂CH₃, 2-methoxyethyl, tetrahydropyran-4-yl-methyl, 1-propylsulfonyl, cyclopropylsulfonyl, phenyl, phenylsulfonyl, 2-(methoxycarbonyl)-phenylsulfonyl; 2-(hydroxycarbonyl)-phenylsulfonyl, 1-methyl-1H-imidazol-4-yl-sulfonyl, (5-methylisoxazol-4-yl)sulfonyl, morpholin-4-ylcarbonyl, 4-amino-phenyl, —CH₂—C(═O)—N(CH₃)₂, —C(═O)—N(CH₃)₂, —S(═O)₂—N(CH₃)₂, —S(═O)₂—NHCH₂CH₃, —C(═O)—CH₂CH₂CH₃, —CH₂—C(═O)—OCH₃, —CH₂—C(═O)—OCH₂CH₃, —CH₂—CO₂H, benzyl, 4-aminobenzyl, 4-nitrobenzyl, 4-methylsulfonyl-benzyl, 4-methylthio-benzyl, 4-acetylamino-benzyl, 4-methoxy-benzyl, 4-ethoxy-benzyl, 2,6-difluorobenzyl, (6-chloro-1,3-benzodioxol-5-yl)methyl, (5-ethoxycarbonyl)-fur-2-yl-methyl, (2-methyl-1,3-thiazol-4-yl)-methyl, (5-methyl-isoxazol-4-yl)-methyl, pyridin-2-ylmethyl, cyclobutylmethyl, and cyclopropylmethyl; and

R³ is selected from ethyl, isopropyl, propyl, 2-methyl-propyl, 1-butyl, 1-pentyl, 1-acetyl-piperidin-4-yl, tetrahydrothien-3-yl, cyclopropylmethyl, cyclobutylmethyl, cyclopentylmethyl, cyclobutyl, cyclopentyl, cyclohexyl, 4-tetrahydro-2H-pyranyl, tetrahydro-thiopyran-4-yl, 1-iminoethyl, 3,4,5,6-tetrahydropyrdin-2-yl, 3,4-dihydro-2H-pyrrol-5-yl, tetrahydrofuran-3-ylmethyl, tetrahydrofuran-2-yl, 1-methyl-4-piperidinyl, 2-(tetrahydro-2H-pyran-4-yl)ethyl, tetrahydro-2H-pyran-4-ylmethyl, 3,3,3-trifluoropropyl, 2,2-difluoroethyl, 2-hydroxycyclopentyl, 3-methylbutyl, cyclohex-3-en-1-ylmethyl, and 2-ethyl-butyl.

In another embodiment, R³ is selected from ethyl, isopropyl, propyl, 2-methyl-propyl, 1-butyl, 1-pentyl, 1-acetyl-piperidin-4-yl, tetrahydrothien-3-yl, cyclopropylmethyl, cyclobutylmethyl, cyclopentylmethyl, cyclobutyl, cyclopentyl, cyclohexyl, 4-tetrahydro-2H-pyranyl, tetrahydro-thiopyran-4-yl, 1-iminoethyl, 3,4,5,6-tetrahydropyrdin-2-yl, 3,4-dihydro-2H-pyrrol-5-yl, tetrahydrofuran-3-ylmethyl, tetrahydrofuran-2-yl, tetrahydrofuran-3-yl, 1-methyl-4-piperidinyl, 2-(tetrahydro-2H-pyran-4-yl)ethyl, tetrahydro-2H-pyran-4-ylmethyl, 3,3,3-trifluoropropyl, 2,2-difluoroethyl, 2-hydroxycyclopentyl, 3-methylbutyl, cyclohex-3-en-1-ylmethyl, and 2-ethyl-butyl.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I. It will further be understood that the present invention encompasses tautomers of the compounds of the Formula I.

It will also be understood that certain compounds of the present invention may exist in solvated, for example hydrated, as well as unsolvated forms. It will further be understood that the present invention encompasses all such solvated forms of the compounds of the Formula I.

Within the scope of the invention are also salts of the compounds of the Formula I. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of Formula I above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as modulators or ligands such as agonists, partial agonists, inverse agonist or antagonists of CB₁ receptors. More particularly, the compounds of the invention exhibit selective activity as agonist of the CB₁ receptors and are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. Additionally, compounds of the present invention are useful in other disease states in which dysfunction of CB₁ receptors is present or implicated. Furthermore, the compounds of the invention may be used to treat cancer, multiple sclerosis, Parkinson's disease, Huntington's chorea, Alzheimer's disease, anxiety disorders, obesity, gastrointestinal disorders and cardiovascular disorders. Even furthermore, the compounds of the invention may be useful in enhancing smoking cessation.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of cannabinoid receptors is present or implicated in that paradigm. This may involve the use of isotopically labelled versions of the compounds of the invention in diagnostic techniques and imaging applications such as positron emission tomography (PET).

Compounds of the invention are useful for the treatment of diarrhoea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastro-intestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, obesity, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

Also within the scope of the invention is the use of any of the compounds according to the Formula I above, for the manufacture of a medicament for the treatment of any of the conditions discussed above.

A further aspect of the invention is a method for the treatment of a subject suffering from any of the conditions discussed above, whereby an effective amount of a compound according to the Formula I above, is administered to a patient in need of such treatment.

Thus, the invention provides a compound of Formula I or pharmaceutically acceptable salt or solvate thereof, as hereinbefore defined for use in therapy.

In a further aspect, the present invention provides the use of a compound of Formula I or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be contrued accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, transdermally, intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid and liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of Formula I as defined above for the manufacture of a medicament.

Also within the scope of the invention is the use of any compound of Formula I for the manufacture of a medicament for the therapy of pain.

Additionally provided is the use of any compound according to Formula I for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the Formula I above, is administered to a patient in need of such therapy.

Additionally, there is provided a pharmaceutical composition comprising a compound of Formula I or a pharmaceutically acceptable salt thereof, in association with a pharmaceutically acceptable carrier.

Particularly, there is provided a pharmaceutical composition comprising a compound of Formula I or a pharmaceutically acceptable salt thereof, in association with a pharmaceutically acceptable carrier for therapy, more particularly for therapy of pain.

Further, there is provided a pharmaceutical composition comprising a compound of Formula I or a pharmaceutically acceptable salt thereof, in association with a pharmaceutically acceptable carrier use in any of the conditions discussed above.

In a further aspect, the present invention provides a method of preparing the compounds of the present invention.

In one embodiment, the invention provides a process for preparing a compound of Formula II, comprising:

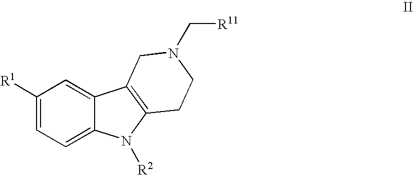

reacting a compound of Formula III with a compound of R¹¹—CHO,

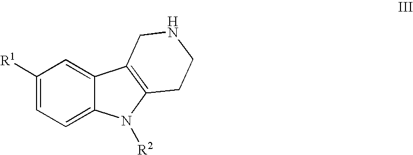

wherein R¹ and R² are as defined above; and

R¹¹ is selected from C₃₋₆heterocycloalkyl, C₃₋₆cycloalkyl and C₁₋₆alkyl, wherein said C₃₋₆heterocycloalkyl, C₃₋₆cycloalkyl and C₁₋₆alkyl used in defining R¹¹ is optionally substituted with one or more groups selected from methoxy, ethoxy, methyl, ethyl and halogen.

Optionally, the step of reacting a compound of formula II with a compound of R¹¹—CHO is carried out in the presence of a reducing agent, such as sodium triacetoxylborohydride, sodium cyanoborohydride or sodium borohydride.

In another embodiment, the invention provides a process for preparing a compound of Formula IV, comprising:

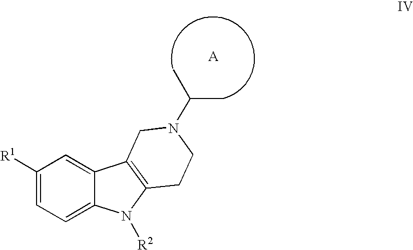

reacting a compound of Formula III with a compound of

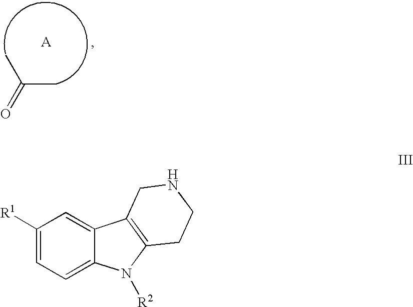

wherein R¹ and R² are as defined above; and

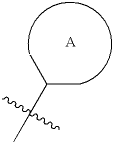

is selected from C₃₋₆heterocycloalkyl and C₃₋₆cycloalkyl, wherein said C₃₋₆heterocycloalkyl and C₃₋₆cycloalkyl are optionally substituted with one or more groups selected from methoxy, ethoxy, methyl, ethyl and halogen.

Optionally, the step of reacting a compound of formula II with a compound of

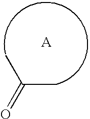

is carried out in the presence of a reducing agent, such as sodium triacetoxylborohydride, sodium cyanoborohydride or sodium borohydride.

In another embodiment, the invention provides a process for preparing a compound of Formula V, comprising

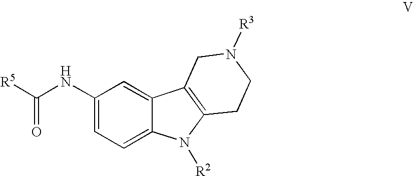

reacting a compound of Formula VI with R⁵—C(═O)—Cl,

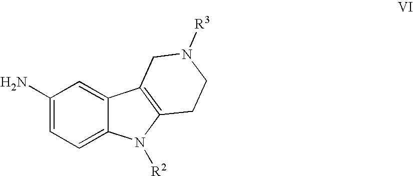

wherein R², R³ and R⁵ are as defined above.

Optionally, the step of reacting a compound of formula VI with R⁵—C(═O)—Cl is carried out in the presence of a base, such as triethylamine.

In another embodiment, the invention provides a process for preparing a compound of Formula VII, comprising

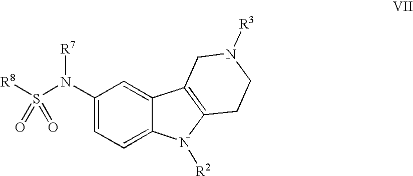

reacting a compound of Formula VIII with R⁸—SO₂—Cl,

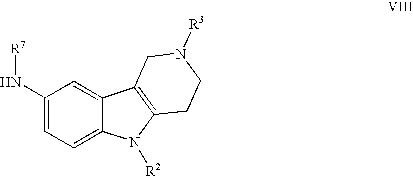

wherein R², R³, R⁵ and R⁷ are as defined above.

Optionally, the step of reacting a compound of formula VIII with R⁸—SO₂—Cl is carried out in the presence of a base, such as triethylamine.

In another embodiment, the invention provides a process for preparing a compound of Formula IX, comprising

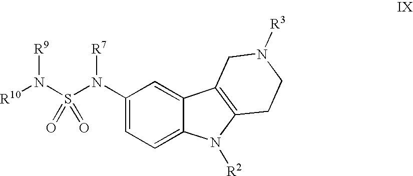

reacting a compound of Formula VIII with R¹⁰R⁹N—SO₂—Cl,

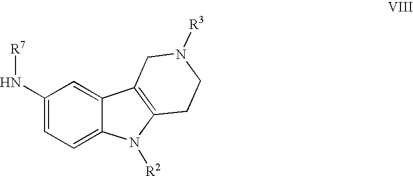

wherein R², R³, R⁷, R⁹ and R¹⁰ are as defined above.

Optionally, the step of reacting a compound of formula VIII with R¹⁰R⁹N—SO₂—Cl is carried out in the presence of a base, such as triethylamine.

In another embodiment, the invention provides a process for preparing a compound of Formula X, comprising

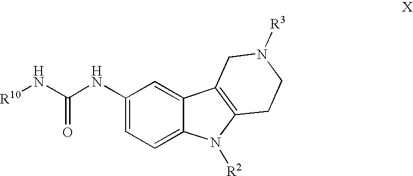

reacting a compound of Formula VI with R⁶NCO,

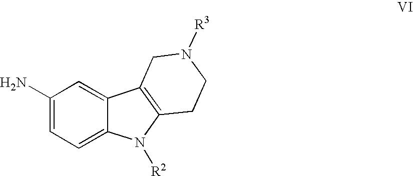

wherein R², R³ and R⁶ are as defined above.

Optionally, the step of reacting a compound of formula VI with R⁶NCO is carried out in the presence of a base, such as triethylamine.

Compounds of the present invention may also be prepared according to the synthetic routes as depicted in Schemes 1-10.

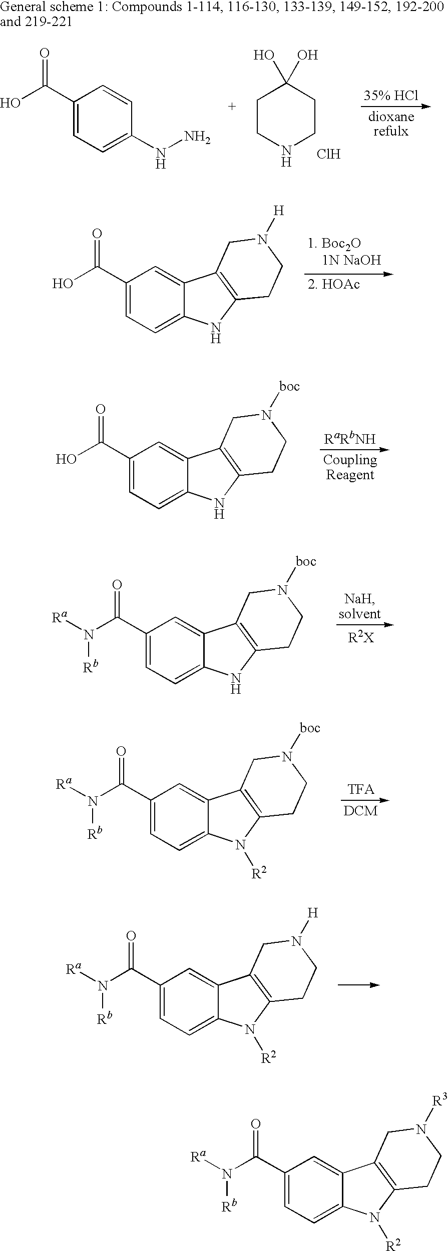

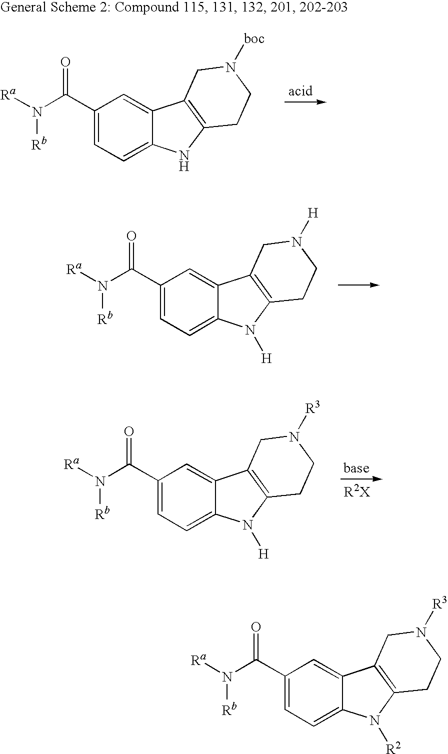

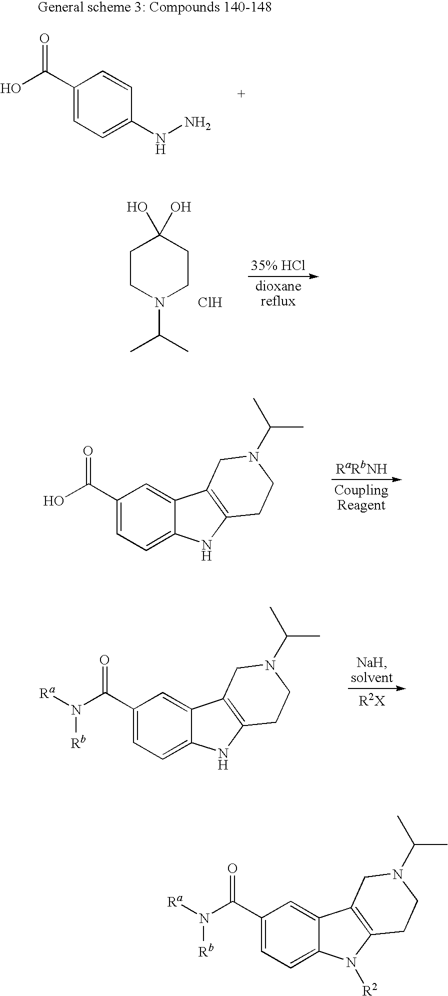

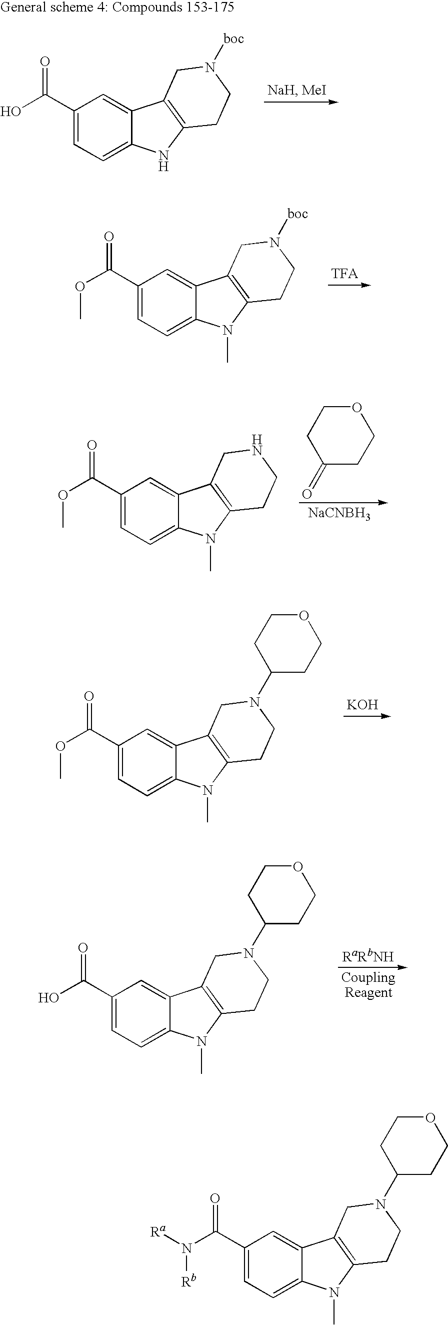

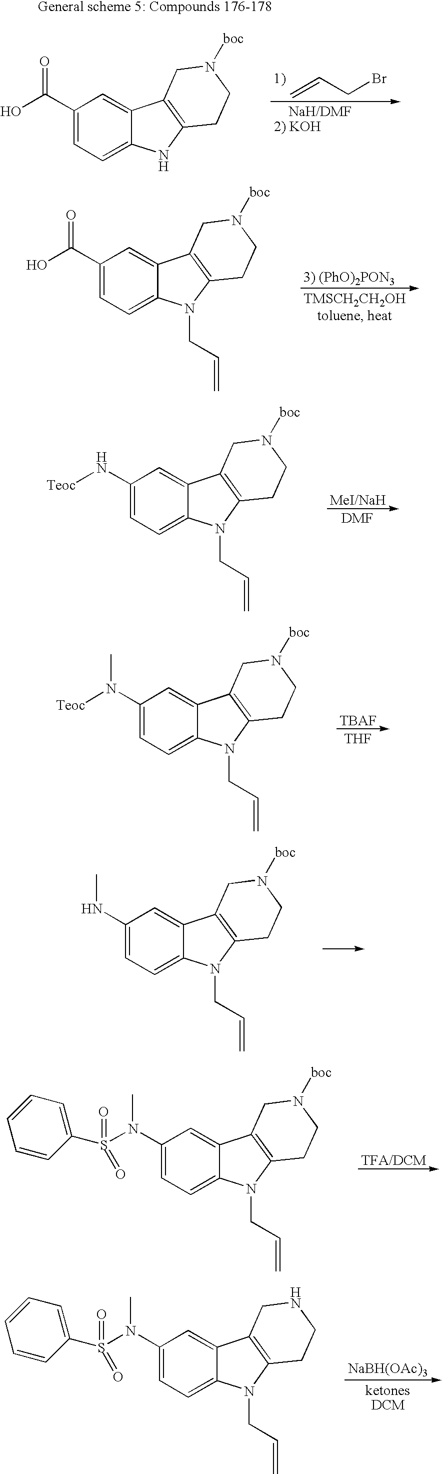

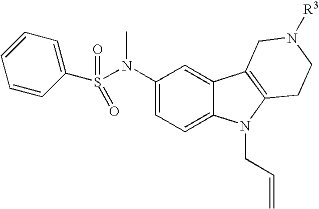

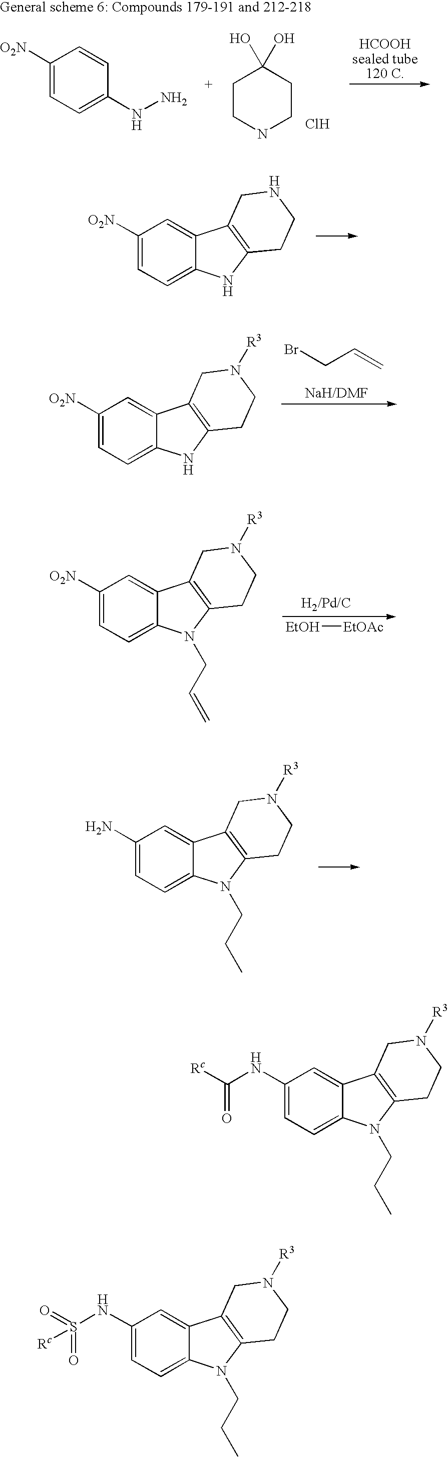

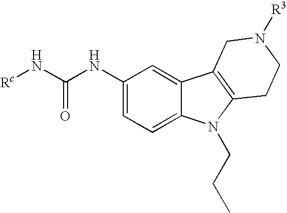

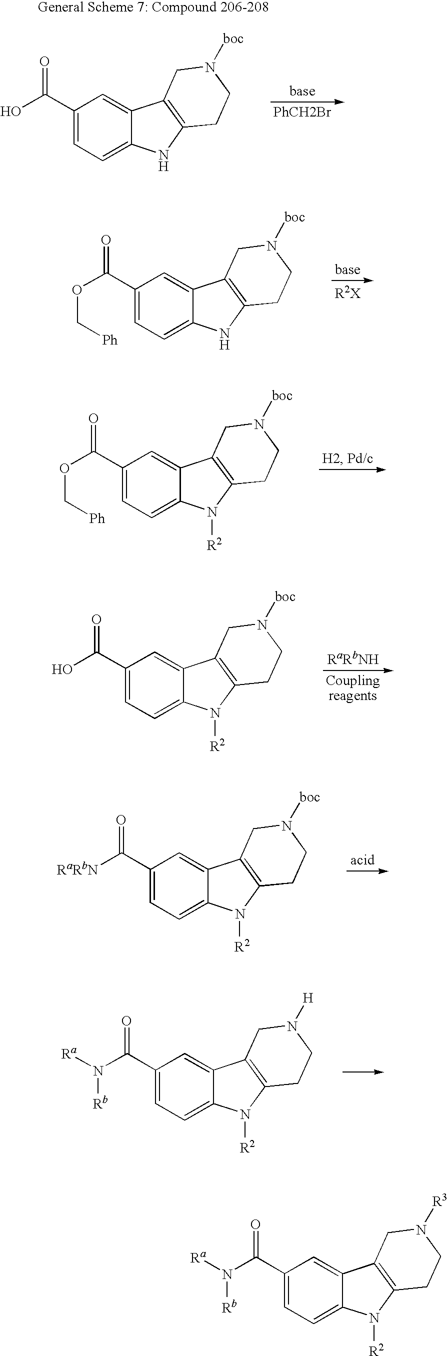

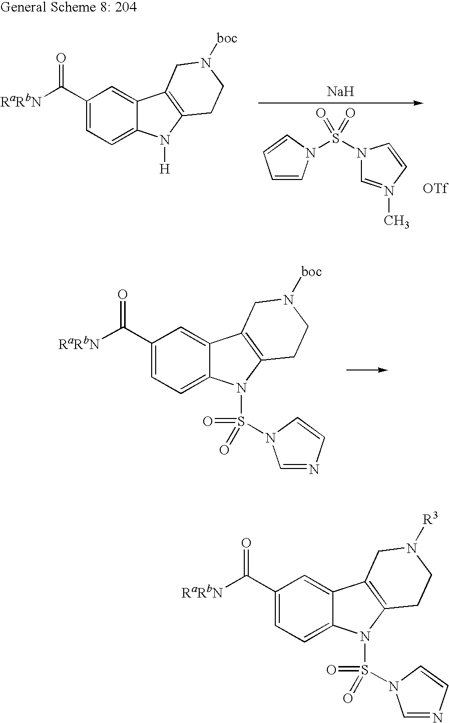

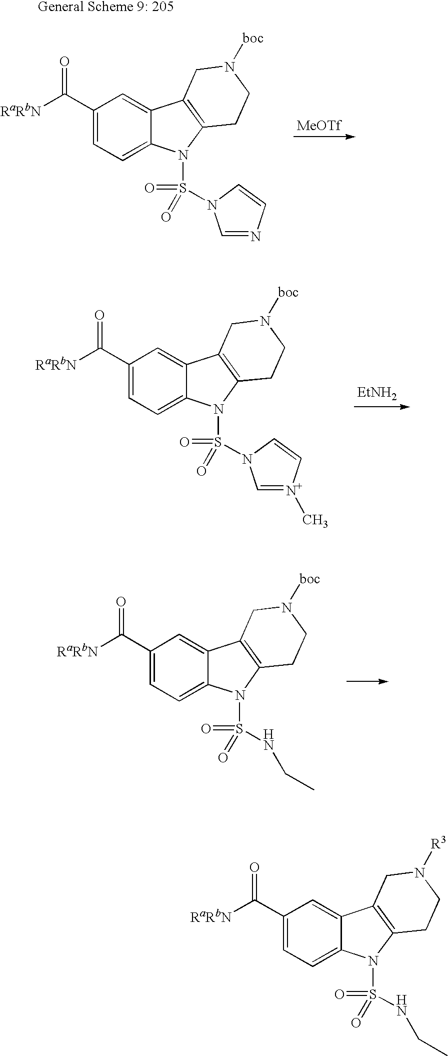

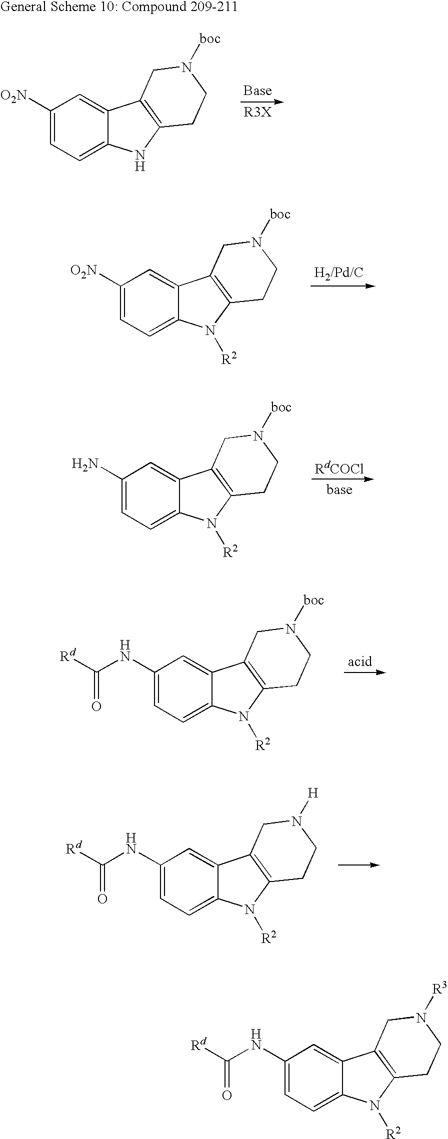

### Biological Evaluation

hCB₁ and hCB₂ Receptor Binding

Human CB₁ receptor from Receptor Biology (hCB₁) or human CB₂ receptor from BioSignal (hCB₂) membranes are thawed at 37° C., passed 3 times through a 25-gauge blunt-end needle, diluted in the cannabinoid binding buffer (50 mM Tris, 2.5 mM EDTA, 5 mM MgCl₂, and 0.5 mg/mL BSA fatty acid free, pH 7.4) and aliquots containing the appropriate amount of protein are distributed in 96-well plates. The IC₅₀ of the compounds of the invention at hCB₁ and hCB₂ are evaluated from 10-point dose-response curves done with ³H-CP55,940 at 20000 to 25000 dpm per well (0.17-0.21 nM) in a final volume of 300 μl. The total and non-specific binding are determined in the absence and presence of 0.2 μM of HU210 respectively. The plates are vortexed and incubated for 60 minutes at room temperature, filtered through Unifilters GF/B (presoaked in 0.1% polyethyleneimine) with the Tomtec or Packard harvester using 3 mL of wash buffer (50 mM Tris, 5 mM MgCl₂, 0.5 mg BSA pH 7.0). The filters are dried for 1 hour at 55° C. The radioactivity (cpm) is counted in a TopCount (Packard) after adding 65 μl/well of MS-20 scintillation liquid.

hCB₁ and hCB₂ GTPγS binding

Human CB₁ receptor from Receptor Biology (hCB₁) or human CB₂ receptor membranes (BioSignal) are thawed at 37° C., passed 3 times through a 25-gauge blunt-end needle and diluted in the GTPγS binding buffer (50 mM Hepes, 20 mM NaOH, 100 mM NaCl, 1 mM EDTA, 5 mM MgCl₂, pH 7.4, 0.1% BSA). The EC₅₀ and Eₘₐₓ of the compounds of the invention are evaluated from 10-point dose-response curves done in 300 μl with the appropriate amount of membrane protein and 100000-130000 dpm of GTPg³⁵S per well (0.11-0.14 nM). The basal and maximal stimulated binding is determined in absence and presence of 1 μM (hCB₂) or 10 μM (hCB₁) Win 55, 212-2 respectively. The membranes are pre-incubated for 5 minutes with 56.25 μM (hCB2) or 112.5 μM (hCB₁) GDP prior to distribution in plates (15 μM (hCB₂) or 30 μM (hCB₁) GDP final). The plates are vortexed and incubated for 60 minutes at room temperature, filtered on Unifilters GF/B (presoaked in water) with the Tomtec or Packard harvester using 3 ml of wash buffer (50 mM Tris, 5 mM MgCl₂, 50 mM NaCl, pH 7.0). The filters are dried for 1 hour at 55° C. The radioactivity (cpm) is counted in a TopCount (Packard) after adding 65 μl/well of MS-20 scintillation liquid. Antagonist reversal studies are done in the same way except that (a) an agonist dose-response curve is done in the presence of a constant concentration of antagonist, or (b) an antagonist dose-response curve is done in the presence of a constant concentration of agonist.

Based on the above assays, the dissociation constant (Ki) for a particular compound of the invention towards a particular receptor is determined using the following equation:

Ki=IC₅₀/(1+[rad]Kd),

Wherein IC₅₀ is the concentration of the compound of the invention at which 50% displacement has been observed;

[rad] is a standard or reference radioactive ligand concentration at that moment; and

Kd is the dissociation constant of the radioactive ligand towards the particular receptor.

Using the above-mentioned assays, the compounds of the invention are found to be active towards human CB₁ receptors.

### EXAMPLES

The invention will further be described in more detail by the following Examples which describe methods whereby compounds of the present invention may be prepared, purified, analyzed and biologically tested, and which are not to be construed as limiting the invention.

### Example 1

### 5-allyl-2-(cyclopropylmethyl)-8-[(4-methylpiperidin-1-yl)carbonyl]-2,3,4,5-tetrahydro-1H-pyrido[4,3-b]indole

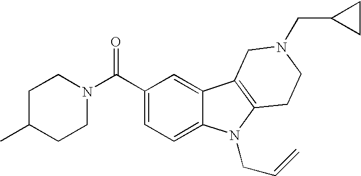

### Step A: The preparation of 2-(tert-butoxycarbonyl)-2,3,4,5-tetrahydro-1H-pyrido[4,3-b]indole-8-carboxylic acid

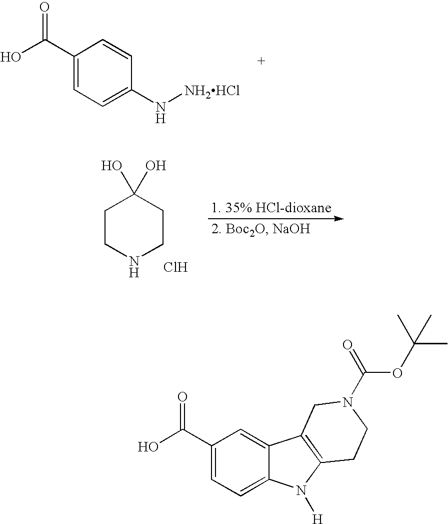

4-hydrazinobenzoic acid hydrochloride salt (18.6 g, 98.6 mmol) and 4-piperidinone monohydrate hydrochloride (17.0 g, 110 mmol) in dioxane (300 ml) and concentrated hydrochloric acid (30 ml) were heated at reflux overnight, allowed to cool to room temperature, and the precipitate was collected, and the filtrate was concentrated in vacuo to dryness, and the residue was treated with water (50 mL), and the solid was collected by filtration. The solid was combined and used without purification. The above solid intermediate was dissolved in methanol (250 mL), and pH was adjusted to ˜9-10 with 5N NaOH, di-tert-butyl dicarbonate (13.0 g, 59.6 mmol) was added and the reaction was stirred for 2 h, then the pH was readjusted to ˜9-10, di-tert-butyl dicarbonate (5.0 g, 22.9 mmol) was added, and the reaction was stirred at room temperature overnight. The pH was again readjusted to ˜9-10 with 2N NaOH, 3 g of di-tert-butyl dicarbonate (5.0 g, 13.7 mmol) was added again and the reaction was stirred for another 2 h. MeOH was concentrated in vacuo, and the residue was diluted with water (100 mL), and extracted with dichloromethane (2×200 mL), and the aqueous phase was acidified with acetic acid to pH˜5 with stirring. The white solid was collected and dried (15.2 g, 48.7% in two steps).

### Step B: The preparation of tert-butyl 8-[(4-methylpiperidin-1-yl)carbonyl]-1,3,4,5-tetrahydro-2H-pyrido[4,3-b]indole-2-carboxylate

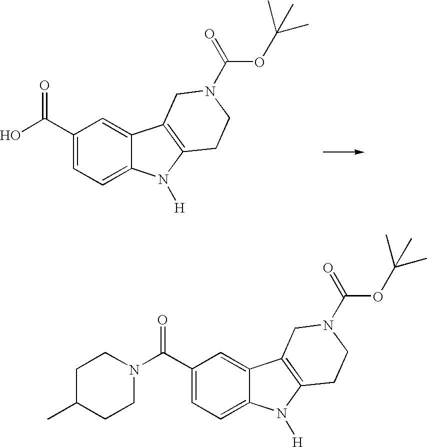

The product from Step A (2-(tert-butoxycarbonyl)-2,3,4,5-tetrahydro-1H-pyrido[4,3-b]indole-8-carboxylic acid, 4.80 g, 15.2 mmol) was dissolved in DMF (80 mL), 4-methylpiperidine (2 mL, 16.9 mmol), diisopropylethylamine (7.0 mL, 38.9 mmol), and HATU (7.60 g, 20.0 mmol) was added successively. The mixture was stirred at room temperature for 1 h, and the reaction was quenched with water (5 mL). The solvent was removed in vacuo to give a light yellow solid, and the solid was treated with water (100 mL) and collected by filtration, then recrystallized in methanol to afford the desired product (4.60 g), the filtrate was concentrated in vacuo and purified on silica gel (0-40% EtOAc in dichloromethane) to give 1.10 g additional desired product, the combined yield was 94.5%. MS: 398.1 (M+1).

### Step C: The preparation of tert-butyl 5-allyl-8-[(4-methylpiperidin-1-yl)carbonyl]-1,3,4,5-tetrahydro-2H-pyrido[4,3-b]indole-2-carboxylate

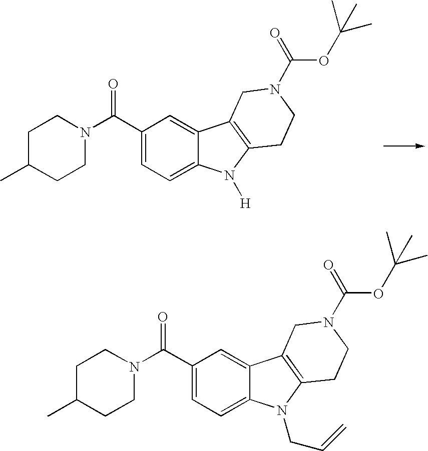

60% NaH (480 mg, 12.0 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (2.42 g, 6.08 mmol) in DMF (50 mL) at 0° C. under nitrogen. The mixture was stirred at room temperature for 30 min, then allyl bromide (1.0 mL, 11.6 mmol) was added at room temperature, and the reaction mixture was stirred at room temperature for 1 h. The solvent was removed in vacuo and the residue was dissolved in dichloromethane (150 mL), washed with water (50 mL), dried over sodium sulfate. Removal of solvent gave the crude product, which was purified on silica gel (0-30% EtOAc in dichloromethane) to give the desired product (2.48 g, 93.5%). MS: 438.1 (M+1).

### Step D: The preparation of 5-allyl-8-[(4-methylpiperidin-1-yl)carbonyl]-2,3,4,5-tetrahydro-1H-pyrido[4,3-b]indole

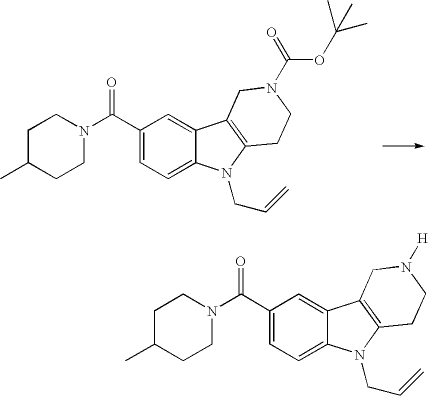

Method A (HCl salt): tert-butyl 5-allyl-8-[(4-methylpiperidin-1-yl)carbonyl]-1,3,4,5-tetrahydro-2H-pyrido[4,3-b]indole-2-carboxylate (315 mg, 0.720 mmol) was added to saturated HCl solution in ethyl acetate (15 mL) at 0° C. The mixture was stirred at 0° C. for 30 min, and removal of solvent gave the desired product 5-allyl-8-[(4-methylpiperidin-1-yl)carbonyl]-2,3,4,5-tetrahydro-1H-pyrido[4,3-b]indole as its hydrochloric acid salt form (235 mg, 87%). MS (M+1): 338.0.
Method B (TFA salt): tert-butyl 5-allyl-8-[(4-methylpiperidin-1-yl)carbonyl]-1,3,4,5-tetrahydro-2H-pyrido[4,3-b]indole-2-carboxylate (2.45 g, 5.60 mmol) was dissolved in dichloromethane (50 mL) and trifluoroacetic acid (10 mL) was added to the reaction mixture. The mixture was stirred at room temperature for 1 h. Removal of excess trifluoroacetic acid in vacuo gave crude 5-allyl-8-[(4-methylpiperidin-1-yl)carbonyl]-2,3,4,5-tetrahydro-1H-pyrido[4,3-b]indole as its trifluoroacetic acid salt form (2.70 g).

### Step E: The preparation of 5-allyl-2-(cyclopropylmethyl)-8-[(4-methylpiperidin-1-yl)carbonyl]-2,3,4,5-tetrahydro-1H-pyrido[4,3-b]indole

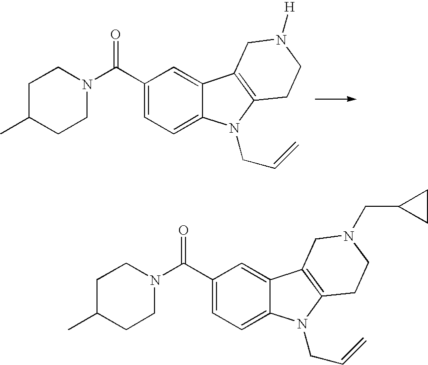

Sodium triacetoxyborohydride (100 mg, 0.474 mmol) was added to 5-allyl-8-[(4-methylpiperidin-1-yl)carbonyl]-2,3,4,5-tetrahydro-1H-pyrido[4,3-b]indole HCl salt (101 mg, 0.271 mmol) from Step D: Method A and cyclopropanecarbaldehyde (50 μL) in dichloromethane (3 mL). The mixture was stirred at room temperature overnight, diluted with dichloromethane (15 mL), washed with saturated NaHCO₃ solution (5 mL), and dried over Na₂SO₄. The dichloromethane solution was concentrated in vacuo and the residue was re-dissolved in ethyl ether (10 mL), and 1 N HCl solution in ethyl ether was added dropwise with stirring. The solid was collected and dried to give the desired product 5-allyl-2-(cyclopropylmethyl)-8-[(4-methylpiperidin-1-yl)carbonyl]-2,3,4,5-tetrahydro-1H-pyrido[4,3-b]indole as its HCl salt (107 mg, 93%). MS (M+1): 392.0; 1H NMR (400 MHz, CD3OD) δ ppm 0.39-0.62 (m, 2H), 0.68-0.91 (m, 2H), 0.99 (d, j=6.44 Hz, 3H), 1.07-1.43 (m, 3H), 1.48-1.97 (m, 3H), 2.81-3.36 (m, 6H), 3.52-3.66 (m, 1H), 3.70-3.92 (m, 1H), 3.93-4.15 (m, 1H), 4.44 (d, J=14.25 Hz, 1H), 4.51-4.70 (m, 1H), 4.75-4.88 (m, 4H), 5.14 (dd, J=10.35, 1.17 Hz, 1H), 5.81-6.16 (m, 1H), 7.13-7.34 (m, 1H), 7.49 (d, J=8.40 Hz, 1H), 7.65 (d, J=1.17 Hz, 1H).

### Example 2

### 5-allyl-8-[(4-methylpiperidin-1-yl)carbonyl]-2-(tetrahydro-2H-pyran-4-yl)-2,3,4,5-tetrahydro-1H-pyrido[4,3-b]indole

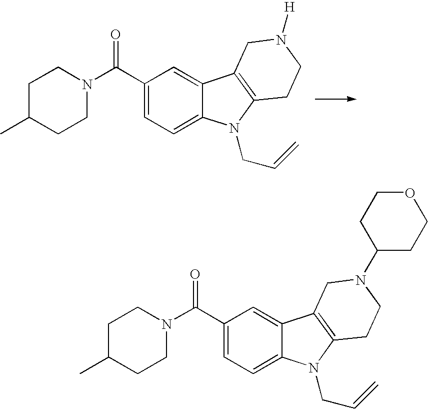

Following the general procedure described in Example 1, Step E: The title compound was prepared from tetrahydro-4H-pyran-4-one (30 mg), 5-allyl-8-[(4-methylpiperidin-1-yl)carbonyl]-2,3,4,5-tetrahydro-1H-pyrido[4,3-b]indole TFA salt (50 mg) prepared from Example 1, Step D (method B). MS (M+1): 422.1. 1H NMR (600 MHz, CD3OD) δ ppm 1.01 (d, J=6.14 Hz, 3H), 1.08-1.32 (m, 2H), 1.55-1.88 (m, 3H), 1.87-2.01 (m, 2H), 2.20 (s, 2H), 2.88 (s, 1H), 3.06-3.18 (m, 1H), 3.19-3.28 (m, 2H), 3.52 (t, J=11.78 Hz, 2H), 3.55-3.64 (m, 1H), 3.67-3.77 (m, 1H), 3.77-3.89 (m, 1H), 4.05 (d, J=10.24 Hz, 1H), 4.10-4.19 (m, 2H), 4.54 (d, J=13.31 Hz, 1H), 4.59-4.67 (m, 1H), 4.76 (d, J=13.31 Hz, 1H), 4.81-4.88 (m, 2H), 4.91-4.93 (m, 1H), 5.17 (d, J=10.24 Hz, 1H), 5.94-6.07 (m, 1H), 7.27 (d, J=8.19 Hz, 1H), 7.48 (d, J=8.19 Hz, 1H), 7.63 (s, 1H). Anal. Calcd for C₂₆H₃₅N₃O₂.2C₂HF₃O₂: C, 55.46; H, 5.74; N, 6.47. Found: C, 55.67; H, 5.59; N, 6.34.
Example 3-10 was prepared by the same method described in Example 2.

TABLE 1

| Examples | Structures | | Data |
|---|---|---|---|
| 3 | 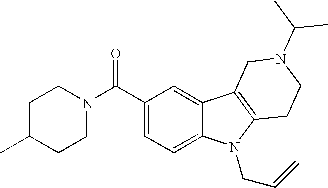 | 5-allyl-2-isopropyl- 8-[(4- methylpiperidin-1- yl)carbonyl]-2,3,4,5- tetrahydro-1H- pyrido[4,3-b]indole | MS (M + 1): 380.0. 1H NMR (400 MHz, METHANOL- D4) δ ppm 0.98 (d, J = 6.25 Hz, 3 H), 1.17 (m, 2 H), 1.49 (dd, J = 6.25, 4.30 Hz, 6 H), 1.57-1.82 (m, 3 H), 2.86 (s, 1 H), 3.08 (s, 1 H), 3.14- 3.25 (m, 2 H), 3.49-3.61 (m, 1 H),3.70-3.94 (m, 3 H), 4.40- 4.68 (m, 3 H), 4.73-4.83 (m, 3 H), 5.14 (d, J = 10.15 Hz, 1 H), 5.79-6.15 (m, 1 H), 7.24 (d, J = 8.59 Hz, 1 H), 7.45 (d, J = 8.59 Hz, 1H), 7.59 (s, 1 H). |
| 4 | 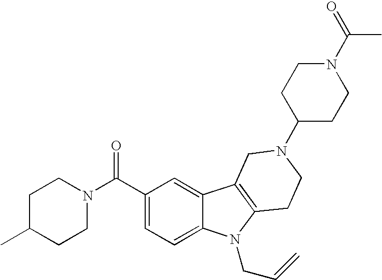 | 2-(1-acetylpiperidin- 4-yl)-5-allyl-8-[(4- methylpiperidin-1- yl)carbonyl]-2,3,4,5- tetrahydro-1H- pyrido[4,3-b]indole | MS (M + 1): 463.1; 1H NMR (400 MHz, METHANOL- D4) δ ppm 0.97 (d, J = 6.45 Hz, 3 H), 1.05-1.30 (m, 2 H), 1.48- 1.96 (m, 4 H), 2.08-2.17 (m, 3 H), 2.17-2.35 (m, 2 H), 2.69 (t, J = 13.09 Hz, 1 H), 2.85 (s, 1 H), 2.99- 3.26 (m, 6 H), 3.39-3.86 (m, 3 H), 3.97 (s, 1 H), 4.08-4.22 (m, 1 H), 4.42-4.85 (m, 6H), 5.10- 5.17 (m, 1H), 5.88-6.07 (m, 1 H), 7.23 (dd, J = 8.01, 0.98 Hz, 1 H), 7.45 (d, J = 8.01 Hz, 1 H), 7.58 (d, J = 0.98 Hz, 1 H). |
| 5 | 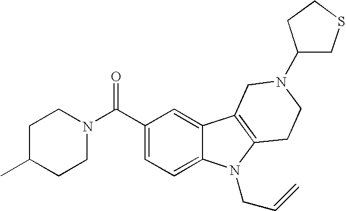 | 5-allyl-8-[(4- methylpiperidin-1- yl)carbonyl]-2- (tetrahydro-3- thienyl)-2,3,4,5- tetrahydro-1H- pyrido[4,3-b]indole | MS (M + 1): 424.0; 1HNMR 1H NMR (600 MHz, CD3OD) δ ppm 1.09 (d, J = 6.14 Hz, 3 H), 1.16-1.42 (m, 2 H), 1.61-1.98 (m, 3 H), 2.28- 2.43 (m, 1 H), 2.76-3.29 (m, 6 H), 3.34 (s, 2 H), 3.43-3.58 (m, 1 H), 3.60-4.05 (m, 2 H), 4.02- 4.35 (m, 2 H), 4.47-4.96 (m, 6 H), 5.25 (d, J = 10.24 Hz, 1 H), 5.94-6.24 (m, 1 H), 7.35 (d, J = 8.19 Hz, 1 H), 7.56 (d, J = 8.19 Hz, 1 H), 7.71 (s, 1 H). |
| 6 | 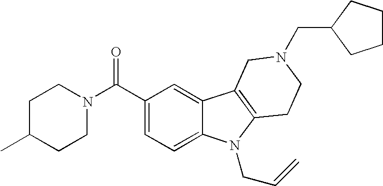 | 5-allyl-2- (cyclopentylmethyl)- 8-[(4- methylpiperidin-1- yl)carbonyl]-2,3,4,5- tetrahydro-1H- pyrido[4,3-b]indole | MS (M + 1): 420.3; 1H NMR (400 MHz, METHANOL- D4) δ ppm 0.99 (d, J = 6.64 Hz, 3 H), 1.07-1.42 (m, 4 H), 1.51- 1.85 (m, 7 H), 1.90-2.07 (m, 2 H), 2.37-2.56 (m, 1 H), 2.78- 2.96 (m, 1 H), 2.99-3.26 (m, 3 H), 3.36 (d, J = 7.42 Hz, 2 H), 3.52-3.66 (m, 1 H), 3.73 -3.87 (m, 1 H), 3.89- 4.01 (m, 1 H), 4.41 (d, J = 14.06 Hz, 1H), 4.50- 4.70 (m, 1H), 4.74-4.84 (m, 4 H), 5.15 (d, J = 10.15 Hz, 1 H), 5.90-6.06 (m, 1 H), 7.20-7.28 (m, 1 H), 7.46 (d, J = 8.59 Hz, 1 H), 7.60 (s, 1 H) |
| 7 | 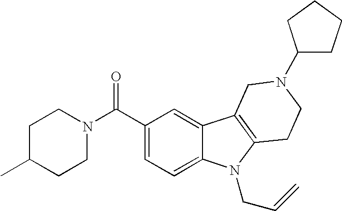 | 5-allyl-2- cyclopentyl-8-[(4- methylpiperidin-1- yl)carbonyl]-2,3,4,5- tetrahydro-1H- pyrido[4,3-b]indole | MS (M + 1): 406.0. 1H NMR (400 MHz, CDCl3) δ ppm 0.97 (d, J = 6.44 Hz, 3 H), 1.08-1.35 (m, 2 H), 1.56-2.22 (m, 11 H) 2.88-3.05 (m, 1 H), 3.31- 3.46 (m, 2 H), 3.47 (s, 3 H), 3.53-3.70 (m, 1 H), 3.89-4.01 (m, 1H), 4.17 (d, J = 13.86 Hz, 1 H), 4.57-4.88 (m, 5 H), 5.14 (d, J =10.35 Hz, 1 H), 5.84-5.93 (m, 1 H), 7.18-7.32 (m, 2H) 7.48 (s, 1H). |
| 8 | 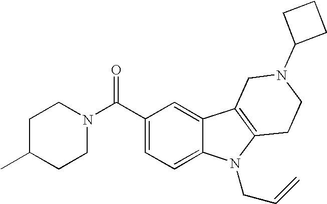 | 5-allyl-2-cyclobutyl- 8-[(4- methylpiperidin-1- yl)carbonyl]-2,3,4,5- tetrahydro-1H- pyrido[4,3-b]indole | MS (M + 1): 392.0. 1H NMR (400 MHz, CDCl3) δ ppm 0.97 (d, J = 6.44 Hz, 3 H), 1.04- 1.36 (m, 2 H), 1.52-1.87 (m, 4 H), 1.95 (q, J = 9.96 Hz, 1 H), 2.23-2.41 (m, 2 H), 2.48-2.70 (m, 2H), 2.76- 3.12 (m, 2 H), 2.90 (d, J = 16.60 Hz, 1 H), 3.11- 3.35 (m, 2H), 3.55-3.87 (m, 3 H), 3.95 (d, J = 14.25 Hz, 1 H), 4.52-4.71 (m, 4 H), 4.76 (d, J = 17.18 Hz, 1 H), 5.14 (d, J = 10.54 Hz, 1 H), 5.81- 5.94 (m, 1 H), 7.16-7.28 (m, 2 H), 7.45 (s, 1 H). |
| 9 | 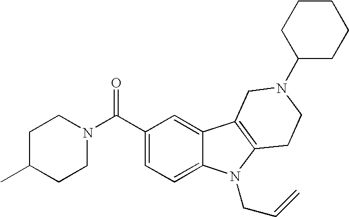 | 5-allyl-2- cyclohexyl-8-[(4- methylpiperidin-1- yl)carbonyl]-2,3,4,5- tetrahydro-1H- pyrido[4,3-b]indole | MS (M + 1): 420.0. 1H NMR (600 MHz, CDC13) δ ppm 0.99 (d, J = 6.40 Hz, 3 H), 1.09- 1.32 (m, 3 H), 1.33-1.47 (m, 2 H) 1.54-1.72 (m, 4 H), 1.76 (d, J = 13.06 Hz, 2 H), 1.98 (d, J = 10.50 Hz, 2 H), 2.21 (m, 2H), 2.74- 3.10 (m, 2 H), 2.93 (d, J = 15.36 Hz, 1 H), 3.33- 3.50 (m, 3 H), 3.74-3.93 (m, 2 H), 4.26 (d, J = 13.82 Hz, 1 H), 4.58 (d, J = 13.82 Hz, 1 H), 4.61- 4.72 (m, 3 H), 4.77 (d, J = 17.15 Hz, 1 H), 5.15 (d, J = 10.50 Hz, 1 H), 5.85-5.95 (m, 1 H), 7.18-7.31 (m, 2 H) 7.48 (s, 1 H). |
| 10 | 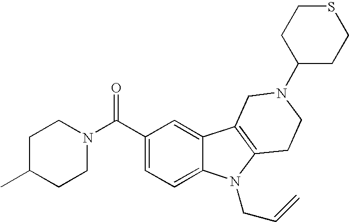 | 5-allyl-8-[(4- methylpiperidin-1- yl)carbonyl]-2- (tetrahydro-2H- thiopyran-4-yl)- 2,3,4,5-tetrahydro- 1H-pyrido[4,3- b]indole | MS (M + 1): 438.0. 1H NMR (600 MHz, CDCl3) δ ppm 0.99 (d, J = 6.40 Hz, 3 H), 1.06- 1.35 (m, 2 H), 1.53-1.90 (m, 3 H), 1.93-2.09 (m, 2 H), 2.48- 2.61 (m, 2 H), 2.75-3.13 (m, 6 H), 3.31-3.46 (m, 2 H), 3.50- 3.62 (m, 1 H), 3.71-3.90 (m, 2 H), 4.26-4.36 (m, 1H), 4.49 (bs, 1 H), 4.56- 4.71 (m, 1 H), 4.63 (s, 3 H), 4.77 (d, J = 17.15 Hz, 1 H), 5.15 (d, J = 9.99 Hz, 1 H), 5.84-5.96 (m, 1 H), 7.19 (d, J = 8.20 Hz, 1 H), 7.25 (d, J = 8.20 Hz, 1 H), 7.43 (s, 1 H). |

### Example 11-36

### General Procedure

The compounds in Example 11-36 were prepared in plate format: A solution of 5-allyl-8-[(4-methylpiperidin-1-yl)carbonyl]-2,3,4,5-tetrahydro-1H-pyrido[4,3-b]indole TFA salt in dichloromethane (˜0.16 M, 0.5 mL, 80 umol) per well, and the corresponding aldehyde (˜0.5M, 0.25 mL, 125 umol) per well was dispensed to the plate using Tecan, and NaBH(OAc)3 (˜30 mg) per well was dispensed to the well by solid dispenser. The plate was shaken overnight, 1 N NaOH (0.5 mL) per well was added, and the plate was shaken for 2 min, the organic phase was transferred to a new plate, the aqueous phase was extracted with dichloromethane (0.5 mL) per well, the combined organic solutions in each well were dried by passing through Hydromatrix (˜0.5 g per well). The solution was removed in vacuo by Genevac evaporator. The plate was purified by preparative LCMS, and the compound purity was obtained by analytical LCMS (10-95% CH₃CN, 3 min)

TABLE 2

| | | | | Reten- |
|---|---|---|---|---|
| | | | | tion |
| Example | | | MS | time |
| No. | Structure | Name | (M + 1) | (min) |
| 11 | 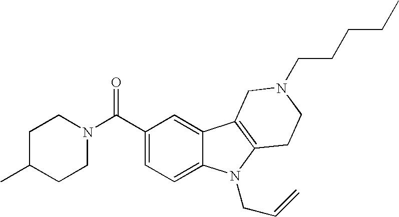 | 5-allyl-8-[(4- methylpiperidin-1- yl)carbonyl]-2-pentyl- 2,3,4,5-tetrahydro-1H- pyrido[4,3-b]indole | 408.3 | 1.53 |
| 12 | 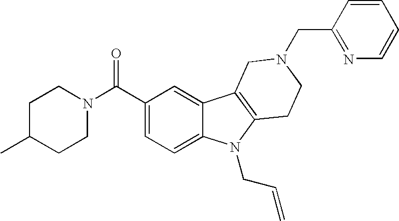 | 5-allyl-8-[(4- methylpiperidin-1- yl)carbonyl]-2- (pyridin-2-ylmethyl)- 2,3,4,5-tetrahydro-1H- pyrido[4,3-b]indole | 429.3 | 1.43 |
| 13 | 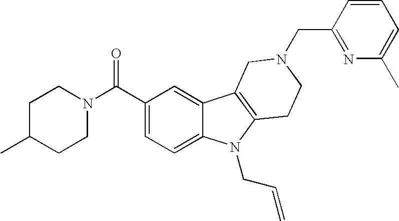 | 5-allyl-8-[(4- methylpiperidin-1- yl)carbonyl]-2-[(6- methylpyridin-2- yl)methyl]-2,3,4,5- tetrahydro-1H- pyrido[4,3-b]indole | 443.3 | 1.38 |
| 14 | 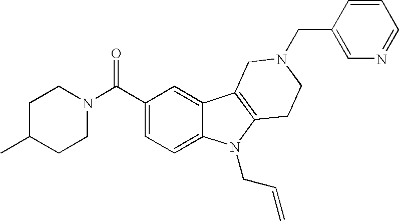 | 5-allyl-8-[(4- methylpiperidin-1- yl)carbonyl]-2- (pyridin-3-ylmethyl)- 2,3,4,5-tetrahydro-1H- pyrido[4,3-b]indole | 429.3 | 1.20 |
| 15 | 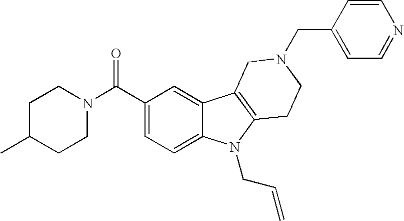 | 5-allyl-8-[(4- methylpiperidin-1- yl)carbonyl]-2- (pyridin-4-ylmethyl)- 2,3,4,5-tetrahydro-1H- pyrido[4,3-b]indole | 429.3 | 1.20 |
| 16 | 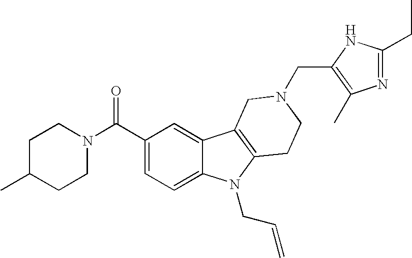 | 5-allyl-2-[(2-ethyl-4- methyl-1H-imidazol-5- yl)methyl]-8-[(4- methylpiperidin-1- yl)carbonyl]-2,3,4,5- tetrahydro-1H- pyrido[4,3-b]indole | 460.3 | 1.23 |
| 17 | 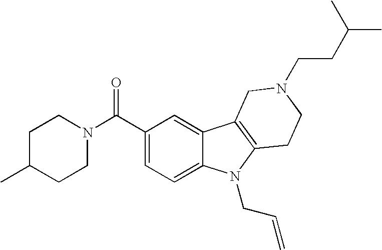 | 5-allyl-2-(3- methylbutyl)-8-[(4- methylpiperidin-1- yl)carbonyl]-2,3,4,5- tetrahydro-1H- pyrido[4,3-b]indole | 408.4 | 1.52 |
| 18 | 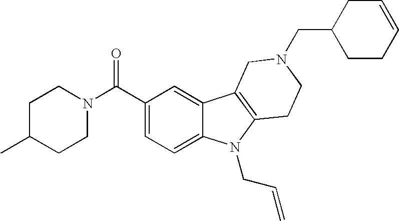 | 5-allyl-2-(cyclohex-3- en-1-ylmethyl)-8-[(4- methylpiperidin-1- yl)carbonyl]-2,3,4,5- tetrahydro-1H- pyrido[4,3-b]indole | 432.4 | 1.55 |
| 19 | 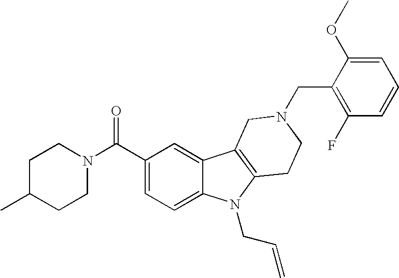 | 5-allyl-2-(2-fluoro-6- methoxybenzyl)-8-[(4- methylpiperidin-1- yl)carbonyl]-2,3,4,5- tetrahydro-1H- pyrido[4,3-b]indole | 476.4 | 1.55 |
| 20 | 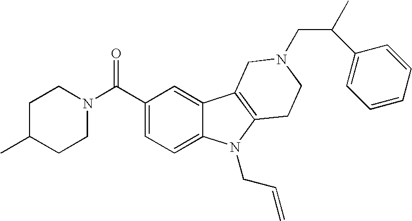 | 5-allyl-8-[(4- methylpiperidin-1- yl)carbonyl]-2-(2- phenylpropyl)-2,3,4,5- tetrahydro-1H- pyrido[4,3-b]indole | 456.4 | 1.58 |
| 21 | 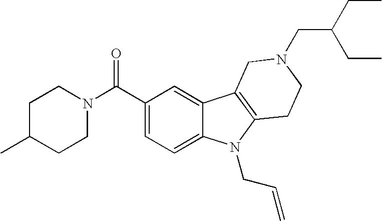 | 5-allyl-2-(2- ethylbutyl)-8-[(4- methylpiperidin-1- yl)carbonyl]-2,3,4,5- tetrahydro-1H- pyrido[4,3-b]indole | 422.4 | 1.57 |
| 22 | 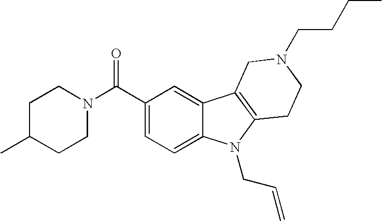 | 5-allyl-2-butyl-8-[(4- methylpiperidin-1- yl)carbonyl]-2,3,4,5- tetrahydro-1H- pyrido[4,3-b]indole | 394.3 | 1.47 |
| 23 | 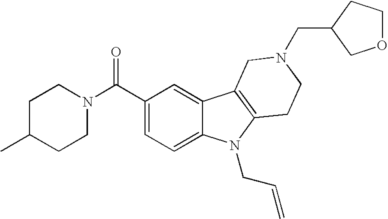 | 5-allyl-8-[(4- methylpiperidin-1- yl)carbonyl]-2- (tetrahydrofuran-3- ylmethyl)-2,3,4,5- tetrahydro-1H- pyrido[4,3-b]indole | 422.3 | 1.33 |
| 24 | 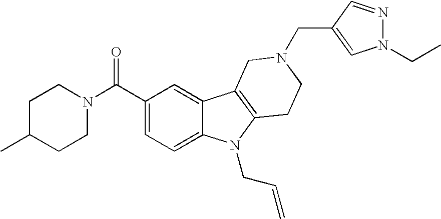 | 5-allyl-2-[(1-ethyl-1H- pyrazol-4-yl)methyl]-8- [(4-methylpiperidin-1- yl)carbonyl]-2,3,4,5- tetrahydro-1H- pyrido[4,3-b]indole | 446.3 | 1.38 |
| 25 | 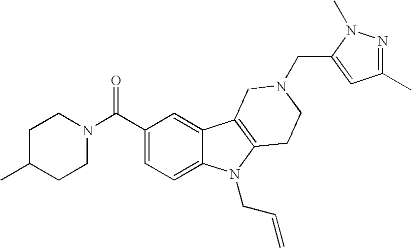 | 5-allyl-2-[(1,3- dimethyl-1H-pyrazol- 5-yl)methyl]-8-[(4- methylpiperidin-1- yl)carbonyl]-2,3,4,5- tetrahydro-1H- pyrido[4,3.-b]indole | 446.3 | 1.37 |
| 26 | 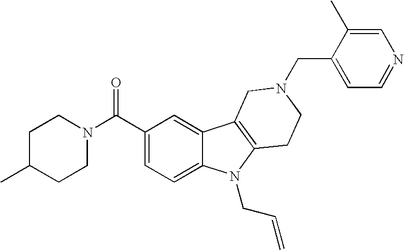 | 5-allyl-8-[(4- methylpiperidin-1- yl)carbonyl]-2-[(3- methylpyridin-4- yl)methyl]-2,3,4,5- tetrahydro-1H- pyrido[4,3-b]indole | 443.3 | 1.22 |
| 27 | 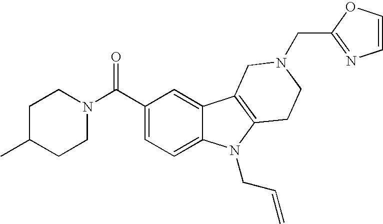 | 5-allyl-8-[(4- methylpiperidin-1- yl)carbonyl]-2-(1,3- oxazol-2-ylmethyl)- 2,3,4,5-tetrahydro-1H- pyrido[4,3-b]indole | 419.3 | 1.32 |
| 28 | 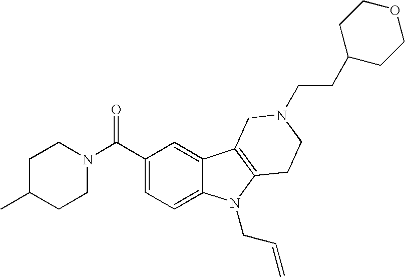 | 5-allyl-8-[(4- methylpiperidin-1- yl)carbonyl]-2-[2- (tetrahydro-2H-pyran- 4-yl)ethyl]-2,3,4,5- tetrahydro-1H- pyrido[4,3-b]indole | 450.3 | 1.41 |
| 29 | 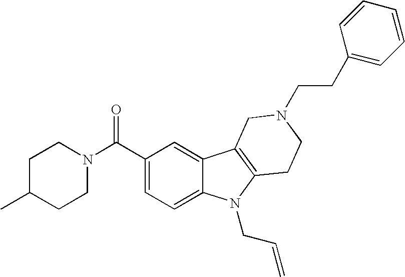 | 5-allyl-8-[(4- methylpiperidin-1- yl)carbonyl]-2-(2- phenylethyl)-2,3,4,5- tetrahydro-1H- pyrido[4,3-b]indole | 442.3 | 1.57 |
| 30 | 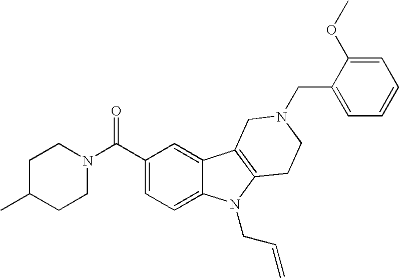 | 5-allyl-2-(2- methoxybenzyl)-8-[(4- methylpiperidin-1- yl)carbonyl]-2,3,4,5- tetrahydro-1H- pyrido[4,3-b]indole | 458.4 | 1.55 |
| 31 | 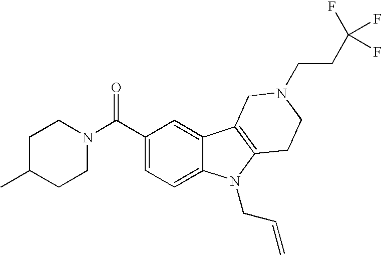 | 5-allyl-8-[(4- methylpiperidin-1- yl)carbonyl]-2-(3,3,3- trifluoropropyl)- 2,3,4,5-tetrahydro-1H- pyrido[4,3-b]indole | 434.3 | 1.47 |
| 32 | 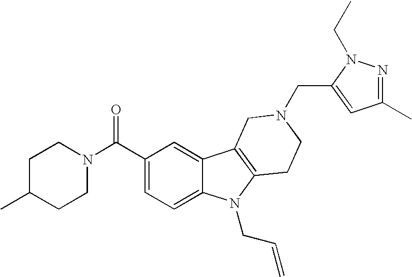 | 5-allyl-2-[(1-ethyl-3- methyl-1H-pyrazol-5- yl)methyl]-8-[(4- methylpiperidin-1- yl)carbonyl]-2,3,4,5- tetrahydro-1H- pyrido[4,3-b]indole | 460.4 | 1.40 |
| 33 | 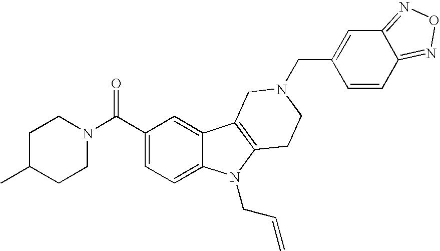 | 5-allyl-2-(2,1,3- benzoxadiazol-5- ylmethyl)-8-[(4- methylpiperidin-1- yl)carbonyl]-2,3,4,5- tetrahydro-1H- pyrido[4,3-b]indole | 470.3 | 1.52 |
| 34 | 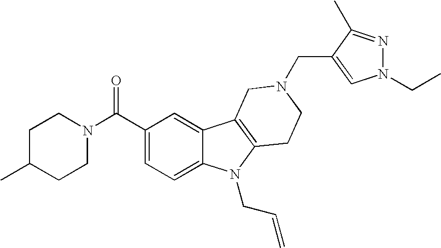 | 5-allyl-2-[(1-ethyl-3- methyl-1H-pyrazol-4- yl)methyl]-8-[(4- methylpiperidin-1- yl)carbonyl]-2,3,4,5- tetrahydro-1H- pyrido[4,3-b]indole | 460.4 | 1.40 |
| 35 | 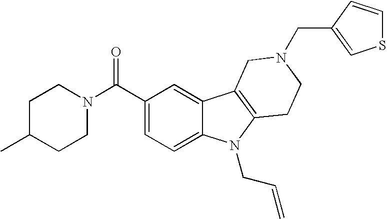 | 5-allyl-8-[(4- methylpiperidin-1- yl)carbonyl]-2-(3- thienylmethyl)-2,3,4,5- tetrahydro-1H- pyrido[4,3-b]indole | 434.3 | 1.50 |
| 36 | 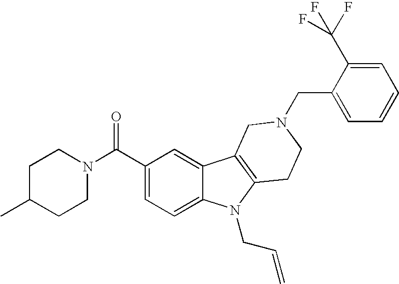 | 5-allyl-8-[(4- methylpiperidin-1- yl)carbonyl]-2-[2- (trifluoromethyl)benzyl]- 2,3,4,5-tetrahydro- 1H-pyrido[4,3-b]indole | 496.3 | 1.60 |

### Example 37

### 5-allyl-8-[(4-methylpiperidin-1-yl)carbonyl]-2-piperidin-4-yl-2,3,4,5-tetrahydro-1H-pyrido[4,3-b]indole

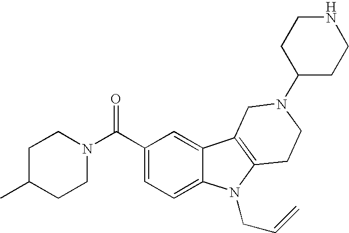

### Step A: The preparation of tert-butyl 4-{5-allyl-8-[(4-methylpiperidin-1-yl)carbonyl]-1,3,4,5-tetrahydro-2H-pyrido[4,3-b]indol-2-yl}piperidine-1-carboxylate

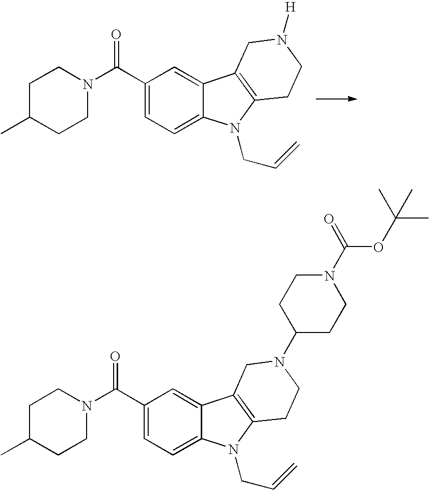

Following the general reductive amination procedure as described in Example 2. NaBH(OAc)₃ (200 mg, 0.94 mmol) was added to a solution of tert-butyl 4-oxopiperidine-1-carboxylate (122 mg, 0.61 mmol), 5-allyl-8-[(4-methylpiperidin-1-yl)carbonyl]-2,3,4,5-tetrahydro-1H-pyrido[4,3-b]indole TFA salt (111 mg, 0.196 mmol) in dichloromethane (5 mL) at room temperature. The reaction was stirred at room temperature overnight, diluted with dichloromethane (20 mL), and 1 N NaOH (2 mL) was added. The mixture was stirred for 5 min, then passed through a prepacked hydromatrix column (10 g), the hydromatrix column was washed with dichloromethane. Removal of dichloromethane gave crude Boc-protected intermediate, which was used for next step without purification.

### Step B: The preparation of 5-allyl-8-[(4-methylpiperidin-1-yl)carbonyl]-2-piperidin-4-yl-2,3,4,5-tetrahydro-1H-pyrido[4,3-b]indole

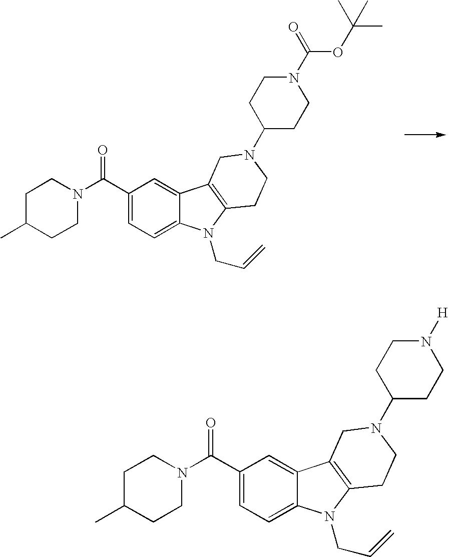

The intermediate from Step A was dissolved in dichloromethane (10 mL) and trifluoroacetic acid (3 mL) was added. The mixture was stirred at room temperature for 30 min, then the excess TFA was evaporated in vacuo. The residue was purified by preparative HPLC to give the desired product (62 mg) as TFA salt. MS (M+1): 421.3. 1H NMR (600 MHz, CD3OD) δ ppm 1.09 (d, J=6.66 Hz, 3H), 1.14-1.39 (m, 21H), 1.63-1.97 (m, 3H), 2.16-2.30 (m, 2H), 2.62 (d, J=13.31 Hz, 2H), 2.88-3.05 (m, 1H), 3.13-3.23 (m, 1H), 3.23-3.31 (m, 2H), 3.36 (t, J=5.63 Hz, 2H), 3.76 (d, J=13.31 Hz, 2H), 3.82-4.02 (m, 4H), 4.62-4.85 (m, 3H), 4.88-4.96 (m, 3H), 5.25 (d, J=9.22 Hz, 1H), 6.04-6.16 (m, 1H), 7.36 (d, J=8.70 Hz, 1H), 7.57 (d, J=8.70 Hz, 1H), 7.70 (s, 1H). Anal. Calcd for C₂₈H₃₈N₄O₂.2C₂HF₃O₂.H₂O: C, 54.23; H, 5.97; N, 7.91. Found, C, 54.49; H, 5.79; N, 7.91.

### Example 38

### 5-allyl-8-[(4-methylpiperidin-1-yl)carbonyl]-2-(tetrahydro-2H-pyran-4-ylmethyl)-2,3,4,5-tetrahydro-1H-pyrido[4,3-b]indole

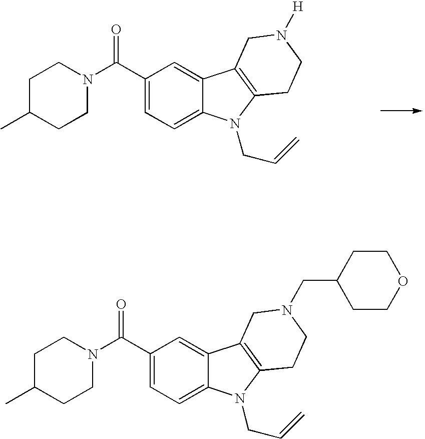

4-(bromomethyl)tetrahydro-2H-pyran (50 uL) was added to a degassed solution of 5-allyl-8-[(4-methylpiperidin-1-yl)carbonyl]-2,3,4,5-tetrahydro-1H-pyrido[4,3-b]indole TFA salt (53 mg, 0.093 mmol) and Cs₂CO₃ (200 mg) in DMF under N₂. The mixture was heated at 110° C. for 1.5 h in a sealed tube, the mixture was allowed to cooled to room temperature, diluted with dichloromethane and water, passed through a short hydromatrix cartridge. The organic solution was concentrated in vacuo and the residue was purified on HPLC (10-65% CH₃CN, 30 min) to give the desired product (16 mg) as its TFA salt, MS (M+1): 436.1. 1H NMR (600 MHz, CD3OD) δ ppm 1.09 (d, J=6.66 Hz, 3H), 1.15-1.37 (m, 2H), 1.38-1.61 (m, 3H), 1.63-1.98 (m, 5H), 2.34-2.48 (m, 1H), 2.82-3.04 (m, 1H), 3.10-3.39 (m, 4H), 3.47-3.54 (m, 1H), 3.56-3.65 (m, 2H), 3.65-3.74 (m, 1H), 3.81-4.13 (m, 5H), 4.46-4.56 (m, 1H), 4.62-4.78 (m, 1H), 4.85-4.97 (m, 3H), 5.25 (d, J=11.26 Hz, 1H), 6.10 (d, 1H), 7.35 (d, J=8.70 Hz, 1H), 7.56 (d, J=8.70 Hz, 1H), 7.71 (s, 1H).

### Example 39

### 5-allyl-2-(2,2-difluoropropanoyl)-8-[(4-methylpiperidin-1-yl)carbonyl]-2,3,4,5-tetrahydro-1H-pyrido[4,3-b]indole

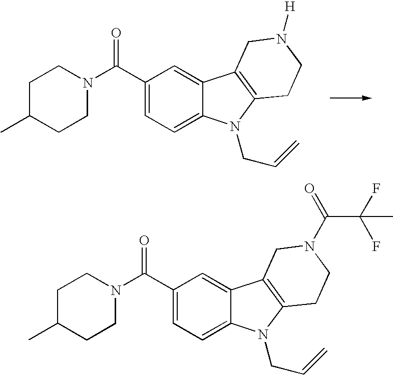

HATU (430 mg, 1.13 mg) was added to a mixture of 5-allyl-8-[(4-methylpiperidin-1-yl)carbonyl]-2,3,4,5-tetrahydro-1H-pyrido[4,3-b]indole diTFA salt (470 mg, 0.83 mmol), 2,2-difluoropropanoic acid (147 mg, 1.33 mmol), diisopropylethylamine (0.8 mL) in DMF (5 mL) at room temperature. The reaction was stirred at room temperature for 30 min, concentrated in vacuo, and water (10 mL) was added. The mixture was extracted with dichloromethane (40 mL) and dried. Removal of solvent gave the desired product (365 mg). A fraction of the crude product was purified on HPLC (25-85% CH₃CN) to afford the pure product for characterization. MS (M+1): 430.1. Based on NMR data, the title compound is a ˜3:1 mixture of two rotomers incurred from the amide bond.

### Example 40

### 5-allyl-2-(cyclopentylcarbonyl)-8-[(4-methylpiperidin-1-yl)carbonyl]-2,3,4,5-tetrahydro-1H-pyrido[4,3-b]indole

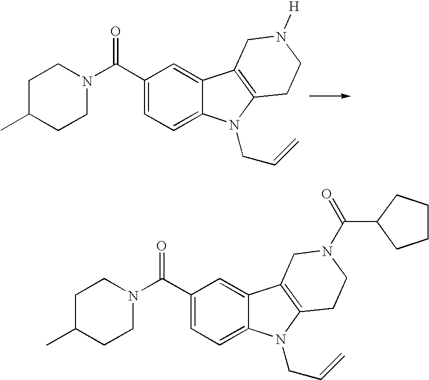

Cyclopentanecarbonyl chloride (0.1 mL) was added to a solution of 8-[(4-methylpiperidin-1-yl)carbonyl]-5-allyl-2,3,4,5-tetrahydro-1H-pyrido[4,3-b]indole diTFA salt (0.1 mmol) and diisopropylethylamine (0.3 mL) in dichloromethane (2 mL) at room temperature. The mixture was stirred at room temperature for 1 h, diluted with dichloromethane (10 mL), and 1N NaOH (1 mL) was added. The mixture was then passed through a short pad of hydromatrix, the organic solution was concentrated in vacuo and the residue was purified by preparative HPLC (10-75% CH₃CN) and lipholization to afford the title compound (21 mg). MS (M+1): 434.4. Based on NMR data, the title compound is a mixture of two rotomers from the amide bond in the ratio of ˜1:3 at room temperature.

### Example 41

### 5-allyl-8-[(4-methylpiperidin-1-yl)carbonyl]-2-(tetrahydro-2H-pyran-4-ylcarbonyl)-2,3,4,5-tetrahydro-1H-pyrido[4,3-b]indole

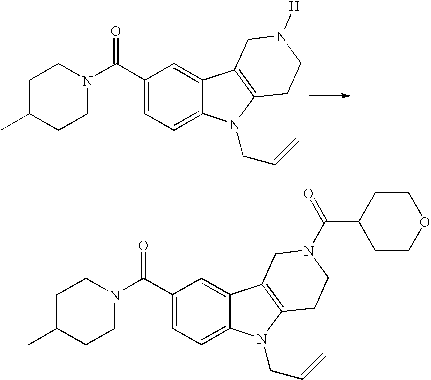

To a solution of 8-[(4-methylpiperidin-1-yl)carbonyl]-5-allyl-2,3,4,5-tetrahydro-1H-pyrido[4,3-b]indole dihydrochloride salt from Example 1D (82 mg, 0.20 mmol), diisopropylethylamine (0.15 mL), tetrahydro-2H-pyran-4-carboxylic acid (32.5 mg, 0.25 mmol) in DMF (3 mL) was added HATU (95 mg, 0.25 mmol) in one portion. The mixture was stirred at room temperature for 30 min. DMF was evaporated and the residue was dissolved in dichloromethane and washed with water. The dichloromethane solution was concentrated in vacuo, and the residue was purified by preparative HPLC (10-70% CH₃CN) to give the title compound as its TFA salt (68 mg). MS (M+1): 450.0. Based on NMR data, the title compound is a mixture of two rotomers from the amide bond in the ratio of ˜1:3 at room temperature.

### Example 42

### 5-allyl-2-(cyclopropykarbonyl)-8-[(4-methylpiperidin-1-yl)carbonyl]-2,3,4,5-tetrahydro-1H-pyrido[4,3-b]indole

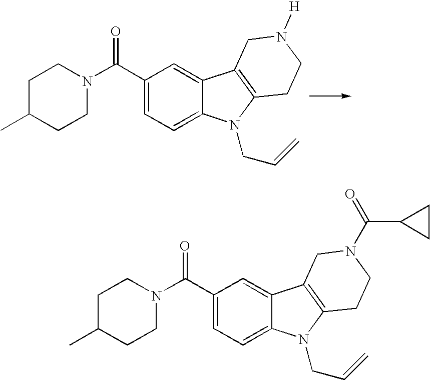

Following the procedure of Example 41: The title compound was prepared from 8-[(4-methylpiperidin-1-yl)carbonyl]-5-allyl-2,3,4,5-tetrahydro-1H-pyrido[4,3-b]indole dihydrochloride salt and cyclopropanecarboxylic acid. MS (M+1): 406.0. Based on NMR data, the title compound is a mixture of two rotomers from the amide bond in the ratio of ˜1:3 at room temperature.

### Example 43

### 5-allyl-2-butyryl-8-[(4-methylpiperidin-1-yl)carbonyl]-2,3,4,5-tetrahydro-1H-pyrido[4,3-b]indole

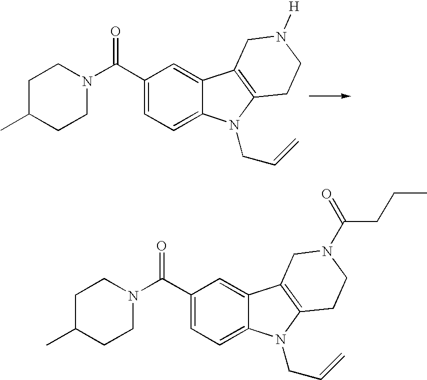

Following the procedure of Example 41: The title compound was prepared from 8-[(4-methylpiperidin-1-yl)carbonyl]-5-allyl-2,3,4,5-tetrahydro-1H-pyrido[4,3-b]indole dihydrochloride salt and butyric acid. MS (M+1): 408.0. Based on NMR data, the title compound is a mixture of two rotomers from the amide bond in the ratio of ˜1:3.

### Example 44

### 5-allyl-N-ethyl-8-[(4-methylpiperidin-1-yl)carbonyl]-1,3,4,4a,5,9b-hexahydro-2H-pyrido[4,3-b]indole-2-carboxamide

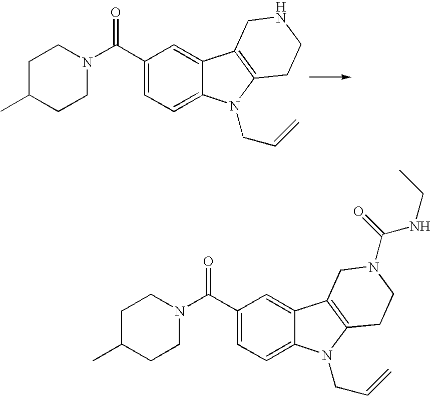

To a solution of 5-allyl-8-[(4-methylpiperidin-1-yl)carbonyl]-2,3,4,5-tetrahydro-1H-pyrido[4,3-b]indole (0.15 mmol) from Example 1D, method B in dry DMA (2 mL) was added ethyl isocyanate (0.15 mmol). The mixture was stirred at room temperature for 2 h. The solution was concentrated in vacuo and the residue was purified with reverse phase HPLC to give the desired product as a TFA salt (27 mg, 35%). MS (M+1): 409.0. 1H NMR (400 MHz, CDCl3) δ ppm 1.00 (d, J=6.25 Hz, 3H), 1.19 (t, J=7.23 Hz, 3H), 1.20-1.30 (m, 2H), 1.62-1.85 (m, 2H), 2.80 (t, J=5.37 Hz, 2H), 2.88-3.10 (m, 2H), 3.35 (q, J=7.23 Hz, 2H), 3.90 (t, J=5.37 Hz, 2H), 4.54 (s, 2H), 4.62-4.71 (m, 2H), 4.86 (d, J=17.18 Hz, 1H), 5.15 (d, J=10.54 Hz, 1H), 5.29-5.68 (m, 3H), 5.83-5.96 (m, 1H), 7.18-7.30 (m, 2H), 7.61 (s, 1H).

### Example 45

### 5-allyl-N-isopropyl-8-[(4-methylpiperidin-1-yl)carbonyl]-1,3,4,4a,5,9b-hexahydro-2H-pyrido[4,3-b]indole-2-carboxamide

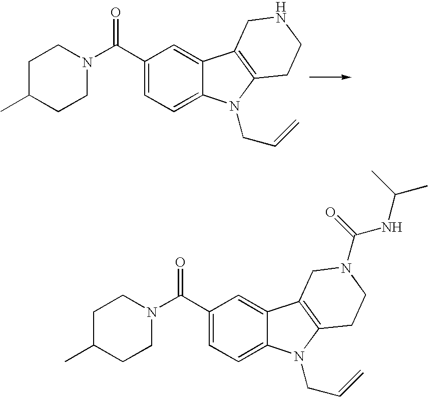

Following the same procedure as Example 44: The title compound was prepared from 5-allyl-8-[(4-methylpiperidin-1-yl)carbonyl]-2,3,4,5-tetrahydro-1H-pyrido[4,3-b]indole and isopropylisocyanate. MS (M+1): 423.0. 1H NMR (400 MHz, CDCl3) δ ppm 1.00 (d, J=6.25 Hz, 3H), 1.15-1.30 (m, 2H), 1.21 (d, J=6.44 Hz, 6H), 1.69 (m, 3H), 2.80 (t, J=5.08 Hz, 2H), 2.96 (s, 2H), 3.91 (m, 2H), 4.04 (m, 1H), 4.52 (s, 2H), 4.66 (m, 2H), 4.87 (d, J=16.99 Hz, 1H), 5.14 (t, J=10.06 Hz, 1H), 5.95-6.00 (m, 4H), 7.18-7.31 (m, 2H), 7.62 (s, 1H).

### Example 46

### 5-allyl-2-(cyclopropylsulfonyl)-8-[(4-methylpiperidin-1-yl)carbonyl]-2,3,4,5-tetrahydro-1H-pyrido[4,3-b]indole

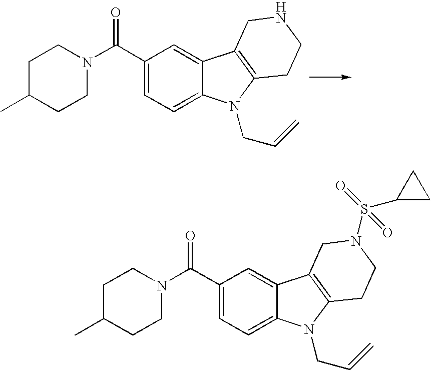

To a solution of 5-allyl-8-[(4-methylpiperidin-1-yl)carbonyl]-2,3,4,5-tetrahydro-1H-pyrido[4,3-b]indole (0.15 mmol) in dry dichloromethane (2 mL) was added triethylamine (0.18 mmol) followed by cyclopropanesulfonyl chloride (0.18 mmol). The mixture was stirred at room temperature for 2 h. The solution was concentrated in vacuo and the residue was purified with reverse phase HPLC to give the desired product (28 mg). MS (M+1): 442.0. 1H NMR (400 MHz, CDCl3) δ ppm 0.92-1.05 (m, 3H), 1.00 (d, J=6.25 Hz, 3H), 1.20-1.35 (m, 3H), 1.58-1.80 (m, 2H), 2.34 (m, 1 H), 2.91 (t, J=5.62 Hz, 2H), 3.01-3.12 (m 1H), 3.77 (t, J=5.62 Hz, 2H), 3.82-3.98 (m, 1H), 4.39-4.59 (m, 3H), 4.62 (s, 2H), 4.67 (m, 2H), 4.83 (d, J=16.99 Hz, 1H), 5.15 (d, J=10.35 Hz, 1H), 5.91 (m, 1H), 7.20-7.30 (m, 2H), 7.52 (s, 1H).

### Example 47

### 5-allyl-2-(ethylsulfonyl)-8-[(4-methylpiperidin-1-yl)carbonyl]-2,3,4,5-tetrahydro-1H-pyrido[4,3-b]indole

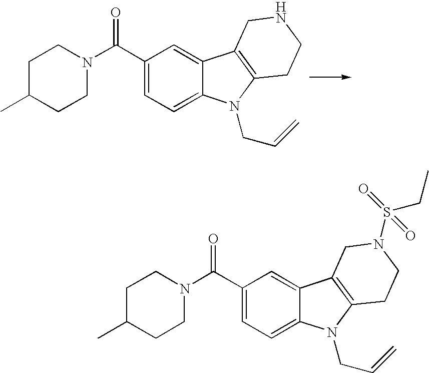

To a solution of 5-allyl-8-[(4-methylpiperidin-1-yl)carbonyl]-2,3,4,5-tetrahydro-1H-pyrido[4,3-b]indole (0.15 mmol) in dry dichloromethane (2 mL) was added triethylamine (0.18 mmol) followed by ethanesulfonyl chloride (0.18 mmol). The mixture was stirred at room temperature for 2 h. The solution was concentrated in vacuo and the residue was purified with reverse phase HPLC to give the desired product (31 mg). MS (M+1): 430.0. 1H NMR (400 MHz, CDCL3) δ ppm 1.00 (d, J=6.25 Hz, 3H), 1.28 (m, 2H), 1.38 (t, J=7.35 Hz, 3H), 1.71 (m, 3H), 2.88 (t, J=5.56 Hz, 2H), 3.05 (q, J=7.35 Hz, 2H), 3.54 (s, 4H), 3.78 (t, J=5.56 Hz, 2H), 4.60 (s, 2H), 4.67 (m, 2H), 4.84 (d, J=17.18 Hz, 1H), 5.15 (d, J=10.35 Hz, 1H), 5.92 (m, 1H), 7.25 (m, 2H), 7.51 (s, 1H).

### Example 48

### 5-(2-methoxyethyl)-8-[(4-methylpiperidin-1-yl)carbonyl]-2-(tetrahydro-2H-pyran-4-yl)-2,3,4,5-tetrahydro-1H-pyrido[4,3-b]indole

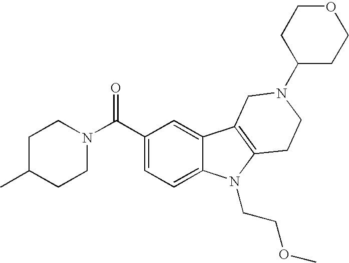

### Step A: The preparation of tert-butyl 5-(2-methoxyethyl)-8-[(4-methylpiperidin-1-yl)carbonyl]-1,3,4,5-tetrahydro-2H-pyrido[4,3-b]indole-2-carboxylate

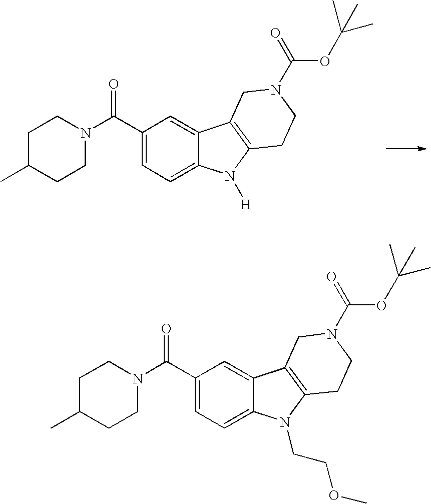

Following the general procedure described in Example 1, Step C: tert-butyl 542-methoxyethyl)-8-[(4-methylpiperidin-1-yl)carbonyl]-1,3,4,5-tetrahydro-2H-pyrido[4,3-b]indole-2-carboxylate (1.35 g, 49%) was prepared from tert-butyl 8-[(4-methylpiperidin-1-yl)carbonyl]-1,3,4,5-tetrahydro-2H-pyrido[4,3-b]indole-2-carboxylate (2.40 g) and 2-bromoethyl methyl ether (1.0 mL). MS (M+1): 456.1.

### Step B: The preparation of 5-(2-methoxyethyl)-8-[(4-methylpiperidin-1-yl)carbonyl]-2,3,4,5-tetrahydro-1H-pyrido[4,3-b]indole diTFA salt

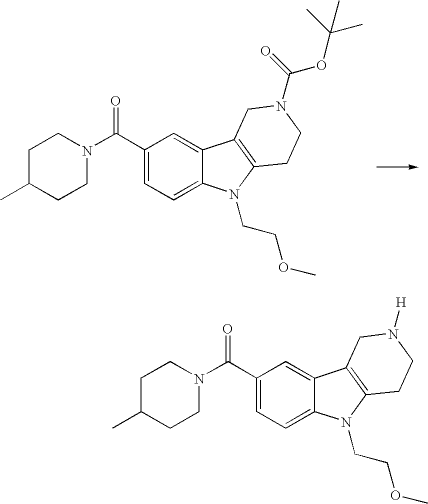

Following the same procedure of Example 1, Step D (Method B): The title compound as its diTFA salt (1.45 g) was prepared from tert-butyl 5-(2-methoxyethyl)-8-[(4-methylpiperidin-1-yl)carbonyl]-1,3,4,5-tetrahydro-2H-pyrido[4,3-b]indole-2-carboxylate (1.35 g) from Step A.

### Step C: The preparation of 5-(2-methoxyethyl)-8-[(4-methylpiperidin-1-yl)carbonyl]-2-(tetrahydro-2H-pyran-4-yl)-2,3,4,5-tetrahydro-1H-pyrido[4,3-b]indole

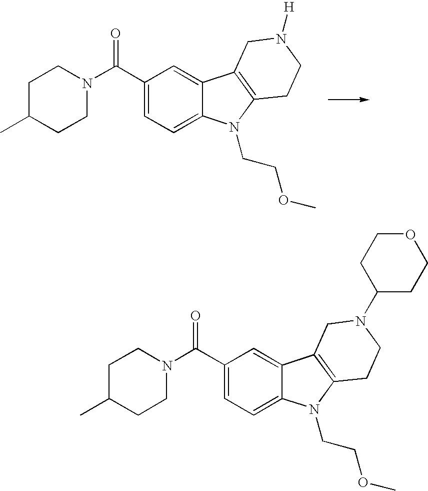

Following the similar procedure as described in Example 2: the title compound as its TEA salt was prepared from 5-(2-methoxyethyl)-8-[(4-methylpiperidin-1-yl)carbonyl]-2,3,4,5-tetrahydro-1H-pyrido[4,3-b]indole and tetrahydro-4H-pyran-4-one. MS (M+1): 440.0. 1H NMR (600 MHz, CD3OD) δ ppm 0.83 (d, J=6.66 Hz, 3H), 0.92-1.15 (m, 2H), 1.39-1.70 (m, 3H), 1.71-1.83 (m, 2H), 2.02 (m, 2H), 2.63-2.78 (m, 1H), 2.88-3.02 (m, 1H), 3.08 (s, 3H), 3.15 (bs, 2H), 3.28-3.42 (m, 3H), 3.46-3.59 (m, 3H), 3.60-3.71 (m, 1H), 3.80-3.91 (m, 1H), 3.96 (dd, J=11.52, 3.84 Hz, 2H), 4.23-4.13 (m, 2H), 4.31 (d, J=13.57 Hz, 1H), 4.52 (d, J=13.57 Hz, 2H), 7.09 (d, J=8.45 Hz, 1H), 7.34 (d, J=8.45 Hz, 1H), 7.42 (s, 1H).

### Example 49-64

The following examples (Table 3) were prepared from 5-(2-methoxyethyl)-8-[(4-methylpiperidin-1-yl)carbonyl]-2,3,4,5-tetrahydro-1H-pyrido[4,3-b]indole diTFA salt by the same plate method described in Example 11-36. The LCMS condition: 10-95% CH₃CN, 3 min.

| | | | | Reten- |
|---|---|---|---|---|
| | | | MS | tion |
| Example | Structures | IUPAC Name | (M + 1) | time |
| 49 | 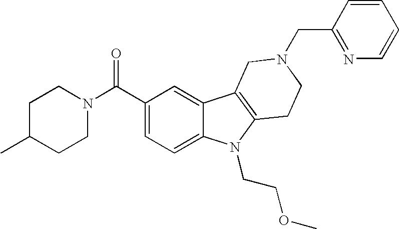 | 5-(2-methoxyethyl)- 8-[(4- methylpiperidin-1- yl)carbonyl]-2- (pyridin-2- ylmethyl)-2,3,4,5- tetrahydro-1H- pyrido[4,3-b]indole | 447.3 | 1.33 |
| 50 | 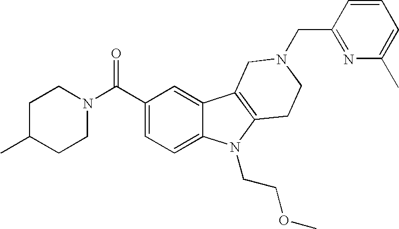 | 5-(2-methoxyethyl)- 8-[(4- methylpiperidin-1- yl)carbonyl]-2-[(6- methylpyridin-2- yl)methyl]-2,3,4,5- tetrahydro-1H- pyrido[4,3-b]indole | 461.3 | 1.32 |
| 51 | 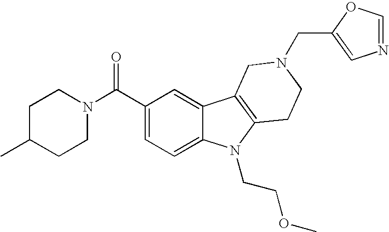 | 5-(2-methoxyethyl)- 8-[(4- methylpiperidin-1- yl)carbonyl]-2-(1,3- oxazol-5-ylmethyl)- 2,3,4,5-tetrahydro- 1H-pyrido[4,3- b]indole | 437.3 | 1.22 |
| 52 | 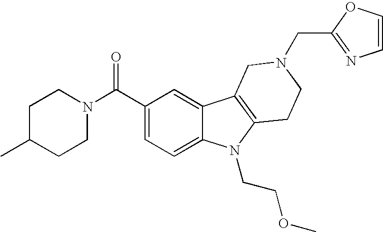 | 5-(2-methoxyethyl)- 8-[(4- methylpiperidin-1- yl)carbonyl]-2-(1,3- oxazol-2-ylmethyl)- 2,3,4,5-tetrahydro- 1H-pyrido[4,3- b]indole | 437.3 | 1.23 |
| 53 | 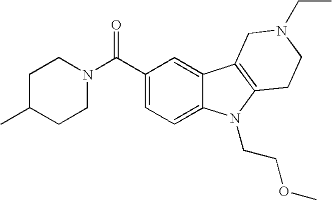 | 2-ethyl-5-(2- methoxyethyl)-8-[(4- methylpiperidin-1- yl)carbonyl]-2,3,4,5- tetrahydro-1H- pyrido[4,3-b]indole | 384.3 | 1.23 |
| 54 | 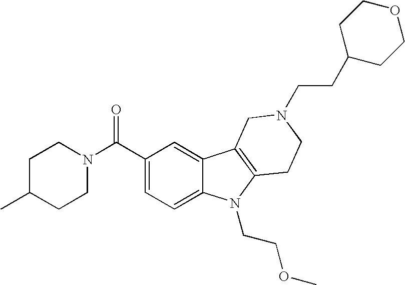 | 5-(2-methoxyethyl)- 8-[(4- methylpiperidin-1- yl)carbonyl]-2-[2- (tetrahydro-2H- pyran-4-yl)ethyl]- 2,3,4,5-tetrahydro- 1H-pyrido[4,3- b]indole | 468.3 | 1.33 |
| 55 | 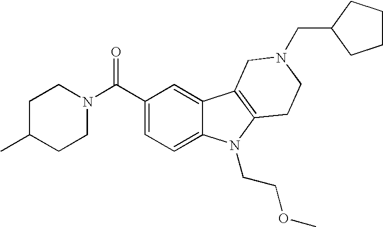 | 2- (cyclopentylmethyl)- 5-(2-methoxyethyl)- 8-[(4- methylpiperidin-1- yl)carbonyl]-2,3,4,5- tetrahydro-1H- pyrido[4,3-b]indole | 438.3 | 1.48 |
| 56 | 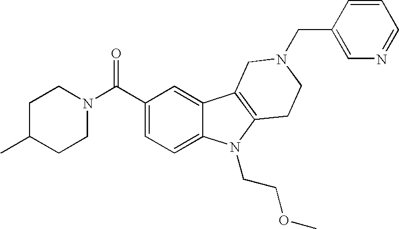 | 5-(2-methoxyethyl)- 8-[(4- methylpiperidin-1- yl)carbonyl]-2- (pyridin-3- ylmethyl)-2,3,4,5- tetrahydro-1H- pyrido[4,3-b]indole | 447.3 | 1.16 |
| 57 | 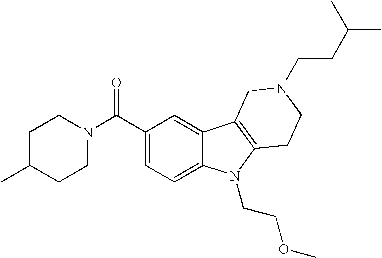 | 5-(2-methoxyethyl)- 2-(3-methylbutyl)-8- [(4-methylpiperidin- 1-yl)carbonyl]- 2,3,4,5-tetrahydro- 1H-pyrido[4,3- b]indole | 426.3 | 1.45 |
| 58 | 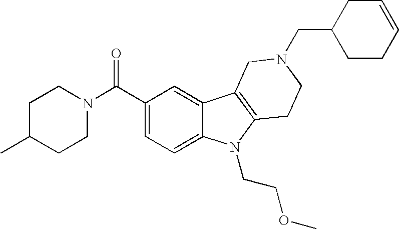 | 2-(cyclohex-3-en-1- ylmethyl)-5-(2- methoxyethyl)-8-[(4- methylpiperidin-1- yl)carbonyl]-2,3,4,5- tetrahydro-1H- pyrido[4,3-b]indole | 450.3 | 1.48 |
| 59 | 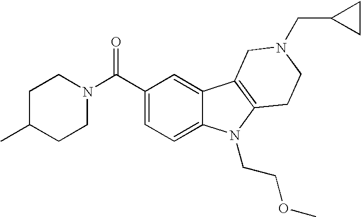 | 2- (cyclopropylmethyl)- 5-(2-methoxyethyl)- 8-[(4- methylpiperidin-1- yl)carbonyl]-2,3,4,5- tetrahydro-1H- pyrido[4,3-b]indole | 410.3 | 1.33 |
| 60 | 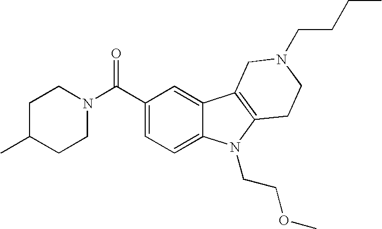 | 2-butyl-5-(2- methoxyethyl)-8-[(4- methylpiperidin-1- yl)carbonyl]-2,3,4,5- tetrahydro-1H- pyrido[4,3-b]indole | 412.3 | 1.40 |
| 61 | 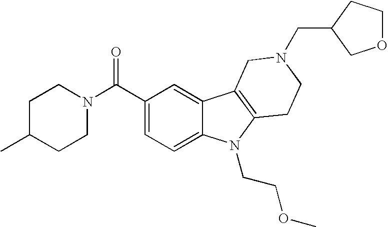 | 5-(2-methoxyethyl)- 8-[(4- methylpiperidin-1- yl)carbonyl]-2- (tetrahydrofuran-3- ylmethyl)-2,3,4,5- tetrahydro-1H- pyrido[4,3-b]indole | 440.3 | 1.25 |
| 62 | 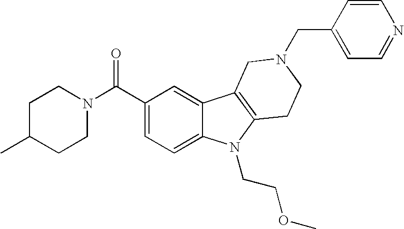 | 5-(2-methoxyethyl)- 8-[(4- methylpiperidin-1- yl)carbonyl]-2- (pyridin-4- ylmethyl)-2,3,4,5- tetrahydro-1H- pyrido[4,3-b]indole | 447.3 | 1.12 |
| 63 | 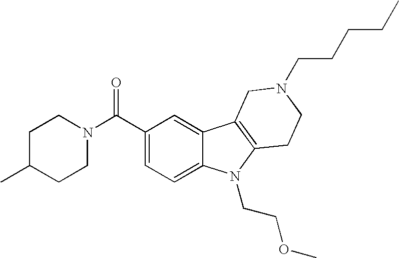 | 5-(2-methoxyethyl)- 8-[(4- methylpiperidin-1- yl)carbonyl]-2- pentyl-2,3,4,5- tetrahydro-1H- pyrido[4,3-b]indole | 426.3 | 1.48 |
| 64 | 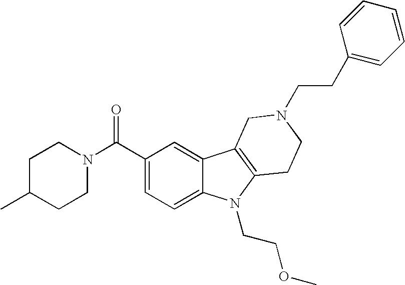 | 5-(2-methoxyethyl)- 8-[(4- methylpiperidin-1- yl)carbonyl]-2-(2- phenylethyl)-2,3,4,5- tetrahydro-1H- pyrido[4,3-b]indole | 460.3 | 1.52 |

### Example 65

### 2-cyclopentyl-8-[(4-methylpiperidin-1-yl)carbonyl]-5-propyl-2,3,4,5-tetrahydro-1H-pyrido[4,3-b]indole

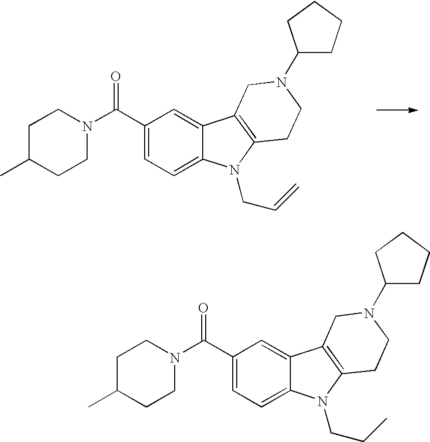

To a solution of 5-allyl-2-cyclopentyl-8-[(4-methylpiperidin-1-yl)carbonyl]-2,3,4,5-tetrahydro-1H-pyrido[4,3-b]indole TFA salt (56 mg, 0.14 mmol) in MeOH (6 mL) was added Pd/C (10 mg) and the mixture placed under H₂ (30 psi). The mixture stirred over night at room temperature and then filtered and condensed. The obtained oil (60 mg) purified by reverse phase HPLC (5-75% CH₃CN) to provide the title compound as its TFA salt (30 mg). MS (M+1): 408.0. 1H NMR (400 MHz, CDCl3) δ ppm 0.89 (t, J=14.84 Hz, 3H), 0.98 (d, J=6.44 Hz, 3H), 1.05-1.37 (m, 2H), 1.55-1.94 (m, 9H), 1.95-2.23 (m, 4H), 2.57-3.11 (m, 3H), 3.30-3.49 (m, 2H), 3.52-3.67 (m, 1H), 3.69-4.08 (m, 4H), 4.17 (d, J=13.86 Hz, 1H), 4.71 (d, J=14.25 Hz, 1H), 4.57-4.78 (m, 1H), 7.21 (d, J=8.60 Hz, 1H), 7.29 (d, J=8.60 Hz, 1H), 7.47 (s, 1H).

### Example 66

### 8-[(4-methylpiperidin-1-yl)carbonyl]-5-propyl-2-(3,3,3-trifluoropropyl)-2,3,4,5-tetrahydro-1H-pyrido[4,3-b]indole

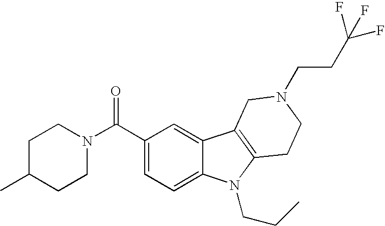

### Step A: The preparation of tert-butyl 8-[(4-methylpiperidin-1-yl)carbonyl]-5-propyl-1,3,4,5-tetrahydro-2H-pyrido[4,3-b]indole-2-carboxylate

### Method A: Hydrogenation Method

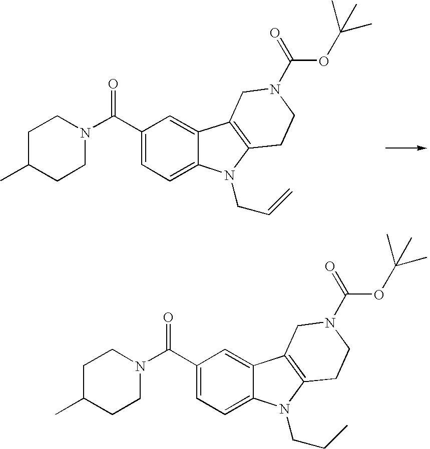

tert-butyl 5-allyl-8-[(4-methylpiperidin-1-yl)carbonyl]-1,3,4,5-tetrahydro-2H-pyrido[4,3-b]indole-2-carboxylate from Example 1, Step C (1.10 g, 2.52 mmol) was dissolved in methanol (40 mL), and 10% Pd/C (100 mg) was added The mixture was hydrogenated at 30 psi H₂ for 2 h. Pd/C was filtered off and removal of solvent gave the desired product (998 mg, 90.3%). MS (M+1): 440.1.

### Method B: Alkylation Method

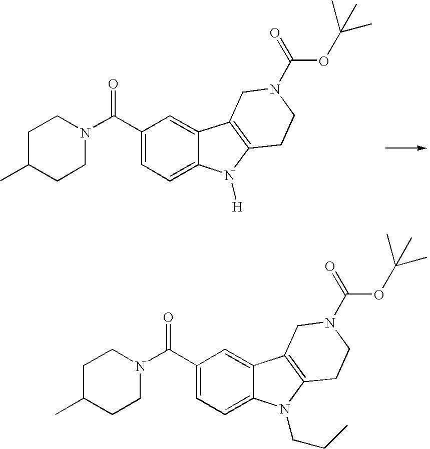

60% NaH (120 mg, 3.0 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (325 mg, 0.819 mmol) in DMF (10 mL) at 0° C. under nitrogen. The mixture was stirred at room temperature for 30 min, then 1-iodopropane (402 mg, 2.36 mmol) was added at room temperature, and the reaction mixture was stirred at room temperature for 1 h. The solvent was removed in vacuo and the residue was suspended in water (10 mL), the light yellow solid was collected, washed with water (20 mL) to give crude product (365 mg). The crude product was dissolved in methanol, and then methanol was removed in vacuo to afford the desired product (346 mg, 96%). MS (M+1): 440.1.

### Step B: The preparation of 8-[(4-methylpiperidin-1-yl)carbonyl]-5-propyl-2,3,4,5-tetrahydro-1H-pyrido[4,3-b]indole

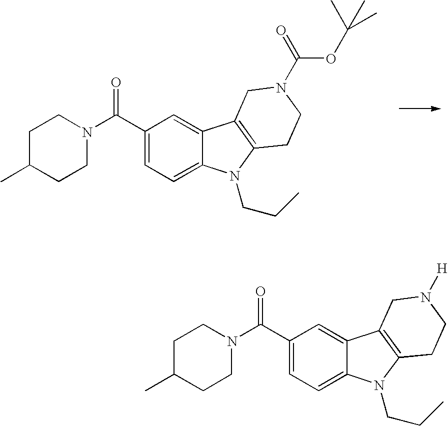

tert-butyl 8-[(4-methylpiperidin-1-yl)carbonyl]-5-propyl-1,3,4,5-tetrahydro-2H-pyrido[4,3-b]indole-2-carboxylate (346 mg, 0.788 mmol) was dissolved in dichloromethane (8 mL) and trifluoroacetic acid (2 mL) was added. The mixture was stirred at room temperature for 30 min. Dichloromethane and excess trifluoroacetic acid (TFA) were removed in vacuo, and the residue was lipholized to afford the desired product (371 mg) as its TFA salt. MS (M+1): 340.0.

### Step C: The preparation of 8-[(4-methylpiperidin-1-yl)carbonyl]-5-propyl-2-(3,3,3-trifluoropropyl)-2,3,4,5-tetrahydro-1H-pyrido[4,3-b]indole

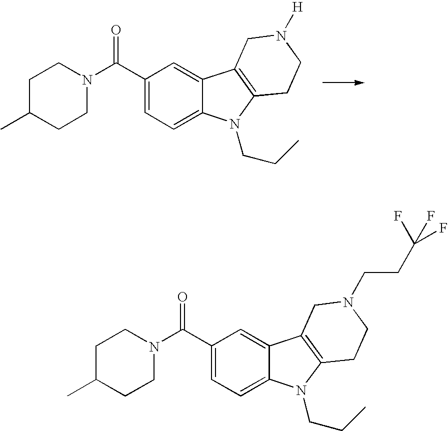

Following the general procedure of Example 1, Step E: the desired product as its TFA salt was prepared from 8-[(4-methylpiperidin-1-yl)carbonyl]-5-propyl-2,3,4,5-tetrahydro-1H-pyrido[4,3-b]indole TFA salt and 3,3,3-trifluoropropanal. MS (M+1): 436.4. 1HNMR (600 Hz, CD3OD): δ ppm 1.02 (t, J=7.1 Hz, 3H), 1.07 (d, J=6.40 Hz, 3H), 1.26 (m, 2H), 1.60-1.95 (m, 5H), 2.85-3.25 (m, 4H), 3.37 (s, 2H), 3.79 (t, J=7.70 Hz, 2H), 3.95 (m, 3H), 4.24 (s, 2H), 4.70 (m, 3H), 7.35 (d, J=8.2 Hz, 1H), 7.61 (d, J=8.2 Hz, 1H), 7.65 (s, 1H).

### Example 67

### 8-[(4-methylpiperidin-1-yl)carbonyl]-5-propyl-2-(tetrahydro-2H-pyran-4-yl)-2,3,4,5-tetrahydro-1H-pyrido[4,3-b]indole

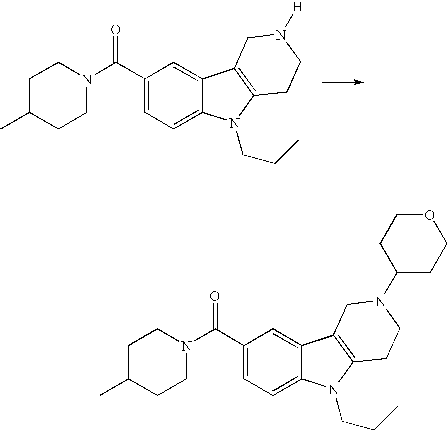

Following the general procedure of Example 1, Step E: the desired product as its TFA salt was prepared from 8-[(4-methylpiperidin-1-yl)carbonyl]-5-propyl-2,3,4,5-tetrahydro-1H-pyrido[4,3-b]indole TFA salt and tetrahydro-4H-pyran-4-one. MS (M+1): 424.11. 1H NMR (600 MHz, CD3OD) δ ppm 0.81 (t, J=7.17 Hz, 3H), 0.87 (d, J=6.14 Hz, 3H), 0.96-1.15 (m, 2H), 1.40-1.87 (m, 7H), 1.97-2.12 (m, 2H), 2.74 (s, 1H), 2.98 (s, 1H), 3.10-3.17 (m, 2H), 3.26-3.78 (m, 5H), 3.82-4.11 (m, 5H), 4.30-4.67 (m, 3H), 7.13 (d, J=9.22 Hz, 1H), 7.39 (d, J=9.22 Hz, 1H), 7.47 (s, 1H).

### Example 68

### 8-[(4-methylpiperidin-1-yl)carbonyl]-5-propyl-2-(tetrahydrofuran-3-ylmethyl)-2,3,4,5-tetrahydro-1H-pyrido[4,3-b]indole

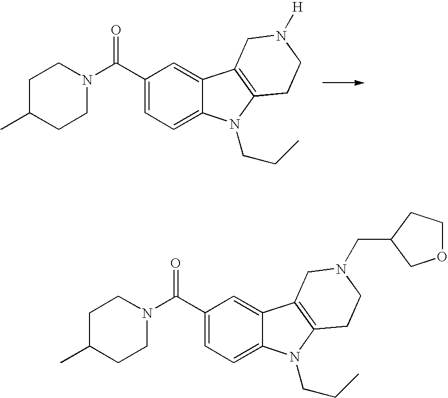

Following the general procedure of Example 1, Step E: the desired product as its TFA salt was prepared from 8-[(4-methylpiperidin-1-yl)carbonyl]-5-propyl-2,3,4,5-tetrahydro-1H-pyrido[4,3-b]indole diTFA salt and tetrahydrofuran-3-carbaldehyde. MS (M+1): 424.46. 1H NMR (400 MHz, CD3OD) δ ppm 0.91 (t, J=7.42 Hz, 3H), 0.96 (d, J=6.25 Hz, 3H), 1.01-1.27 (m, 2H), 1.50-1.88 (m, 6H), 2.15-2.33 (m, 1H), 2.76-2.95 (m, 2H), 3.08 (d, J=10.16 Hz, 1H), 3.18-3.27 (m, 2H), 3.43 (d, J=7.42 Hz, 2H), 3.47-3.65 (m, 2H), 3.68-4.03 (m, 5H), 4.12 (t, J=7.03 Hz, 2H), 4.25-4.46 (m, 1H), 4.50-4.67 (m, 1H), 4.67-4.85 (m, 1H), 7.22 (dd, J=8.59, 1.56 Hz, 1H), 7.48 (d, J=8.59 Hz, 1H), 7.54 (s, 1H).

### Example 69

### 2-(2,2-difluoroethyl)-8-[(4-methylpiperidin-1-yl)carbonyl]-5-propyl-2,3,4,5-tetrahydro-1H-pyrido[4,3-b]indole

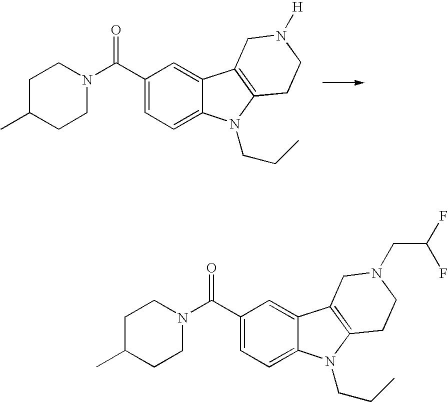

1,1-difluoro-2-iodoethane (50 uL) was added to a degassed solution of 5-propyl-8-[(4-methylpiperidin-1-yl)carbonyl]-2,3,4,5-tetrahydro-1H-pyrido[4,3-b]indole diTFA salt (100 mg, 0.176 mmol) and Cs₂CO₃ (200 mg) in DMF (3 mL) under N₂. The mixture was heated at 140° C. for 4 h in a sealed tube, the mixture was allowed to cooled to room temperature, diluted with dichloromethane and water, passed through hydromatrix column. The organic solution was concentrated in vacuo and the residue was purified on HPLC (10-75% CH₃CN, 30 min) to give the title compound (12 mg) as its TFA salt. MS (M+1): 404.1. 1H NMR (400 MHz, CD3OD) δ ppm 0.92 (t, J=7.42 Hz, 3H), 0.96 (d, J=6.44 Hz, 3H), 1.03-1.27 (m, 2H), 1.49-1.92 (m, 5H), 2.73-3.17 (m, 3H), 3.26 (s, 1H), 3.81-3.97 (m, 5H), 4.13 (t, J=7.23 Hz, 2H), 4.47-4.65 (m, 1H), 4.68 (s, 2H), 6.51 (tt, J=53.50, 3.60 Hz, 1H), 7.24 (dd, J=8.59, 1.17 Hz, 1H), 7.50 (d, J=8.59 Hz, 1H), 7.53 (d, J=1.17 Hz, 1H).

### Example 70

### (1R*,2R*)-2-{8-[(4-methylpiperidin-1-yl)carbonyl]-5-propyl-1,3,4,5-tetrahydro-2H-pyrido[4,3-b]indol-2-yl}cyclopentanol

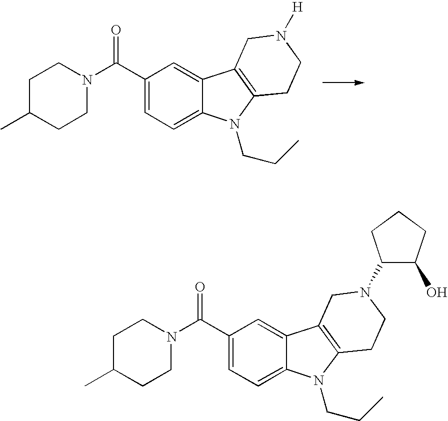

6-oxabicyclo[3.1.0]hexane (0.1 mL) was added to a degassed mixture of 5-propyl-8-[(4-methylpiperidin-1-yl)carbonyl]-2,3,4,5-tetrahydro-1H-pyrido[4,3-b]indole diTFA salt (63 mg, 0.11 mmol) and Cs₂CO₃ (270 mg) in DMF (3 mL) under N₂. The mixture was heated at 140° C. overnight, allowed to cool to room temperature, diluted with ethyl ether, washed with water. The ethereal solution was concentrated in vacuo, and the residue was purified by preparative HPLC (5-65% CH₃CN) to give the title compound as its TFA salt (13 mg). MS (M+1): 424.1. ¹HNMR (600 MHz, CD₃OD): δ ppm 1.03 (t, J=7.2 Hz, 3H), 1.07 (d, J=6.1 Hz, 3H), 1.30 (m, 2H), 1.52-2.00 (m, 11H), 2.22 (m, 1H), 2.46 (m, 1H), 2.95 (brs, 1H), 3.19 (brs, 1H), 3.58-4.05 (m, 3H), 4.23 (m, 2H), 4.46-4.75 (m, 5H), 7.34 (d, J=8.2 Hz, 1H), 7.63 (m, 2H).

### Example 71

### 8-[(4-methylpiperidin-1-yl)carbonyl]-5-propyl-2-pyridin-2-yl-2,3,4,5-tetrahydro-1H-pyrido[4,3-b]indole

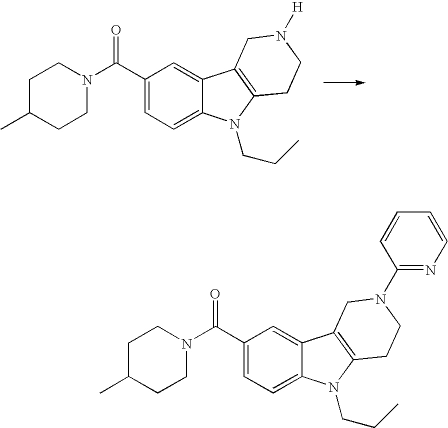

The degassed mixture of 2-bromopyridine (0.1 mL), 5-propyl-8-[(4-methylpiperidin-1-yl)carbonyl]-2,3,4,5-tetrahydro-1H-pyrido[4,3-b]indole diTFA salt (77 mg; 0.135 mmol) and Cs₂CO₃ (100 mg) in DMF (2 mL) were heated at 145° C. under N₂ in a sealed tube overnight. Similar work as in Example 62 and purification by preparative HPLC (15-70% CH₃CN) to give the title compound as its TFA salt (16 mg). MS (M+1): 417.10; 1H NMR (400 MHz, CD3OD) δ ppm 0.91 (t, J=7.42 Hz, 3H), 1.00 (d, J=6.25 Hz, 3H), 1.08-1.30 (m, 2H), 1.52-1.93 (m, 5H), 2.72-3.22 (m, 4H), 3.87 (s, 1H), 4.07-4.25 (m, 4H), 4.62 (s, 1H), 4.92 (s, 2H), 7.01 (t, J=6.64 Hz, 1 H), 7.20-7.26 (m, 1H), 7.48 (d, J=8.59 Hz, 1H), 7.55-7.65 (m, 2H), 7.88-8.03 (m, 1H), 8.03-8.14 (m, 1H).

### Example 72

### 8-[(4-methylpiperidin-1-yl)carbonyl]-5-propyl-2-(3,4,5,6-tetrahydropyridin-2-yl)-2,3,4,5-tetrahydro-1H-pyrido[4,3-b]indole

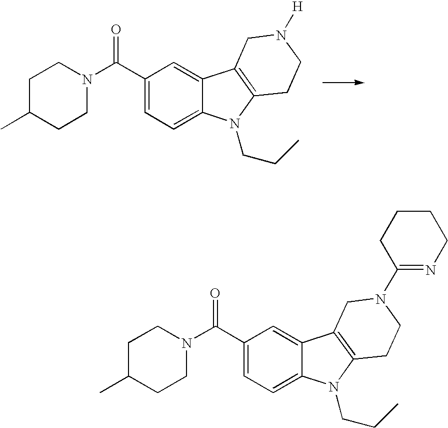

6-methoxy-2,3,4,5-tetrahydropyridine (0.3 mL) was added to the solution of 5-propyl-8-[(4-methylpiperidin-1-yl)carbonyl]-2,3,4,5-tetrahydro-1H-pyrido[4,3-b]indole tetraTFA salt (110 mg, 138 mg) in methanol (1 mL) at room temperature. The mixture was stirred at room temperature overnight and purified by preparative HPLC (10-70% CH3CN) to afford the title compound as its TFA salt (35 mg). MS (M+1): 421.10. 1H NMR (400 MHz, CD3OD) δ ppm 0.89 (t, J=7.42 Hz, 3H), 0.97 (d, J=6.45 Hz, 3H), 1.01-1.29 (m, 2H), 1.48-2.01 (m, 10H), 2.74-2.94 (m, 3H), 2.97-3.12 (m, 3H), 3.48 (q, J=6.31 Hz, 2H), 3.82 (s, 1H), 3.98 (t, J=5.66 Hz, 2H), 4.05-4.16 (m, 2H), 4.58 (s, 1H), 4.74 (s, 1H), 7.15-7.24 (m, 1H), 7.41-7.57 (m, 2H).

### Example 73

### 2-(3,4-dihydro-2H-pyrrol-5-yl)-8-[(4-methylpiperidin-1-yl)carbonyl]-5-propyl-2,3,4,5-tetrahydro-1H-pyrido[4,3-b]indole

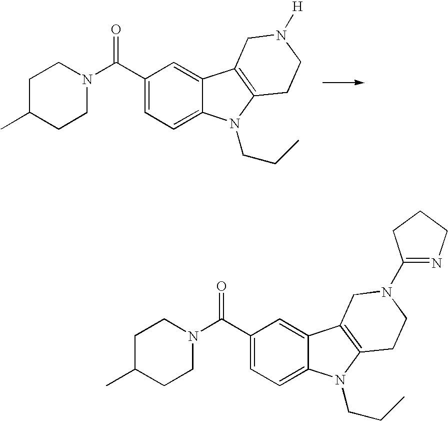

5-methoxy-3,4-dihydro-2H-pyrrole (0.3 mL) was added to the solution of 5-propyl-8-[(4-methylpiperidin-1-yl)carbonyl]-2,3,4,5-tetrahydro-1H-pyrido[4,3-b]indole tetraTFA salt (110 mg, 138 mg) in methanol (1 mL) at room temperature. The mixture was stirred at room temperature overnight and purified by preparative HPLC (10-70% CH₃CN) to afford the title compound as its TFA salt (31 mg). MS (M+1): 407.1. ¹H NMR (400 MHz, CD3OD) δ ppm 0.91 (t, J=7.42 Hz, 3H), 0.99 (d, J=6.44 Hz, 3H), 1.06-1.28 (m, 2H), 1.55-1.89 (m, 5H), 2.14-2.39 (m, 2H), 2.89 (s, 1H), 3.00-3.21 (m, 4H), 3.67-3.92 (m, 3H), 3.94-4.06 (m, 2H), 4.08-4.18 (m, 2H), 4.60 (s, 1H), 4.79-4.89 (m, 2H), 7.17-7.27 (m, 1H), 7.41-7.60 (m, 2H).

### Example 74

### 2-(cyclobutylcarbonyl)-8-[(4-methylpiperidin-1-yl)carbonyl]-5-propyl-2,3,4,5-tetrahydro-1H-pyrido[4,3-b]indole

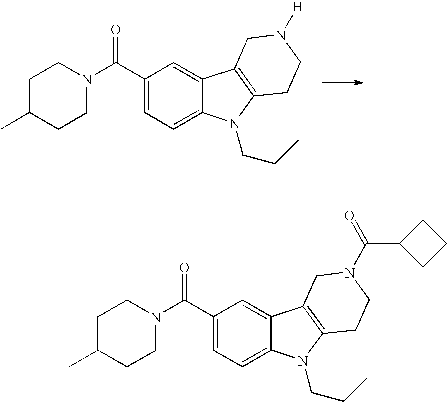

To a solution of 8-[(4-methylpiperidin-1-yl)carbonyl]-5-propyl-2,3,4,5-tetrahydro-1H-pyrido[4,3-b]indole diTFA salt (80 mg, 0.14 mmol) and diisopropylethylamine (0.2 mL) in dichloromethane (5 mL) was added cyclobutanecarbonyl chloride (0.1 mL). The mixture was stirred at room temperature for 3 min, and 1 N NaOH (1 mL) was added, and the mixture was passed through hydromatrix cartridge (10 g) with dichloromethane as eluent. The solvent was evaporated in vacuo and the residue was purified by preparative HPLC (20-75% CH₃CN) to provide the title compound (65 mg). MS (M+1): 422.1. Based on NMR spectra, the title compound is a 2:1 mixture of rotomers incurred from the amide bond.

### Example 75

### 2-(cyclopentylcarbonyl)-8-[(4-methylpiperidin-1-yl)carbonyl]-5-propyl-2,3,4,5-tetrahydro-1H-pyrido[4,3-b]indole

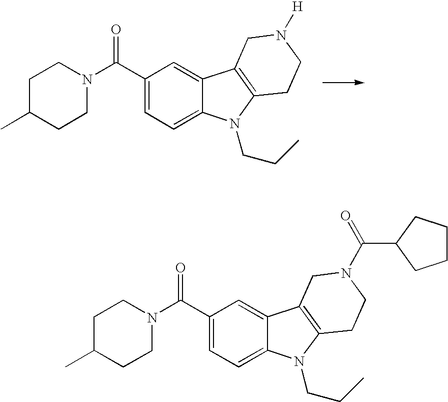

Following the general procedure of Example 74: The title compound was prepared from 8-[(4-methylpiperidin-1-yl)carbonyl]-5-propyl-2,3,4,5-tetrahydro-1H-pyrido[4,3-b]indole diTFA salt and cyclopentanecarbonyl chloride. MS (M+1): 436.1. Based on NMR spectra, the title compound is a ˜3:1 mixtures of rotomers incurred from the amide bond.

### Example 76

### 2-(2,2-difluoropropanoyl)-8-[(4-methylpiperidin-1-yl)carbonyl]-5-propyl-2,3,4,5-tetrahydro-1H-pyrido[4,3-b]indole

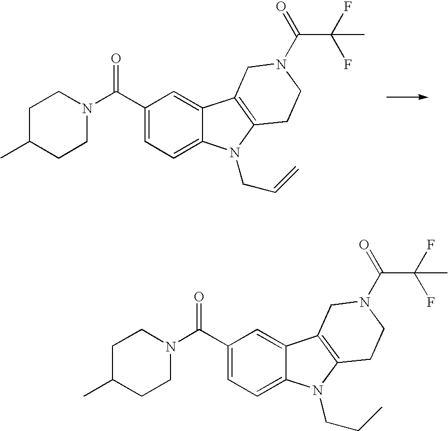

The mixture of 5-allyl-2-(2,2-difluoropropanoyl)-8-[(4-methylpiperidin-1-yl)carbonyl]-2,3,4,5-tetrahydro-1H-pyrido[4,3-b]indole (crude product from Example 39, 301 mg), 10% Pd/C (40 mg) in methanol (30 mL) was hydrogenated at 30 psi overnight. Pd/C was removed by filtration, and removal of methanol gave the crude product (301 mg). MS (M+1): 432.0. ¹H NMR (600 MHz, CD3OD) δ ppm 1.00 (t, J=7.42 Hz, 3H), 1.08 (d, J=6.40 Hz, 3H), 1.16-1.41 (m, 2H), 1.62-2.06 (m, 8H), 2.84-3.28 (m, 4H), 3.95 (s, 1H), 4.10-4.28 (m, 4H), 4.70 (s, 1H), 4.92 (s, 1H), 5.02-5.09 (m, 1H), 7.29 (d, J=8.45 Hz, 1H), 7.54 (d, J=8.45 Hz, 1H), 7.58-7.65 (m, 1H).

### Example 77

### 2-(cyclopropylmethyl)-5-(4-ethoxybenzyl)-8-[(4-methylpiperidin-1-yl)carbonyl]-2,3,4,5-tetrahydro-1H-pyrido[4,3-b]indole

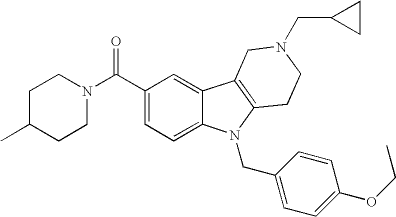

### Step A: The preparation of tert-butyl 5-(4-ethoxybenzyl)-8-[(4-methylpiperidin-1-yl)carbonyl]-1,3,4,5-tetrahydro-W-pyrido[4,3-b]indole-2-carboxylate

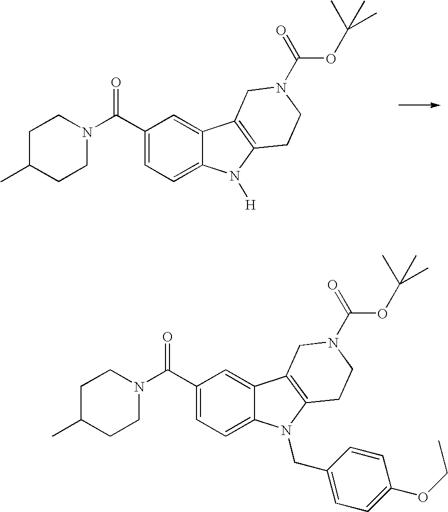

60% NaH (300 mg, 7.5 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (1.58 g, 3.98 mmol) in DMF (30 mL) at room temperature under nitrogen. The mixture was stirred at room temperature for 20 min, then a solution of 4-ethoxybenzyl chloride in DMF (10 mL) (freshly prepared from 4-ethoxybenzyl alcohol (1.10 g, 7.23 mmol) and SOCl₂ (1.0 mL) in dichloromethane at 0° C.) was added at room temperature, and the reaction mixture was stirred at room temperature for 1 h. The solvent was removed in vacuo and the residue was dissolved in dichloromethane and washed with water, dried over sodium sulfate. Removal of solvent gave the crude product, which was purified on silica gel (0-50% EtOAc in dichloromethane) to give the desired product (1.40 mg, 66%). MS (M+1): 532.2.

### Step B: The preparation of 5-(4-ethoxybenzyl)-8-[(4-methylpiperidin-1-yl)carbonyl]-2,3,4,5-tetrahydro-1H-pyrido[4,3-b]indole

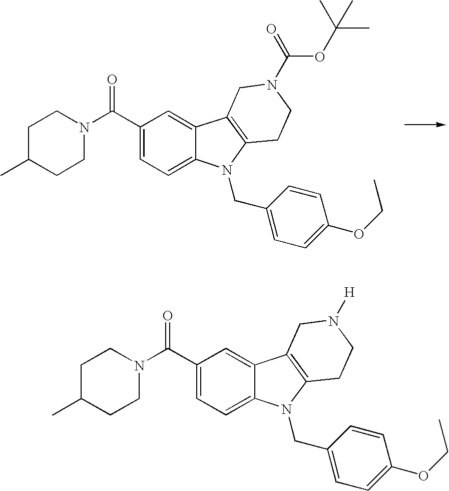

### Method A: the Preparation of Hydrochloride Salt

tert-Butyl 5-(4-ethoxybenzyl)-8-[(4-methylpiperidin-1-yl)carbonyl]-1,3,4,5-tetrahydro-2H-pyrido[4,3-b]indole-2-carboxylate (600 mg, 1.12 mmol) was dissolved in ethyl acetate (10 mL) and 2 N HCl solution in ethyl ether was added, and the mixture was stirred at room temperature overnight. Removal of solvent gave the crude product of the title compounds (562 mg) as its HCl salt.

### Method B: the Preparation of TFA Salt

tert-Butyl 5-(4-ethoxybenzyl)-8-[(4-methylpiperidin-1-yl)carbonyl]-1,3,4,5-tetrahydro-2H-pyrido[4,3-b]indole-2-carboxylate (1.35 g, 2.53 mmol) was dissolved in dichloromethane (20 mL) and TFA (4 mL) was added, the mixture was stirred at room temperature for 1 h. Removal of TFA gave the desired product as its TFA salt (1.85 g).

### Step C: The preparation of 2-(cyclopropylmethyl)-5-(4-ethoxybenzyl)-8-[(4-methylpiperidin-1-yl)carbonyl]-2,3,4,5-tetrahydro-1H-pyrido[4,3-b]indole

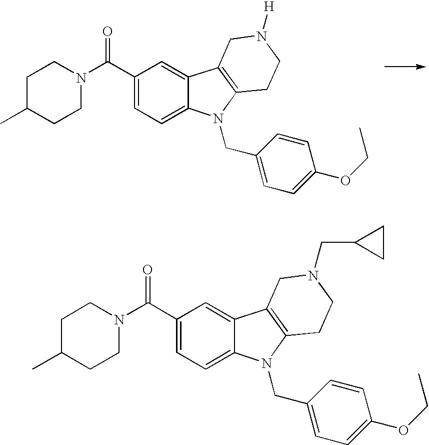

NaBH(OAc)₃ (160 mg, 0.758 mmol) was added in one portion to a solution of 5-(4-ethoxybenzyl)-8-[(4-methylpiperidin-1-yl)carbonyl]-2,3,4,5-tetrahydro-1H-pyrido[4,3-b]indole dihydrochloride (230 mg, 0.453 mmol), cyclopropanecarbaldehyde (0.1 mL) in dichloromethane (5 mL) at room temperature. The mixture was stirred at room temperature for 30 min, diluted with dichloromethane and washed with 1 N NaOH solution. The organic layer was dried and concentrated in vacuo. The residue was purified by preparative HPLC (10-70% CH₃CN) to give the title compound as its TFA salt (136 mg). MS (M+1): 486.1. 1H NMR (400 MHz, CD3OD) δ ppm 0.43-0.53 (m, 2H), 0.72-0.88 (m, 2H), 1.00 (d, J=6.40 Hz, 3H), 1.05-1.30 (m, 3H), 1.33 (t, J=7.03 Hz, 3H), 1.52-1.86 (m, 3H), 2.75-3.22 (m, 5H), 3.26 (d, J=7.42 Hz, 2H), 3.49-3.64 (m, 1H), 3.81 (s, 1H), 3.97 (q, J=6.96 Hz, 2H), 4.43 (d, J=14.45 Hz, 1H), 4.60 (s, 1H), 4.79-4.87 (m, 1H), 5.30 (d, J=16.40 Hz, 1H), 5.41 (d, J=16.40 Hz, 1H), 6.81 (d, J=8.80 Hz, 2H), 7.02 (d, J=8.80 Hz, 2H), 7.25 (dd, J=8.49, 1.56 Hz, 1H), 7.53 (d, J=8.49 Hz, 1H), 7.61 (d, J=1.56 Hz, 1H).

Anal. Calculated for C₃₁H₃₉N₃O₂.1.5C₂HF₃O₂: C, 62.18; H, 6.21; N, 6.40. Found: C, 61.80; H, 6.31; N, 6.46.

### Example 78

### 5-(4-ethoxybenzyl)-2-isobutyl-8-[(4-methylpiperidin-1-yl)carbonyl]-2,3,4,5-tetrahydro-1H-pyrido[4,3-b]indole

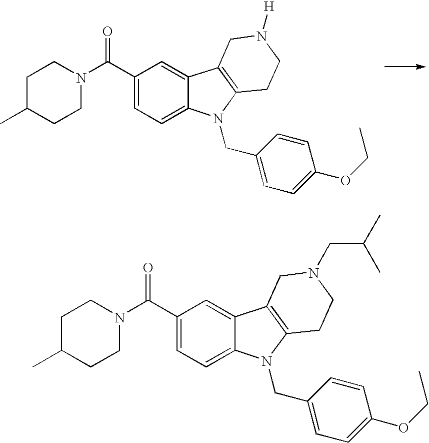

Using similar procedure as described in Example 77, Step C. The title compound was prepared from 5-(4-ethoxybenzyl)-8-[(4-methylpiperidin-1-yl)carbonyl]-2,3,4,5-tetrahydro-1H-pyrido[4,3-b]indole dihydrochloride and 2-methylpropanal. MS (M+1): 488.1. ¹H NMR (400 MHz, CD3OD) δ ppm 0.99 (d, J=6.44 Hz, 3H), 1.09 (d, J=3.71 Hz, 6H), 1.12-1.27 (m, 2H), 1.33 (t, J=7.03 Hz, 3H), 1.46-1.91 (m, 3H), 2.18-2.39 (m, 1H), 2.74-3.26 (m, 6H), 3.45-3.63 (m, 1H), 3.69-3.93 (m, 2H), 3.97 (q, J=7.03 Hz, 2H), 4.26-4.47 (m, 1H), 4.60 (s, 1H), 4.79 (d, J=14.45 Hz, 1H), 5.30 (d, J=16.40 Hz, 1H), 5.40 (d, J=16.40 Hz, 1H), 6.82 (d, J=8.80 Hz, 2H), 7.02 (d, J=8.80 Hz, 2H), 7.24 (dd, J=8.40, 1.56 Hz, 1H), 7.52 (d, J=8.40 Hz, 1H), 7.61 (d, J=1.17 Hz, 1H).

### Example 79

### 5-(4-ethoxybenzyl)-8-[(4-methylpiperidin-1-yl)carbonyl]-2-propyl-2,3,4,5-tetrahydro-1H-pyrido[4,3-b]indole

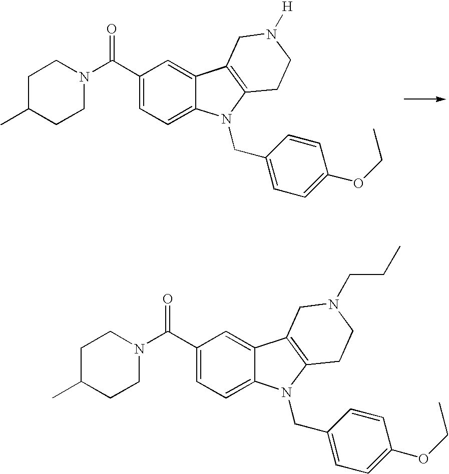

Using similar procedure described in Example 77, Step C: The title compound was prepared from 5-(4-ethoxybenzyl)-8-[(4-methylpiperidin-1-yl)carbonyl]-2,3,4,5-tetrahydro-1H-pyrido[4,3-b]indole TFA and propanal. MS (M+1): 474.0. ¹H NMR (400 MHz, CD3OD) δ ppm 0.98 (d, J=6.44 Hz, 3H), 1.07 (t, J=7.42 Hz, 3H), 1.10-1.26 (m, 2H), 1.33 (t, J=7.03 Hz, 3H), 1.52-1.99 (m, 5H), 2.75-3.22 (m, 4H), 3.25-3.36 (m, 2H), 3.45-3.63 (m, 1H), 3.75-3.94 (m, 2H), 3.96 (q, J=7.03 Hz, 2H), 4.37 (d, J=14.45 Hz, 1H), 4.61 (s, 1H), 4.77 (d, J=14.25 Hz, 1H), 5.29 (d, J=16.20 Hz, 1H), 5.39 (d, J=16.20 Hz, 1H), 6.81 (d, J=8.79 Hz, 2H), 7.01 (d, J=8.79 Hz, 2H), 7.24 (dd, J=8.40, 1.56 Hz, 1H), 7.51 (d, J=8.40 Hz, 1H), 7.59 (d, J=1.56 Hz, 1H).

### Example 80

### 2-cyclobutyl-5-(4-ethoxybenzyl)-8-[(4-methylpiperidin-1-yl)carbonyl]-2,3,4,5-tetrahydro-1H-pyrido[4,3-b]indole

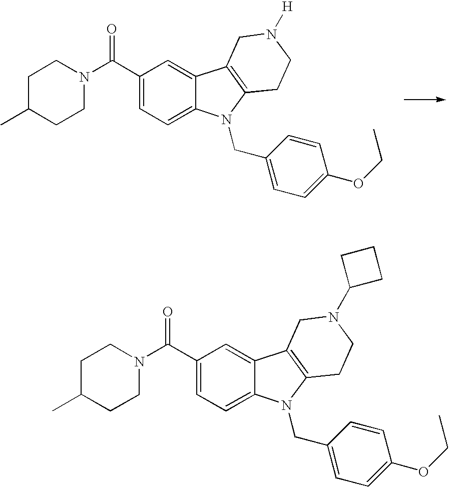

Sodium triacetoxyborohydride (65 mg, 0.31 mmol) was added to 5-(4-ethoxybenzyl)-8-[(4-methylpiperidin-1-yl)carbonyl]-2,3,4,5-tetrahydro-1H-pyrido[4,3-b]indole which was freshly prepared from its TFA salt with MP-carbonate and cyclobutanone (28 μL, 0.37 mmol) in dichloromethane (2.5 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt as a white solid. (30 mg, 17% for three steps). MS (M+1): 486.0. 1H NMR (400 MHz, CDCl3) δ ppm 0.97 (d, J=6.25 Hz, 3H), 1.02-1.30 (m, 2H), 1.36 (t, J=7.03 Hz, 3H), 1.49-1.87 (m, 4H), 1.86-2.02 (m, 1H), 2.21-2.37 (m, 2H), 2.51-2.71 (m, 2H), 2.73-3.09 (m, 2H), 2.82 (d, J=16.60 Hz, 1H), 3.10-3.34 (m, 2H), 3.56-3.86 (m, 3H), 3.95 (q, J=6.90 Hz, 3H), 4.59-4.73 (m, 2H), 5.09-5.25 (m, 2H), 6.78 (d, J=8.60, 2H), 6.85 (d, J=8.60 Hz, 2H), 7.14-7.28 (m, 2H), 7.46 (bs, 1H).

### Example 81

### 5-(4-ethoxybenzyl)-8-[(4-methylpiperidin-1-yl)carbonyl]-2-(tetrahydro-2H-pyran-4-yl)-2,3,4,5-tetrahydro-1H-pyrido[4,3-b]indole

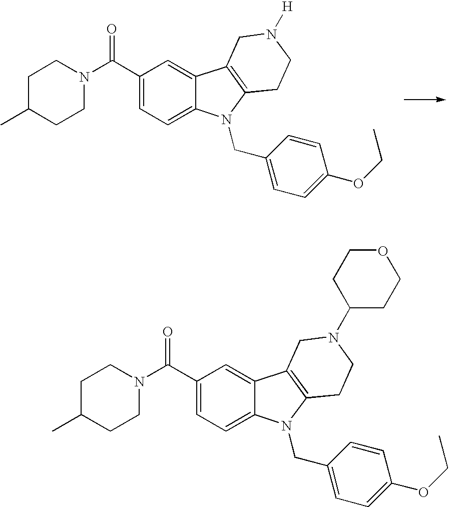

Using similar procedure described in Example 77, Step C: The title compound was prepared from 5-(4-ethoxybenzyl)-8-[(4-methylpiperidin-1-yl)carbonyl]-2,3,4,5-tetrahydro-1H-pyrido[4,3-b]indole TFA and tetrahydro-4H-pyran-4-one. MS (M+1): 516.15. 1H NMR (600 MHz, CD3OD) δ ppm 1.08 (d, J=6.14 Hz, 3H), 1.15 (m, 2H), 1.42 (t, J=7.17 Hz, 3H), 1.50-2.07 (m, 5H), 2.19-2.32 (m, 2H), 2.95 (s, 1H), 3.11-3.35 (m, 2H), 3.49-3.69 (m, 3H), 3.71-3.83 (m, 1H), 3.91 (s, 1H), 4.06 (q, J=7.17 Hz, 2H), 4.21 (d, J=8.19 Hz, 2H), 4.61 (d, J=12.29 Hz, 1H), 4.70 (s, 1H), 4.83 (d, J=12.29 Hz, 1H), 5.39 (d, J=16.40 Hz, 1H), 5.51 (d, J=16.40 Hz, 1H), 5.58 (s, 2H), 6.91 (d, J=8.19 Hz, 2H), 7.11 (d, J=8.19 Hz, 2H), 7.35 (d, J=8.19 Hz, 1H), 7.63 (d, J=8.19 Hz, 1H), 7.72 (s, 1H).

### Example 82

### 2-(cyclobutylcarbonyl)-5-(4-ethoxybenzyl)-8-[(4-methylpiperidin-1-yl)carbonyl]-2,3,4,5-tetrahydro-1H-pyrido[4,3-b]indole

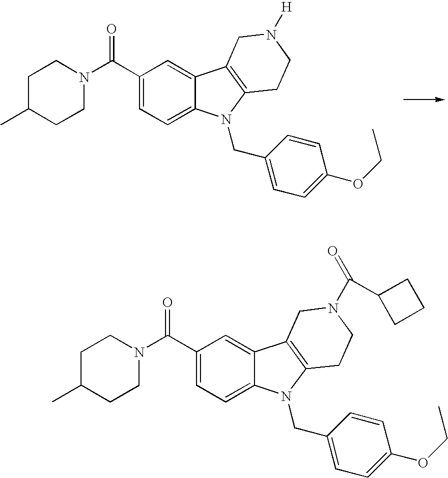

Using similar procedure described in Example 74: The title compound was prepared from 5-(4-ethoxybenzyl)-8-[(4-methylpiperidin-1-yl)carbonyl]-2,3,4,5-tetrahydro-1H-pyrido[4,3-b]indole TFA and cyclobutanecarbonyl chloride. MS (M+1): 514.2. Based on NMR data, the title compound is a ˜2:1 mixture of two rotomers incurred from the amide bond.

### Example 83

### 5-(4-ethoxybenzyl)-8-[(4-methylpiperidin-1-yl)carbonyl]-2-pyrimidin-2-yl-2,3,4,5-tetrahydro-1H-pyrido[4,3-b]indole

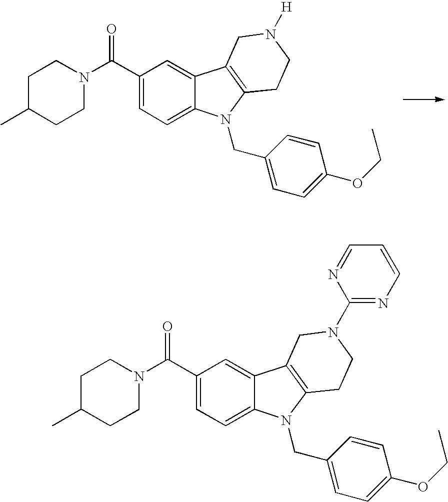

The degassed mixture of 5-(4-Ethoxybenzyl)-8-[(4-methylpiperidin-1-yl)carbonyl]-2,3,4,5-tetrahydro-1H-pyrido[4,3-b]indole TFA salt (100 mg), 2-bromopyrimidine (35 mg), K₂CO₃ (35 mg) in DMF (2 mL) was heated under N₂ in microwave condition (10 min, 100° C.), then additional 2-bromopyrimidine (18 mg), K₂CO₃ (35 mg) were added and heated another 10 min at 100° C. under microwave condition. The process was repeated again. The mixture was allowed to cool to room temperature and diluted with dichloromethane and washed with water. The organic solution was concentrated in vacuo, and the residue was purified by HPLC (20-80% CH₃CN) to afford the title compound as its TFA salt (28 mg). MS (M+1): 510.4. 1H NMR (400 MHz, CD3OD) δ ppm 0.95 (d, J=6.44 Hz, 3H), 1.05-1.21 (m, 2H), 1.28 (t, J=7.03 Hz, 3H), 1.49-1.86 (m, 3H), 2.88 (t, J=5.57 Hz, 2H), 3.22-3.28 (m, 2H), 3.76-3.86 (m, 1H), 3.90 (q, J=7.03 Hz, 2H), 4.25 (t, J=5.76 Hz, 2H), 4.56 (s, 1H), 4.96 (s, 2H), 5.16-5.33 (m, 2H), 6.69-6.79 (m, 3H), 6.91 (d, J=8.79 Hz, 2H), 7.14 (dd, J=8.40, 1.66 Hz, 1H), 7.38 (d, J=8.40 Hz, 1H), 7.55 (d, J=1.66 Hz, 1H), 8.43 (d, J=5.08 Hz, 2H).

### Example 84

### 5-(4-ethoxybenzyl)-8-[(4-methylpiperidin-1-yl)carbonyl]-2-(3,4,5,6-tetrahydropyridin-2-yl)-2,3,4,5-tetrahydro-1H-pyrido[4,3-b]indole

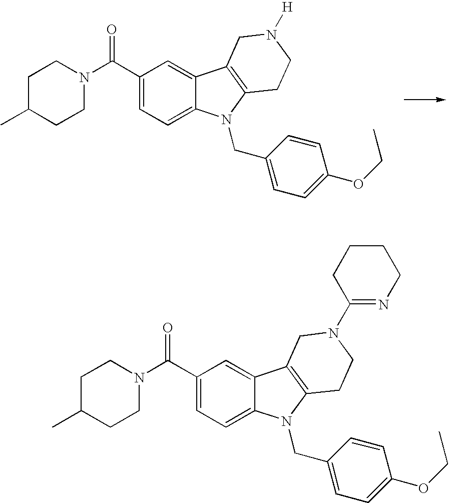

6-methoxy-2,3,4,5-tetrahydropyridine (0.3 mL) was added to the solution of 5-(4-ethoxybenzyl)-8-[(4-methylpiperidin-1-yl)carbonyl]-2,3,4,5-tetrahydro-1H-pyrido[4,3-b]indole TFA salt (100 mg) in methanol (1.5 mL) at room temperature. The mixture was stirred at room temperature overnight and purified by preparative HPLC (10-75% CH₃CN) to afford the title compound as its TFA salt (45 mg). MS (M+1): 513.1. ¹H NMR (400 MHz, CD3OD) δ ppm 0.99 (d, J=6.64 Hz, 3H), 1.06-1.27 (m, 2H), 1.33 (t, J=7.03 Hz, 3H), 1.51-2.01 (m, 6H), 2.86 (t, J=5.66 Hz, 2H), 2.98 (t, J=5.47 Hz, 2H), 3.03-3.15 (m, 1H), 3.30 (s, 2H), 3.43-3.55 (m, 2H), 3.74-3.92 (m, 1H), 3.96 (m, 4H), 4.50-4.69 (m, 1H), 4.78 (s, 1H), 4.90 (s, 1H), 5.29-5.35 (m, 2H), 6.78-6.84 (m, 2H), 6.98 (d, J=8.59 Hz, 2H), 7.21 (t, J=7.23 Hz, 1H), 7.44-7.50 (m, 1H), 7.53-7.62 (m, 1H).

### Example 85

### 1-{5-(4-ethoxybenzyl)-8-[(4-methylpiperidin-1-yl)carbonyl]-1,3,4,5-tetrahydro-2H-pyrido[4,3-b]indol-2-yl}ethanimine

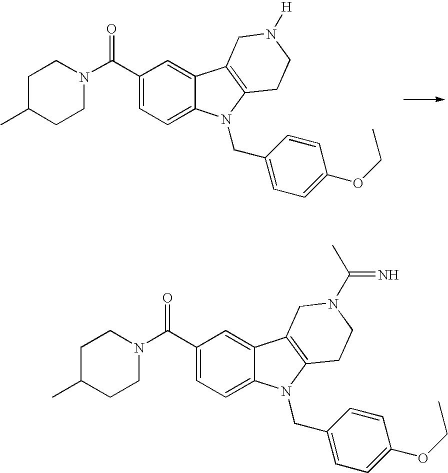

The solution of ethyl ethanimidoate hydrochloride (30 mg), diisopropylethylamine (0.1 mL) and 5-(4-ethoxybenzyl)-8-[(4-methylpiperidin-1-yl)carbonyl]-2,3,4,5-tetrahydro-1H-pyrido[4,3-b]indole dihydrochloride (33 mg) in methanol-CH₃CN (2 mL, 1:1) was heated at 65° C. in a sealed tube for 3 h. The solvent was evaporated in vacuo and the residue was purified by HPLC (10-70% CH₃CN) to afford the title compound as its diTFA salt. MS (M+1): 473.3. 1H NMR (400 MHz, CD3OD) δ ppm 1.00 (d, J=6.25 Hz, 3H), 1.09-1.28 (m, 2H), 1.35 (t, J=7.03 Hz, 3H), 1.55-1.89 (m, 3H), 2.47 (s, 3H), 2.77-3.18 (m, 4H), 3.98 (q, J=7.03 Hz, 2H), 4.03 (t, J=5.47 Hz, 2H), 4.62 (s, 1H), 4.85 (s, 1H), 4.97 (s, 1H), 5.35 (s, 2H), 6.83 (cl, J=8.59 Hz, 2 H), 7.00 (d, J=8.59 Hz, 2H), 7.19-7.28 (m, 1H), 7.44-7.53 (m, 1H), 7.57 (7.60) (s, 1H). Anal Calculated for C₂₉H₃₆N₄O₂.2C₂HF₃O₂: C, 56.56; H, 5.46; N, 7.99. Found: C, 56.99; H, 5,45; N, 7.66.

### Example 86

### 2-(3,4-dihydro-2H-pyrrol-5-yl)-5-(4-ethoxybenzyl)-8-[(4-methylpiperidin-1-yl)carbonyl]-2,3,4,5-tetrahydro-1H-pyrido[4,3-b]indole

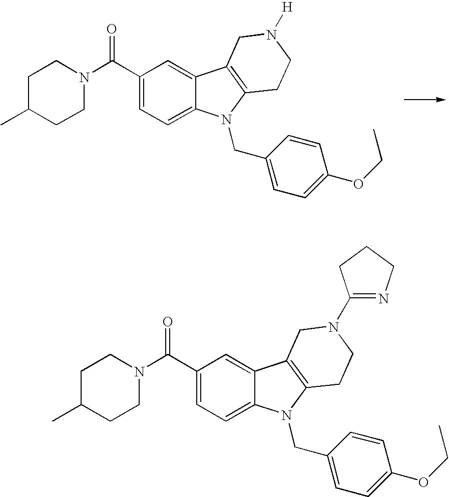

Using similar procedure described in Example 84: The title compound was prepared from 5-(4-ethoxybenzyl)-8-[(4-methylpiperidin-1-yl)carbonyl]-2,3,4,5-tetrahydro-1H-pyrido[4,3-b]indole TFA salt and 5-methoxy-3,4-dihydro-2H-pyrrole. MS (M+1): 499.12. ¹H NMR (400 MHz, CD3OD) δ ppm 0.99 (d, J=6.44 Hz, 3H), 1.06-1.26 (m, 2H), 1.33 (t, J=7.03 Hz, 3H), 1.69 (s, 3H), 2.15-2.36 (m, 2H), 2.93-3.06 (m, 3H), 3.07-3.18 (m, 3H), 3.28-3.32 (m, 2H), 3.72-3.80 (m, 3H), 3.96 (q, J=7.03 Hz, 2H), 4.83 (brs, 2H), 4.91 (brs, 1H), 5.33 (s, 2H), 6.74-6.86 (m, 2H), 6.96-7.01 (m, 2H), 7.15-7.25 (m, 1H), 7.42-7.51 (m, 1H), 7.55 (7.59) (d, J=0.98 Hz, 1H).

### Example 87

### 2-(cyclopropylmethyl)-8-[(4-methylpiperidin-1-yl)carbonyl]-5-(tetrahydro-2H-pyran-4-ylmethyl)-2,3,4,5-tetrahydro-1H-pyrido[4,3-b]indole

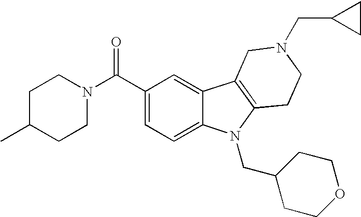

### Step A: The preparation of tert-butyl 8-[(4-methylpiperidin-1-yl)carbonyl]-5-(tetrahydro-2H-pyran-4-ylmethyl)-1,3,4,5-tetrahydro-2H-pyrido[4,3-b]indole-2-carboxylate

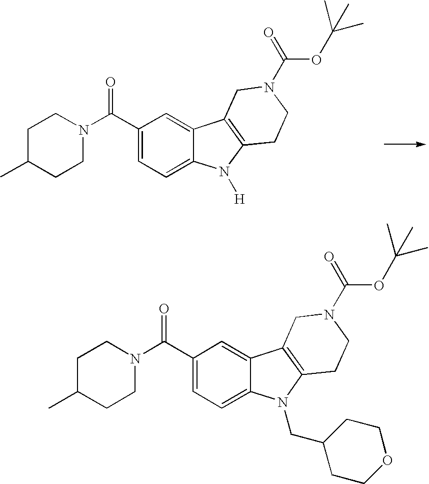

60% NaH (25 mg, 0.62 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (40 mg, 0.10 mmol) in DMF (2 mL) at room temperature under nitrogen. The mixture was stirred at room temperature for 20 min, then a solution of 4-bromomethyltetrahydropyran in DMF (1 mL) was added at room temperature, and the reaction mixture was stirred at room temperature for 2 h. The solvent was removed in vacuo and the residue was dissolved in dichloromethane and washed with water, dried over sodium sulfate. Removal of solvent gave the crude product (49 mg), which was used without purification. MS (M+1): 496.1.

### Step B: The preparation of 8-[(4-methylpiperidin-1-yl)carbonyl]-5-(tetrahydro-2H-pyran-4-ylmethyl)-2,3,4,5-tetrahydro-1H-pyrido[4,3-b]indole

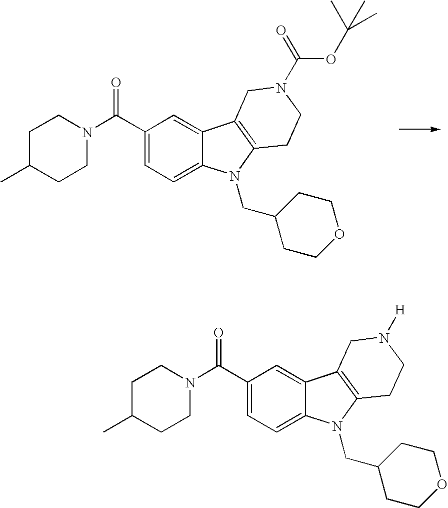

The crude intermediate from step A (49 mg) was dissolved in dichloromethane (5 mL), and TFA (1 mL) was added at room temperature, the mixture was stirred at room temperature for 1.5 h, and concentrated in vacuo. The residue was redissolved in dichloromethane and loaded onto a short pad of silica gel, and washed with dichloromethane (20 mL). Then the compound was washed out from the column by 2 M NH₃ in methanol. Removal of methanol gave the title compound (38 mg, 96% in two steps).

### Step C: The preparation of 2-(cyclopropylmethyl)-8-[(4-methylpiperidin-1-yl)carbonyl]-5-(tetrahydro-2H-pyran-4-ylmethyl)-2,3,4,5-tetrahydro-1H-pyrido[4,3-b]indole

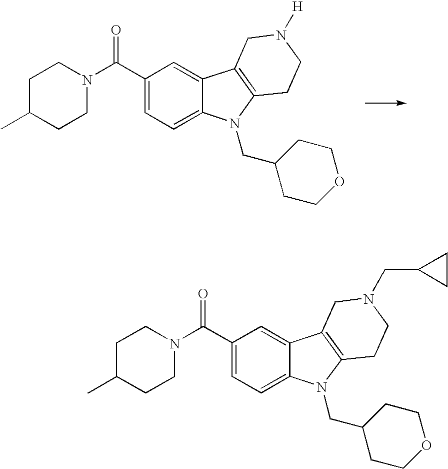

NaBH(OAc)₃ (67 mg, 0.32 mmol) was added to a mixture of 8-[(4-methylpiperidin-1-yl)carbonyl]-5-(tetrahydro-2H-pyran-4-ylmethyl)-2,3,4,5-tetrahydro-1H-pyrido[4,3-b]indole (38 mg) from step B, cyclopropanecarbaldehyde (0.1 mL) in dichloromethane (3 mL) at room temperature. The mixture was stirred at room temperature for 1 h, diluted with dichloromethane, washed with 1 N NaOH, and concentrated in vacuo. The residue was purified by preparative HPLC (15-75% CH₃CN) to afford the title compound as its TFA salt (12 mg). MS (M+1): 450.1. ¹H NMR (400 MHz, CD3OD) δ ppm 0.46-0.55 (m, 2H), 0.79-0.88 (m, 2H), 0.99 (d, J=6.64 Hz, 3H), 1.06-1.34 (m, 4H), 1.37-1.52 (m, 4H), 1.52-1.92 (m, 3H), 2.04-2.26 (m, 1H), 3.22-3.38 (m, 7H), 3.50-4.14 (m, 7H), 4.42 (d, J=14.45 Hz, 1H), 4.49-4.68 (m, 1H), 4.84 (d, J=14.45 Hz, 1H), 7.26 (dd, J=8.40, 1.37 Hz, 1H), 7.49-7.61 (m, 2H).

### Example 88

### 8-[(4-methylpiperidin-1-yl)carbonyl]-5-(propylsulfonyl)-2-(tetrahydro-2H-pyran-4-yl)-2,3,4,5-tetrahydro-1H-pyrido[4,3-b]indole

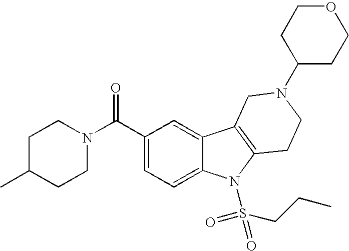

### Step A: The preparation of tert-butyl 8-[(4-methylpiperidin-1-yl)carbonyl]-5-(propylsulfonyl)-1,3,4,5-tetrahydro-2H-pyrido[4,3-b]indole-2-carboxylate

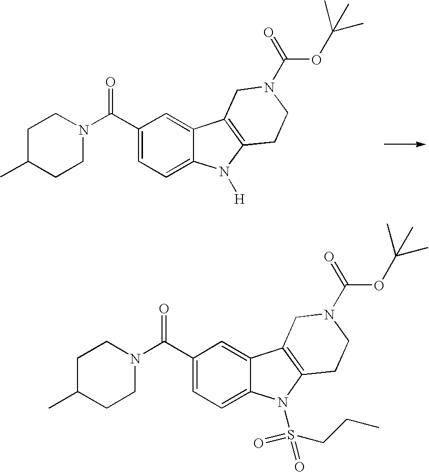

60% NaH (120 mg, 3.0 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (397 mg, 1.00 mmol) in DMF (20 mL) at room temperature under nitrogen. The mixture was stirred at room temperature for 1 h, then propylsulfonyl chloride (0.2 mL) was added dropwise at room temperature, and the reaction mixture was stirred at room temperature for 2 h. The solvent was removed in vacuo and the residue was dissolved in dichloromethane and washed with water, dried over sodium sulfate. Removal of solvent gave the crude product, which was purified by flash chromatography (eluent 0-60% dichloromethane in EtOAc) to afford the title compound (225 mg, 60%). MS (M+1): 504.1.

### Step B: The preparation of 8-[(4-methylpiperidin-1-yl)carbonyl]-5-(propylsulfonyl)-2,3,4,5-tetrahydro-1H-pyrido[4,3-b]indole TFA salt

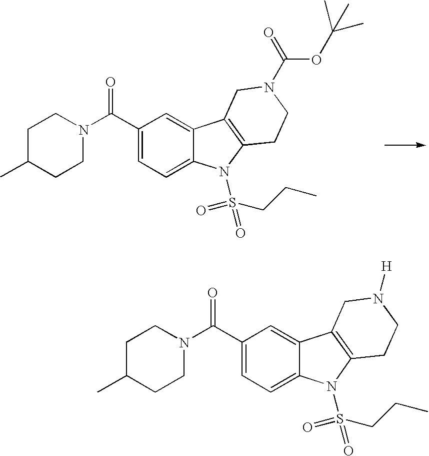

tert-butyl 8-[(4-methylpiperidin-1-yl)carbonyl]-5-(propylsulfonyl)-1,3,4,5-tetrahydro-2H-pyrido[4,3-b]indole-2-carboxylate (225 mg, 0.447 mmol) from step A was dissolved in dichloromethane (2 mL) and TFA (2 mL) was added, and the mixture was stirred at room temperature for 1 h. Excess TFA was removed in vacuo, and the residue was lipholized to afford the title compound as its TFA salt (235 mg).

### Step C: The preparation of 8-[(4-methylpiperidin-1-yl)carbonyl]-5-(propylsulfonyl)-2-(tetrahydro-2H-pyran-4-yl)-2,3,4,5-tetrahydro-1H-pyrido[4,3-b]indole

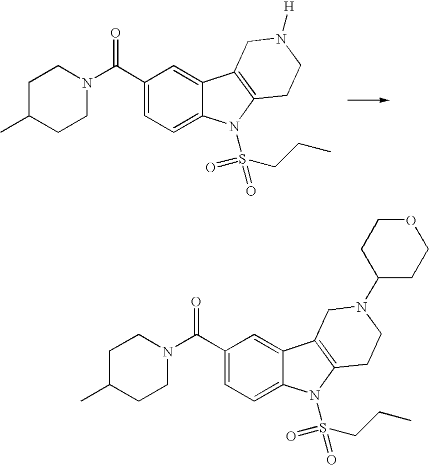

NaBH(OAc)₃ (60 mg, 0.28 mmol) was added to a solution of 8-[(4-methylpiperidin-1-yl)carbonyl]-5-(propylsulfonyl)-2,3,4,5-tetrahydro-1H-pyrido[4,3-b]indole TFA salt (70 mg, 0.135 mmol), tetrahydro-4-pyranone (50 mg, 0.50 mmol) in dichloromethane (3 mL). The mixture was stirred at room temperature for 3 h, diluted with dichloromethane. 1N NaOH (1 ml) was added to the mixture, and the mixture was stirred for 5 min, and loaded on to a 10 g hydromatrix cartridge. The compound was eluted with dichloromethane. Removal of solvent in vacuo gave the crude product, which was purified by preparative HPLC (10-70% CH₃CN) and lipholized to afford the title compound as its TFA salt (48 mg). MS (M+1): 488.1. ¹H NMR (600 MHz, CD3OD) δ ppm 0.97 (t, J=7.42 Hz, 3H), 0.99 (d, J=6.66 Hz, 3H), 1.05-1.30 (m, 2H), 1.44-1.86 (m, 5H), 1.87-2.00 (m, 2H), 2.18 (d, J=10.75 Hz, 2H), 2.78-2.94 (m, 1H), 3.12 (t, J=11.52 Hz, 1H), 3.36-3.62 (m, 7H), 3.65-3.79 (m, 2H), 3.83-4.08 (m, 1H), 4.13 (dd, J=11.65, 3.71 Hz, 2H), 4.62 (d, J=8.70 Hz, 3H), 7.42 (dd, J=8.71, 1.41 Hz, 1H), 7.53-7.77 (m, 1H), 8.05 (d, J=8.71 Hz, 1H). Anal. Calcd for C₂₆H₃₇N₃O₄.1.75C₂HF₃O₂: C, 51.50; H, 5.63; N, 6.11. Found: C, 51.07; H, 5.45; N, 6.07.

### Example 89

### 8-[(4-methylpiperidin-1-yl)carbonyl]-5-(propylsulfonyl)-2-(3,3,3-trifluoropropyl)-2,3,4,5-tetrahydro-1H-pyrido[4,3-b]indole

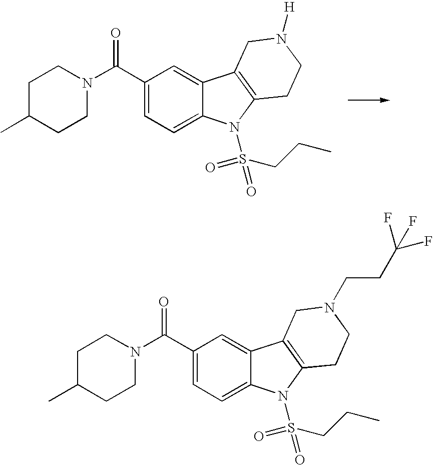

To a solution of crude 8-[(4-methylpiperidin-1-yl)carbonyl]-5-(propylsulfonyl)-2,3,4,5-tetrahydro-1H-pyrido[4,3-b]indole (TFA salt, 30 mg, 0.06 mmol) in dry dichloromethane (2 mL) was added 3,3,3-trifluoropropanal (0.12 mmol) and sodium triacetoxyborohydride (0.09 mmol). The mixture was stirred at room temperature for 3 h, diluted with dichloromethane (15 mL), washed with saturated NaHCO₃ solution (5 mL), dried over Na₂SO₄. The dichloromethane solution was concentrated in vacuo and the residue was purified with reverse phase HPLC to give the desired product as its TFA salt (18 mg, 49%). MS (M+1): 500.0. 1H NMR (600 MHz, CDCL3) δ ppm 1.04 (t, J=7.42 Hz, 3H), 1.09 (d, J=6.40 Hz, 3H), 1.23 (s, br, 1H), 1.35 (s, br, 1H), 1.75 (m, 4H), 1.89 (s, br, 1H), 2.93 (m, 3H), 3.14 (s, br, 1H), 3.27 (m, 2H), 3.51 (m, 2H), 3.58 (s, br, 2H), 3.63 (s, br, 2H), 3.83 (s, br, 1H), 4.42 (s, br, 2H), 4.77 (s, br, 1H), 7.48 (d, J=8.71 Hz, 1H), 7.57 (s, 1H), 8.09 (d, J=8.71 Hz, 1H).

### Example 90

### 2-cyclopentyl-8-[(4-methylpiperidin-1-yl)carbonyl]-5-(propylsulfonyl)-2,3,4,5-tetrahydro-1H-pyrido[4,3-b]indole TFA salt

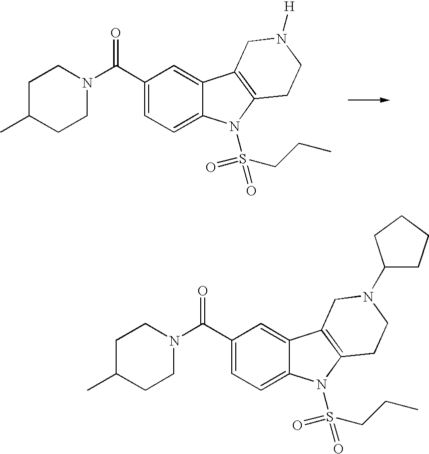

Following the similar procedure as described in Example 89, Step C: The title compound was prepared from 8-[(4-methylpiperidin-1-yl)carbonyl]-5-(propylsulfonyl)-2,3,4,5-tetrahydro-1H-pyrido[4,3-b]indole TFA salt and cyclopentanone. MS (M+1): 472.1. ¹H NMR (400 MHz, CD3OD) δ ppm 0.97 (t, J=7.13 Hz, 3H), 1.00 (d, J=6.25 Hz, 3H), 1.06-1.30 (m, 2H), 1.48-1.99 (m, 11H), 2.23-2.44 (m, 2H), 2.78-2.97 (m, 1H), 3.05-3.20 (m, 1H), 3.38-3.61 (m, 3H), 3.46 (t, J=7.81 Hz, 2H), 3.62-3.89 (m, 2H), 3.92-4.06 (m, 1H), 4.32-4.50 (m, 1H), 4.56-4.71 (m, 1H), 4.82 (m, 1H) 7.42 (dd, J=8.60, 0.98 Hz, 1H), 7.67 (d, J=0.98 Hz, 1H), 8.06 (d, J=8.60 Hz, 1H).

### Example 91

### 8-[(4-methylpiperidin-1-yl)carbonyl]-5-(propylsulfonyl)-2-(tetrahydrofuran-3-yl)-2,3,4,5-tetrahydro-1H-pyrido[4,3-b]indole

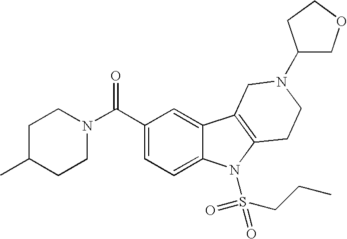

### Step A: The preparation of dihydrofuran-3(2H)-one

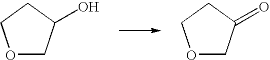

Tetrapropylammonium perruthenate (TPAP, 20 mg, 0.057 mmol) was added to a mixture of tetrahydrofuran-3-ol (616 mg, 7.0 mmol), N-methylmorpholine N-oxide (NMO, 11 mmol), activated molecular sieve powder (3.5 g) in dichloromethane (30 mL) at room temperature, the reaction mixture was stirred at room temperature for 2 h, then filtered through a short pad of Celite under reduced pressure. The dichloromethane solution (˜20 mL) of dihydrofuran-3(2H)-one was used as a stock solution (˜0.3 M).

### Step B: The preparation of 8-[(4-methylpiperidin-1-yl)carbonyl]-5-(propylsulfonyl)-2-(tetrahydrofuran-3-yl)-2,3,4,5-tetrahydro-1H-pyrido[4,3-b]indole

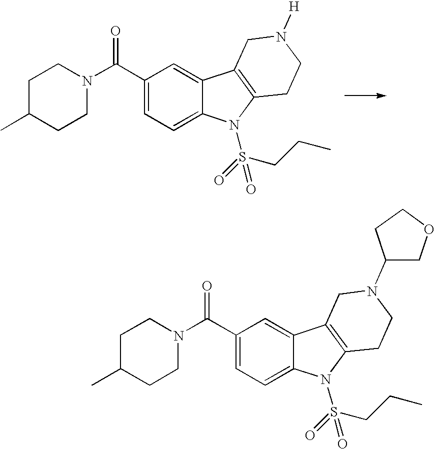

Following the similar procedure described in Example 89, Step C: The title compound was prepared from dihydrofuran-3(2H)-one from step A and 8-[(4-methylpiperidin-1-yl)carbonyl]-5-(propylsulfonyl)-2,3,4,5-tetrahydro-1H-pyrido[4,3-b]indole TFA salt. MS (M+1): 474.0. ¹H NMR (600 MHz, CD3OD) δ ppm 0.80-0.92 (m, 6H), 0.94-1.21 (m, 2H), 1.41-1.77 (m, 5H), 2.16-2.30 (m, 1H), 2.35-2.48 (m, 1H), 2.65-2.83 (m, 1H), 3.01 (d, J=8.19 Hz, 1H), 3.19 (s, 1H), 3.27-3.40 (m, 3H), 3.47-3.76 (m, 4H), 3.80-3.89 (m, 1H), 3.99-4.09 (m, 1H), 4.11-4.25 (m, 2H), 4.37-4.68 (m, 3H), 7.30 (d, J=9.22 Hz, 1H), 7.51 (s, 1H), 7.92 (d, J=9.22 Hz, 1H).

### Example 92

### 2-(3,4-dihydro-2H-pyrrol-5-yl)-8-[(4-methylpiperidin-1-yl)carbonyl]-5-(propylsulfonyl)-2,3,4,5-tetrahydro-1H-pyrido[4,3-b]indole

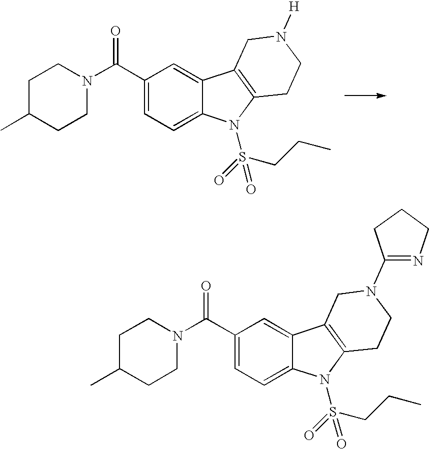

Using similar procedure described in Example 86: The title compound was prepared from 8-[(4-methylpiperidin-1-yl)carbonyl]-5-(propylsulfonyl)-2,3,4,5-tetrahydro-1H-pyrido[4,3-b]indole TFA salt and 5-methoxy-3,4-dihydro-2H-pyrrole. MS (M+1): 471.1. 1H NMR (400 MHz, CD3OD) δ ppm 0.95 (t, J=7.42 Hz, 3H), 0.99 (d, J=6.44 Hz, 3H), 1.05-1.33 (m, 2H), 1.46-1.90 (m, 5H), 2.16-2.40 (m, 2H), 2.73-2.98 (m, 1H), 3.00-3.22 (m, 3H), 3.34-3.46 (m, 2H), 3.63-3.86 (m, 3H), 3.97 (t, J=5.76 Hz, 2H), 4.48-4.68 (m, 1H), 4.78 (s, 2H), 4.85-4.93 (m, 2H), 7.30-7.45 (m, 1H), 7.53 (7.61) s, 1H), 7.99-8.08 (m, 1H).

### Example 93

### 2-cyclopentyl-8-[(4-methylpiperidin-1-yl)carbonyl]-5-(methylsulfonyl)-2,3,4,5-tetrahydro-1H-pyrido[4,3-b]indole

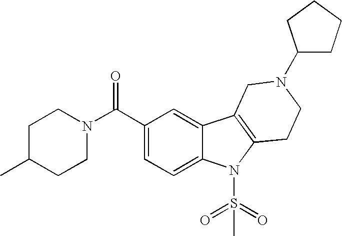

### Step A: The preparation of tert-butyl 8-[(4-methylpiperidin-1-yl)carbonyl]-5-(methylsulfonyl)-1,3,4,5-tetrahydro-2H-pyrido[4,3-b]indole-2-carboxylate

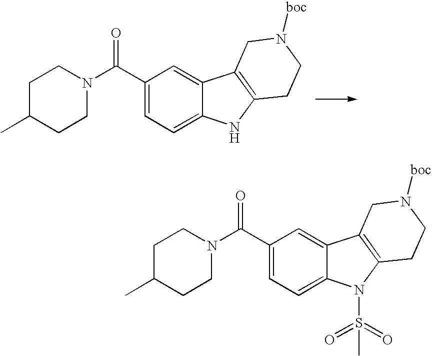

To a solution of 2-(tert-butoxycarbonyl)-2,3,4,5-tetrahydro-1H-pyrido[4,3-b]indole-8-carboxylic acid (596 mg, 1.5 mmol) in dry DMF (20 mL) was added 60% NaH (7.5 mmol) in one portion at 0° C. under nitrogen. The mixture was stirred at room temperature for 30 min, then methanesulfonyl chloride (7.5 mmol) was added slowly at room temperature, and the reaction mixture was stirred at room temperature for 5 h. The solvent was removed in vacuo and the residue was dissolved in dichloromethane (80 mL), washed with water (20 mL), dried over sodium sulfate. Removal of solvent gave the crude product, which was purified on silica gel (5-60% EtOAc in dichloromethane) to give the desired product as yellow oil (520 mg, 73%). MS (M+1): 476.0.

### Step B: The preparation of 8-[(4-methylpiperidin-1-yl)carbonyl]-5-(methylsulfonyl)-2,3,4,5-tetrahydro-1H-pyrido[4,3-b]indole

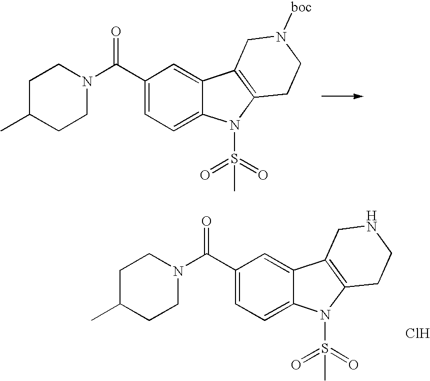

To a solution of tert-butyl 8-[(4-methylpiperidin-1-yl)carbonyl]-5-(methylsulfonyl)-1,3,4,5-tetrahydro-2H-pyrido[4,3-b]indole-2-carboxylate (145 mg, 0.31 mmol) in dioxane (3 mL) was added 4N HCl in dioxane (2 mL). The solution was stirred at room temperature for 3 h. The solvent was removed in vacuo and the residue was washed with diethyl ether, filtered to give the desired product as its HCl salt (112 mg, 96%), which was used without further purification. MS (M+1): 376.0.

### Step C: The preparation of 2-cyclopentyl-8-[(4-methylpiperidin-1-yl)carbonyl]-5-(methylsulfonyl)-2,3,4,5-tetrahydro-1H-pyrido[4,3-b]indole

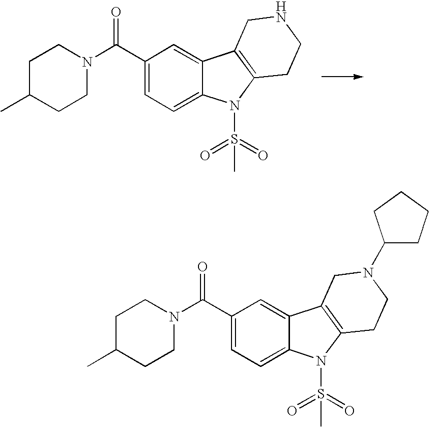

To a solution of 8-[(4-methylpiperidin-1-yl)carbonyl]-5-(methylsulfonyl)-2,3,4,5-tetrahydro-1H-pyrido[4,3-b]indole (HCl salt, 56 mg, 0.13 mmol) in dry dichloromethane (4 mL) was added cyclopentanone (0.26 mmol) and sodium triacetoxyborohydride (0.39 mmol). The mixture was stirred at room temperature overnight, diluted with dichloromethane (15 mL), washed with saturated NaHCO₃ solution (5 mL), and dried over Na₂SO₄. The dichloromethane solution was concentrated in vacuo and the residue was purified with reverse phase HPLC to give the desired product as its TFA salt (28 mg). MS (M+1): 444.0. 1H NMR (600 MHz, CDCL3) δ ppm 1.01 (d, J=6.14 Hz, 3H), 1.15 (m, 1H), 1.30 (m, 1H), 1.59-1.78 (m, 5H), 1.95 (m, 2H), 2.10 (m, 2H), 2.20 (m, 2H), 2.84 (m, 1H), 3.09 (s, 3H), 3.28 (m, 1H), 3.44 (m, 2H), 3.61 (m, 2H), 3.76 (m, 1H), 3.92 (m, 1H), 4.05 (d, J=14.23 Hz, 1H), 4.69 (m, 1H), 4.76 (d, J=14.23 Hz, 1H), 7.38 (d, J=8.25 Hz, 1H), 7.52 (s, 1H), 8.01 (d, J=8.25 Hz, 1H).

### Example 94

### 8-[(4-methylpiperidin-1-yl)carbonyl]-5-(methylsulfonyl)-2-(tetrahydro-2H-pyran-4-yl)-2,3,4,5-tetrahydro-1H-pyrido[4,3-b]indole

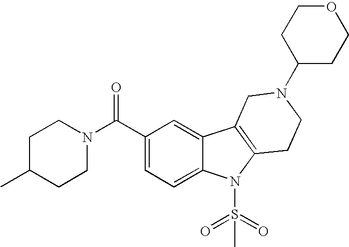

Following the same procedure as Example 93, Step C: The title compound as its TFA salt was prepared from 8-[(4-methylpiperidin-1-yl)carbonyl]-5-(methylsulfonyl)-2,3,4,5-tetrahydro-1H-pyrido[4,3-b]indole (HCl salt, 56 mg, 0.13 mmol) and tetrahydro-4H-pyran-4-one .and .MS (M+1): 460.0. 1H NMR (600 MHz, CDCl3) δ ppm 1.01 (d, J=6.14 Hz, 3H), 1.15 (m, 1H), 1.28 (m, 1H), 1.70 (m, 2H), 1.83 (m, 1H), 2.03 (m, 2H), 2.12 (m, 2H), 2.85 (m, 1H), 3.10 (m, 3H), 3.08 (s, 3H), 3.30 (m, 1H), 3.51 (m, 3H), 3.71 (m, 2H), 3.87 (m, 1H), 4.19 (m, 2H), 4.66 (m, 2H), 7.39 (d, J=8.35 Hz, 1H), 7.53 (s, 1H), 8.02 (d, J=8.35 Hz, 1H).

### Example 95

### 2-cyclobutyl-8-[(4-methylpiperidin-1-yl)carbonyl]-5-(methylsulfonyl)-2,3,4,5-tetrahydro-1H-pyrido[4,3-b]indole

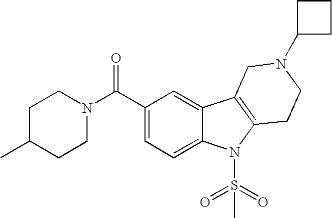

Following the same procedure as Example 93, Step C: The title compound as its TFA salt was prepared from 8-[(4-methylpiperidin-1-yl)carbonyl]-5-(methylsulfonyl)-2,3,4,5-tetrahydro-1H-pyrido[4,3-b]indole and cyclobutanone. MS (M+1): 430.0. 1H NMR (400 MHz, CD3OD) δ ppm 0.96 (d, J=6.45 Hz, 3H), 1.04-1.27 (m, 2H), 1.50-1.58 (m, 1H), 1.64-1.74 (m, 1H), 1.73-1.80 (m, 1H), 1.85-1.99 (m, 2H), 2.26-2.38 (m, 2H), 2.41-2.52 (m, 2H), 2.78-2.90 (m, 1H), 3.05-3.14 (m, 1H), 3.28-3.30 (m, 3H), 3.32-3.57 (m, 3H), 3.63-3.74 (m, 1H), 3.75-3.82 (m, 1H), 3.86-4.02 (m, 1H), 4.12-4.22 (m, 1H), 4.54-4.62 (m, 1H), 4.64-4.75 (m, 1H), 7.40 (d, J=8.59 Hz, 1H), 7.61 (s, 1H), 8.04 (d, J=8.59 Hz, 1H).

### Example 96

### 2-(cyclopropylmethyl)-8-[(4-methylpiperidin-1-yl)carbonyl]-5-(methylsulfonyl)-2,3,4,5-tetrahydro-1H-pyrido[4,3-b]indole

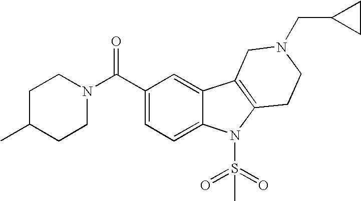

Following the same procedure as Example 93, Step C: The title compound was prepared from 8-[(4-methylpiperidin-1-yl)carbonyl]-5-(methylsulfonyl)-2,3,4,5-tetrahydro-1H-pyrido[4,3-b]indole and cyclopropanecarboxaldehyde. MS (M+1): 430.0. 1H NMR (400 MHz, CD3OD) δ ppm 0.50 (d, J=4.10 Hz, 2H), 0.78-0.87 (m, 2H), 0.97 (d, J=6.45 Hz, 3H), 1.06-1.34 (m, 3H), 1.54-1.62 (m, 1H), 1.64-1.74 (m, 1H), 1.75-1.85 (m, 1H), 2.78-2.90 (m, 1H), 3.01-3.17 (m, 1H), 3.29 (s, 3H), 3.27-3.30 (m, 2H), 3.38-3.50 (m, 2H), 3.51-3.62 (m, 1H), 3.65-3.75 (m, 1H), 3.91-4.02 (m, 1H), 4.35-4.46 (m, 1H), 4.55-4.63 (m, 1H), 4.76-4.84 (m, 1H), 7.41 (dd, J=8.59 Hz, 1H), 7.62 (s, 1H), 8.05 (d, J=8.59 Hz, 1H).

### Example 97

### 8-[(4-methylpiperidin-1-yl)carbonyl]-5-(methylsulfonyl)-2-(3,3,3-trifluoropropyl)-2,3,4,5-tetrahydro-1H-pyrido[4,3-b]indole

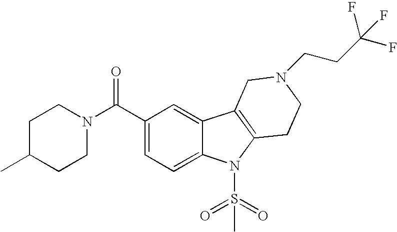

Following the same procedure as Example 93, Step C: The title compound as its TFA salt was prepared from 8-[(4-methylpiperidin-1-yl)carbonyl]-5-(methylsulfonyl)-2,3,4,5-tetrahydro-1H-pyrido[4,3-b]indole and 3,3,3-trifluoropropanal. MS (M+1): 472.0. ¹H NMR (400 MHz, CD3OD) δ ppm 0.96 (d, J=6.45 Hz, 3H), 1.10-1.20 (m, 2H), 1.54-1.87 (m, 2H), 2.84-3.00 (m, 4H), 3.01-3.16 (m, 1H), 3.29 (s, 3H), 3.41-3.50 (m, 2H), 3.64-3.72 (m, 4H), 3.74-3.82 (m, 2H), 4.55-4.66 (m, 2H), 7.41 (d, J=8.59, 1H), 7.59 (s, 1H), 8.04 (d, J=8.59 Hz, 1H).

### Example 98

### 5-(cyclopropylsulfonyl)-8-[(4-methylpiperidin-1-yl)carbonyl]-2-(tetrahydro-2H-pyran-4-yl)-2,3,4,5-tetrahydro-1H-pyrido[4,3-b]indole

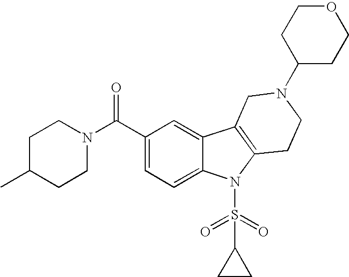

### Step A: The preparation of tert-butyl 5-(cyclopropylsulfonyl)-8-[(4-methylpiperidin-1-yl)carbonyl]-1,3,4,5-tetrahydro-2H-pyrido[4,3-b]indole-2-carboxylate

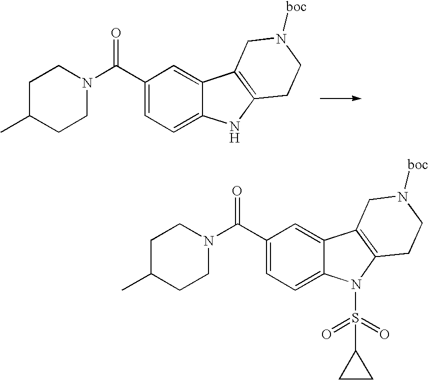

To the solution of 2-(tert-butoxycarbonyl)-2,3,4,5-tetrahydro-1H-pyrido[4,3-b]indole-8-carboxylic acid (0.5 mmol) in dry DMF (4 mL) was added 60% NaH (1.0 mmol) in one portion at 0° C. under nitrogen. The mixture was stirred at room temperature for 30 min, then methanesulfonyl chloride (1.0 mmol) was added slowly at room temperature, and the reaction mixture was stirred at room temperature for overnight. The solvent was removed in vacuo and the residue was dissolved in dichloromethane (20 mL), washed with water (10 mL), dried over sodium sulfate. Removal of solvent gave the crude product, which was used for the next step without further purification. MS (M+1): 502.07.

### Step B: The preparation of 5-(cyclopropylsulfonyl)-8-[(4-methylpiperidin-1-yl)carbonyl]-2,3,4,5-tetrahydro-1H-pyrido[4,3-b]indole

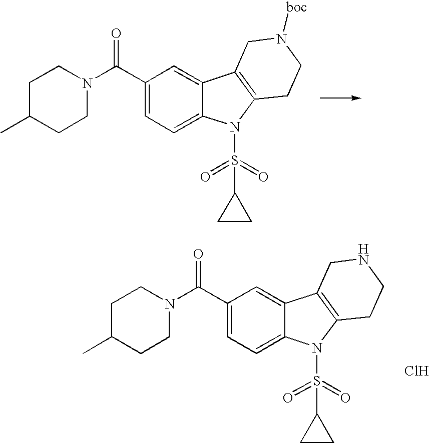

To a solution of crude tert-butyl 5-(cyclopropylsulfonyl)-8-[(4-methylpiperidin-1-yl)carbonyl]-1,3,4,5-tetrahydro-2H-pyrido[4,3-b]indole-2-carboxylate (0.5 mmol) in dioxane (3 mL) was added 4N HCl in dioxane (2 mL). The solution was stirred at room temperature for 3 h. The solvent was removed in vacuo and the residue was used without further purification. MS (M+1): 402.0.

### Step C: The preparation of 5-(cyclopropylsulfonyl)-8-[(4-methylpiperidin-1-yl)carbonyl]-2-(tetrahydro-2H-pyran-4-yl)-2,3,4,5-tetrahydro-1H-pyrido[4,3-b]indole

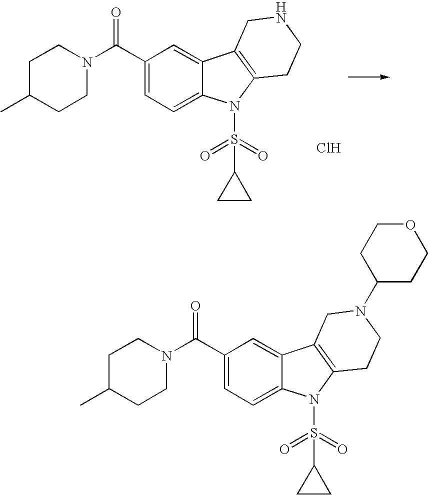

To a solution of crude 5-(cyclopropylsulfonyl)-8-[(4-methylpiperidin-1-yl)carbonyl]-2,3,4,5-tetrahydro-1H-pyrido[4,3-b]indole in dry dichloromethane (4 mL) was added tetrahydro-4H-pyran-4-one (0.5 mmol) and sodium triacetoxyborohydride (1.0 mmol). The mixture was stirred at room temperature overnight, diluted with dichloromethane (15 mL), washed with saturated NaHCO₃ solution (5 mL), dried over Na₂SO₄. The dichloromethane solution was concentrated in vacuo and the residue was purified with reverse phase HPLC to give the desired product as its TFA salt (38 mg, 13% for 3 steps). MS (M+1): 486.0. 1H NMR (600 Mai, CDCl3) δ ppm 1.01 (d, J=6.42 Hz, 3H), 0.97-1.09 (m, 2H), 1.17 (m, 1H), 1.32 (m, 3H), 1.70 (m, 2H), 1.82 (m, 1H), 2.03 (m, 2H), 2.12 (m, 2H), 2.60 (m, 1H), 2.85 (m, 1H), 3.22 (m, 3H), 3.51 (m, 3H), 3.58 (m, 1H), 3.75 (m, 2H), 3.89 (m, 1H), 4.19 (d, J=8.71 Hz, 2H), 4.68 (m, 2H), 7.36 (d, J=8.45 Hz, 1H), 7.52 (s, 1H), 8.03 (d, J=8.45 Hz, 1H).

### Example 99

### 2-cyclopentyl-5-(ethylsulfonyl)-8-[(4-methylpiperidin-1-yl)carbonyl]-2,3,4,5-tetrahydro-1H-pyrido[4,3-b]indole

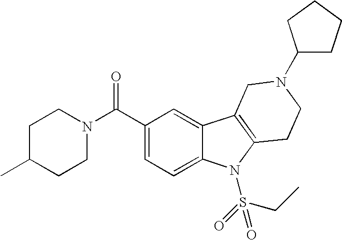

### Step A: The preparation of tert-butyl 5-(ethylsulfonyl)-8-[(4-methylpiperidin-1-yl)carbonyl]-1,3,4,5-tetrahydro-2H-pyrido[4,3-b]indole-2-carboxylate

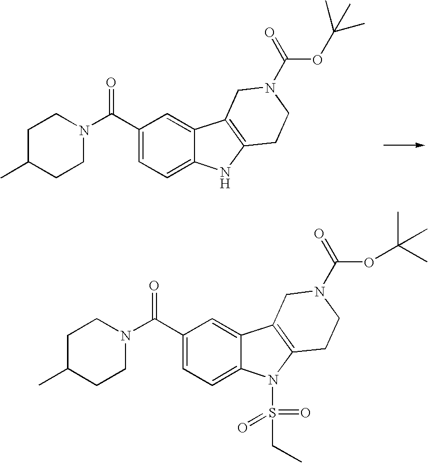

60% NaH (140 mg, 3.50 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (400 mg, 1.00 mmol) in DMF (10 mL) at room temperature under nitrogen. The mixture was stirred for 30 min, ethylsulfonyl chloride (0.12 mL, 1.27 mmol) was added at room temperature, and the reaction mixture was stirred at room temperature for 3 h. The solvent was removed in vacu and the residue was dissolved in dichloromethane and washed with water, dried over sodium sulfate and condensed under reduced pressure. Purification of the crude over reverse phase HPLC using (5-75% CH₃CN) provided (0.36 g, 73%) of the desired compound as its TFA salt. MS (M+1) 490.4.

### Step B: The preparation of 5-(ethylsulfonyl)-8-[(4-methylpiperidin-1-yl)carbonyl]-2,3,4,5-tetrahydro-1H-pyrido[4,3-b]indole

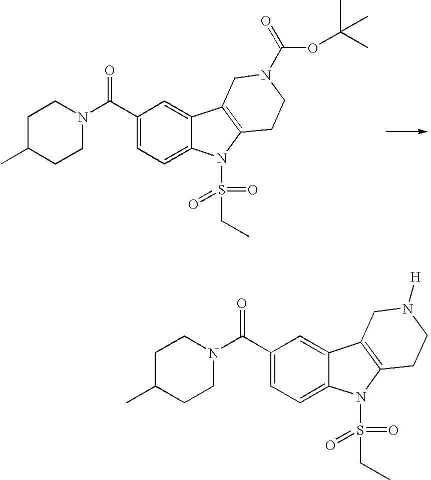

Tert-butyl 5-(ethylsulfonyl)-8-[(4-methylpiperidin-1-yl)carbonyl]-1,3,4,5-tetrahydro-2H-pyrido[4,3-b]indole-2-carboxylate (0.36 g, 0.73 mmol) was dissolved in dichloromethane (5 mL) and trifluoroacetic acid (0.5 mL) was added to the reaction mixture. The mixture was stirred at room temperature until the starting material was disappeared. Removal of excess trifluoroacetic acid in vacuo gave desired product as its trifluoroacetic acid salt form. The salt was then dissolved in methanol (6 mL) and MP carbonate was added (1.52 g). The mixture was stirred for one hour and then filtered and condensed. The free amine was then used for the next step without any further purification. MS (M+1): 390.31.

### Step C: The preparation of 2-cyclopentyl-5-(ethylsulfonyl)-8-[(4-methylpiperidin-1-yl)carbonyl]-2,3,4,5-tetrahydro-1H-pyrido[4,3-b]indole

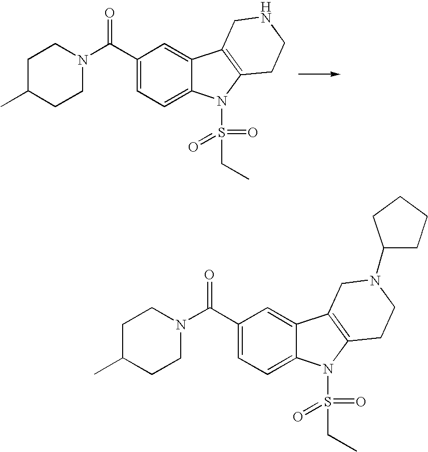

Sodium triacetoxyborohydride (60 mg, 0.28 mmol) was added to 5-(ethylsulfonyl)-8-[(4-methylpiperidin-1-yl)carbonyl]-2,3,4,5-tetrahydro-1H-pyrido[4,3-b]indole (71 mg, 0.18 mmol) and cyclopentanone (33 μL, 0.37 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt (8.5 mg). MS (M+1): 458.0. 1H NMR (600 MHz, CD3OD): δ ppm 0.87 (d, J=6.66 Hz, 3H), 0.94-1.16 (m, 2H), 1.10 (t, J=7.30 Hz, 3 H), 1.44-1.54 (m, 1H), 1.54-1.83 (m, 8H), 2.14-2.27 (m, 2H), 2.75 (t, J=11.65 Hz, 1H), 3.00 (t, J=11.65 Hz, 1H), 3.24-3.48 (m, 3H), 3.39 (q, J=7.34 Hz, 2H), 3.59 (d, J=11.65 Hz, 1H), 3.67-3.75 (m, 1H), 3.81-3.93 (m, 1H), 4.29 (d, J=14.59 Hz, 1H), 4.50 (d, J=11.01 Hz, 1H), 4.68 (d, J=14.08 Hz, 1H), 7.30 (d, J=8.70 Hz, 1H), 7.54 (s, 1H), 7.92 (d, J=8.70 Hz, 1H).

### Example 100

### 5-(ethylsulfonyl)-8-[(4-methylpiperidin-1-yl)carbonyl]-2-(tetrahydro-2H-pyran-4-yl)-2,3,4,5-tetrahydro-1H-pyrido[4,3-b]indole

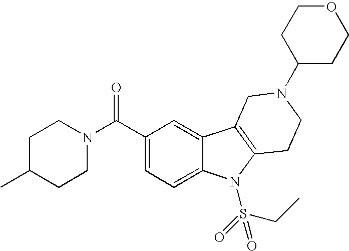

Sodium triacetoxyborohydride (60 mg, 0.28 mmol) was added to 5-(ethylsulfonyl)-8-[(4-methylpiperidin-1-yl)carbonyl]-2,3,4,5-tetrahydro-1H-pyrido[4,3-b]indole (72 mg, 0.18 mmol) and tetrahydro-4H-pyran-4-one (34 μL, 0.37 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt as a white solid (18 mg). MS (M+1): 474.0. 1H NMR (600 MHz, CD3OD) δ ppm 0.89 (d, J=6.40 Hz, 3H), 0.59-1.09 (m, 2H), 1.13 (t, J=7.42 Hz, 3H), 1.47-1.76 (m, 3H), 1.77-1.89 (m, 2H), 2.08 (d, J=12.29 Hz, 2H), 2.70-2.83 (m, 1H), 2.96-3.08 (m, 1H), 3.29-3.47 (m, 5H), 3.42 (q, J=7.25 Hz, 2H), 3.55-3.70 (m, 2H), 3.85-4.00 (m, 1H), 4.03 (dd, J=11.65, 4.48 Hz, 2H), 4.34-4.70 (m, 3H), 7.32 (d, J=8.45 Hz, 1H), 7.56 (s, 1H), 7.95 (d, J=8.45 Hz, 1H).

### Example 101

### 2-cyclobutyl-5-(ethylsulfonyl)-8-[(4-methylpiperidin-1-yl)carbonyl]-2,3,4,5-tetrahydro-1H-pyrido[4,3-b]indole

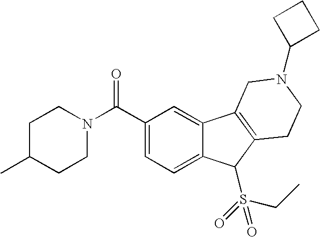

Sodium triacetoxyborohydride (100 mg, 0.47 mmol) was added to 5-(ethylsulfonyl)-8-[(4-methylpiperidin-1-yl)carbonyl]-2,3,4,5-tetrahydro-1H-pyrido[4,3-b]indole.TFA (90 mg, 0.23 mmol) and cyclobutanone (38 μL, 0.51 mmol) in dichloromethane (2 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt (24 mg). MS (M+1): 444.0. ¹HNMR (400 MHz, CDCl3) δ ppm 0.97 (d, J=6.25 Hz, 3H), 1.01-1.31 (m, 2H), 1.15 (t, J=7.32 Hz, 2 H), 1.53-1.90 (m, 4H), 1.97 (q, J=9.96 Hz, 1H), 2.24-2.38 (m, 2H), 2.57-2.71 (m, 2H), 2.73-2.88 (m, 1H), 2.93-3.12 (m, 2H), 3.23 (q, J=6.58 Hz, 2H), 3.28-3.40 (m, 1H), 3.40-3.86 (m, 5H), 4.54-4.73 (m, 2H), 7.32 (d, J=8.40 Hz, 1H), 7.41 (s, 1H), 7.94 (d, J=8.40 Hz, 1H).

### Example 102

### 2-(cyclopropylmethyl)-5-(ethylsulfonyl)-8-[(4-methylpiperidin-1-yl)carbonyl]-2,3,4,5-tetrahydro-1H-pyrido[4,3-b]indole

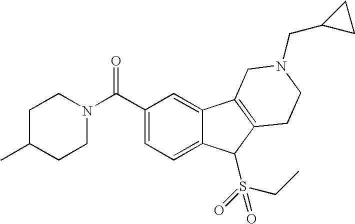

Sodium triacetoxyborohydride (100 mg, 0.47 mmol) was added to 5-(ethylsulfonyl)-8-[(4-methylpiperidin-1-yl)carbonyl]-2,3,4,5-tetrahydro-1H-pyrido[4,3-b]indole.TFA (90 mg, 0.23 mmol) and cyclopropanecarbaldehyde (38 μL, 0.51 mmol) in dichloromethane (2 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt (11 mg). MS (M+1): 444.0. 1H NMR (400 MHz, CDCl3) δ ppm 0.46 (d, J=3.71 Hz, 2H), 0.76-0.87 (m, 2H), 0.82 (m, 2H), 0.97 (d, J=6.45 Hz, 3H), 1.05-1.31 (m, 2H), 1.17 (t, J=7.32 Hz, 3H), 1.55-1.86 (m, 3H), 2.57-2.90 (m, 1H), 2.93-3.17 (m, 2H), 3.17-3.44 (m, 4H), 3.60-3.43 (m, 1H), 3.63-3.79 (m, 1H), 3.88-4.01 (m, 1H), 4.09 (d, J=7.32 Hz, 1H), 4.66 (bs, 1H), 4.78 (d, J=14.65 Hz, 1H), 7.33 (d, J=8.40 Hz, 1H), 7.47 (s, 1H), 7.95 (d, J=8.40 Hz, 1H).

### Example 103

### 5-(ethylsulfonyl)-8-[(4-methylpiperidin-1-yl)carbonyl]-2-(tetrahydrofuran-3-yl)-2,3,4,5-tetrahydro-1H-pyrido[4,3-b]indole

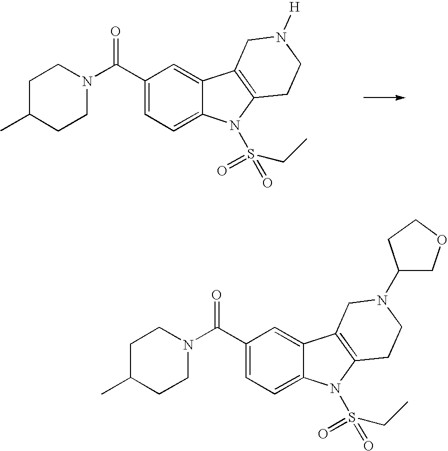

Following the similar procedure of Example 94, Step C: the title compound was prepared from 5-(ethylsulfonyl)-8-[(4-methylpiperidin-1-yl)carbonyl]-2,3,4,5-tetrahydro-1H-pyrido[4,3-b]indole.TFA and dihydrofuran-3(2H)-one. MS (M+1): 459.98. 1H NMR (400 MHz, CD3OD) δ ppm 0.99 (d, J=6.44 Hz, 3H), 1.07-1.19 (m, 2H), 1.21 (t, J=7.32 Hz, 3H), 1.45-1.92 (m, 3H), 2.21-2.43 (m, 1H), 2.42-2.62 (m, 1H), 2.74-2.96 (m, 1H), 3.00-3.20 (m, 1H), 3.38-3.56 (m, 4H), 3.58-3.85 (m, 2H), 3.65 (s, 1H), 3.79 (q, J=8.01 Hz, 2H), 3.95 (dd, J=10.94, 6.05 Hz, 1 H), 4.02-4.19 (m, 1H), 4.19-4.38 (m, 2H), 4.60 (s, 2H), 7.41 (dd, J=8.59, 1.56 Hz, 1H), 7.64 (d, J=1.17 Hz, 1H), 8.04 (d, J=8.59 Hz, 1H). Anal. Calcd for C₂₄H₃₃N₃O₄S.2C₂HF₃O₂.0.5H₂O: C, 48.27; H, 5.21; N, 6.03. Found: C, 48.44; H, 4.81; N, 6.01.

### Example 104

### 2-(2,2-difluoroethyl)-5-(ethylsulfonyl)-8-[(4-methylpiperidin-1-yl)carbonyl]-2,3,4,5-tetrahydro-1H-pyrido[4,3-b]indole

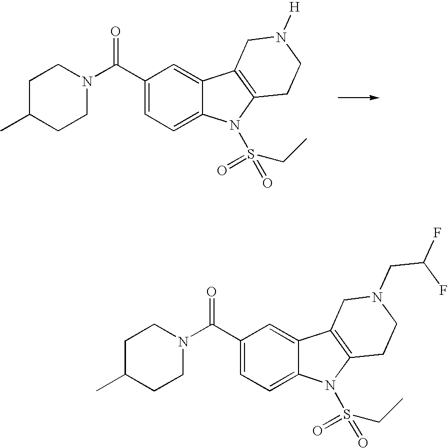

The mixture 5-(ethylsulfonyl)-8-[(4-methylpiperidin-1-yl)carbonyl]-2,3,4,5-tetrahydro-1H-pyrido[4,3-b]indole TFA salt (0.179 mmol), K₂CO₃ (110 mg, 0.797 mmol) in DMF (2.5 mL) was degassed under vacuo and then purged with nitrogen, the degassing and purging process was repeated for 3 times, then 1,1-difluoro-2-iodoethane (50 uL) was added. The mixture was heated at 140° C. in a sealed tube for 2 h, allowed to cool to room temperature, diluted with dichloromethane (50 mL) and water (5 mL). The mixture was stirred for 10 min, passed through a 10 g prepacked hydromatrix cartridge. Removal of solvent gave the crude product, which was purified by preparative HPLC (10-70% CH₃CN) to afford the title compound as its TFA salt (31 mg). MS (M+1): 454.0. 1H NMR (600 MHz, CD3OD) δ ppm 0.99 (d, J=6.40 Hz, 3H), 1.04-1.17 (m, 2H), 1.22 (t, J=7.30 Hz, 3H), 1.53-1.64 (m, 1H), 1.65-1.75 (m, 1H), 1.75-1.88 (m, 1H), 2.79-2.93 (m, J=8.96 Hz, 1H), 3.04-3.19 (m, 1H), 3.41 (s, 2H), 3.49 (q, J=7.30 Hz, 2H), 3.64-3.86 (m, 5H), 4.54 (s, 2H), 4.61 (d, J=11.78 Hz, 1H), 6.46 (dt, J=53.89, 3.07 Hz, 1H), 7.40 (dd, J=8.45, 1.41 Hz, 1H), 7.59 (s, 1H), 8.05 (d, J=8.45 Hz, 1H).

### Example 105

### 5-(isopropylsulfonyl)-8-[(4-methylpipericlin-1-yl)carbonyl]-2-(tetrahydro-2H-pyran-4-yl)-2,3,4,5-tetrahydro-1H-pyrido[4,3-b]indole

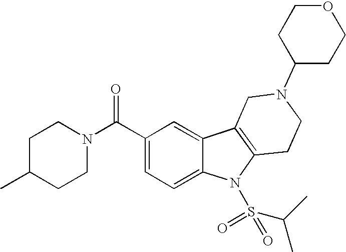

### Step A: The preparation of tert-butyl 5-(isopropylsulfonyl)-8-[(4-methylpiperidin-1-yl)carbonyl]-1,3,4,5-tetrahydro-2H-pyrido[4,3-b]indole-2-carboxylate

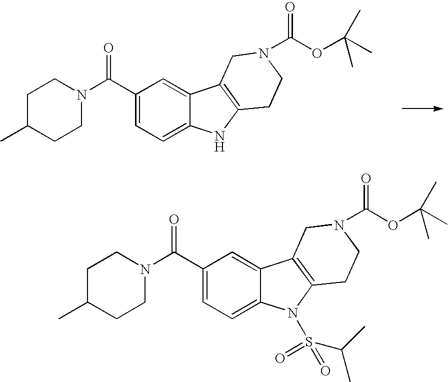

60% NaH (56 mg, 1.40 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (150 mg, 0.38 mmol) in DMF (6 mL) at room temperature under nitrogen. The mixture was stirred for 30 min, propane-2-sulfonyl chloride (55 μL 0.49 mmol) was added and the reaction mixture was stirred at room temperature for 2 h. The solvent was removed in vacuo and the residue was dissolved in dichloromethane and washed with water dried over sodium sulfate. Removal of solvent gave the crude product, which was purified using preparative HPLC (5-75% CH₃CN) to provide the desired product (50 mg). MS (M+1): 504.4.

### Step B: 5-(isopropylsulfonyl)-8-[(4-methylpiperidin-1-yl)carbonyl]-2,3,4,5-tetrahydro-1H-pyrido[4,3-b]indole

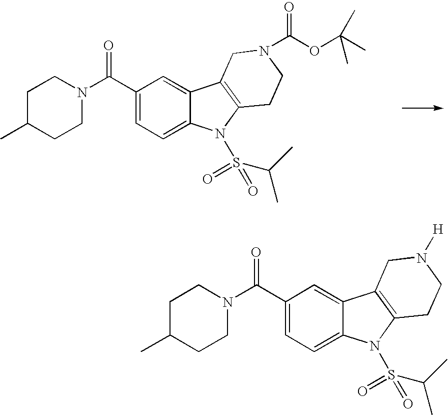

tert-butyl 5-(isopropylsulfonyl)-8-[(4-methylpiperidin-1-yl)carbonyl]-1,3,4,5-tetrahydro-2H-pyrido[4,3-b]indole-2-carboxylate (50 mg, 0.08 mmol) was dissolved in dichloromethane (2 mL) and trifluoroacetic acid (0.2 mL) was added to the reaction mixture. The mixture was stirred until disappearance of starting material. Removal of excess trifluoroacetic acid in vacuo gave crude product as its trifluoroacetic acid salt form (47 mg). MS (M+1+CH₃CN): 445.40. The salt was then neutralized with MP carbonate: 0.18 g MP carbonate was added to a solution of above salt in MeOH (1 mL) and the mixture was stirred for one hour then filtered and condensed.

### Step C: 5-(isopropylsulfonyl)-8-[(4-methylpiperidin-1-yl)carbonyl]-2-(tetrahydro-2H-pyran-4-yl)-2,3,4,5-tetrahydro-1H-pyrido[4,3-b]indole

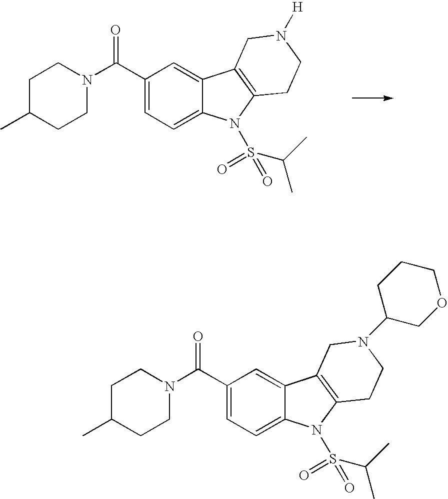

Sodium triacetoxyborohydride (37 mg, 0.17 mmol) was added to 5-(isopropylsulfonyl)-8-[(4-methylpiperidin-1-yl)carbonyl]-2,3,4,5-tetrahydro-1H-pyrido[4,3-b]indole (47 mg, 0.12 mmol) and tetrahydro-4H-pyran-4-one (13 μL, 0.14 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt (8 mg). MS (M+1): 488.0. 1H NMR (600 MHz, CD3OD) δ ppm 0.88 (d, J=6.66 Hz, 3H), 0.93-1.23 (m, 3H) 1.18 (d, J=6.40 Hz, 6H), 1.44-1.55 (m, 1H), 1.55-1.64 (m, 1H), 1.65-1.74 (m, 1H), 1.77-1.88 (m, 2H), 2.07 (d, J=11.01 Hz, 2H), 2.69-2.81 (m, 1H), 3.01 (t, J=12.29 Hz, 2H), 3.27-3.50 (m, 2H), 3.40 (t, J=11.65 Hz, 2H), 3.53-3.67 (m, 3 H), 4.02 (dd, J=11.65, 4.22 Hz, 2H), 4.33-4.72 (m, 2H), 4.51 (d, J=10.75 Hz, 1H), 7.30 (d, J=8.70 Hz, 1H), 7.55 (s, 1H), 7.92 (d, J=8.70 Hz, 1H).

### Example 106

### N,N-dimethyl-8-[(4-methylpiperidin-1-yl)carbonyl]-2-(tetrahydro-2H-pyran-4-yl)-1,2,3,4-tetrahydro-5H-pyrido[4,3-b]indole-5-sulfonamide

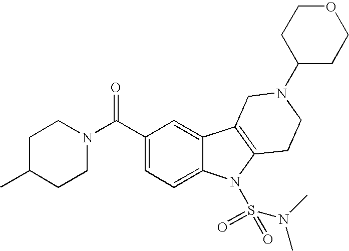

### Step A: The preparation of tert-butyl 5-[(dimethylamino)sulfonyl]-8-[(4-methylpiperidin-1-yl)carbonyl]-1,3,4,5-tetrahydro-2H-pyrido[4,3-b]indole-2-carboxylate

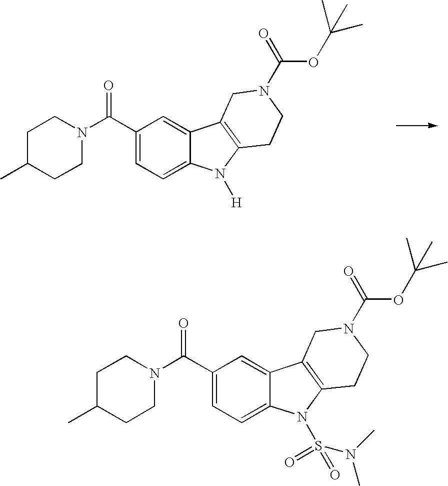

60% NaH (463 mg, 11.6 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (823 mg, 2.07 mmol) in DMF (20 mL) at room temperature under nitrogen. The mixture was stirred at room temperature for 1 h, then dimethylaminosulfonyl chloride (1.10 g, 7.69 mmol) was added dropwise at room temperature, and the reaction mixture was stirred at room temperature for 2 h, quenched with 1N NaOH (6 mL). The solvent was removed in vacuo and the residue was dissolved in dichloromethane and washed with water, dried over sodium sulfate. Removal of solvent gave the crude product (1.04 g), which was used for next step without purification. MS (M+1): 505.0.

### Step B: The preparation of N,N-dimethyl-8-[(4-methylpiperidin-1-yl)carbonyl]-1,2,3,4-tetrahydro-5H-pyrido[4,3-b]indole-5-sulfonamide TFA salt

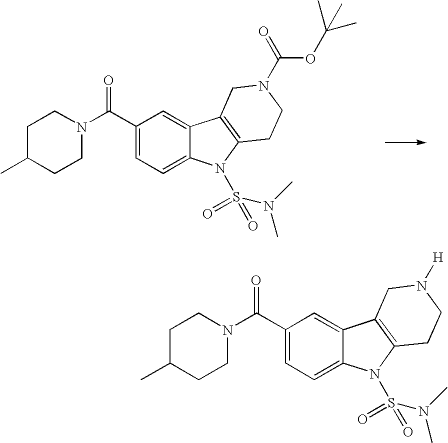

tert-butyl 5-[(dimethylamino)sulfonyl]-8-[(4-methylpiperidin-1-yl)carbonyl]-1,3,4,5-tetrahydro-2H-pyrido[4,3-b]indole-2-carboxylate (1.01 g, 2.00 mmol) from step A was dissolved in TFA (10 mL), and the mixture was stirred at room temperature for 1 h, then TEA was removed in vacuo and the residue was coevaporated with methanol to afford the crude product (1.60 g). The crude product was dissolved in water-acetonitrile and lipholized to give the title compound as its TFA salt (1.07 g).

### Step C: The preparation of N,N-dimethyl-8-[(4-methylpiperidin-1-yl)carbonyl]-2-(tetrahydro-2H-pyran-4-yl)-1,2,3,4-tetrahydro-5H-pyrido[4,3-b]indole-5-sulfonamide

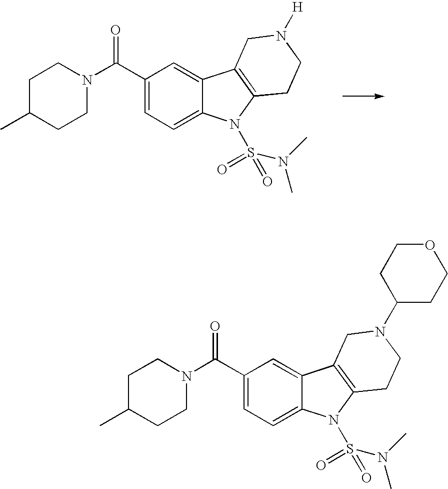

Using the similar method as described in Example 93, Step C: the title compound was prepared from N,N-dimethyl-8-[(4-methylpiperidin-1-yl)carbonyl]-1,2,3,4-tetrahydro-5H-pyrido[4,3-b]indole-5-sulfonamide TFA salt and tetrahydro-4-pyranone. MS (M+1): 489.0. 1H NMR (600 MHz, CD3OD) δ ppm 1.01 (t, J=6.91 Hz, 3H), 1.06-1.33 (m, 2H), 1.61 (s, 1H), 1.73 (s, 1H), 1.82 (s, 1H), 1.89-2.03 (m, 2H), 2.11-2.29 (m, 2H), 2.89 (s, 3H), 2.91 (s, 3H), 3.14 (s, 1H), 3.37-3.65 (m, 6H), 3.67-3.81 (m, 2H), 3.95-4.10 (m, 1H), 4.11-4.20 (m, 2H), 4.53 (s, 1H), 4.64 (s, 1H), 4.75 (s, 1H), 7.37-7.47 (m, 1H), 7.65 (7.67) (s, 1H), 8.09 (8.10) (d, J=8.32 Hz, 1H).

### Example 107

### N,N-dimethyl-8-[(4-methylpiperidin-1-yl)carbonyl]-2-(3,3,3-trifluoropropyl)-1,2,3,4-tetrahydro-5H-pyrido[4,3-b]indole-5-sulfonamide

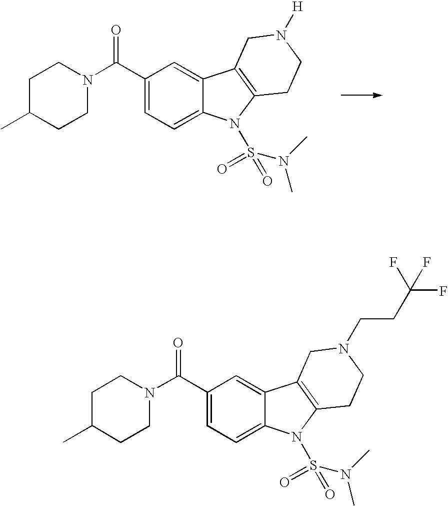

Using similar method as described in Example 93, Step C: the title compound was prepared from N,N-dimethyl-8-[(4-methylpiperidin-1-yl)carbonyl]-1,2,3,4-tetrahydro-5H-pyrido[4,3-b]indole-5-sulfonamide TFA salt and 3,3,3-trifluoropropanal. MS (M+1): 501.0. ¹H NMR (400 MHz, CD3OD) δ ppm 0.96 (d, J=6.25 Hz, 3H), 1.04-1.36 (m, 2H), 1.48-1.86 (m, 3H), 2.85 (s, 6H), 2.86-3.17 (m, 4H), 3.44 (t, J=5.86 Hz, 2H), 3.59-3.86 (m, 5H), 4.59 (s, 3H), 7.37 (dd, J=8.59, 1.17 Hz, 1H), 7.57 (d, J=1.17 Hz, 1H), 8.05 (d, J=8.59 Hz, 1H).

### Example 108

### 2-cyclopentyl-N,N-dimethyl-8-[(4-methylpiperidin-1-yl)carbonyl]-1,2,3,4-tetrahydro-5H-pyrido[4,3-b]indole-5-sulfonamide

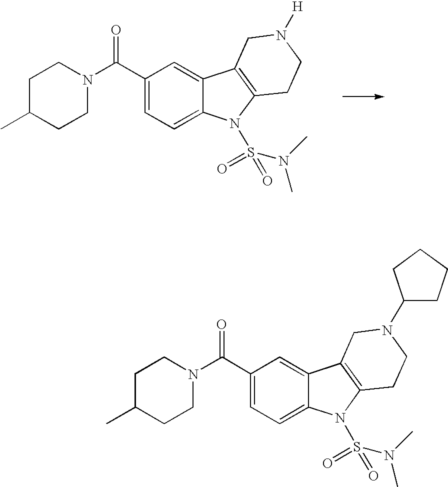

Using similar method as described in Example 93, Step C: the title compound was prepared from N,N-dimethyl-8-[(4-methylpiperidin-1-yl)carbonyl]-1,2,3,4-terrahydro-5H-pyrido[4,3-b]indole-5-sulfonamide TFA salt and cyclopentanone. MS (M+1): 473.01. 1H NMR (400 MHz, CD3OD) δ ppm 0.96 (d, J=6.64 Hz, 3H), 1.02-1.30 (m, 2H), 1.49-1.96 (m, 9H), 2.22-2.36 (m, 2H), 2.79-2.90 (m, 1H), 2.85 (s, 6H), 2.97-3.17 (m, 1H), 3.32-4.03 (m, 6H), 4.32-4.81 (m, 3H), 7.36 (dd, J=8.59, 1.56 Hz, 1H), 7.62 (dd, J=1.56, 0.78 Hz, 1H), 8.04 (dd, J=8.59, 0.78 Hz, 1H). Anal. Calcd for C₂₅H₃₆N₄O₃S.1.25C₂HF₃O₂.H₂O: C, 52.16; H, 6.25; N, 8.84. Found: C, 52.14; H, 6.46; N, 8.51.

### Example 109

### N,N-dimethyl-8-[(4-methylpiperidin-1-yl)carbonyl]-2-(tetrahydrofuran-3-yl)-1,2,3,4-tetrahydro-5H-pyrido[4,3-b]indole-5-sulfonamide

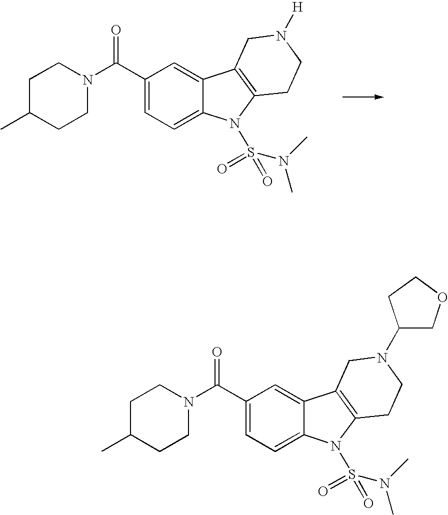

Using similar method as described in Example 93, Step C: the title compound as its TFA salt was prepared from N,N-dimethyl-8-[(4-methylpiperidin-1-yl)carbonyl]-1,2,3,4-tetrahydro-5H-pyrido[4,3-b]indole-5-sulfonamide TFA salt and dihydrofuran-3 (2H)-one. MS (M+1): 475.02. 1H NMR (400 MHz, CD3OD) δ ppm 0.97 (d, J=6.64 Hz, 3H), 1.04-1.29 (m, 2H), 1.50-1.87 (m, 3H), 2.22-2.38 (m, 1H), 2.43-2.59 (m, 1H), 2.80 (d, J=0.78 Hz, 1H), 2.84-2.87 (m, 6H), 2.99-3.17 (m, 1H), 3.38-3.51 (m, 2H), 3.63-3.84 (m, 4H), 3.93 (dd, J=11.13, 6.05 Hz, 1H), 4.09-4.18 (m, 1H), 4.19-4.33 (m, 2H), 4.43-4.75 (m, 3H), 7.37 (dd, J=8.59, 1.56 Hz, 1H), 7.60 (d, J=1.56 Hz, 1H), 8.05 (d, J=8.59 Hz, 1H).

### Example 110

### 2-cyclobutyl-N,N-dimethyl-8-[(4-methylpiperidin-1-yl)carbonyl]-1,2,3,4-tetrahydro-5H-pyrido[4,3-b]indole-5-sulfonamide

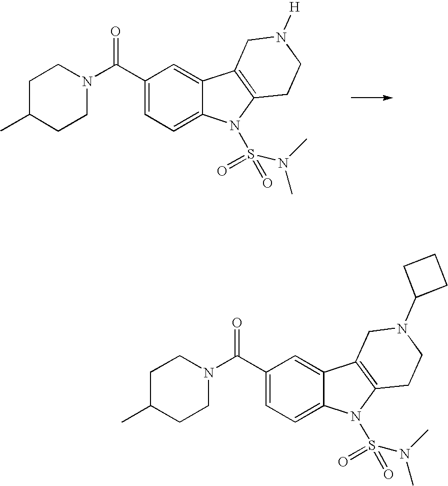

Using similar method as described in Example 93, Step C: the title compound was prepared from N,N-dimethyl-8-[(4-methylpiperidin-1-yl)carbonyl]-1,2,3,4-tetrahydro-5H-pyrido[4,3-b]indole-5-sulfonamide TFA salt and cyclobutanone. MS (M+1): 459.01. 1H NMR (400 MHz, CD3OD) δ ppm 0.96 (d, J=6.64 Hz, 3H), 1.00-1.29 (m, 2H), 1.41-2.05 (m, 5H), 2.13-2.56 (m, 4H), 2.69-2.96 (m, 1H), 2.85 (s, 6H), 2.95-3.19 (m, 1H), 3.30-3.53 (m, 3H,) 3.58-4.01 (m, 3H), 4.05-4.29 (m, 1H), 4.35-4.79 (m, 2H), 7.36 (dd, J=8.01, 1.17 Hz, 1H), 7.59 (d, J=1.17 Hz, 1H), 8.04 (d, J=8.01 Hz, 1H). Anal. Calcd for C₂₄H₃₄N₄O₃S. 1.75C₂HF₃O₂C, 50.18; H, 5.47; N, 8.51. Found: C, 50.00; H, 4.33; N, 8.09.

### Example 111

### N,N-dimethyl-8-[(4-methylpiperidin-1-yl)carbonyl]-2-(tetrahydro-2H-pyran-4-yl)-1,2,3,4-tetrahydro-5H-pyrido[4,3-b]indole-5-carboxamide

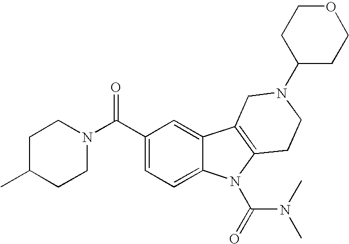

### Step A: The preparation tert-butyl 5-[(dimethylamino)carbonyl]-8-[(4-methylpiperidin-1-yl)carbonyl]-1,3,4,5-tetrahydro-2H-pyrido[4,3-b]indole-2-carboxylate

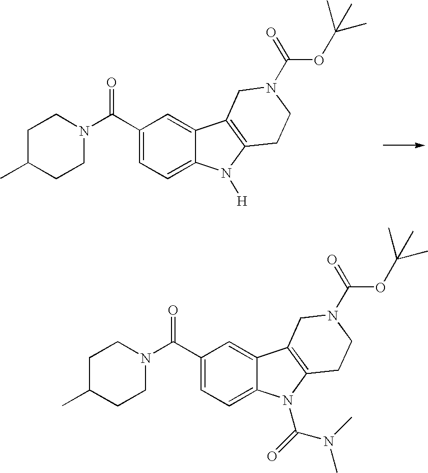

Following the similar procedure of Example 105, Step A: The title compound was prepared from tert-butyl 8-[(4-methylpiperidin-1-yl)carbonyl]-1,3,4,5-tetrahydro-2H-pyrido[4,3-b]indole-2-carboxylate and dimethylcarbamic chloride. MS (M+1): 469.10.

### Step B: N,N-dimethyl-8-[(4-methylpiperidin-1-yl)carbonyl]-1,2,3,4-tetrahydro-5H-pyrido[4,3-b]indole-5-carboxamide TFA salt

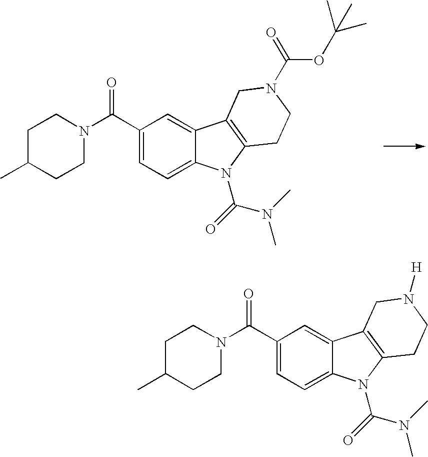

Following similar procedure of Example 93, Step B: The TFA salt of the title compound was prepared from tert-butyl 5-[(dimethylamino)carbonyl]-8-[(4-methylpiperidin-1-yl)carbonyl]-1,3,4,5-tetrahydro-2H-pyrido[4,3-b]indole-2-carboxylate and TFA in dichloromethane. MS (M+1): 369.1.

### Step C: The preparation of N,N-dimethyl-8-[(4-methylpiperidin-1-yl)carbonyl]-2-(tetrahydro-2H-pyran-4-yl)-1,2,3,4-tetrahydro-5H-pyrido[4,3-b]indole-5-carboxamide

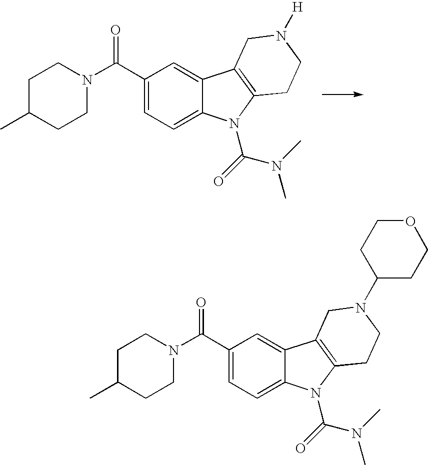

Following the similar procedure of Example 93, Step C: The title compound as its TFA salt was prepared from N,N-dimethyl-8-[(4-methylpiperidin-1-yl)carbonyl]-1,2,3,4-tetrahydro-5H-pyrido[4,3-b]indole-5-carboxamide TFA salt and tetrahydro-4-pyranone. MS (M+1): 453.1. 1H NMR (600 MHz, CD3OD) δ ppm 1.01 (d, J=6.66 Hz, 3H), 1.08-1.31 (m, 2H), 1.58-1.88 (m, 3H), 1.89-2.02 (m, 2H), 2.20 (d, J=11.26 Hz, 2H), 2.89 (s, 1H), 3.02-3.18 (m, 2H), 3.10 (s, 6H), 3.23-3.40 (m, 1H), 3.50-3.56 (t, J=11.52 Hz, 2H), 3.54-3.65 (m, 1H), 3.69-3.87 (m, 2H), 4.02 (s, 1H), 4.15 (dd, J=11.26, 4.10 Hz, 2H), 4.47-4.80 (m, 3H), 7.39 (d, J=8.70 Hz, 1H), 7.48 (d, J=8.70 Hz, 1H), 7.67 (s, 1H). Anal. Calcd for C₂₆H₃₆N₄O₃.1.75C₂H F₃O₂: C, 54.33; H, 5.83; N, 8.59. Found: C, 54.42; H, 5.81; N, 8.42

### Example 112

### 2-cyclobutyl-N,N-dimethyl-8-[(4-methylpiperidin-1-yl)carbonyl]-1,2,3,4-tetrahydro-5H-pyrido[4,3-b]indole-5-carboxamide

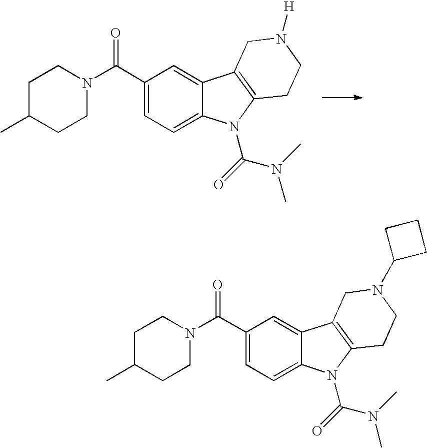

Following the similar procedure of Example 111: The title compound as its TFA salt was prepared from N,N-dimethyl-8-[(4-methylpiperidin-1-yl)carbonyl]-1,2,3,4-tetrahydro-5H-pyrido[4,3-b]indole-5-carboxamide TFA salt and cyclobutanone. MS (M+1): 423.0 (M+1). 1HNMR (400 MHz, CD3OD) δ ppm 0.97 (d, J=6.45 Hz, 3H), 1.04-1.33 (m, 2H), 1.36-2.07 (m, 5H), 2.18-2.57 (m, 4H), 2.73-2.96 (m, 1H), 2.95-3.16 (m, 2H), 3.06 (s, 6H), 3.16-3.43 (m, 2H), 3.65-3.85 (m, 2H), 3.86-4.03 (m, 1H), 4.08-4.27 (m, 1H), 4.48-4.78 (m, 2H), 7.35 (dd, J=0.98, 8.40, 1H), 7.42 (d, J=8.40 Hz, 1H), 7.61 (d, J=0.98 Hz, 1H).

### Example 113

### 5-butyryl-2-cyclobutyl-8-[(4-methylpiperidin-1-yl)carbonyl]-2,3,4,5-tetrahydro-1H-pyrido[4,3-b]indole

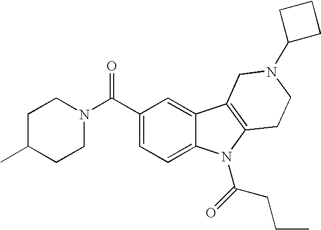

### Step A: tert-butyl 5-butyryl-8-[(4-methylpiperidin-1-yl)carbonyl]-1,3,4,5-tetrahydro-2H-pyrido[4,3-b]indole-2-carboxylate

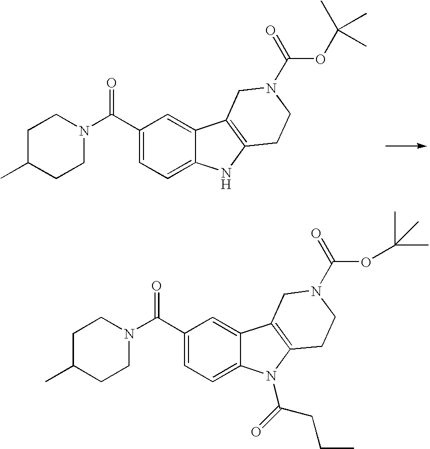

60% NaH (58 mg, 1.45 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (400 mg, 1 mmol) in DMF (10 mL) at room temperature under nitrogen. The mixture was stirred for one hour, butanoyl chloride (0.12 mL, 1.20 mmol) was added dropwise at −20° C. and the reaction mixture was allowed to rise to room temperature. The solvent was removed in vacuo and the residue was dissolved in dichloromethane and washed with water dried over sodium sulfate. Removal of the solvent gave the crude product, which was purified using preparative HPLC (5-80% CH₃CN) to provide the title compound (363 mg, 62%). MS (M+1): 468.4.

### Step B: 5-butyryl-8-[(4-methylpiperidin-1-yl)carbonyl]-2,3,4,5-tetrahydro-1H-pyrido[4,3-b]indole

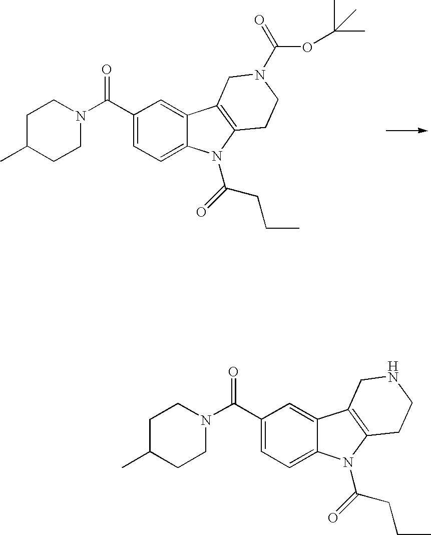

tert-butyl 5-butyryl-8-[(4-methylpiperidin-1-yl)carbonyl]-1,3,4,5-tetrahydro-2H-pyrido[4,3-b]indole-2-carboxylate was dissolved in dichloromethane (8 mL) and trifluoroacetic acid (0.8 mL) was added to the reaction mixture. The mixture was stirred until disappearance of starting material. Removal of excess trifluoroacetic acid in vacuo gave crude product. MS (M+1): 368.5. The salt was then neutralized with MP carbonate: 1.5 g MP carbonate was added to a solution of above salt in MeOH (6 mL) and the mixture was stirred for one hour then filtered and condensed.

### Step C: 5-butyryl-2-cyclobutyl-8-[(4-methylpiperidin-1-yl)carbonyl]-2,3,4,5-tetrahydro-1H-pyrido[4,3-b]indole

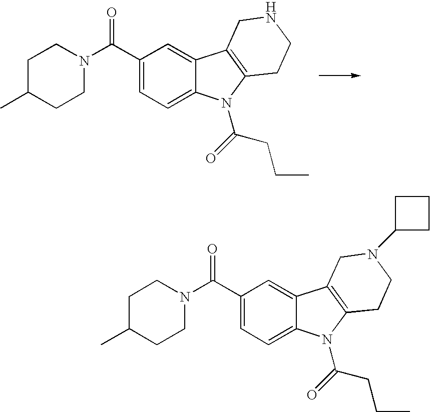

Sodium triacetoxyborohydride (51 mg, 0.24 mmol) was added to 5-butyryl-8-[(4-methylpiperidin-1-yl)carbonyl]-2,3,4,5-tetrahydro-1H-pyrido[4,3-b]indole (59 mg, 0.16 mmol) and cyclobutanone (24 μL, 0.32 mmol) in dichloromethane (2 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt (34.5 mg). MS (M+1): 422.0. ¹H NMR (600 MHz, CDCL3) δppm 0.96 (d, J=6.66 Hz, 3H), 1.02-1.18 (m, 1H), 1.15-1.32 (m; 1H), 1.07 (t, J=7.30 Hz, 3H), 1.51-1.72 (m, 2H), 1.73-1.90 (m, 4H), 1.96 (q, J=9.81 Hz, 1H), 2.75-2.39 (m, 2H), 2.47-2.67 (m, 2H), 2.75-3.04 (m, 4H), 3.11-3.22 (m, 1H), 3.24-3.39 (m, 1H), 3.42-3.55 (m, 1H), 3.56-3.76 (m, 3H), 3.81-3.93 (m, 1H), 4.52-4.71 (m, 2H), 7.23-7.32 (m, 1H), 7.38 (s, 1H), 7.82 (d, J=8.19 Hz, 1H).

### Example 114

### 5-butyryl-8-[(4-methylpiperidin-1-yl)carbonyl]-2-(tetrahydro-2H-pyran-4-yl)-2,3,4,5-tetrahydro-1H-pyrido[4,3-b]indole

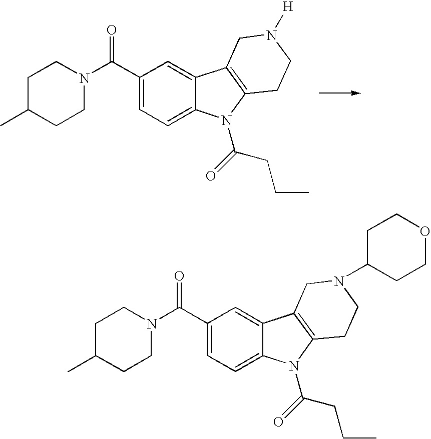

Sodium triacetoxyborohydride (70 mg, 0.33 mmol) was added to 5-butyryl-8-[(4-methylpiperidin-1-yl)carbonyl]-2,3,4,5-tetrahydro-1H-pyrido[4,3-b]indole (80 mg, 0.22 mmol) and tetrahydro-4H-pyran-4-one (40 μL, 0.43 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt (40 mg). MS (M+1): 452.0. 1H NMR (600 MHz, CDCl3) δ ppm 0.98 (d, J=6.40 Hz, 3H), 1.06-1.15 (m, 1H), 1.08 (t, J=7.42 Hz, 3H), 1.81-1.31 (m, 1H), 1.55-1.73 (m, 2H), 1.74-1.84 (m, 1H), 1.85-1.90 (m, 2H), 1.95-2.15 (m, 2H), 2.44-2.91 (m, 6H), 2.96 (t, J=7.17 Hz, 2H), 2.93-3.11 (m, 1H), 3.24-3.39 (m, 1H), 3.45 (t, J=11.65 Hz, 3H), 3.76-3.88 (m, 1H), 4.10-4.28 (m, 1H), 4.15 (dd, J=11.65, 3.71 Hz, 2H), 4.53-4.74 (m, 2H), 7.34 (d, J=8.70 Hz, 1H), 7.45 (s, 1H), 7.88 (d, J=8.70 Hz, 1H).

### Example 115

### 2-cyclopentyl-8-[(4-methylpiperidin-1-yl)carbonyl]-2,3,4,5-tetrahydro-1H-pyrido[4,3-b]indole

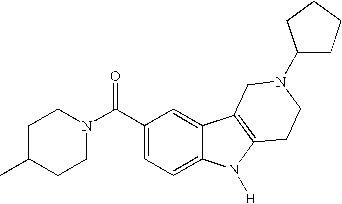

### Step A: The preparation of 8-[(4-methylpiperidin-1-yl)carbonyl]-2,3,4,5-tetrahydro-1H-pyrido[4,3-b]indole

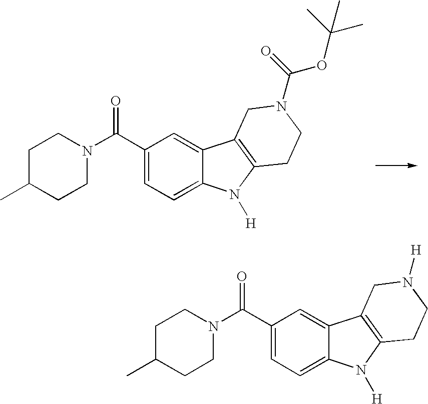

To a solution of tert-butyl 8-[(4-methylpiperidin-1-yl)carbonyl]-1,3,4,5-tetrahydro-2H-pyrido[4,3-b]indole-2-carboxylate (100 mg, 0.25 mmol) in methanol (1 mL) was added a solution of HCl in ether (2M, 3 mL) at room temperature. The mixture stirred until no more starting material was observed and then condensed under reduced pressure. The salt was then neutralized with MP carbonate: 0.51 g MP carbonate was added to a solution of above salt in MeOH (2 mL) and the mixture was stirred for one hour then filtered and condensed.

### Step B: The preparation of 2-cyclopentyl-8-[(4-methylpiperidin-1-yl)carbonyl]-2,3,4,5-tetrahydro-1H-pyrido[4,3-b]indole

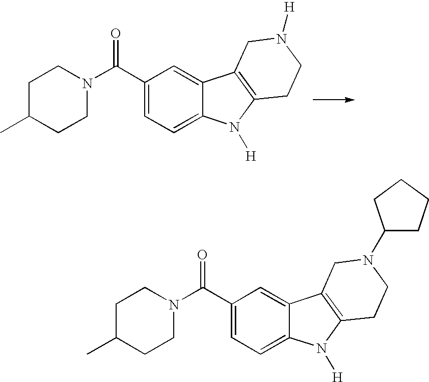

Sodium triacetoxyborohydride (15 mg, 0.24 mmol) was added to 8-[(4-methylpiperidin-1-yl)carbonyl]-2,3,4,5-tetrahydro-1H-pyrido[4,3-b]indole (50 mg, 0.17 mmol) and cyclopentanone (30 μL, mmol) in dichloromethane (2 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt (15 mg). MS (M+1): 366.0. 1H NMR (400 MHz, CDCl3) δ ppm 0.95 (d, J=6.44 Hz, 3H), 0.99-1.35 (m, 2H), 1.44-2.22 (m, 12H), 2.57-3.17 (m, 4H), 2.83 (d, J=15.82 Hz, 1H), 3.44-3.72 (m, 3H), 3.90 (d, J=15.82 Hz, 1H), 4.15-4.36 (m, 1H), 4.52-4.70 (m, 1H), 6.89-7.23 (m, 3H).

### Example 116

### 2-cyclopentyl-5-methyl-8-[(4-methylpiperidin-1-yl)carbonyl]-2,3,4,5-tetrahydro-1H-pyrido[4,3-b]indole

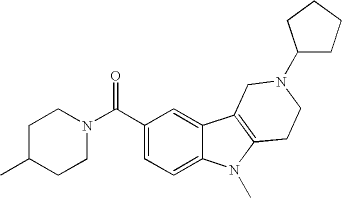

### Step A: tert-butyl 5-methyl-8-[(4-methylpiperidin-1-yl)carbonyl]-1,3,4,5-tetrahydro-2H-pyrido[4,3-b]indole-2-carboxylate

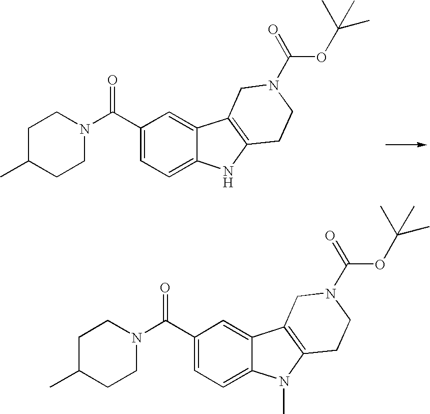

60% NaH (74 mg, 1.85 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (200 mg, 0.503 mmol) in DMF (5 mL) at room temperature under nitrogen. The mixture was stirred for 30 min, methyliodide (62 μL, 0.99 mmol) was added at room temperature, and the reaction mixture was stirred at room temperature for 2 h. The solvent was removed in vacu and the residue was dissolved in dichloromethane and washed with water dried over sodium sulfate. Removal of solvent gave the crude product. MS (M+1): 412.1.

### Step B: 5-methyl-8-[(4-methylpiperidin-1-yl)carbonyl]-2,3,4,5-tetrahydro-1H-pyrido[4,3-b]indole

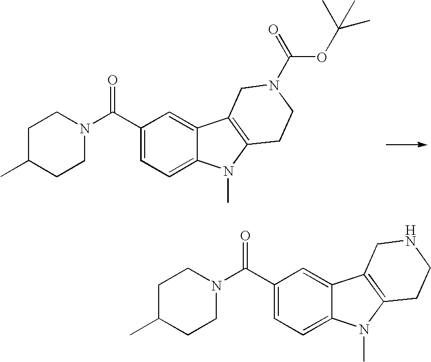

To the crude tert-butyl 5-methyl-8-[(4-methylpiperidin-1-yl)carbonyl]-1,3,4,5-tetrahydro-2H-pyrido[4,3-b]indole-2-carboxylate in MeOH (3 mL) was added a saturated HCl solution in ether (2M, 2 mL) at room temperature. The mixture was stirred until disappearance of starting material. Removal of solvent gave the desired product as its hydrochloric acid salt form. MS (M+1): 312.3.

### Step C: 2-cyclopentyl-5-methyl-8-[(4-methylpiperidin-1-yl)carbonyl]-2,3,4,5-tetrahydro-1H-pyrido[4,3-b]indole

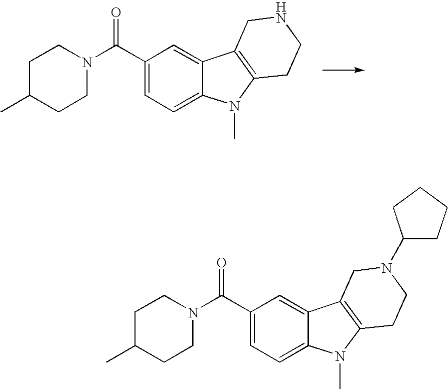

Sodium triacetoxyborohydride (136 mg, 0.64 mmol) was added to 5-methyl-8-[(4-methylpiperidin-1-yl)carbonyl]-2,3,4,5-tetrahydro-1H-pyrido[4,3-b]indole (133 mg, 0.35 mmol) and cyclopentanone (76 μL, 0.86 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt (46.6 mg). MS (M+1): 380.0. 1H NMR (600 MHz, CDCl3) d ppm 0.98 (d, J=6.40 Hz, 3H), 1.56-1.71 (m, 4H), 1.84-1.94 (m, 2H), 2.01-2.17 (m, 5H), 2.93-3.18 (m, 6H), 3.36-3.46 (m, 2H), 3.53-3.63 (m, 1H), 3.66 (s, 3H), 3.72-3.85 (m, 1H), 3.93 (dd, J=11.14, 4.99 Hz, 1H), 4.17 (d, J=14.34 Hz, 1H), 4.74 (d, J=14.34 Hz, 1H), 7.13-7.38 (m, 2H), 7.47 (s, 1H).

### Example 117

### 5-methyl-8-[(4-methylpiperidin-1-yl)carbonyl]-2-(tetrahydro-2H-pyran-4-yl)-2,3,4,5-tetrahydro-1H-pyrido[4,3-b]indole

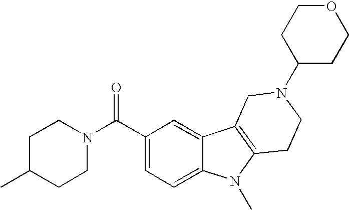

Sodium triacetoxyborohydride (85 mg, 0.40 mmol) was added to 5-methyl-8-[(4-methylpiperidin-1-yl)carbonyl]-2,3,4,5-tetrahydro-1H-pyrido[4,3-b]indole (80 mg, 0.25 mmol) and tetrahydro-4H-pyran-4-one (47 μL, 0.51 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt (20 mg). MS (M+1): 396.0. 1H NMR (600 MHz, CDCl3) δ ppm 0.97 (d, J=6.14 Hz, 3H), 1.17-1.31 (m, 1H), 1.58-1.81 (m, 2H), 1.91-2.15 (m, 6H), 2.98 (d, J=12.29 Hz, 2H), 3.30-3.51 (m, 4H), 3.59-3.81 (m, 3H), 3.63 (s, 3H), 3.84-3.93 (m, 1H), 4.13 (d, J=11.01 Hz, 2H), 4.24 (d, J=14.34 Hz, 1H), 4.51-4.72 (m, 2H), 7.21 (d, J=7.42 Hz, 2H), 7.40-7.44 (m, 1H).

### Example 118

### 2-cyclopentyl-5-ethyl-8-[(4-methylpiperidin-1-yl)carbonyl]-2,3,4,5-tetrahydro-1H-pyrido[4,3-b]indole

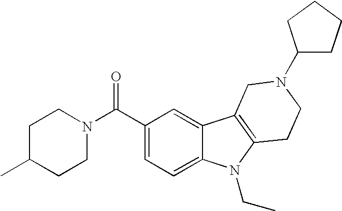

### Step A: The preparation of tert-butyl 5-ethyl-8-[(4-methylpiperidin-1-yl)carbonyl]-1,3,4,5-tetrahydro-2H-pyrido[4,3-b]indole-2-carboxylate

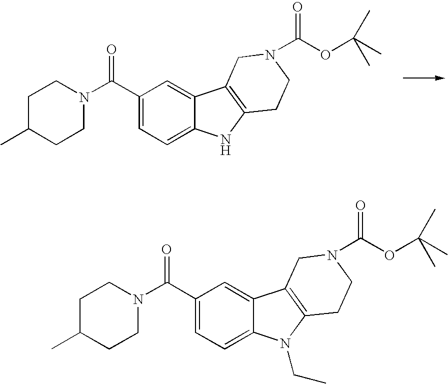

60% NaH (60 mg, 1.50 mmol) was added in one portion to a suspension of tent-butyl 8-[(4-methylpiperidin-1-yl)carbonyl]-1,3,4,5-tetrahydro-2H-pyrido[4,3-b]indole-2-carboxylate (160 mg, 0.40 mmol) in DMF (2 mL) at room temperature under nitrogen. The mixture was stirred for 30 min, ethyliodide (64 μL, 0.80 mmol) was added and the reaction mixture was stirred at room temperature for 2 h. The solvent was removed in vacuo and the residue was dissolved in dichloromethane and washed with water dried over sodium sulfate. Removal of solvent gave the crude product. MS (M+1): 426.1.

### Step B: The preparation of 5-ethyl-8-[(4-methylpiperidin-1-yl)carbonyl]-2,3,4,5-tetrahydro-1H-pyrido[4,3-b]indole

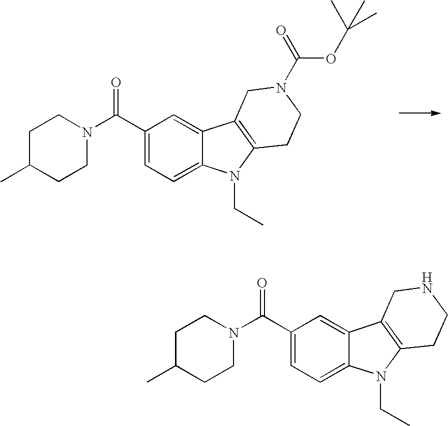

To the crude tert-butyl 5-ethyl-8-[(4-methylpiperidin-1-yl)carbonyl]-1,3,4,5-tetrahydro-2H-pyrido[4,3-b]indole-2-carboxylate in MeOH (2 mL) was added a saturated HCl solution in ether (2M, 2 mL) at room temperature. The mixture was stirred until disappearance of starting material. Removal of solvent gave the desired product as its hydrochloric acid salt form. The salt was then neutralized with MP carbonate: 0.6 g MP carbonate was added to a solution of above salt in MeOH (2 mL) and the mixture was stirred for one hour then filtered and condensed. MS (M+1): 326.3.

### Step C: The preparation of 2-cyclopentyl-5-ethyl-8-[(4-methylpiperidin-1-yl)carbonyl]-2,3,4,5-tetrahydro-1H-pyrido[4,3-b]indole

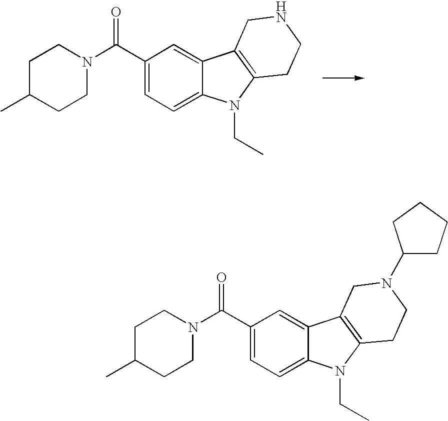

Sodium triacetoxyborohydride (120 mg, 0.57 mmol) was added to 5-ethyl-8-[(4-methylpiperidin-1-yl)carbonyl]-2,3,4,5-tetrahydro-1H-pyrido[4,3-b]indole and cyclopentanone (71 μL, 0.95 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its di-TFA salt as a yellow solid. (101 mg, 64% for three steps) MS (M+1): 394.0. 1H NMR (400 MHz, CDCL3) δ ppm 0.98 (d, J=6.44 Hz, 3H), 1.05-1.28 (m, 2H), 1.34 (t, J=7.23 Hz, 3H), 1.56-1.80 (m, 5H), 1.81-1.97 (m, 2H), 1.98-2.22 (m, 4H), 2.76-2.92 (m, 1H), 2.99 (d, J=16.21 Hz, 1H), 3.31-3.48 (m, 2H), 3.52-3.68 (m, 1H), 3.86-4.02 (m, 2H), 4.03-4.23 (m, 2H), 4.17 (d, J=13.70 Hz, 1H), 4.74 (d, J=13.70 Hz, 1H), 4.80-5.02 (m, 3H), 7.23 (d, J=8.40 Hz, 1H), 7.30 (d, J=8.40 Hz, 1H), 7.48 (s, 1H).

### Example 119

### 5-ethyl-8-[(4-methylpiperidin-1-yl)carbonyl]-2-(tetrahydro-2H-pyran-4-yl)-2,3,4,5-tetrahydro-1H-pyrido[4,3-b]indole

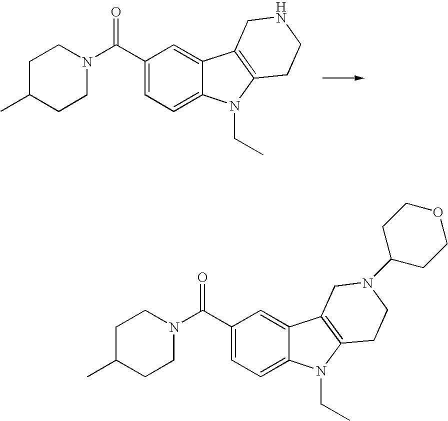

Following the similar procedure of Example 118, Step C: The title compound as its TFA salt was prepared from 5-ethyl-8-[(4-methylpiperidin-1-yl)carbonyl]-2,3,4,5-tetrahydro-1H-pyrido[4,3-b]indole and tetrahydro-4H-pyran-4-one. MS (M+1): 410.0. 1H NMR (600 MHz, CD3OD) δ ppm 0.83-0.91 (m, 3H), 0.95-1.16 (m, 2H), 1.18-1.27 (m, 3H), 1.33-1.40 (m, 2H), 1.42-1.75 (m, 3H), 1.81 (q, J=11.35 Hz, 1H), 2.02-2.12 (m, 1H), 2.68-2.84 (m, 1H), 2.92-3.06 (m, 1H), 3.09-3.23 (m, 3H), 3.28-3.51 (m, 1H), 3.39 (t, J=11.52 Hz, 1H), 3.64-3.77 (m, 1H), 3.55-3.64 (m, 1H), 3.64-3.77 (m, 1H), 3.83 (d, J=10.24 Hz, 0.5H), 3.87-3.97 (m, 0.5H), 4.01 (d, J=11.52 Hz, 1H), 4.06-4.16 (m, 2H), 4.21 (d, J=14.34 Hz, 0.5H), 4.32-4.42 (m, 0.5H), 4.43-4.55 (m, 1H), 4.55-4.67 (m, 0.5H), 4.63 (d, J=14.34 Hz, 0.5H), 7.13 (d, J=8.19 Hz, 1H), 7.39 (d, J=8.19 Hz, 1H), 7.45 (d, J=12.80 Hz, 1H).

### Example 120

### 2-cyclopentyl-5-(cyclopropylmethyl)-8-[(4-methylpiperidin-1-yl)carbonyl]-2,3,4,5-tetrahydro-1H-pyrido[4,3-b]indole

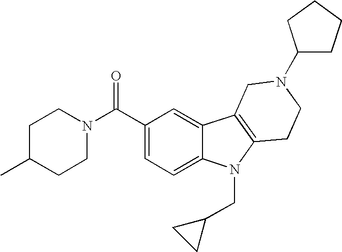

### Step A: tert-butyl 5-(cyclopropylmethyl)-8-[(4-methylpiperidin-1-yl)carbonyl]-1,3,4,5-tetrahydro-2H-pyrido[4,3-b]indole-2-carboxylate

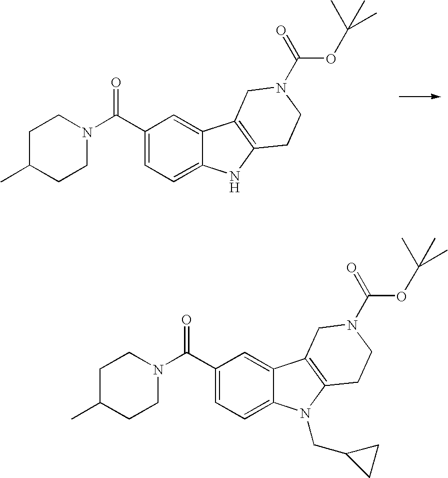

60% NaH (60 mg, 1.50 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (160 mg, 0.40 mmol) in DMF (2 mL) at room temperature under nitrogen. The mixture was stirred for 30 min, (chloromethyl)cyclopropane (78 μL, 0.84 mmol) was added at room temperature, and the reaction mixture was stirred at room temperature for 2 h. The solvent was removed in vacuo and the residue was dissolved in dichloromethane and washed with water dried over sodium sulfate. Removal of solvent gave the crude product. MS (M+1): 452.1.

### Step B: 5-(cyclopropylmethyl)-8-[(4-methylpiperidin-1-yl)carbonyl]-2,3,4,5-tetrahydro-1H-pyrido[4,3-b]indole

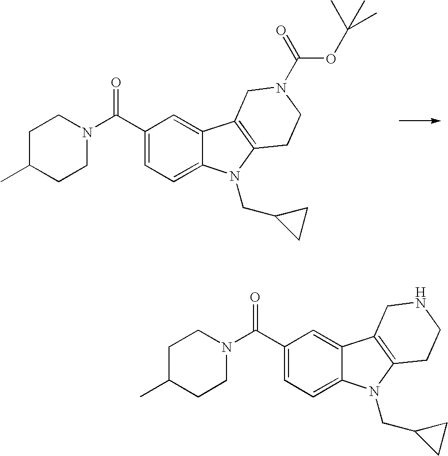

To the crude of tert-butyl 5-(cyclopropylmethyl)-8-[(4-methylpiperidin-1-yl)carbonyl]-1,3,4,5-tetrahydro-2H-pyrido[4,3-b]indole-2-carboxylate in MeOH (2 mL) was added a saturated HCl solution in ether (2M, 2 mL) at room temperature. The mixture was stirred until disappearance of starting material. Removal of solvent gave the desired product as its hydrochloric acid salt form. MS (M+1): 352.36. The salt was then neutralized with MP carbonate: 0.62 g MP carbonate was added to a solution of above salt in MeOH (2 mL) and the mixture was stirred for one hour then filtered and condensed.

### Step C: 2-cyclopentyl-5-(cyclopropylmethyl)-8-[(4-methylpiperidin-1-yl)carbonyl]-2,3,4,5-tetrahydro-1H-pyrido[4,3-b]indole

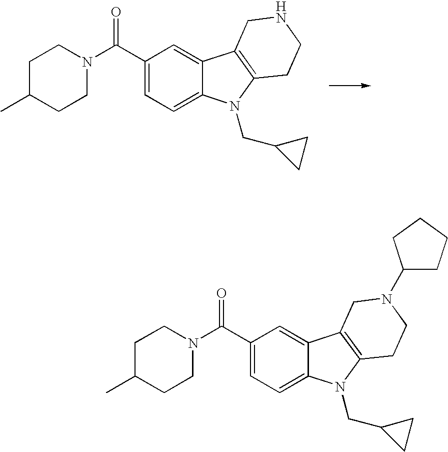

Sodium triacetoxyborohydride (120 mg, 0.57 mmol) was added to 5-(cyclopropylmethyl)-8-[(4-methylpiperidin-1-yl)carbonyl]-2,3,4,5-tetrahydro-1H-pyrido[4,3-b]indole and cyclopentanone (71 μL, 0.80 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its di-TFA salt as a yellow solid (171 mg, 66% for three steps) MS (M+1): 420.15. 1H NMR (400 MHz, CDCl3) δ ppm 0.29 (m, 2H), 0.53-0.63 (m, 2H), 0.98 (d, J=6.44 Hz, 3H), 1.05-1.19 (m, 1H), 1.58-1.73 (m, 4H), 1.84-1.94 (m, 2H), 2.01-2.18 (m, 6H), 2.94-3.07 (m, 1H), 3.34-3.52 (m, 3H), 3.54-3.66 (m, 1H), 3.85-4.04 (m, 4H,) 4.17 (d, J=14.84 Hz, 1H), 4.73 (d, J=14.84 Hz, 1H), 7.23 (d, J=8.40 Hz, 1H), 7.32 (d, J=8.40 Hz, 1H), 7.46 (s, 1H).

### Example 121

### 5-(cyclobutylmethyl)-2-cyclopentyl-8-[(4-methylpiperidin-1-yl)carbonyl]-2,3,4,5-tetrahydro-1H-pyrido[4,3-b]indole

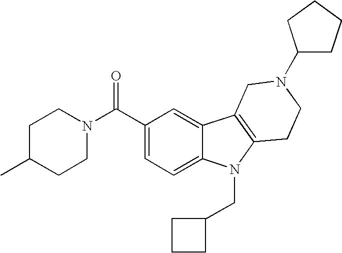

### Step A: tert-butyl 5-(cyclobutylmethyl)-8-[(4-methylpiperidin-1-yl)carbonyl]-1,3,4,5-tetrahydro-2H-pyrido[4,3-b]indole-2-carboxylate

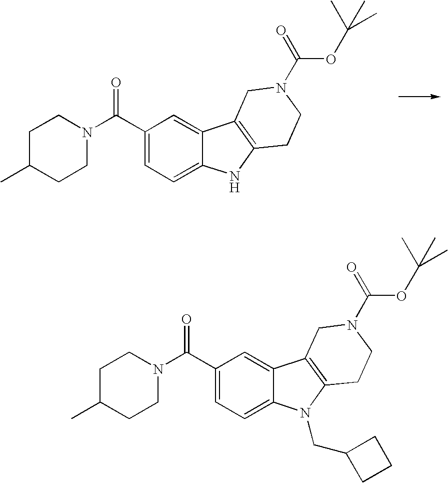

60% NaH (60 mg, 1.50 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (160 mg, 0.40 mmol) in DMF (2 mL) at room temperature under nitrogen. The mixture was stirred for 30 min, (bromomethyl)cyclobutane (80 μL, 0.71 mmol) was added at room temperature, and the reaction mixture was stirred at room temperature for 2 h. The solvent was removed in vacuo and the residue was dissolved in dichloromethane and washed with water dried over sodium sulfate. Removal of solvent gave the crude product. MS (M+1): 466.1.

### Step B: 5-(cyclobutylmethyl)-8-[(4-methylpiperidin-1-yl)carbonyl]-2,3,4,5-tetrahydro-1H-pyrido[4,3-b]indole

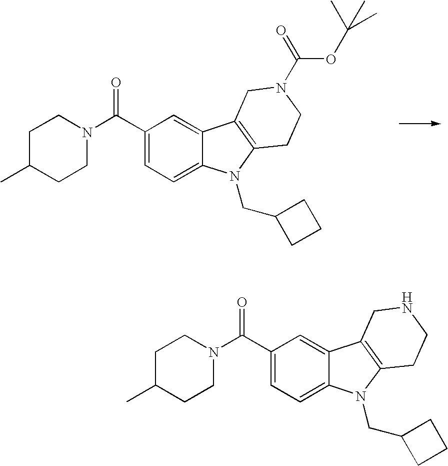

To the crude tert-butyl 5-(cyclobutylmethyl)-8-[(4-methylpiperidin-1-yl)carbonyl]-1,3,4,5-tetrahydro-2H-pyrido[4,3-b]indole-2-carboxylate in MeOH (2 mL) was added a saturated HCl solution in ether (2M, 2 mL) at room temperature. The mixture was stirred until disappearance of starting material. Removal of solvent gave the desired product as its hydrochloric acid salt form. MS (M+1): 366.38. The salt was then neutralized with MP carbonate: 0.75 g MP carbonate was added to a solution of above salt in MeOH (3 mL) and the mixture was stirred for one hour then filtered and condensed.

### Step C: 5-(cyclobutylmethyl)-2-cyclopentyl-8-[(4-methylpiperidin-1-yl)carbonyl]-2,3,4,5-tetrahydro-1H-pyrido[4,3-b]indole

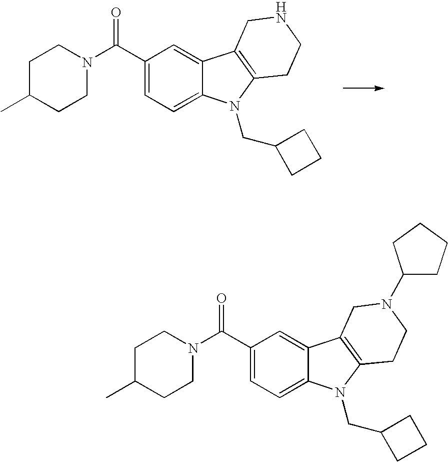

Sodium triacetoxyborohydride (120 mg, 0.57 mmol) was added to 5-(cyclobutylmethyl)-8-[(4-methylpiperidin-1-yl)carbonyl]-2,3,4,5-tetrahydro-1H-pyrido[4,3-b]indole and cyclopentanone (71 μL, 0.80 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt (128 mg, 48% for three steps). MS (M+1): 434.2. 1H NMR (400 MHz, CDCl3) δ ppm 0.98 (d, J=6.25 Hz, 3H), 1.59-1.93 (m, 12H), 1.93-2.24 (m, 7H), 2.67-2.78 (m, 1H), 2.94-3.08 (m, 1H), 3.31-3.50 (m, 3H), 3.54-3.68 (m, 1H), 3.90-4.11 (m, 3H), 4.17 (d, J=14.65 Hz, 1H), 4.74 (d, J=14.65 Hz, 1H), 7.21 (d, J=8.20 Hz, 1H), 7.31 (d, J=8.20 Hz, 1H), 7.48 (s, 1H).

### Example 122

### 2-cyclopentyl-5-[(5-methylisoxazol-4-yl)methyl]-8-[(4-methylpiperidin-1-yl)carbonyl]-2,3,4,5-tetrahydro-1H-pyrido[4,3-b]indole

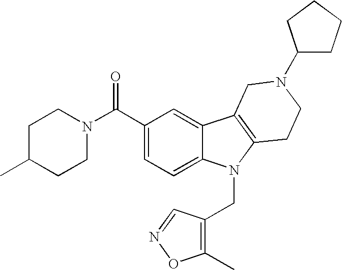

### Step A: 5-[(5-methylisoxazol-4-yl)methyl]-8-[(4-methylpiperidin-1-yl)carbonyl]-2,3,4,5-tetrahydro-1H-pyrido[4,3-b]indole

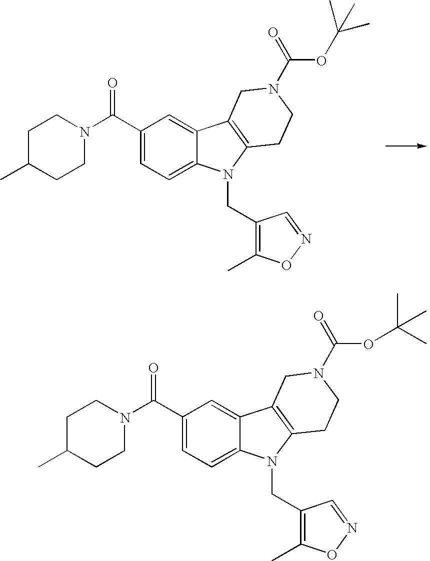

60% NaH (74 mg, 1.85 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (200 mg, 0.503 mmol) in DMF (6 mL) at room temperature under nitrogen. The mixture was stirred for 30 min, 4-(chloromethyl)-5-methylisoxazole (87 mg, 0.66 mmol) was added and the reaction mixture was stirred at room temperature for 2 h. The solvent was removed in vacuo and the residue was dissolved in dichloromethane and washed with water dried over sodium sulfate. Removal of solvent gave the crude product. MS (M+1): 493.1.

### Step B: 5-[(5-methylisoxazol-4-yl)methyl]-8-[(4-methylpiperidin-1-yl)carbonyl]-2,3,4,5-tetrahydro-1H-pyrido[4,3-b]indole

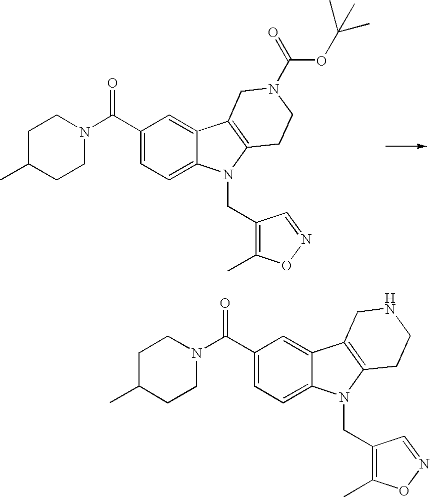

To the crude of tert-butyl 5-[(5-methylisoxazol-4-yl)methyl]-8-[(4-methylpiperidin-1-yl)carbonyl]-1,3,4,5-tetrahydro-2H-pyrido[4,3-b]indole-2-carboxylate in MeOH (2 mL) was added a saturated HCl solution in ether (2M, 2 mL) at room temperature. The mixture was stirred until disappearance of starting material. Removal of solvent gave the desired product as its hydrochloric acid salt form. MS (M+1): 393.1. The salt was then neutralized with MP carbonate: 0.75 g MP carbonate was added to a solution of above salt in MeOH (3 mL) and the mixture was stirred for one hour then filtered and condensed.

### Step C: 2-cyclopentyl-5-[(5-methylisoxazol-4-yl)methyl]-8-[(4-methylpiperidin-1-yl)carbonyl]-2,3,4,5-tetrahydro-1H-pyrido[4,3-b]indole

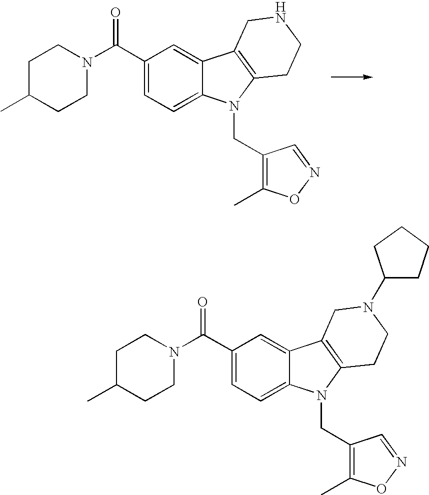

Sodium triacetoxyborohydride (180 mg, 0.85 mmol) was added to 5-[(5-methylisoxazol-4-yl)methyl]-8-[(4-methylpiperidin-1-yl)carbonyl]-2,3,4,5-tetrahydro-1H-pyrido[4,3-b]indole and cyclopentanone (90 μL, 1.02 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt (20 mg, 6% for three steps). MS (M+1): 461.1. 1H NMR (400 MHz, CDCl3) δ ppm 0.97 (d, J=6.25 Hz, 3H), 1.03-1.34 (m, 2H), 1.53-2.22 (m, 10H), 2.29 (m, 3H), 2.72-3.08 (m, 1H), 3.04 (d, J=13.47 Hz, 1H), 3.24-3.51 (m, 2H), 3.24-3.51 (m, 2H), 3.53-3.66 (m, 1H), 3.67-3.85 (m, 1H), 3.85-3.98 (m, 1H), 4.10 (d, J=13.47 Hz, 1H), 4.55-4.78 (m, 2H), 5.10-5.30 (m, 2H), 5.65 (s, 1H), 7.20 (d, J=8.20 Hz, 1H), 7.31 (d, J=8.20 Hz, 1H), 7.45 (s, 1H).

### Example 123

### 2-cyclopentyl-8-[(4-methylpiperidin-1-yl)carbonyl]-5-[(2-methyl-1,3-thiazol-4-yl)methyl]-2,3,4,5-tetrahydro-1H-pyrido[4,3-b]indole

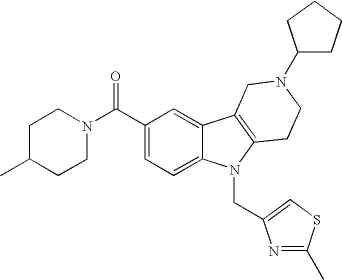

### Step A: tert-butyl 8-[(4-methylpiperidin-1-yl)carbonyl]-5-[(2-methyl-1,3-thiazol-4-yl)methyl]-1,3,4,5-tetrahydro-2H-pyrido[4,3-b]indole-2-carboxylate

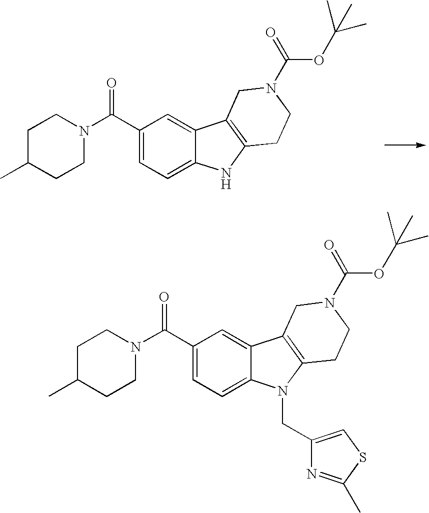

60% NaH (74 mg, 1.85 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (200 mg, 0.503 mmol) in DMF (7 mL) at room temperature under nitrogen. The mixture was stirred for 30 min, 4-(chloromethyl)-2-methyl-1,3-thiazole (120 mg, 0.81 mmol) was added at room temperature, and the reaction mixture was stirred at room temperature for 2 h. The solvent was removed in vacu and the residue was dissolved in dichloromethane and washed with water dried over sodium sulfate. Removal of solvent gave the crude product. MS (M+1): 509.1.

### Step B: 8-[(4-methylpiperidin-1-yl)carbonyl]-5-[(2-methyl-1,3-thiazol-4-yl)methyl]-2,3,4,5-tetrahydro-1H-pyrido[4,3-b]indole

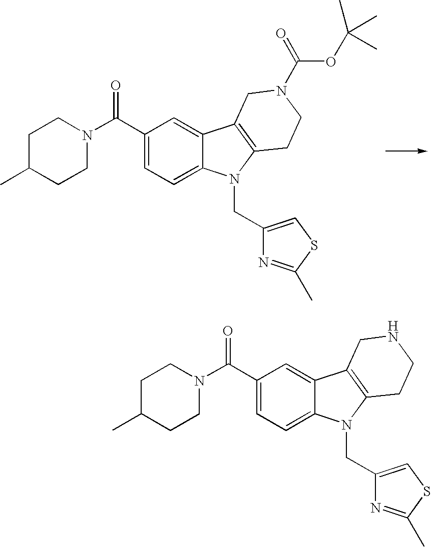

To the crude tert-butyl 8-[(4-methylpiperidin-1-yl)carbonyl]-5-[(2-methyl-1,3-thiazol-4-yl)methyl]-1,3,4,5-tetrahydro-2H-pyrido[4,3-b]indole-2-carboxylate in MeOH (2 mL) was added a saturated HCl solution in ether (2M, 3 mL) at room temperature. The mixture was stirred until disappearance of starting material. Removal of solvent gave the desired product as its hydrochloric acid salt form. MS (M+1): 409.1 The salt was then neutralized with MP carbonate: 0.75 g MP carbonate was added to a solution of above salt in MeOH (3 mL) and the mixture was stirred for one hour then filtered and condensed.

### Step C: 2-cyclopentyl-8-[(4-methylpiperidin-1-yl)carbonyl]-5-[(2-methyl-1,3-thiazol-4-yl)methyl]-2,3,4,5-tetrahydro-1H-pyrido[4,3-b]indole

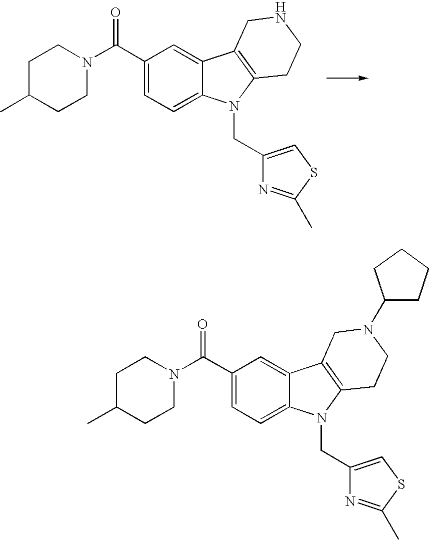

Sodium triacetoxyborohydride (180 mg, 0.85 mmol) was added to 8-[(4-methylpiperidin-1-yl)carbonyl]-5-[(2-methyl-1,3-thiazol-4-yl)methyl]-2,3,4,5-tetrahydro-1H-pyrido[4,3-b]indole and cyclopentanone (90 μL, 1.02 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) followed by recristallisation provided the desired product as its TFA salt as a yellow solid (224 mg, 63% for three steps). MS (M+1): 477.0. 1H NMR (400 MHz, CDCl3) δ ppm 0.99 (d, J=6.25 Hz, 3H), 1.48-1.91 (m, 12H), 1.96-2.10 (m, 2H), 2.70 (s, 3 H), 2.79-3.02 (m, 8H), 3.80 (s, 2H), 5.31 (d, J=0.98 Hz, 2H), 6.37 (s, 1H), 7.16-7.20 (m, 1H), 7.21-7.29 (m, 1H), 7.54 (s, 1H).

### Example 124

### 2-{2-cyclobutyl-8-[(4-methylpiperidin-1-yl)carbonyl]-1,2,3,4-tetrahydro-5H-pyrido[4,3-b]indol-5-yl}-N,N-dimethylacetamide

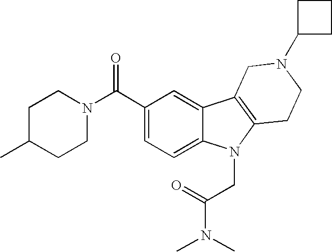

### Step A: tert-butyl 5-[2-(dimethylamino)-2-oxoethyl]-8-[(4-methylpiperidin-1-yl)carbonyl]-1,3,4,5-tetrahydro-2H-pyrido[4,3-b]indole-2-carboxylate

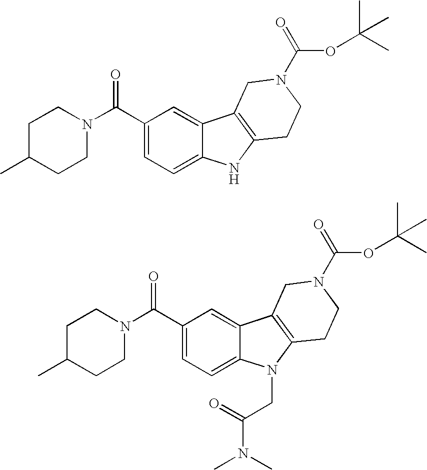

60% NaH (87 mg, 2.17 mmol) was added in one portion to a suspension of tert-butyl 8-[(4-methylpiperidin-1-yl) carbonyl]-1,3,4,5-tetrahydro-2H-pyrido[4,3-b]indole-2-carboxylate (400 mg, 1 mmol) in DMF (10 mL) at room temperature under nitrogen. The mixture was stirred for 30 min, 2-chloro-N,N-dimethylacetamide (180 μL, 1.75 mmol) was added and the reaction mixture was stirred at room temperature for 2 h. The solvent was removed in vacu and the residue was dissolved in dichloromethane and washed with water dried over sodium sulfate. Removal of solvent gave the crude product. MS (M+1): 483.2

### Step B: N,N-dimethyl-2-{8-[(4-methylpiperidin-1-yl)carbonyl]-1,2,3,4-tetrahydro-5H-pyrido[4,3-b]indol-5-yl}acetamide

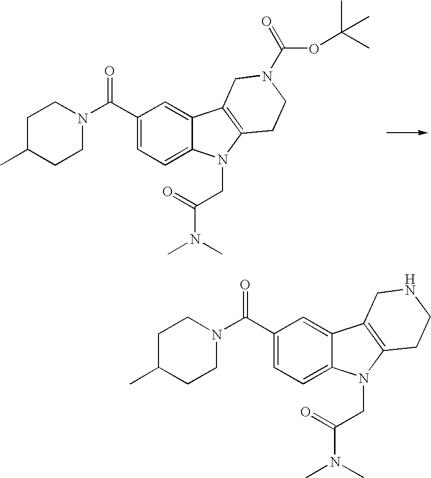

Following the similar procedure of Example 123, Step B: The title compound was prepared from tert-butyl 5-[2-(dimethylamino)-2-oxoethyl]-8-[(4-methylpiperidin-1-yl)carbonyl]-1,3,4,5-tetrahydro-2H-pyrido[4,3-b]indole-2-carboxylate. MS (M+1): 383.4.

### Step C: 2-{2-cyclobutyl-8-[(4-methylpiperidin-1-yl)carbonyl]-1,2,3,4-tetrahydro-5H-pyrido[4,3-b]indol-5-yl}-N,N-dimethylacetamide

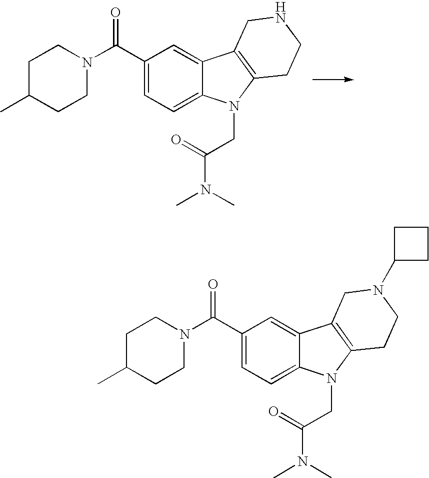

Sodium triacetoxyborohydride (86 mg, 0.40 mmol) was added to N,N-dimethyl-2-{8-[(4-methylpiperidin-1-yl)carbonyl]-1,2,3,4-tetrahydro-5H-pyrido[4,3-b]indol-5-yl}acetamide (103 mg, 0.27 mmol) and cyclobutanone (40 μL, 0.53 mmol) in dichloromethane (2 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt as a yellow solid (50 mg, 28%). MS (M+1): 437.0. 1H NMR (400 MHz, CDCL3) δ ppm 0.94 (d, J=6.44 Hz, 10H), 0.98-1.30 (m, 2H), 1.46-1.84 (m, 4H), 1.84-1.99 (m, 1H), 2.36-2.61 (m, 2H), 2.71-3.18 (m, 2H), 2.93 (s, 3H), 3.13 (s, 3H), 3.47-3.77 (m, 3H), 2.29 (d, J=7.23 Hz, 5H), 4.10-4.37 (m, 1H), 3.69 (d, J=14.45 Hz, 4H), 4.66 (d, J=17.18 Hz, 1H), 4.88 (d, J=17.38 Hz, 1H), 4.53-7.43 (m, 1H), 7.04-7.31 (m, 3H).

### Example 125

### 2-{2-cyclopentyl-8-[(4-methylpiperidin-1-yl)carbonyl]-1,2,3,4-tetrahydro-5H-pyrido[4,3-b]indol-5}-N,N-dimethylacetamide

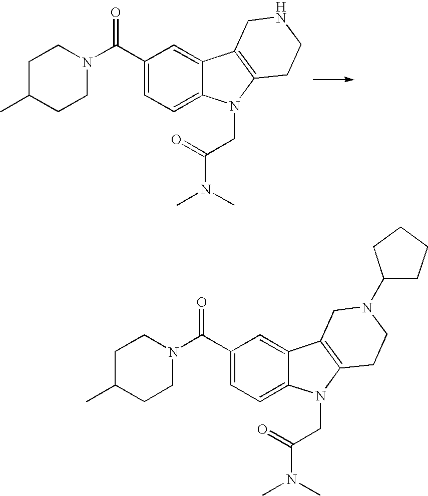

Sodium triacetoxyborohydride (86 mg, 0.40 mmol) was added to N,N-dimethyl-2-{8-[(4-methylpiperidin-1-yl)carbonyl]-1,2,3,4-tetrahydro-5H-pyrido[4,3-b]indol-5-yl}acetamide (103 mg, 0.27 mmol) and cyclopentanone (46 μL, 0.52 mmol) in dichloromethane (4 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt as a white solid (49 mg, 27%). MS (M+1): 451.0. 1H NMR (400 MHz, CDCl3) δ ppm 0.94 (d, J=6.30 Hz, 3H), 1.01-1.27 (m, 2H), 1.48-2.19 (m, 11H), 2.73-2.99 (m, 8H), 3.09-3.27 (m, 4H), 3.50 (t, J=5.9 Hz, 1H), 3.61-3.77 (m, 1H), 3.89-4.02 (m, 1H), 4.31-4.49 (m, 1H), 4.51-4.75 (m, 1H), 4.65 (d, J=17.38 Hz, 1H), 4.84 (d, J=17.38 Hz, 1H), 6.39-6.61 (m, 1H), 7.11 (s, 1H), 7.29 (s, 1H).

### Example 126

### 2-{2-cyclohexyl-8-[(4-methylpiperidin-1-yl)carbonyl]-1,2,3,4-tetrahydro-5/H-pyrido[4,3-b]indol-5-yl}-N,N-dimethylacetamide

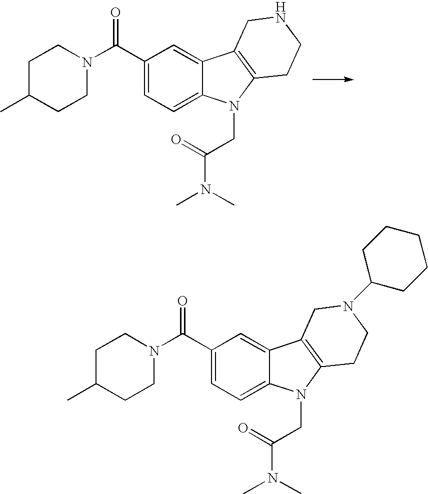

Sodium triacetoxyborohydride (86 mg, 0.40 mmol) was added to N,N-dimethyl-2-{8-[(4-methylpiperidin-1-yl)carbonyl]-1,2,3,4-tetrahydro-5H-pyrido[4,3-b]indol-5-yl}acetamide (103 mg, 0.27 mmol) and cyclohexanone (56 μL, 0.54 mmol) in dichloromethane (4 mL). The mixture was stirred at room temperature over night. More Sodium triacetoxyborohydride (60 mg, 0.28 mmol) and cyclohexanone (30 μL, 0.29 mmol) were added and the mixture stirred until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its TFA salt as a yellow-pale solid (49.7 mg, 27%). MS (MS+1): 465.0. 1H NMR (600 MHz, CDCl3) δ ppm 0.96 (d, J=6.40 Hz, 3H), 1.14-1.29 (m, 2H), 1.31-1.45 (m, 2H), 1.51-1.70 (m, 4H), 1.75 (d, J=13.06 Hz, 1H), 1.97 (d, J=13.57 Hz, 2H), 2.18 (t, J=12.42 Hz, 2H), 2.85-2.93 (m, 1H), 2.97 (s, 3H), 3.13 (s, 3H), 3.21-3.44 (m, 9H), 3.76 (d, J=7.94 Hz, 1H), 4.18 (d, J=13.82 Hz, 1H), 4.59-4.72 (m, 1H), 4.68 (d, J=17.41 Hz, 1H), 4.91 (d, J=17.41 Hz, 1H), 7.14 (d, J=12.00 Hz, 1H), 7.18 (d, J=12 Hz, 1H), 7.38 (s, 1H).

### Example 127

### methyl {2-cyclobutyl-8-[(4-methylpiperidin-1-yl)carbonyl]-1,2,3,4-tetrahydro-5H-pyrido[4,3-b]indol-5-yl}acetate

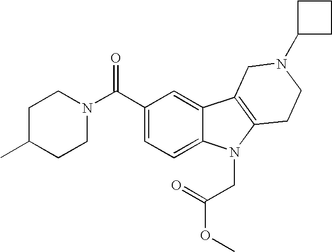

### Step A: The preparation of tert-butyl 5-(2-ethoxy-2-oxoethyl)-8-[(4-methylpiperidin-1-yl)carbonyl]-1,3,4,5-tetrahydro-2H-pyrido[4,3-b]indole-2-carboxylate

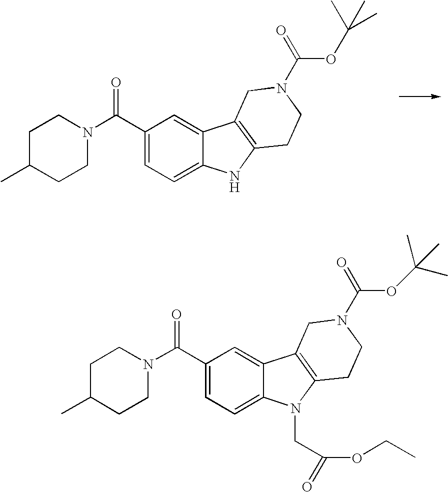

60% NaH (28 mg, 0.7 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (200 mg, 0.50 mmol) in DMF (7 mL) at room temperature under nitrogen. The mixture was stirred for 30 min, ethyl bromoactate (0.1 mL, 0.58 mmol) was added and the reaction mixture was stirred at room temperature for 2 h. The solvent was removed in vacu and the residue was dissolved in dichloromethane and washed with water dried over sodium sulfate. Removal of solvent gave the desired product. MS (M+1): 484.2.

### Step B: The preparation of Ethyl {8-[(4-methylpiperidin-1-yl)carbonyl]-1,2,3,4-tetrahydro-5H-pyrido[4,3-b]indol-5-yl}acetate

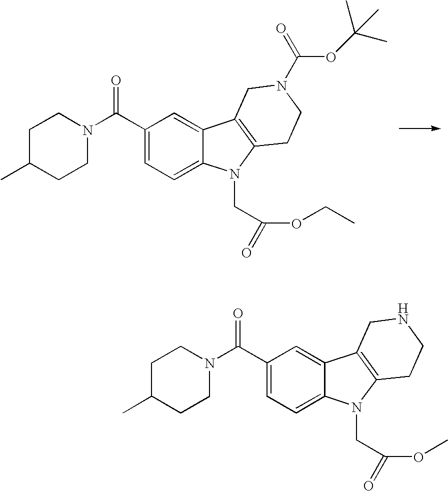

To the crude tert-butyl 5-(2-ethoxy-2-oxoethyl)-8-[(4-methylpiperidin-1-yl)carbonyl]-1,3,4,5-tetrahydro-2H-pyrido[4,3-b]indole-2-carboxylate in MeOH (2 mL) was added a saturated HCl solution in ether (2M, 2 mL) at room temperature. The mixture was stirred until disappearance of starting material. Removal of solvent gave the desired product as its hydrochloric acid salt form (during the reaction, the ethyl group exchanged with MeOH to give methylic ester). MS (M+1): 370.1. The salt was then neutralized with MP carbonate: 0.75 g MP carbonate was added to a solution of above salt in MeOH (3 mL) and the mixture was stirred for one hour then filtered and condensed.

### Step C: The preparation of Methyl {2-cyclobutyl-8-[(4-methylpiperidin-1-yl)carbonyl]-1,2,3,4-tetrahydro-5H-pyrido[4,3-b]indol-5-yl}acetate

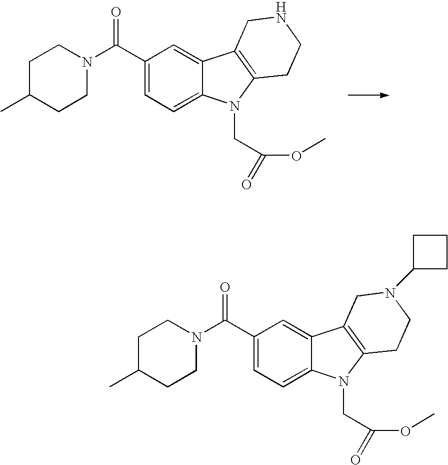

Sodium triacetoxyborohydride (46 mg, 0.22 mmol) was added to ethyl {8-[(4-methylpiperidin-1-yl)carbonyl]-1,2,3,4-tetrahydro-5H-pyrido[4,3-b]indol-5-yl}acetate (53 mg, 0.14 mmol) and cyclobutanone (21 μL, 0.28 mmol) in dry dichloromethane (2 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its di-TFA salt as a yellow solid (19.6 mg, 22%). MS (MS+1): 424.0. 1H NMR (400 MHz, CDCl3) δ ppm 0.97 (d, J=6.44 Hz, 3H), 1.03-1.31 (m, 2H), 1.47-1.89 (m, 4H), 1.86-2.04 (m, 1H), 2.22-2.38 (m, 2H), 2.47-2.67 (m, 2H), 2.72-3.10 (m, 3H), 3.14-3.34 (m, 2H), 3.56-3.82 (m, 3H), 3.74 (s, 3H), 3.87 (d, J=14.65 Hz, 1H), 4.44-4.86 (m, 4H), 7.16-7.27 (m, 2H), 7.38 (bs, 1H).

### Example 128

### methyl {2-cyclopentyl-8-[(4-methylpiperidin-1-yl)carbonyl]-1,2,3,4-tetrahydro-5H-pyrido[4,3-b]indol-5-yl}acetate

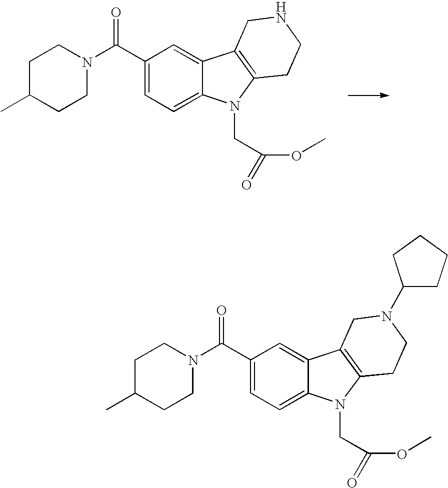

Sodium triacetoxyborohydride (46 mg, 0.22 mmol) was added to ethyl {8-[(4-methylpiperidin-1-yl)carbonyl]-1,2,3,4-tetrahydro-5H-pyrido[4,3-b]indol-5-yl}acetate (53 mg, 0.14 mmol) and cyclopentanone (25 μL, 0.28 mmol) in dry dichloromethane (2 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over reverse phase HPLC (5-75% CH₃CN) provided the desired product as its TFA salt as a white solid (22 mg, 24%). MS (M+1): 438.0. 1H NMR (400 MHz, CDCl3) δ ppm 0.96 (d, J=6.25 Hz, 3H), 1.02-1.31 (m, 2H), 1.52-2.21 (m, 13H), 2.71-3.08 (m, 3H), 3.19-3.42 (m, 2H), 3.48-3.64 (m, 1H), 3.74 (s, 3H), 3.80-3.94 (m, 1H), 4.09 (d, J=13.28 Hz, 1H), 4.48-4.87 (m, 3H), 7.20 (s, 2H), 7.41 (s, 1H).

### Example 129

### {2-cyclopentyl-8-[(4-methylpiperidin-1-yl)carbonyl]-1,2,3,4-tetrahydro-5H-pyrido[4,3-b]indol-5-yl}acetic acid

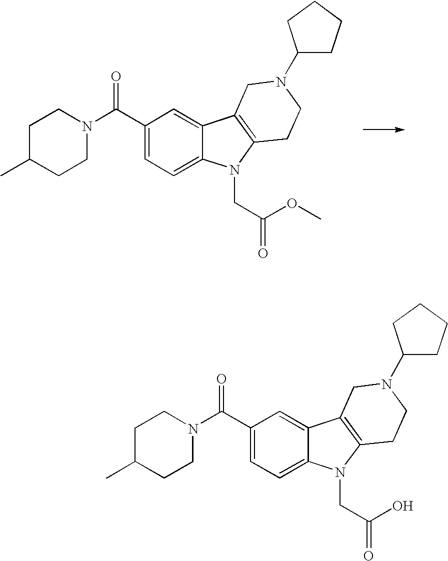

To a solution of methyl {2-cyclopentyl-8-[(4-methylpiperidin-1-yl)carbonyl]-1,2,3,4-tetrahydro-5H-pyrido[4,3-b]indol-5-yl}acetate (obtained from 0.2 g of tert-butyl 5-(2-ethoxy-2-oxoethyl)-8-[(4-methylpiperidin-1-yl)carbonyl]-1,3,4,5-tetrahydro-2H-pyrido[4,3-b]indole-2-carboxylate, as described above) was added to a mixture of MeOH/H₂O/THE (3 mL of each) and NaOH (96 mg, 0.24 mmol) and the mixture was stirred at room temperature. The solvents were then removed under reduced pressure and the PH of residue adjusted to 5. The formed precipitates were recovered. Purification of this later over reverse phase HPLC (5-75% CH₃CN) provided a yellow solid (24 mg, 7.3% for four steps). MS (M+1): 424.0. 1H NMR (600 MHz, CDCl3) δ ppm 0.94 (bs, 3H), 1.11-1.22 (m, 1H), 1.23-1.42 (m, 2H), 1.54-2.10 (m, 9H), 2.13-2.31 (m, 2H), 2.55-2.62 (m, 1H), 2.74-2.97 (m, 2H), 2.98-3.30 (m, 3H), 3.53-3.66 (m, 1H), 3.68-3.87 (m, 2H), 3.94-4.15 (m, 1H), 4.56-4.84 (m, 2H), 7.01-7.28 (m, 2H), 8.21 (m, 1H), 11.06-11.18 (m, 1H).

### Example 130

### Methyl {2-cyclohexyl-8-[(4-methylpiperidin-1-yl)carbonyl]-1,2,3,4-tetrahydro-5H-pyrido[4,3-b]indol-5-yl}acetate

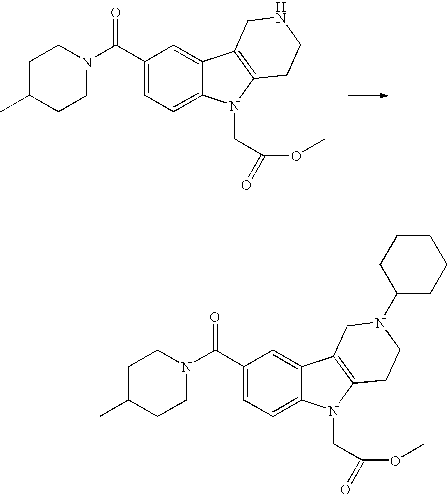

Sodium triacetoxyborohydride (80 mg, 0.38 mmol) was added to ethyl {8-[(4-methylpiperidin-1-yl)carbonyl]-1,2,3,4-tetrahydro-5H-pyrido[4,3-b]indol-5-yl}acetate (93 mg, 0.24 mmol) and cyclohexanone (60 μL, 0.58 mmol) in dry dichloromethane (3 mL). The mixture was stirred at room temperature. More sodium triacetoxyborohydride (60 mg, 0.28 mmol) and cyclohexanone (30 μL, 0.29 mmol) were added but still the conversion was not complete and about 20% of starting material left. The mixture was then diluted with dichloromethane and washed with saturated NaHCO₃ solution, dried over Na₂SO₄ and condensed under reduced pressure. Purification of the crude over reverse phase HPLC (5-75% CH₃CN) provided the desired product as its TFA salt as a white solid (18 mg, 11%). MS (M+1): 452.0. 1H NMR (600 MHz, CDCL3) δ ppm 0.97 (d, J=6.40 Hz, 3H), 1.03-1.32 (m, 3H), 1.33-1.46 (m, 2H), 1.52-1.70 (m, 4H), 1.75 (d, J=9.73 Hz, 2H), 1.97 (d, J=12.80 Hz, 2H), 2.14-2.25 (m, 2H), 2.75-3.08 (m, 3H), 3.34-3.47 (m, 2 H), 3.73 (m, 3H), 3.79-3.89 (m, 1H), 4.23 (d, J=13.82 Hz, 1H) 4.45-4.84 (m, 3H) 5.08-5.40 (m, 3H), 7.22 (m, 2H), 7.44 (m, 1H).

### Example 131

### methyl 5-({2-cyclopentyl-8-[(4-methylpiperidin-1-yl)carbonyl]-1,2,3,4-tetrahydro-5H-pyrido[4,3-b]indol-5-yl}methyl)-2-furoate

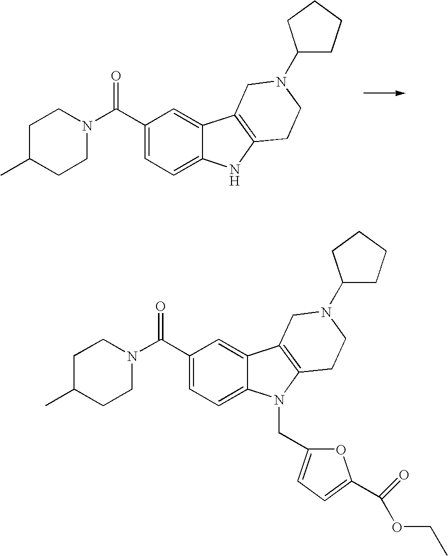

60% NaH (17 mg, 0.42 mmol) was added in one portion to a suspension of the crude of 2-cyclopentyl-8-[(4-methylpiperidin-1-yl)carbonyl]-2,3,4,5-tetrahydro-1H-pyrido[4,3-b]indole in DMF (3 mL) at room temperature under nitrogen. The mixture was stirred for 30 min, ethyl 5-(chloromethyl)-2-furoate (66 μL, 0.43 mmol) was added and the reaction mixture was stirred at room temperature for 2 h. The solvent was removed in vacu and the residue was dissolved in dichloromethane and washed with water, dried over sodium sulfate then condensed under reduced pressure. The purification of the crude by reverse phase HPLC (5-75% CH₃CN) provided the desired compound as a white solid as its di-TFA salt (97.86 mg, 17% for three steps). MS (M+1): 518.0. 1H NMR (400 MHz, CDCl3) δ ppm 0.95 (d, J=6.25 Hz, 3H), 0.99-1.26 (m, 2H), 1.30 (t, J=7.13 Hz, 3H), 1.48-2.08 (m, 9H), 2.09-2.22 (m, 2H), 2.67-3.04 (m, 2H), 3.05-3.20 (m, 1H), 3.23-3.43 (m, 2H), 3.54-3.78 (m, 2H), 3.82-3.94 (m, 1H), 4.11 (d, J=14.06 Hz, 1H), 4.27 (q, J=7.03 Hz, 2H), 4.50-4.72 (m, 2H), 5.18 (s, 2H), 6.16 (s, 1H), 7.00 (d, J=3.40 Hz, 1H), 7.12-7.20 (m, 1H), 7.24-7.30 (m, 1H), 7.37 (bs, 1H).

### Example 132

### 5-({2-cyclopentyl-8-[(4-methylpiperidin-1-yl)carbonyl]-1,2,3,4-tetrahydro-5H-pyrido[4,3-b]indol-5-yl}methyl)-2-furoic acid

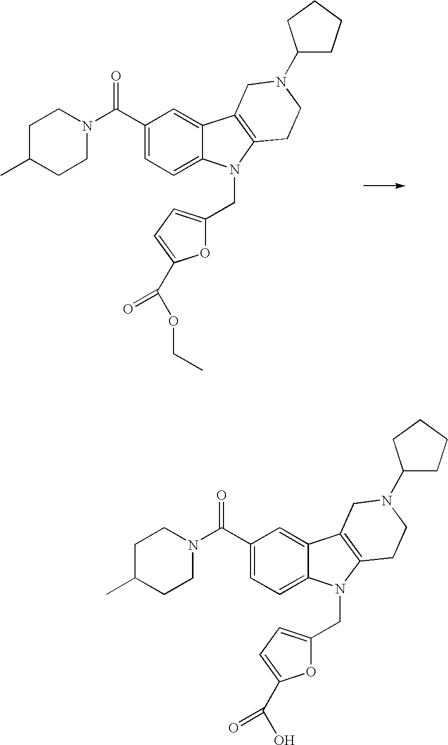

To a solution of methyl 5-({2-cyclopentyl-8-[(4-methylpiperidin-1-yl)carbonyl]-1,2,3,4-tetrahydro-5H-pyrido[4,3-b]indol-5-yl}methyl)-2-furoate (33 mg, 0.04 mmol) in a mixture of MeOH/THF/H₂O (1 mL of each) was added NaOH (17 mg 0.42 mmol) and the mixture stirred over night at room temperature. The solvent was then removed and the Ph of residue adjusted to 5. The precipitate was collected and then purified by reverse phase HPLC (5-75% CH₃CN) to provide the desired compound as its TFA salt (8 mg, 25%). MS (MS+1): 490.0.

### Example 133

### 2-cyclopentyl-8-[(4-methylpiperidin-1-yl)carbonyl]-5-[4-(methylthio)benzyl]-2,3,4,5-tetrahydro-1H-pyrido[4,3-b]indole

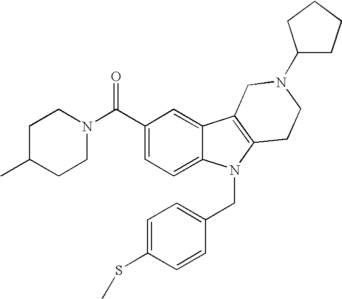

### Step A: Preparation of 1-(chloromethyl)-4-(methylthio)benzene

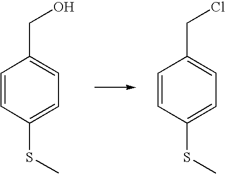

To a solution of 4-(methylthio)benzyl alcohol (1 g, 6.48 mmol) in dry dichloromethane (2 mL) was added drop by drop thionyl chloride (0.2 mL) and the mixture stirred at room temperature for 2 hours and then condensed. The chloride was used without any further purification (0.6 g, 54%). 1H NMR (400 MHz, CDCl3) δ ppm 2.49 (s, 3H) 4.57 (s, 2H) 7.22-7.28 (m, 2H) 7.29-7.36 (m, 2H).

### Step B: tert-butyl 8-[(4-methylpiperidin-1-yl)carbonyl]-5-[4-(methylthio)benzyl]-1,3,4,5-tetrahydro-2H-pyrido[4,3-b]indole-2-carboxylate

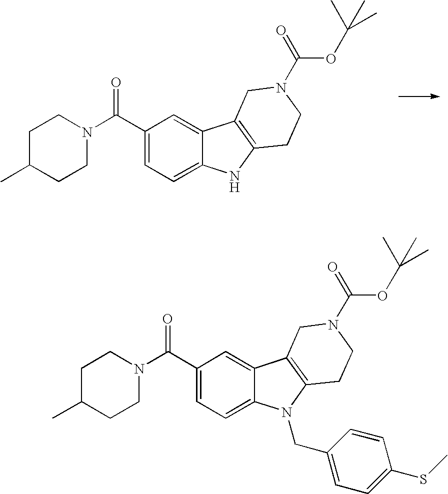

60% NaH (74 mg, 1.85 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (200 mg, 0.50 mmol) in DMF (7 mL) at room temperature under nitrogen. The mixture was stirred for 30 min, 1-(chloromethyl)-4-(methylthio)benzene (100 mg, 0.58 mmol) was added at room temperature, and the reaction mixture was stirred at room temperature for 2 h. The solvent was removed in vacuo and the residue was dissolved in dichloromethane and washed with water dried over sodium sulfate. Removal of solvent gave the crude product. MS (M+1): 534.13.

### Step C: 8-[(4-methylpiperidin-1-yl)carbonyl]-5-[4-(methylthio)benzyl]-2,3,4,5-tetrahydro-1H-pyrido[4,3-b]indole

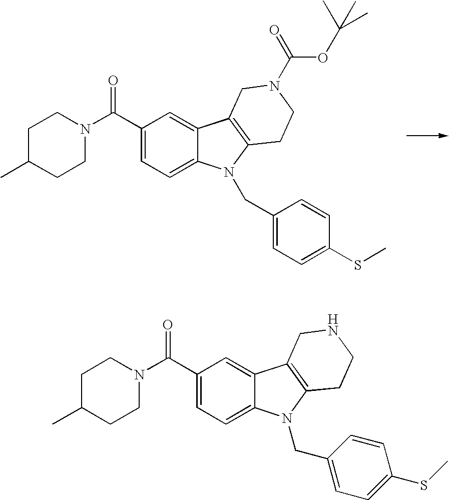

To the crude of tert-butyl 8-[(4-methylpiperidin-1-yl)carbonyl]-5-[4-(methylthio)benzyl]-1,3,4,5-tetrahydro-2H-pyrido[4,3-b]indole-2-carboxylate in MeOH (2 mL) was added a saturated HCl solution in ether (2M, 2 mL) at room temperature. The mixture was stirred until disappearance of starting material. Removal of solvent gave the desired product 8-[(4-methylpiperidin-1-yl)carbonyl]-5-[4-(methylthio)benzyl]-2,3,4,5-tetrahydro-1H-pyrido[4,3-b]indole as its hydrochloric acid salt form. MS (M+1): 434.09. The salt was then neutralized with MP carbonate: 0.76 g MP carbonate was added to a solution of above salt in MeOH (3 mL) and the mixture was stirred for one hour then filtered and condensed.

### Step D: 2-cyclopentyl-8-[(4-methylpiperidin-1-yl)carbonyl]-5-[4-(methylthio)benzyl]-2,3,4,5-tetrahydro-1H-pyrido[4,3-b]indole

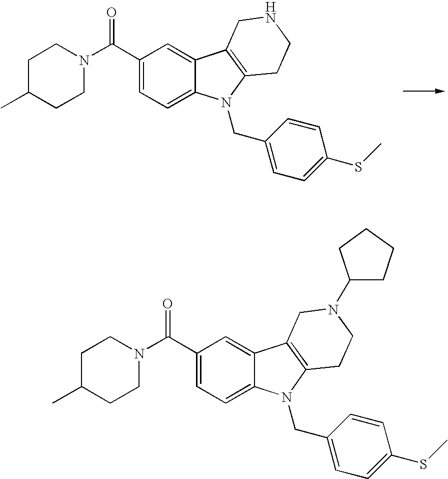

Sodium triacetoxyborohydride (128 mg, 0.604 mmol) was added to 8-[(4-methylpiperidin-1-yl)carbonyl]-5-[4-(methylthio)benzyl]-2,3,4,5-tetrahydro-1H-pyrido[4,3-b]indole and cyclopentanone (89 μL, 1.01 mmol) in dry dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its di-TFA salt (43 mg, 14% for three steps). MS (M+1): 502.15. 1H NMR (400 MHz, CDCl3) δ ppm 0.96 (d, J=6.25 Hz, 3H), 1.52-2.21 (m, 11H), 2.89 (s, 3H), 2.84 (d, J=12.89 Hz, 2H), 3.21-3.36 (m, 2H), 3.50-3.62 (m, 1H), 3.67-3.90 (m, 2H), 4.14 (d, J=13.86 Hz, 1H), 4.52-4.76 (m, 2H), 5.08-5.23 (m, 2 H), 6.83 (d, J=8.20 Hz, 2H), 7.12 (d, J=8.20 Hz, 2H), 7.08-7.22 (m, 2H), 7.46 (s, 1 H).

### Example 134

### 2-cyclopentyl-8-[(4-methylpiperidin-1-yl)carbonyl]-5-[4-(methylthio)benzyl]-2,3,4,5-tetrahydro-1H-pyrido[4,3-b]indole 2-oxide

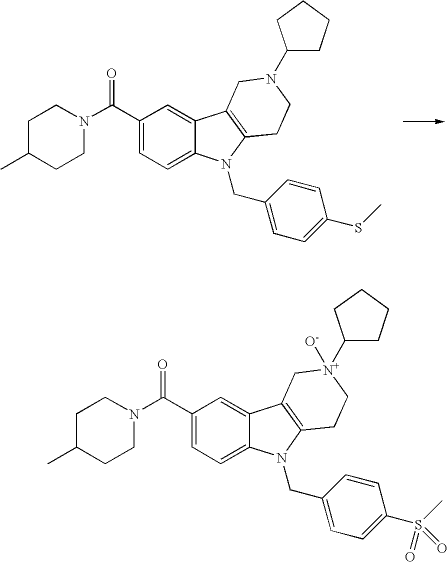

To a solution of 2-cyclopentyl-8-[(4-methylpiperidin-1-yl)carbonyl]-5-[4-(methylthio)benzyl]-2,3,4,5-tetrahydro-1H-pyrido[4,3-b]indole (12 mg, 0.019 mmol) in MeOH (2 mL) was added oxone (54 mg) and the mixture was stirred over night at room temperature. The mixture was then filtered and condensed under reduced pressure and then purified by reverse phase HPLC (5-75% CH₃CN) to provide the title compound as its TFA salt (4 mg). MS (M+1): 550.1. 1H NMR (400 MHz, CDCl3) δ ppm 0.98 (d, J=6.25 Hz, 3H), 1.56-2.19 (m, 11H), 2.36-2.59 (m, 1H), 3.05 (s, 3H), 3.14-3.67 (m, 10H), 3.92 (t, J=9.57 Hz, 1H), 4.00 (d, J=14.65 Hz, 1 H), 4.82-5.05 (m, 2H), 6.51 (d, J=8.59 Hz, 1H), 7.34 (d, J=8.20 Hz, 2H), 7.54 (dd, J=8.49, 1.66 Hz, 1H), 7.72 (s, 1H), 7.88 (d, J=8.20 Hz, 2H).

### Example 135

### 2-cyclopentyl-8-[(4-methylpiperidin-1-yl)carbonyl]-5-[4-(methylsulfonyl)benzyl]-2,3,4,5-tetrahydro-1H-pyrido[4,3-b]indole

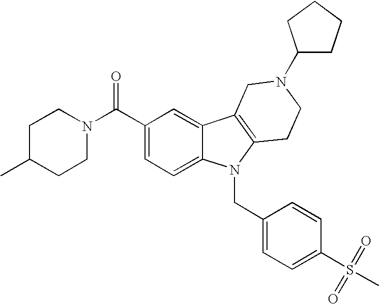

### Step A: tert-butyl 8-[(4-methylpiperidin-1-yl)carbonyl]-5-[4-(methylsulfonyl)benzyl]-1,3,4,5-tetrahydro-2H-pyrido[4,3-b]indole-2-carboxylate

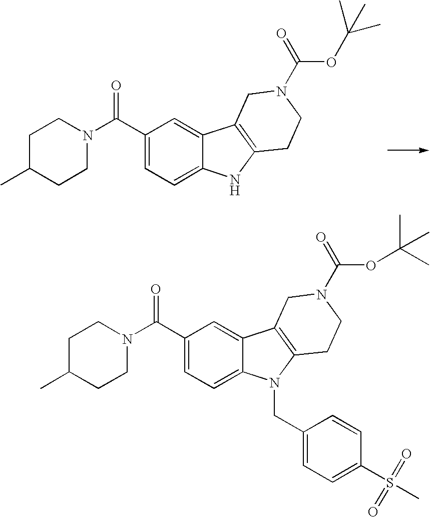

60% NaH (74 mg, 1.85 mmol) was added in one portion to a suspension of tert-butyl 8-[(4-methylpiperidin-1-yl)carbonyl]-1,3,4,5-tetrahydro-2H-pyrido[4,3-b]indole-2-carboxylate (200 mg, 0.503 mmol) in DMF (7 mL) at room temperature under nitrogen. The mixture was stirred for 30 min, 1-(chloromethyl)-4-(methylsulfonyl)benzene (123 mg, 0.60 mmol) was added at room temperature, and the reaction mixture was stirred at room temperature for 2 h. The solvent was removed in vacuo and the residue was dissolved in dichloromethane and washed with water dried over sodium sulfate. Removal of solvent gave the crude product. MS (M+1): 566.48.

### Step B: 8-[(4-methylpiperidin-1-yl)carbonyl]-5-[4-(methylsulfonyl)benzyl]-2,3,4,5-tetrahydro-1H-pyrido[4,3-b]indole

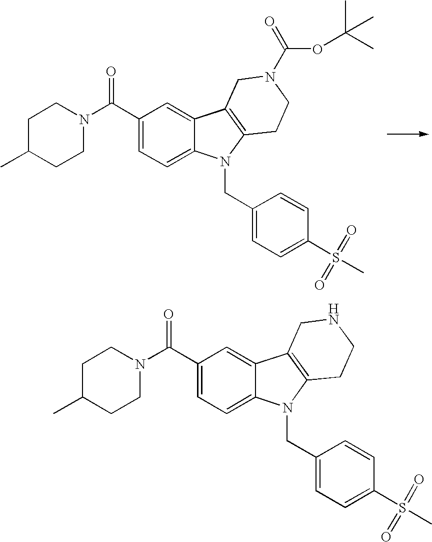

The crude tert-butyl 8-[(4-methylpiperidin-1-yl)carbonyl]-5-[4-ethylsulfonyl)benzyl]-1,3,4,5-tetrahydro-2H-pyrido[4,3-b]indole-2-carboxylate was dissolved in dichloromethane (3 mL) and trifluoroacetic acid (1 mL) was added to the reaction mixture. The mixture was stirred until disappearance of starting material. Removal of excess trifluoroacetic acid in vacuo gave crude 8-[(4-methylpiperidin-1-yl)carbonyl]-5-[4-(methylsulfonyl)benzyl]-2,3,4,5-tetrahydro-1H-pyrido[4,3-b]indole as its trifluoroacetic acid salt form. The salt was then neutralized with MP carbonate: 0.75 g MP carbonate was added to a solution of above salt in MeOH (3 mL) and the mixture was stirred for one hour then filtered and condensed. MS (M+1): 466.4.

### Step C: 2-cyclopentyl-8-[(4-methylpiperidin-1-yl)carbonyl]-5-[4-(methylsulfonyl)benzyl]-2,3,4,5-tetrahydro-1H-pyrido[4,3-b]indole

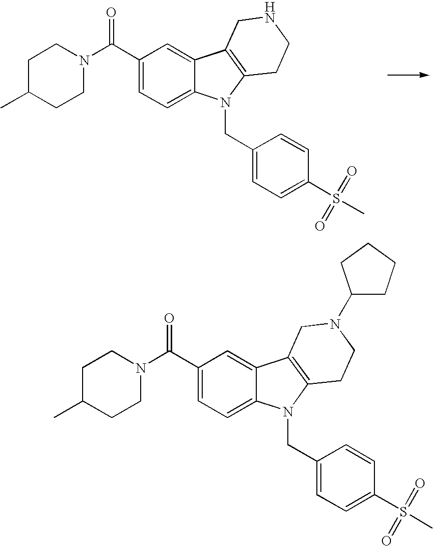

Sodium triacetoxyborohydride (80 mg, 0.38 mmol) was added to half of the crude of 8-[(4-methylpiperidin-1-yl)carbonyl]-5-[4-(methylsulfonyl)benzyl]-2,3,4,5-tetrahydro-1H-pyrido[4,3-b]indole and cyclopentanone (50 μL, 56 mmol) in dichloromethane (2 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75% CH₃CN) provided the desired product as its di-TFA salt as a yellow-pale solid (40 mg, 10% for three steps). MS (M+1): 534.0. 1H NMR (600 MHz, CDCL3) δ ppm 1.07 (d, J=6.40 Hz, 3H), 1.17-1.40 (m, 2H), 1.63-2.06 (m, 8H), 2.06-2.38 (m, 4H), 2.96 (d, J=18.18 Hz, 2H), 3.09 (s, 3H), 3.32-3.52 (m, 2H), 3.64-3.78 (m, 1H), 3.89 (bs, 1H), 3.99-4.09 (m, 1H), 4.25 (d, J=13.82 Hz, 1H), 4.76 (bs, 1H), 4.86 (d, J=13.31 Hz, 1H), 5.42 (q, J=17.6 Hz, 2H), 7.15 (d, J=7.94 Hz, 2H), 7.20-7.32 (m, 1H), 7.35 (bs, 1H), 7.62 (s, 1H), 7.90 (d, J=7.94 Hz, 2H).

### Example 136

### 8-[(4-methylpiperidin-1-yl)carbonyl]-5-(4-nitrophenyl)-2-(tetrahydro-2H-pyran-4-yl)-2,3,4,5-tetrahydro-1H-pyrido[4,3-b]indole

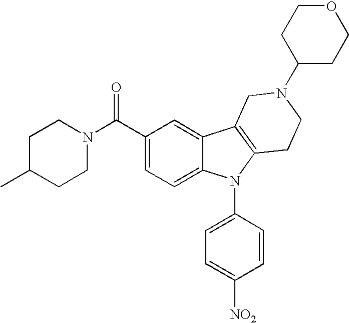

### Step A: tert-butyl 8-[(4-methylpiperidin-1-yl)carbonyl]-5-(4-nitrophenyl)-1,3,4,5-tetrahydro-2H-pyrido[4,3-b]indole-2-carboxylate

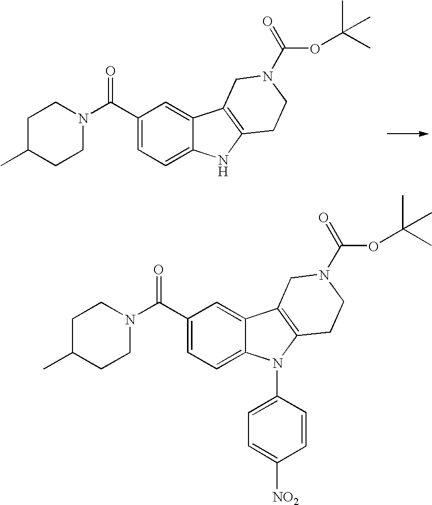

60% NaH (70 mg, 1.75 mmol) was added in one portion to a suspension of tent-butyl 8-[(4-methylpiperidin-1-yl)carbonyl]-1,3,4,5-tetrahydro-2H-pyrido[4,3-b]indole-2-carboxylate (200 mg, 0.503 mmol) in DMF (5 mL) at room temperature under nitrogen. The mixture was stirred for 30 min, 1-fluoro-4-nitrobenzene (70 μL, 0.66 mmol) was added and the reaction mixture was stirred at room temperature for 2 h. The solvent was removed in vacuo and the residue was dissolved in dichloromethane and washed with water dried over sodium sulfate. Removal of solvent gave the crude product. MS (M+1): 519.5.

### Step B: 8-[(4-methylpiperidin-1-yl)carbonyl]-5-(4-nitrophenyl)-2,3,4,5-tetrahydro-1H-pyrido[4,3-b]indole

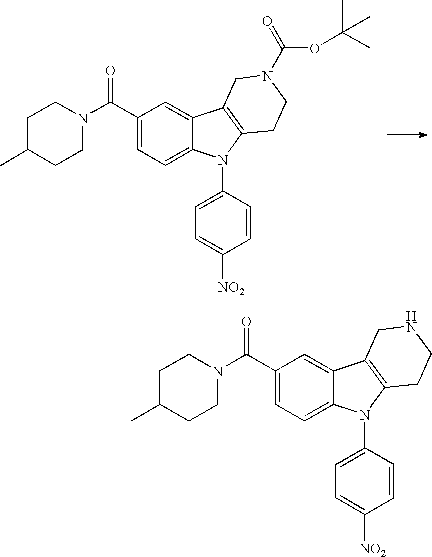

tert-butyl 8-[(4-methylpiperidin-1-yl)carbonyl]-5-(4-nitrophenyl)-1,3,4,5-tetrahydro-2H-pyrido[4,3-b]indole-2-carboxylate was dissolved in dichloromethane (5 mL) and trifluoroacetic acid (0.5 mL) was added to the reaction mixture. The mixture was stirred until disappearance of starting material. Removal of excess trifluoroacetic acid in vacuo gave crude product as its trifluoroacetic acid salt form. The salt was then neutralized with MP carbonate: 0.75 g MP carbonate was added to a solution of above salt in MeOH (3 mL) and the mixture was stirred for one hour then filtered and condensed (0.4 g). MS (M+1): 419.4.

### Step C: 8-[(4-methylpiperidin-1-yl)carbonyl]-5-(4-nitrophenyl)-2-(tetrahydro-2H-pyran-4-yl)-2,3,4,5-tetrahydro-1H-pyrido[4,3-b]indole

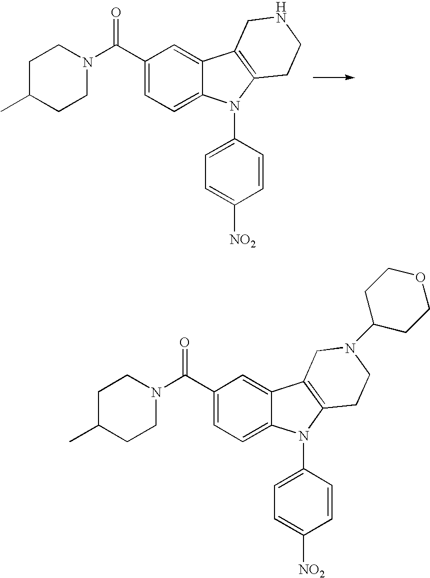

Sodium triacetoxyborohydride (80 mg, 0.38 mmol) was added to 8-[(4-methylpiperidin-1-yl)carbonyl]-5-(4-nitrophenyl)-2,3,4,5-tetrahydro-1H-pyrido[4,3-b]indole (80 mg, 0.19 mmol) and tetrahydro-4H-pyran-4-one (35 μL, 0.38 mmol) in dichloromethane (4 mL). The mixture was stirred at room temperature until no more starting material was observed. The mixture was then diluted with dichloromethane and washed with saturated NaHCO₃ solution, dried over Na₂SO₄ and condensed under reduced pressure. Purification of the crude over preparative HPLC (5-75%) provided the desired product as its TFA salt (18 mg, 14%). MS (M+1): 503.5. 1H NMR (600 MHz, CDCl3): δ ppm 0.98 (d, J=6.40 Hz, 3H), 1.05-1.31 (m, 2H), 1.51 (t, J=6.91 Hz, 1H), 1.56-1.86 (m, 3H), 1.95-2.18 (m, 4H), 2.73-2.92 (m, 2H), 2.97-3.11 (m, 1H), 3.53-3.39 (m, 1H), 3.48 (t, J=11.65 Hz, 2H), 3.53-3.65 (m, 1H), 3.68-3.82 (m, 2H), 3.83-3.95 (m, 1H), 4.16 (d, J=9.22 Hz, 2H), 4.60-4.77 (m, 2H), 7.20-7.33 (m, 2H), 7.51-7.63 (m, 3H), 8.43 (d, J=8.19 Hz, 2H).

### Example 137

### {4-[8-[(4-methylpiperidin-1-yl)carbonyl]-2-(tetrahydro-2H-pyran-4-yl)-1,2,3,4-tetrahydro-5H-pyrido[4,3-b]indol-5-yl]phenyl}amine

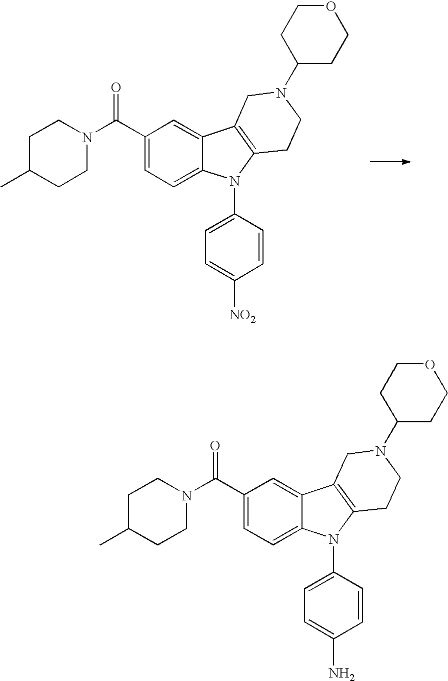

To a solution of 8-[(4-methylpiperidin-1-yl)carbonyl]-5-(4-nitrophenyl)-2-(tetrahydro-2H-pyran-4-yl)-2,3,4,5-tetrahydro-1H-pyrido[4,3-b]indole.2TFA (170 mg, 0.24 mmol) in MeOH (8 mL) was added Pd/C and the mixture placed under H₂ (30 psi) and stirred over night at room temperature. The mixture was then filtered and condensed under reduced pressure and was purified by reversed-phase HPLC (5-75% CH3CN) to provide the desired compound as its TFA salt (153 mg, 94%). MS (M+1): 474.36. 1H NMR (400 MHz, CD3OD) δ ppm 0.84-0.92 (m, 1H), 0.88 (d, J=6.45 Hz, 3H), 0.95-1.19 (m, 3H), 1.41-1.74 (m, 3H), 1.76-1.91 (m, 1H), 2.08 (d, J=12.11 Hz, 2H), 2.66-3.12 (m, 4H), 3.35-3.53 (m, 1H), 3.41 (t, J=11.32 Hz, 2H), 3.57-3.94 (m, 2H), 4.03 (dd, J=11.62, 4.00 Hz, 2H), 4.40-4.57 (m, 1H), 4.63-4.79 (m, 2H), 6.95-7.03 (m, 2H), 7.08-7.18 (m, 4H), 7.54-7.5 (m, 1H).

### Example 138

### N-{-4-[8-[(4-methylpiperidin-1-yl)carbonyl]-2-(tetrahydro-2H-pyran-4-yl)-1,2,3,4-tetrahydro-5H-pyrido[4,3-b]indol-5-yl]phenyl}acetamide

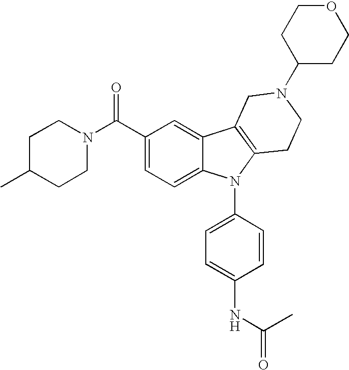

{4-[8-[(4-methylpiperidin-1-yl)carbonyl]-2-(tetrahydro-2H-pyran-4-yl)-1,2,3,4-tetrahydro-5H-pyrido[4,3-b]indol-5-yl]phenyl}amine (33 mg, 0.05 mmol) was dissolved in acetic anhydride (0.5 mL) and pyridine (0.5 mL) and the mixture stirred at room temperature until no more starting material was observed. The mixture was then condensed under reduced pressure and then diluted in dichloromethane and was washed with NaHCO₃, water and a saturated solution of NaCl. After drying and condensation under reduced pressure the crude was purified by reversed-phase HPLC (5-75%) to provide the desired compound as its TFA salt (11.62 mg, 34%). MS (M+1): 515.5. 1H NMR (600 MHz, CDCl3) δ ppm 0.97 (d, J=6.14 Hz, 3H), 0.79-1.37 (m, 4H), 1.45-1.89 (m, 3H), 1.92-2.34 (m, 7H), 2.59-2.71 (m, 1H), 2.76-2.92 (m, 1H), 2.93-3.07 (m, 1H), 3.08-3.28 (m, 2H), 3.42-3.56 (m, 2H), 3.61-3.83 (m, 3H), 4.09-4.22 (m, 2H), 4.28 (d, J=12.54 Hz, 1H), 4.57-4.80 (m, 2H), 5.88-6.27 (m, 3H), 6.86-7.10 (m, 4H), 7.42-7.72 (m, 3H).

### Example 139

### 4-{2-cyclopentyl-8-[(4-methylpiperidin-1-yl)carbonyl]-1,2,3,4-tetrahydro-5H-pyrido[4,3-b]indol-5-yl}phenyl)amine

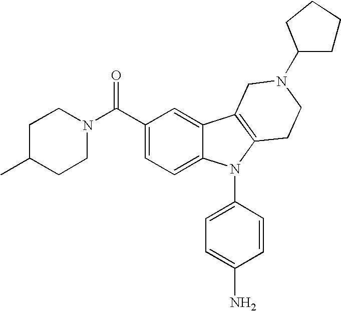

### Step A: 2-cyclopentyl-8-[(4-methylpiperidin-1-yl)carbonyl]-5-(4-nitrophenyl)-2,3,4,5-tetrahydro-1H-pyrido[4,3-b]indole

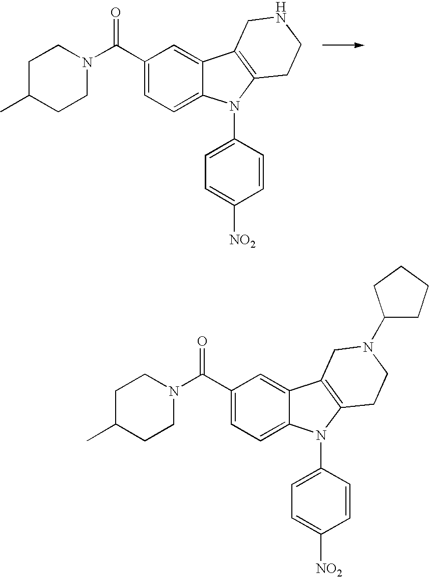

Sodium triacetoxyborohydride (81 mg, 0.38 mmol) was added to 8-[(4-methylpiperidin-1-yl)carbonyl]-5-(4-nitrophenyl)-2,3,4,5-tetrahydro-1H-pyrido[4,3-b]indole (80 mg, 0.19 mmol) cyclopentanone (34 μL, 0.38 mmol) in dichloromethane (3 mL). The mixture was stirred at room temperature until no more starting material was observed. The mixture was then diluted with dichloromethane and washed with saturated NaHCO₃ solution, dried over Na₂SO₄ and condensed under reduced pressure. MS (M+1): 487.5.

### Step B: The preparation of 4-{2-cyclopentyl-8-[(4-methylpiperidin-1-yl)carbonyl]-1,2,3,4-tetrahydro-5H-pyrido[4,3-b]indol-5-yl}phenyl)amine

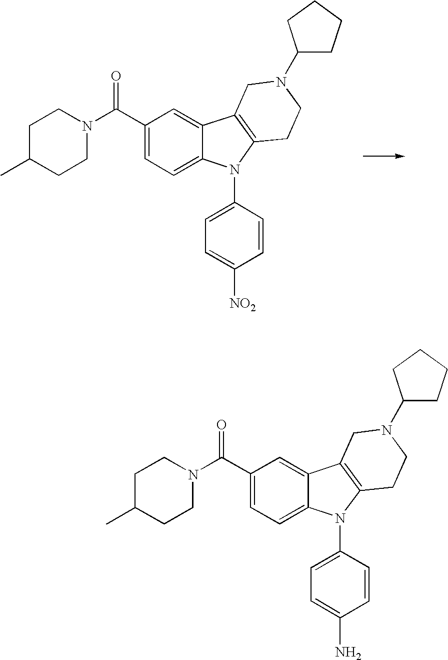

To a solution of above 2-cyclopentyl-8-[(4-methylpiperidin-1-yl)carbonyl]-5-(4-nitrophenyl)-2,3,4,5-tetrahydro-1H-pyrido[4,3-b]indole in MeOH (6 mL) was added Pd/C and the mixture placed under H₂ (30 psi) and stirred over night at room temperature. The mixture was then filtered and condensed under reduced pressure and was purified by reversed-phase HPLC (5-75%) to provide the desired compound as its TFA salt (15.69 mg, 12% for two steps). MS (M+1): 457.48. 1H NMR (600 MHz, CD3OD) δ ppm 0.83 (d, J=6.70 Hz, 3H), 0.94-1.16 (m, 2H), 1.42-1.52 (m, 1H), 1.52-1.84 (m, 9H), 2.10-2.26 (m, 2H), 2.68-2.80 (m, 1H), 2.80-2.90 (m, 1H), 2.90-3.10 (m, 2H,) 3.33-3.45 (m, 1H), 3.62-3.76 (m, 2H), 3.77-3.90 (m, 1H), 4.34 (d, J=14.08 Hz, 1H), 4.43-4.54 (m, 1H), 4.73 (d, J=14.59 Hz, 1H), 7.10 (s, 2H), 7.17 (d, J=8.70 Hz, 2H), 7.26 (d, J=8.45 Hz, 2H), 7.53 (s, 1H).

### Example 140

### 2-isopropyl-8-[(4-methylpiperidin-1-yl)carbonyl]-2,3,4,5-tetrahydro-1H-pyrido[4,3-b]indole

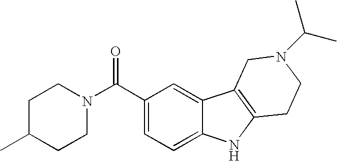

### Step A: 2-isopropyl-2,3,4,5-tetrahydro-1H-pyrido[4,3-b]indole-8-carboxylic acid

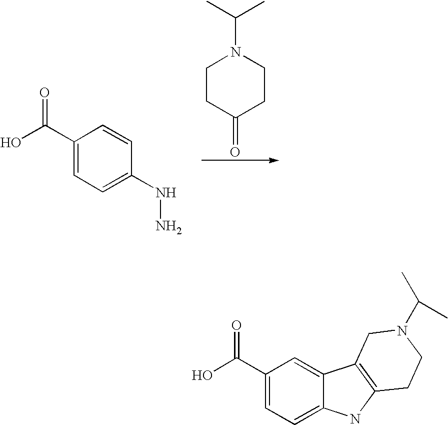

To a solution of 4-hydrazinobenzoic acid (5 g, 32 mmol) and 1-isopropyl-4-piperidone (4.6 g, 32 mmol) in dioxane (100 mL) was added conc. HCl (10 mL) and the mixture was heated at 100° C. overnight. The solvents were then removed and the yellow solid (12 g) was used for the next step without any further purification, the crude product was complexed with dioxane (˜2:1). MS (M+1): 259.00.

### Step B: 2-isopropyl-8-[(4-methylpiperidin-1-yl)carbonyl]-2,3,4,5-tetrahydro-1H-pyrido[4,3-b]indole

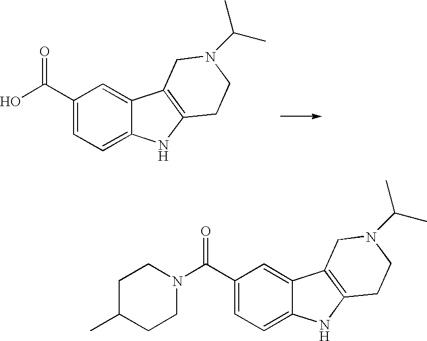

Following the similar procedure of Example 1, Step C: The title compound was prepared from crude 2-isopropyl-2,3,4,5-tetrahydro-1H-pyrido[4,3-b]indole-8-carboxylic acid and 4-methylpiperidine. MS (M+1): 340.0.

### Example 141

### 5-benzyl-2-isopropyl-8-[(4-methylpiperidin-1-yl)carbonyl]-2,3,4,5-tetrahydro-1H-pyrido[4,3-b]indole

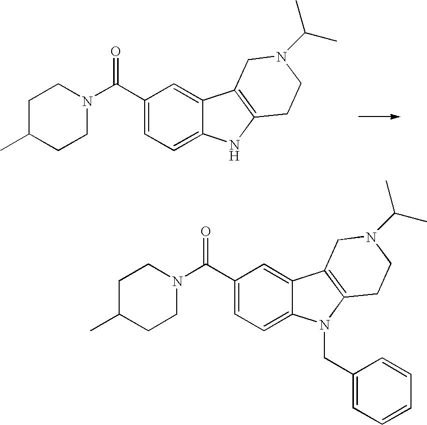

60% NaH (32 mg, 0.80 mmol) was added in one portion to a suspension of 2-isopropyl-8-[(4-methylpiperidin-1-yl)carbonyl]-2,3,4,5-tetrahydro-1H-pyrido[4,3-b]indole (75 mg, 0.22 mmol) in DMF (2 mL) at room temperature under nitrogen. The mixture was stirred for 30 min, (bromomethyl)benzene (31 μL, 0.26 mmol) was added and the reaction mixture was stirred at room temperature for 2 h. The solvent was removed in vacuo and the residue was dissolved in dichloromethane and washed with water dried over sodium sulfate. Removal of solvent gave the crude product, which was purified by reverse phase HPLC (5-75% CH₃CN) to provide the title compound as a white solid (24 mg). MS (M+1): 430.0 1H NMR (400 MHz, CDCl3) δ ppm 0.97 (d, J=6.25 Hz, 3H), 1.05-1.35 (m, 1H), 1.47 (dd, J=19.53, 6.64 Hz, 5H), 1.56-1.87 (m, 1H), 2.51-3.13 (m, 5H), 2.87 (d, J=17.77 Hz, 1H), 3.18-3.53 (m, 2H), 3.78-3.94 (m, 3H), 4.20 (d, J=13.28 Hz, 1H), 4.55-4.80 (m, 1H), 4.60 (d, J=13.86 Hz, 2H), 5.26 (d, J=7.42 Hz, 2H), 6.93 (d, J=6.05 Hz, 2H), 7.07-7.36 (m, 5H), 7.52 (s, 1H).

### Example 142

### 2-isopropyl-5-(4-methoxybenzyl)-8-[(4-methylpiperidin-1-yl)carbonyl]-2,3,4,5-tetrahydro-1H-pyrido[4,3-b]indole

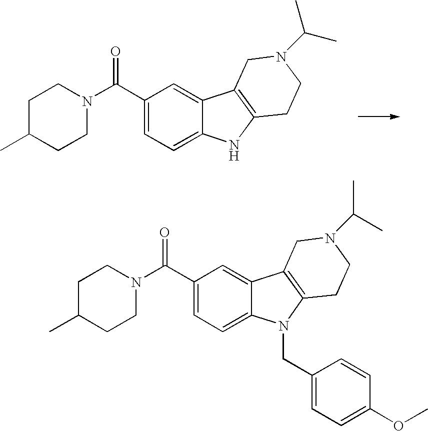

60% NaH (28 mg, 0.70 mmol) was added in one portion to a suspension of 2-isopropyl-8-[(4-methylpiperidin-1-yl)carbonyl]-2,3,4,5-tetrahydro-1H-pyrido[4,3-b]indole (73 mg, 0.21 mmol) in DMF (1.5 mL) at room temperature under nitrogen. The mixture was stirred for 30 min, 1-(chloromethyl)-4-methoxybenzene (58 μL, 0.43 mmol) was added and the reaction mixture was stirred at room temperature for 2 h. The solvent was removed in vacu and the residue was dissolved in dichloromethane and washed with water dried over sodium sulfate. Removal of solvent gave the crude product, which was purified over reverse phase HPLC (5-75% CH₃CN) to provide the title compound as its TFA salt (8 mg). MS (M+1): 460.0. 1H NMR (400 MHz, CDCl3) δ ppm 0.98 (d, J=6.44 Hz, 3H), 1.05-1.33 (m, 3H), 1.47 (dd, J=17.87, 6.54 Hz, 6H), 1.55-1.86 (m, 3H), 2.75-3.10 (m, 1H), 2.86 (d, J=15.43 Hz, 1H), 3.17-3.48 (m, 2H), 3.74 (s, 3H), 3.72-3.92 (m, 2H), 4.20 (d, J=16.01 Hz, 1H), 4.60 (d, J=13.28 Hz, 1H), 4.53-4.82 (m, 2H), 5.12-5.25 (m, 2H), 6.75-6.91 (m, 4H), 7.14-7.28 (m, 2H), 7.52 (s, 1H).

### Example 143

### 2-isopropyl-8-[(4-methylpiperidin-1-yl)carbonyl]-5-pentyl-2,3,4,5-tetrahydro-1H-pyrido[4,3-b]indole

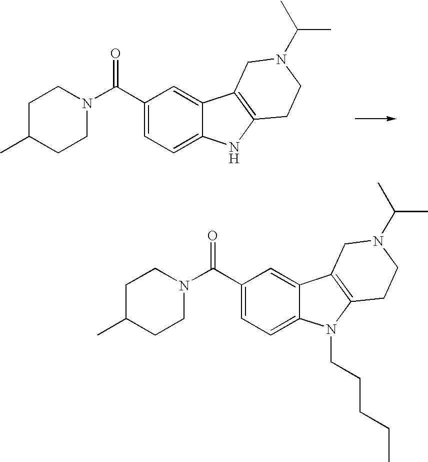

60% NaH (32 mg, 0.80 mmol) was added in one portion to a suspension of 2-isopropyl-8-[(4-methylpiperidin-1-yl)carbonyl]-2,3,4,5-tetrahydro-1H-pyrido[4,3-b]indole (75 mg, 0.22 mmol) in DMF (2 mL) at room temperature under nitrogen. The mixture was stirred for 30 min, 1-bromopentane (55 μL, 0.44 mmol) was added and the reaction mixture was stirred at room temperature for 2 h. The solvent was removed in vacuo and the residue was dissolved in dichloromethane and washed with water dried over sodium sulfate. Removal of solvent gave the crude product, which was purified over reverse phase HPLC (5-75% CH₃CN) to provide the title compound as its TFA salt (13 mg). MS (M+1): 410.2. 1H NMR (400 MHz, CD3OD) δ ppm 1H NMR (400 MHz, CD3OD) δ ppm 0.79 (t, J=7.03 Hz, 3H), 0.89 (d, J=6.44 Hz, 3H), 0.94-1.32 (m, 4H), 1.40 (d, J=6.45 Hz, 6H), 1.45-1.74 (m, 3H), 3.16 (t, J=5.66 Hz, 2H), 3.37-3.55 (m, 2H), 3.63-3.86 (m, 2H), 4.04-4.14 (m, 2H), 4.36-4.58 (m, 2H), 7.16 (d, J=8.59 Hz, 1H), 7.40 (d, J=8.59 Hz, 1H), 7.49 (s, 1H).

### Example 144

### 2-isopropyl-8-[(4-methylpiperidin-1-yl)carbonyl]-5-propyl-2,3,4,5-tetrahydro-1H-pyrido[4,3-b]indole

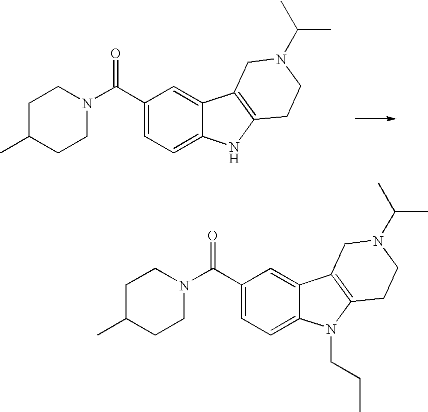

60% NaH (33 mg, 0.82 mmol) was added in one portion to a suspension of 2-isopropyl-8-[(4-methylpiperidin-1-yl)carbonyl]-2,3,4,5-tetrahydro-1H-pyrido[4,3-b]indole (75 mg, 0.22 mmol) in DMF (1.5 mL) at room temperature under nitrogen. The mixture was stirred for 30 min, 1-bromopropane (40 μL, 0.44 mmol) was added and the reaction mixture was stirred at room temperature for 2 h. The solvent was removed in vacuo and the residue was dissolved in dichloromethane and washed with water dried over sodium sulfate. Removal of solvent gave the crude product, which was purified over reverse phase HPLC to provide the title compound as its TFA salt (8 mg). MS (M+1): 382.0. 1H NMR (400 MHz, CDCl3) δ ppm 0.88 (t, J=7.42 Hz, 3 H), 0.98 (d, J=6.44 Hz, 3H), 1.07-1.33 (m, 2H), 1.47 (dd, J=17.48, 6.54 Hz, 6H), 1.57-1.84 (m, 6H), 2.74-3.08 (m, 2H), 2.96 (d, J=16.79 Hz, 1H), 3.22-3.40 (m, 1 H), 3.41-3.58 (m, 1H), 3.75-4.10 (m, 5H), 4.18 (d, J=13.67 Hz, 1H), 4.54 (d, J=13.86 Hz, 1H), 7.17-7.33 (m, 2H), 7.47 (s, 1H).

### Example 145

### 5-[(6-chloro-1,3-benzodioxol-5-yl)methyl]-2-isopropyl-8-[(4-methoxypiperidin-1-yl)carbonyl]-2,3,4,5-tetrahydro-1H-pyrido[4,3-b]indole

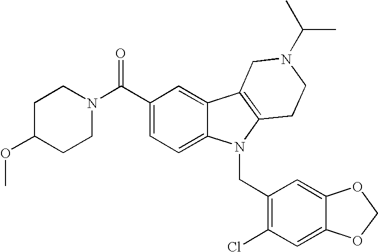

### Step A: The preparation of 2-isopropyl-8-[(4-methoxypiperidin-1-yl)carbonyl]-2,3,4,5-tetrahydro-1H-pyrido[4,3-b]indole

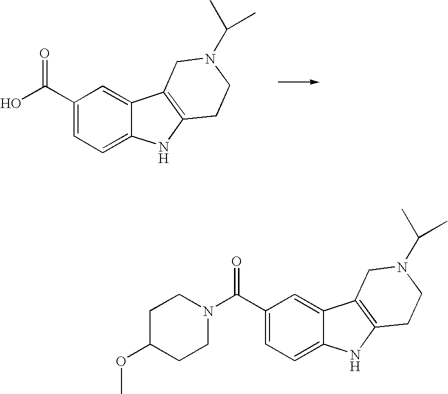

To a solution of 2-isopropyl-2,3,4,5-tetrahydro-1H-pyrido[4,3-b]indole-8-carboxylic acid (1.65 mmol) dissolved in DMF (5 mL) was added 4-methoxyl piperidine (1.65 mmol), diisopropylethylamine (1.65 mmol), and HATU (522 mg, 1.65 mmol) successively. The mixture was stirred at room temperature for overnight. The solvent was removed in vacuo and the residue was treated with water (10 mL) and extracted with dichloromethane (2×20 mL), dried over Na₂SO₄ and evaporated. The residue was purified by flash chromatography to give the desired product (230 mg, 39%). MS (M+1): 356.3.
Step B. The preparation of 5-[(6-chloro-1,3-benzodioxol-5-yl)methyl]-2-isopropyl-8-[(4-methoxypiperidin-1-yl)carbonyl]-2,3,4,5-tetrahydro-1H-pyrido[4,3-b]indole

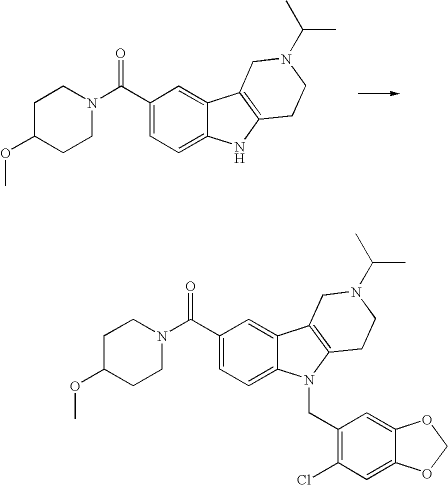

To a solution of 2-isopropyl-8-[(4-methoxypiperidin-1-yl)carbonyl]-2,3,4,5-tetrahydro-1H-pyrido[4,3-b]indole (75 mg, 0.21 mmol) in dry DMF (3 mL) was added 60% NaH (0.42 mmol). The mixture was stirred at 0° C. for 30 min and then 5-chloro-6-(chloromethyl)-1,3-benzodioxole (62 mg, 0.3 mmol) was added. After being stirred at room temperature for overnight, the solvent was removed in vacuo and the residue was dissolved in dichloromethane (20 mL), washed with water (10 mL), dried over sodium sulfate. Removal of solvent gave the crude product, which was purified with reverse phase HPLC to give the desired product as its TFA salt (32 mg, 24%). MS (M+1): 524.0. 1H NMR (400 MHz, CD3OD) δ ppm 1.44-1.54 (m, 9H), 1.83 (m, 3H), 3.16 (m, 3H), 3.37 (s, 3H), 3.53 (m, 3H), 3.78 (m, 1H), 3.87 (m, 1H), 4.53 (m, 1H), 4.66 (m, 1H), 5.42 (m, 2H), 5.90 (s, 1H), 5.91 (s, 2H), 6.97 (s, 1H), 7.27 (d, J=8.54 Hz, 1H), 7.41 (d, J=8.54 Hz, 1H), 7.67 (s, 1H).

### Example 146

### 5-(4-ethoxybenzyl)-2-isopropyl-8-[(4-methoxypiperidin-1-yl)carbonyl]-2,3,4,5-tetrahydro-1H-pyrido[4,3-b]indole

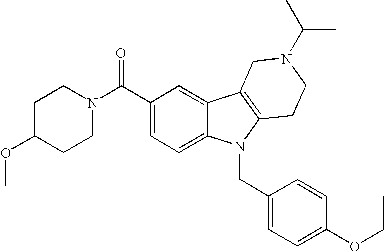

To a solution of 2-isopropyl-8-[(4-methoxypiperidin-1-yl)carbonyl]-2,3,4,5-tetrahydro-1H-pyrido[4,3-b]indole (75 mg, 0.21 mmol) in dry DMF (3 mL) was added 60% NaH (0.42 mmol). The mixture was stirred at 0° C. for 30 min and then 1-(chloromethyl)-4-ethoxybenzene (0.3 mmol) was added. After being stirred at room temperature for overnight, the solvent was removed in vacuo and the residue was dissolved in dichloromethane (20 mL), washed with water (10 mL), dried over sodium sulfate. Removal of solvent gave the crude product, which was purified with reverse phase HPLC to give the desired product as its TFA salt (46 mg, 36%). MS (M+1): 490.0. 1H NMR (400 MHz, CD3OD) δ ppm 1.33 (t, J=7.03 Hz, 3H), 1.40 (t, J=6.83 Hz, 4H), 1.48 (dd, J=6.06, 1.17 Hz, 6H), 2.85 (s, 3H), 3.19 (m, 1H), 3.36 (s, 3H), 3.53 (m, 2H), 3.81 (m, 2H), 3.96 (q, J=7.03 Hz, 2H), 4.07 (m, 2H), 4.46 (s, 2H), 4.59 (m, 1H), 5.35 (m, 1H), 6.81 (d, J=8.59 Hz, 1H), 7.03 (m, 3H), 7.45 (d, J=8.59 Hz, 2H), 7.64 (s, 1H).

### Example 147

### 5-(2,6-difluorobenzyl)-2-isopropyl-8-[(4-methoxypiperidin-1-yl)carbonyl]-2,3,4,5-tetrahydro-1H-pyrido[4,3-b]indole

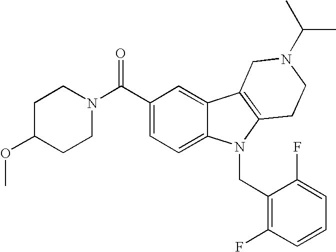

To a solution of 2-isopropyl-8-[(4-methoxypiperidin-1-yl)carbonyl]-2,3,4,5-tetrahydro-1H-pyrido[4,3-b]indole (75 mg, 0.21 mmol) in dry DMF (3 mL) was added 60% NaH (0.42 mmol). The mixture was stirred at 0° C. for 30 min and then 2-(bromomethyl)-1,3-difluorobenzene (0.3 mmol) was added. After being stirred at room temperature for overnight, the solvent was removed in vacuo and the residue was dissolved in dichloromethane (20 mL), washed with water (10 mL), dried over sodium sulfate. Removal of solvent gave the crude product, which was purified with reverse phase HPLC to give the desired product as its TFA salt (53 mg, 42%). MS (M+1): 482.0. 1H NMR (400 MHz, CD3OD) δ ppm 1.49 (dd, J=6.45, 2.74 Hz, 6H), 1.52-2.06 (m, 4H), 3.18-3.38 (m, 3H), 3.36 (s, 3H), 3.47-3.72 (m, 4H), 3.77 (m, 1H), 3.90 (m, 1H), 4.00 (m, 1H), 4.53 (m, 2H), 5.48 (m, 2H), 7.03 (m, 2H), 7.25 (dd, J=8.50, 1.37 Hz, 1H), 7.40 (m, 1H), 7.56 (d, J=8.50 Hz, 1H), 7.59 (s, 1H).

### Example 148

### 5-(4-ethoxybenzyl)-8-[(4-ethoxypiperidin-1-yl)carbonyl]-2-isopropyl-2,3,4,5-tetrahydro-1H-pyrido[4,3-b]indole

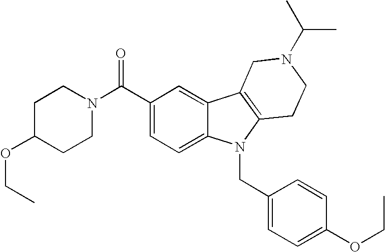

### Step A: The preparation of 8-[(4-ethoxypiperidin-1-yl)carbonyl]-2-isopropyl-2,3,4,5-tetrahydro-1H-pyrido[4,3-b]indole

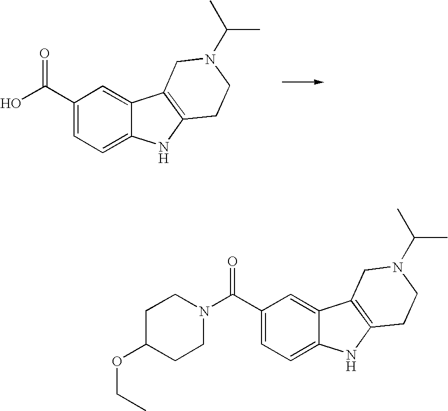

To a solution of 2-isopropyl-2,3,4,5-tetrahydro-1H-pyrido[4,3-b]indole-8-carboxylic acid (1.2 mmol) in DMF (3 mL) was added 4-ethoxyl piperidine (1.2 mmol, prepared following the procedure from the literature, see: Grob, C. A.; Krasnobajew, V. Helvetica Chimica Acta 1964, 47, 2145), diisopropylethylamine (1.2 mmol) and HATU (1.44 mmol) successively. The mixture was stirred at room temperature for overnight. The solvent was removed in vacuo and the residue was treated with water (10 mL) and extracted with dichloromethane (2×20 mL), dried over Na₂SO₄ and evaporated. The residue was purified by flash chromatography to give the desired product (70 mg, 16%). MS (M+1): 370.4.

### Step B: The preparation of 5-(4-ethoxybenzyl)-8-[(4-ethoxypiperidin-1-yl)carbonyl]-2-isopropyl-2,3,4,5-tetrahydro-1H-pyrido[4,3-b]indole

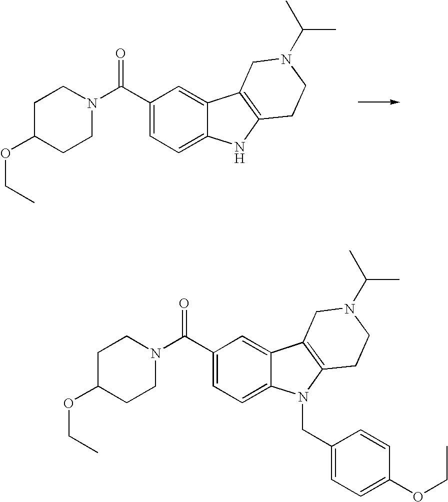

To a solution of 8-[(4-ethoxypiperidin-1-yl)carbonyl]-2-isopropyl-2,3,4,5-tetrahydro-1H-pyrido[4,3-b]indole (70 mg, 0.19 mmol) in dry DMF (3 mL) was added 60% NaH (0.38 mmol). The mixture was stirred at 0° C. for 30 min and then 1-(chloromethyl)-4-ethoxybenzene (0.28 mmol) was added. After being stirred at room temperature for overnight, the solvent was removed id vacuo and the residue was dissolved in dichloromethane (20 mL), washed with water (10 mL), dried over sodium sulfate. Removal of solvent gave the crude product, which was purified with reverse phase HPLC to give the desired product as its TFA salt (63 mg, 54%). MS (M+1): 504.0. 1H NMR (400 MHz, CD3OD) δ ppm 1.19 (t, J=7.03 Hz, 3H), 1.33 (t, J=6.83 Hz, 3H), 1.48 (dd, J=6.05, 0.97 Hz, 6H), 1.75-2.05 (m, 4H), 3.15-3.25 (m, 2H), 3.50-3.68 (m, 6H), 3.80 (m, 2H), 3.97 (q, J=7.03 Hz, 2H), 4.49 (m, 1H), 4.63 (m, 1H), 5.28-5.46 (m, 4H), 6.81 (d, J=8.72 Hz, 2H), 7.03 (d, J=8.72 Hz, 2H), 7.26 (dd, J=8.45, 1.27 Hz, 1H), 7.54 (d, J=8.45 Hz, 1H), 7.64 (s, 1H).

### Example 149

### 2-cyclopentyl-8-[(4,4-difluoropiperidin-1-yl)carbonyl]-5-methyl-2,3,4,5-tetrahydro-1H-pyrido[4,3-b]indole

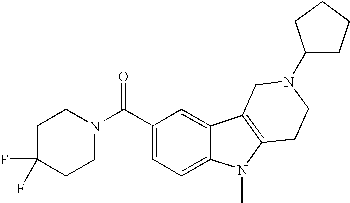

### Step A. The preparation of tert-butyl 8-[(4,4-difluoropiperidin-1-yl)carbonyl]-1,3,4,5-tetrahydro-2H-pyrido[4,3-b]indole-2-carboxylate

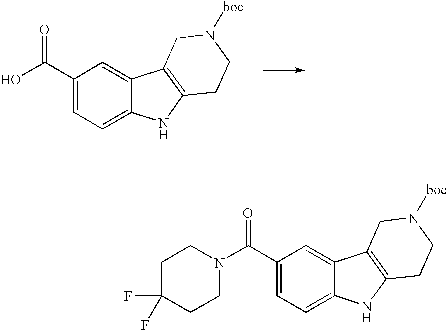

2-(tert-butoxycarbonyl)-2,3,4,5-tetrahydro-1H-pyrido[4,3-b]indole-8-carboxylic acid, 284 mg, 0.9 mmol) was dissolved in DMF (3 mL), 3-methylpiperidine (1.35 mmol), diisopropylethylamine (1.08 mmol), and HATU (410 mg, 1.08 mmol) was added successively. The mixture was stirred at room temperature for 5 h. The solvent was removed in vacuo and the residue was treated with water (10 mL) and extracted with dichloromethane (2×20 mL), dried over Na₂SO₄, evaporated to give the crude product (361 mg, 96%), which was used for the next step without purification. MS (M+1): 420.1.

### Step B: The preparation of tert-butyl 8-[(4,4-difluoropiperidin-1-yl)carbonyl]-5-methyl-1,3,4,5-tetrahydro-2H-pyrido[4,3-b]indole-2-carboxylate

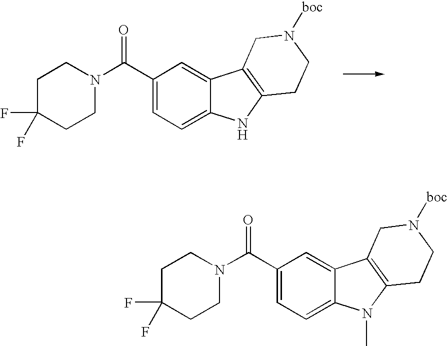

The crude product tert-butyl 8-[(4,4-difluoropiperidin-1-yl)carbonyl]-1,3,4,5-tetrahydro-2H-pyrido[4,3-b]indole-2-carboxylate (361 mg, 0.86 mmol) was treated with 60% NaH (1.72 mmol) in DMF (10 mL) at 0° C. for 30 min and then added iodomethane (1.03 mmol). After being stirred at room temperature for 2 h, the solvent was removed in vacuo and the residue was dissolved in dichloromethane (50 mL), washed with water (20 mL), dried over sodium sulfate. Removal of solvent gave the crude product (372 mg, 99%), which was used without further purification. MS (M+1): 434.4.

### Step C. The preparation of 8-[(4,4-difluoropiperidin-1-yl)carbonyl]-5-methyl-2,3,4,5-tetrahydro-1H-pyrido[4,3-b]indole

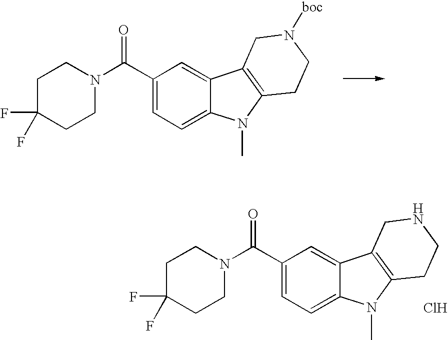

To a solution of tert-butyl 8-[(4,4-difluoropiperidin-1-yl)carbonyl]-5-methyl-1,3,4,5-tetrahydro-2H-pyrido[4,3-b]indole-2-carboxylate (372 mg, 0.86 mmol) in dioxane (5 mL) was added 4N HCl in dioxane (4 mL). The solution was stirred at room temperature for 5 h. The solvent was removed in vacuo and the residue was washed with diethyl ether, filtered to give the desired product as its HCl salt (305 mg, 96%), which was used without further purification. MS (M+1): 334.0.

### Step D: The preparation of 2-cyclopentyl-8-[(4,4-difluoropiperidin-1-yl)carbonyl]-5-methyl-2,3,4,5-tetrahydro-1H-pyrido[4,3-b]indole

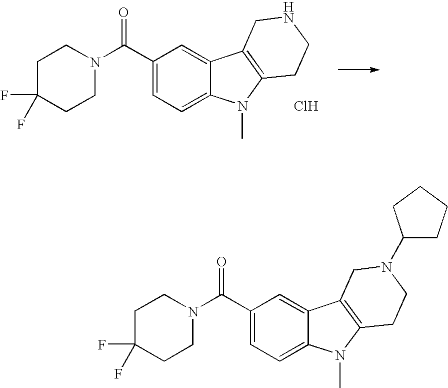

The crude 8-[(4,4-difluoropiperidin-1-yl)carbonyl]-5-methyl-2,3,4,5-tetrahydro-1H-pyrido[4,3-b]indole (HCl salt, 0.2 mmol, 74 mg) was treated with cyclopentanone (0.4 mmol) and sodium triacetoxyborohydride (0.3 mmol) in dichloromethane (4 mL). The mixture was stirred at room temperature overnight, diluted with dichloromethane (15 mL), washed with saturated NaHCO₃ solution (5 mL), dried over Na₂SO₄. The dichloromethane solution was concentrated in vacuo and the residue was purified with reverse phase HPLC to give the desired product as its TFA salt (28 mg, 27%). MS (M+1): 402.0. 1H NMR (600 MHz, CDCl3) δ ppm 1.69 (m, 2H), 1.92 (m, 2H), 1.98-2.22 (m, 8H), 3.04 (m, 1H), 3.44 (m, 2H), 3.50 (s, 2H), 3.65 (m, 2H), 3.70 (s, 3H), 4.00 (m, 2H), 4.20 (d, J=14.08 Hz, 1H), 4.81 (d, J=14.08 Hz, 1H), 7.28 (d, J=8.45 Hz, 1H), 7.34 (d, J=8.45 Hz, 1H), 7.55 (s, 1H).

### Example 150

### 8-[(4,4-difluoropiperidin-1-yl)carbonyl]-5-methyl-2-(tetrahydro-2H-pyran-4-yl)-2,3,4,5-tetrahydro-1H-pyrido[4,3-b]indole

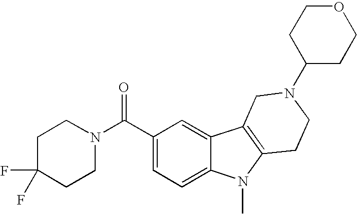

The crude 8-[(4,4-difluoropiperidin-1-yl)carbonyl]-5-methyl-2,3,4,5-tetrahydro-1H-pyrido[4,3-b]indole (HCl salt, 0.2 mmol, 74 mg) was treated with tetrahydro-4H-pyran-4-one (0.4 mmol) and sodium triacetoxyborohydride (0.3 mmol) in dichloromethane (4 mL). The mixture was stirred at room temperature overnight, diluted with dichloromethane (15 mL), washed with saturated NaHCO₃ solution (5 mL), dried over Na₂SO₄. The dichloromethane solution was concentrated in vacuo and the residue was purified with reverse phase HPLC to give the desired product as its TFA salt (52 mg, 49%). MS (M+1): 418.0. 1H NMR (600 MHz, CDCl3) δ ppm 1.89-2.22 (m, 8H), 3.04 (m, 1H), 3.31-3.54 (m, 4H), 3.68 (s, 3H), 3.75 (m, 3H), 3.95 (m, 1H), 4.17 (d, J=10.75 Hz, 2H), 4.29 (d, J=13.31 Hz, 1H), 4.67 (d, J=13.31 Hz, 1H), 5.30 (m, 2H), 7.28 (d, J=8.19 Hz, 1H), 7.33 (d, J=8.19 Hz, 1H), 7.54 (s, 1H).

### Example 151

### 8-[(4,4-difluoropiperidin-1-yl)carbonyl]-5-methyl-2-(tetrahydrofuran-3-yl)-2,3,4,5-tetrahydro-1H-pyrido[4,3-b]indole

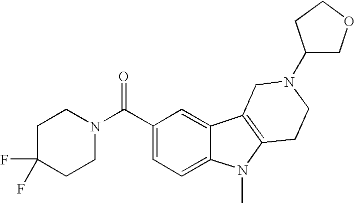

Following the same procedure as Example 1, Step E, crude 8-[(4,4-difluoropiperidin-1-yl)carbonyl]-5-methyl-2,3,4,5-tetrahydro-1H-pyrido[4,3-b]indole (HCl salt, 0.15 mmol, 55 mg) was treated with dihydrofuran-3 (2H)-one (0.3 mmol) and sodium triacetoxyborohydride (0.23 mmol) in dichloromethane (3 mL). The mixture was stirred at room temperature overnight, diluted with dichloromethane (15 mL), washed with saturated NaHCO₃ solution (5 mL), dried over Na₂SO₄. The dichloromethane solution was concentrated in vacuo and the residue was purified with reverse phase HPLC to give the desired product as its TFA salt (23 mg, 30%). MS (M+1): 404.0. 1H NMR (600 MHz, CDCl3) δ ppm 1.89-2.16 (m, 4H), 2.45 (m, 2H), 3.41 (m, 2H), 3.50 (s, 2H), 3.69 (s, 3H), 3.73-4.05 (m, 7H), 4.19 (m, 1H), 4.25 (m, 1H), 4.35 (dd, J=11.01, 2.30 Hz, 1H), 4.66 (m, 1H), 7.29 (d, J=8.45 Hz, 1H), 7.33 (d, J=8.45 Hz, 1H), 7.53 (s, 1H).

### Example 152

### 5-allyl-2-(cyclobutyl)-8-[(3-methylpiperidin-1-yl)carbonyl]-2,3,4,5-tetrahydro-1H-pyrido[4,3-b]indole

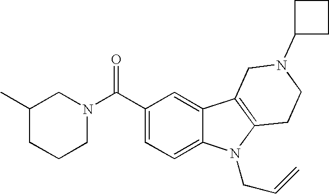

### Step A: The preparation of tert-butyl 8-[(3-methylpiperidin-1-yl)carbonyl]-1,3,4,4a,5,9b-hexahydro-2H-pyrido[4,3-b]indole-2-carboxylate

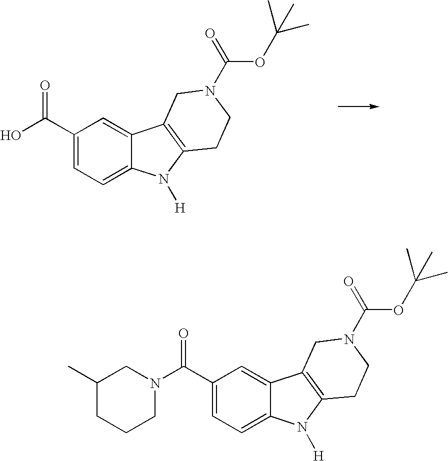

2-(tert-butoxycarbonyl)-2,3,4,5-tetrahydro-1H-pyrido[4,3-b]indole-8-carboxylic acid (316 mg, 1.0 mmol) was dissolved in DMF (3 mL), 3-methylpiperidine (1.5 mmol), diisopropylethylamine (1.2 mmol), and HATU (456 mg, 1.2 mmol) was added successively. The mixture was stirred at room temperature for 3 h. The solvent was removed in vacuo and the residue was treated with water (10 mL) and extracted with dichloromethane (2×20 mL), dried over Na₂SO₄, evaporated to give the crude product as yellow solid. The crude was used for the next step without purification. MS (M+1): 398.1.

### Step B: The preparation of tert-butyl 5-allyl-8-[(3-methylpiperidin-1-yl)carbonyl]-1,3,4,5-tetrahydro-2H-pyrido[4,3-b]indole-2-carboxylate

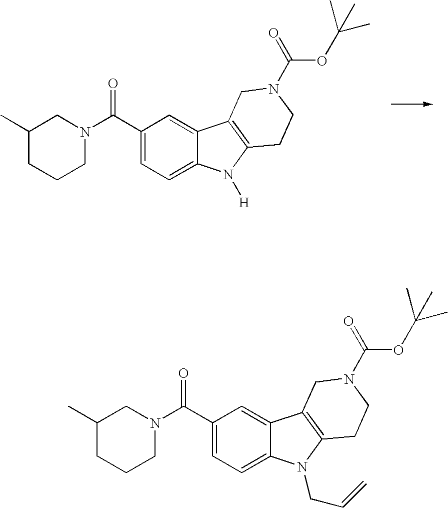

The crude product tert-butyl 8-[(3-methylpiperidin-1-yl)carbonyl]-1,3,4,4a,5,9b-hexahydro-2H-pyrido[4,3-b]indole-2-carboxylate (1.0 mmol) was treated with 60% NaH (2 mmol) in DMF at 0° C. for 30 mm and then added allyl bromide (1.5 mmol). After being stirred at room temperature for 2 h, the solvent was removed in vacuo and the residue was dissolved in dichloromethane (20 mL), washed with water (10 mL), dried over sodium sulfate. Removal of solvent gave the crude product, which was purified with a short pad of silica gel (0-30% EtOAc in dichloromethane) to give the desired product (328 mg, 75% for two steps). MS (M+1): 438.1.

### Step C: The preparation of 5-allyl-8-[(3-methylpiperidin-1-yl)carbonyl]-2,3,4,5-tetrahydro-1H-pyrido[4,3-b]indole

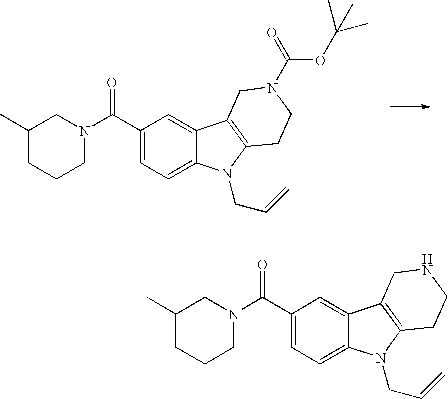

To the solution of tert-butyl 5-allyl-8-[(3-methylpiperidin-1-yl)carbonyl]-1,3,4,5-tetrahydro-2H-pyrido[4,3-b]indole-2-carboxylate (328 mg) in dichloromethane (5 mL) was added trifluoroacetic acid (20 eq.) at room temperature for 1 h. Removal of excess trifluoroacetic acid in vacuo gave crude 5-allyl-8-[(3-methylpiperidin-1-yl)carbonyl]-2,3,4,5-tetrahydro-1H-pyrido[4,3-b]indole as its trifluoroacetic acid salt form.

### Step D: The preparation of 5-allyl-2-(cyclobutyl)-8-[(3-methylpiperidin-1-yl)carbonyl]-2,3,4,5-tetrahydro-1H-pyrido[4,3-b]indole

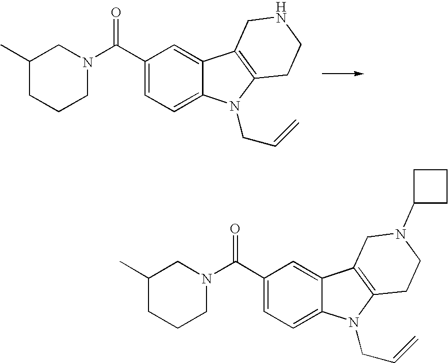

Crude 5-allyl-8-[(3-methylpiperidin-1-yl)carbonyl]-2,3,4,5-tetrahydro-1H-pyrido[4,3-b]indole (TFA salt, 0.175 mmol, 60 mg) was treated with cyclobutanone (0.35 mmol) and sodium triacetoxyborohydride (0.26 mmol) in dichloromethane. The mixture was stirred at room temperature overnight, diluted with dichloromethane (15 mL), washed with saturated NaHCO₃ solution (5 mL), dried over Na₂SO₄. The dichloromethane solution was concentrated in vacuo and the residue was purified with reverse phase HPLC to give the desired product 5-allyl-2-(cyclopropylmethyl)-8-[(4-methylpiperidin-1-yl)carbonyl]-2,3,4,5-tetrahydro-1H-pyrido[4,3-b]indole as its TFA salt (28 mg, 32%). MS (M+1): 392.0. 1H NMR (400 MHz, CDCl3) δ ppm 0.70-1.07 (m, 3H), 1.10-1.40 (m, 3H), 1.48-2.03 (m, 6H), 2.23-2.42 (m, 2H), 2.50-2.76 (m, 2H), 2.85-3.01 (m, 2H), 3.20-3.29 (m, 2H), 3.60-4.00 (m, 5H), 4.50-4.75 (m, 2H), 4.79 (d, J=17.18 Hz, 1H), 5.17 (d, J=10.35 Hz, 1H), 5.85-6.00 (m, 1H), 7.20-7.32 (m, 2H), 7.49 (s, 1H).

### Example 153

### Methyl 5-methyl-2-(tetrahydro-2H-pyran-4-yl)-2,3,4,5-tetrahydro-1H-pyrido[4,3-b]indole-8-carboxylate

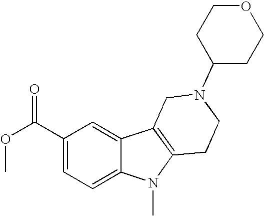

### Step A: The preparation of 2-tert-butyl 8-methyl 5-methyl-1,3,4,5-tetrahydro-2H-pyrido[4,3-b]indole-2,8-dicarboxylate

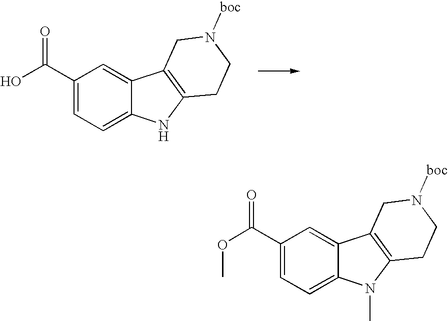

To a solution of 2-(tert-butoxycarbonyl)-2,3,4,4a,5,9b-hexahydro-1H-pyrido[4,3-b]indole-8-carboxylic acid (3.16 g, 10 mmol) in dry DMF (50 mL) was added 60% NaH (80 mmol) in one portion at 0° C. under nitrogen. The mixture was stirred at room temperature for 30 min, then iodomethane (80 mmol) was added at room temperature, and the reaction mixture was stirred at room temperature for overnight. The solvent was removed in vacuo and the residue was dissolved in dichloromethane (200 mL), washed with water (50 mL), dried over sodium sulfate. Removal of solvent gave the crude product (3.14 g, 91%), which was used without further purification. MS (M+1): 345.3.

### Step B: The preparation of methyl 5-methyl-2,3,4,5-tetrahydro-1H-pyrido[4,3-b]indole-8-carboxylate

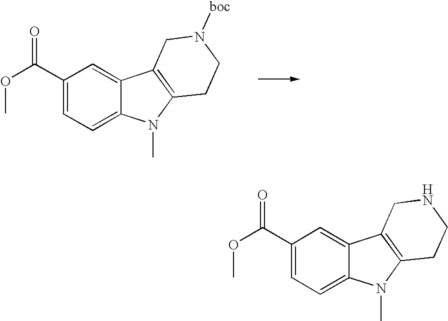

Following the same procedure as Example 1, Step D (Method B), 2-tert-butyl 8-methyl 5-methyl-1,3,4,5-tetrahydro-2H-pyrido[4,3-b]indole-2,8-dicarboxylate (3.14 g, 9.13 mmol) was treated with trifluoroacetic acid in dichloromethane at room temperature for 1 h. Removal of excess trifluoroacetic acid in vacuo gave crude as its trifluoroacetic acid salt form (3.44 g, 96%). MS (M+1): 244.9.

### Step C: The preparation of methyl 5-methyl-2-(tetrahydro-2H-pyran-4-yl)-2,3,4,5-tetrahydro-1H-pyrido[4,3-b]indole-8-carboxylate

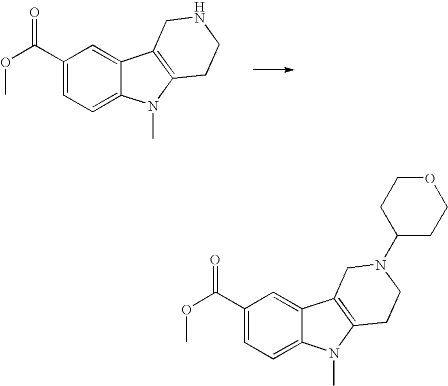

To a solution of methyl 5-methyl-2,3,4,5-tetrahydro-1H-pyrido[4,3-b]indole-8-carboxylate (TFA salt, 2.83 g, 7.9 mmol) in dichloromethane/metanol (1:1 v/v, 80 mL) was added triethylamine (7.9 mmol) followed by tetrahydro-4H-pyran-4-one (11.9 mmol) and sodium cyanoborohydride (11.9 mmol). The mixture was stirred at 50° C. for overnight, followed by the addition of another portion of tetrahydro-4H-pyran-4-one (7.9 mmol) and sodium cyanoborohydride (7.9 mmol). After being stirred at 50° C. for another 24 h, the solvents were removed in vacuo and the residue was taken up into dichloromethane (100 mL), washed with saturated NaHCO₃ solution (50 mL), dried over Na₂SO₄. The dichloromethane solution was concentrated in vacuo and the residue was purified by passing through a short pad of silica gel to give the desired product as white solid (2.41 g, 93%). MS (M+1): 329.0. 1H NMR (600 MHz, CD3OD) δ ppm 1.88-2.00 (m, 2H), 2.13-2.25 (m, 2H), 3.24-3.35 (m, 2H), 3.46-3.65 (m, 3H), 3.71-3.75 (m, 1H), 3.77 (s, 3H), 3.92 (s, 3H), 4.00-4.09 (m, 1H), 4.10-4.19 (m, 2H), 4.55 (d, J=12.03 Hz, 1H), 4.70-4.82 (m, 1H), 7.50 (d, J=8.70 Hz, 1H), 7.92 (d, J=8.70 Hz, 1H), 8.30 (s, 1H).

### Example 154

### 1-{[5-methyl-2-(tetrahydro-2H-pyran-4-yl)-2,3,4,5-tetrahydro-1H-pyrido[4,3-b]indol-8-yl]carbonyl}piperidine-4-carboxamide

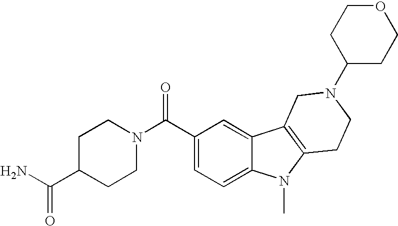

### Step A: The preparation of 5-methyl-2-(tetrahydro-2H-pyran-4-yl)-2,3,4,5-tetrahydro-1H-pyrido[4,3-b]indole-8-carboxylic acid

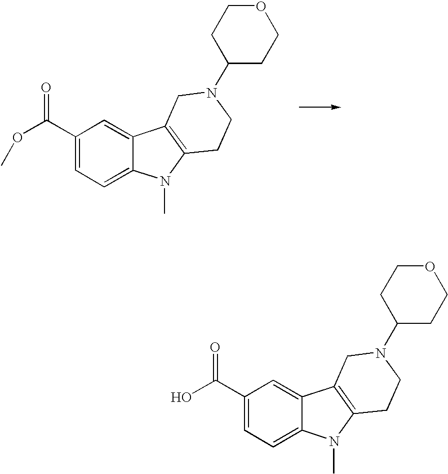

To a solution of methyl 5-methyl-2-(tetrahydro-2H-pyran-4-yl)-2,3,4,5-tetrahydro-1H-pyrido[4,3-b]indole-8-carboxylate (580 mg, 1.77 mmol) in tetrahydrofuran/methanol/water (1:1:1 v/v, 15 mL) was added KOH (297 mg, 5.31 mmol). The reaction mixture was stirred at 80° C. for 3 h. After cooled to room temperature, the solution was neutralized with 1M HCl to pH˜5.0, concentrated, the solid was refluxed in dichloromethane/methaol (v/v 1:1, 80 mL) for 5 min, filtered through Celite, the filtrate was concentrated to give desired product as white solid (420 mg, 76%). MS (M+1): 315.0.

### Step B: The preparation of 1-{[5-methyl-2-(tetrahydro-2H-pyran-4-yl)-2,3,4,5-tetrahydro-1H-pyrido[4,3-b]indol-8-yl]carbonyl}piperidine-4-carboxamide

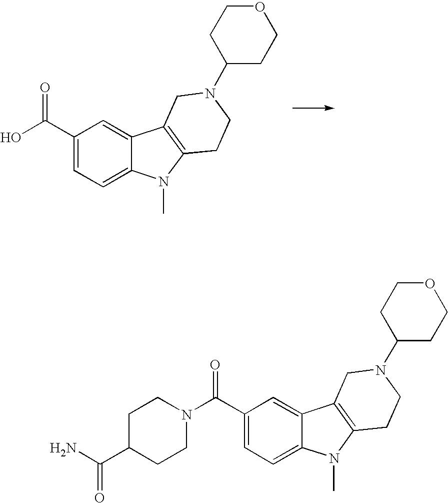

To a solution of (2-(tert-butoxycarbonyl)-2,3,4,5-tetrahydro-1H-pyrido[4,3-b]indole-8-carboxylic acid, 57 mg, 0.18 mmol) was dissolved in DMF (3 mL), isonipecotamide (0.22 mmol), disiopropylethylamine (0.22 mmol), and HATU (84 mg, 0.22 mmol) was added successively. The mixture was stirred at room temperature for 3 h. The solvent was removed in vacuo and the residue was treated with water (10 mL) and extracted with dichloromethane (2×20 mL), dried over Na₂SO₄, evaporated. The residue was purified with reverse phase HPLC to give the desired product as its TFA salt (31 mg, 32%). MS (M+1): 425.0. 1H NMR (600 MHz, CD3OD) δ ppm 1.66-1.79 (m, 2H), 1.89-1.99 (m, 2H), 2.00-2.07 (m, 2H), 2.15-2.25 (m, 2H), 2.53-2.61 (m, 1H), 3.27-3.32 (m, 4H), 3.47-3.56 (m, 3H), 3.57-3.64 (m, 1H), 3.69-3.76 (m, 2H), 3.78 (s, 3H), 4.02-4.09 (m, 1H), 4.11-4.19 (m, 2H), 4.50-4.58 (m, 1H), 4.72-4.79 (m, 1H), 7.31 (d, J=8.45 Hz, 1H), 7.52 (d, J=8.45 Hz, 1H), 7.64 (s, 1H).

### Example 155-171

The following examples (Table 3) were prepared by the same method described in Example 154, Step B.

TABLE 3

| Exam- | | | |
|---|---|---|---|
| ples | Structures | | Data |
| 155 | 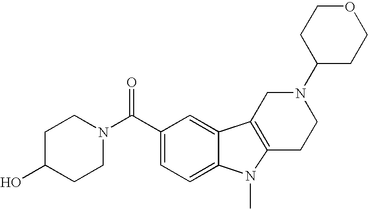 | 1-{[5-methyl-2- (tetrahydro-2H-pyran- 4-yl)-2,3,4,5- tetrahydro-1H- pyrido[4,3-b]indol-8- yl]carbonyl}piperidin- 4-ol | MS (M + 1): 398.0. 1H NMR (600 MHz, CD3OD) δ ppm 1.41-1.64 (m, 2 H), 1.77- 1.88 (m, 1 H), 1.90-2.02 (m, 3 H), 2.19-2.21 (m, 2H), 2.83 (s, 2 H), 3.24-3.31 (m, 2H), 3.48-3.56 (m, 2 H), 3.57- 3.63 (m, 1 H), 3.69-3.75 (m, 2 H), 3.77 (s, 3 H), 3.87-3.96 (m, 1 H), 4.02-4.10 (m, 1 H), 4.11-4.18 (m, 2 H), 4.19- 4.29 (m, 1 H), 4.49-4.57 (m, 1 H), 4.70-4.78 (m, 1 H), 7.30 (d, J = 8.45 Hz, 1 H), 7.52 (d, J = 8.45 Hz, 1 H), 7.63 (s, 1 H). |
| 156 | 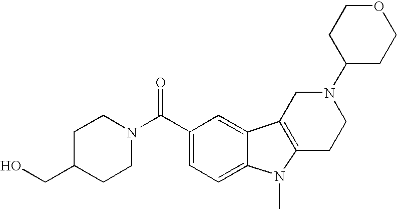 | (1-{[5-methyl-2- (tetrahydro-2H-pyran- 4-yl)-2,3,4,5- tetrahydro-1H- pyrido[4,3-b]indol-8- yl]carbonyl}piperidin- 4-yl)methanol | MS (M + 1): 412.0. 1H NMR (600 MHz, CD3OD) δ ppm 1.17-1.41 (m, 2 H), 1.66- 1.84 (m, 2 H), 1.86-1.99 (m, 3 H), 2.13-2.25 (m, 2 H), 2.84-2.99 (m, 1 H), 3.08- 3.20 (m, 1 H), 3.24-3.30 (m, 2 H), 3.42-3.49 (m, 2 H), 3.52 (d, J = 9.73 Hz, 2 H), 3.57-3.62 (m, 1 H), 3.70-3.75 (m, 1 H), 3.77 (s, 3 H), 3.82-3.96 (m, 1 H), 4.03-4.09 (m, 1 H), 4.11- 4.18 (m, 2 H), 4.32 (d, J = 6.14 Hz, 1H), 4.54 (d, J = 13.58 Hz, 1 H), 4.75 (d, J = 13.58 Hz, 1 H), 7.29 (d, J = 8.45 Hz, 1 H), 7.50 (d, J = 8.45 Hz, 1 H), 7.62 (s, 1 H). |
| 157 | 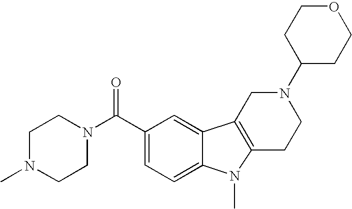 | 5-methyl-8-[(4- methylpiperazin-1- yl)carbonyl]-2- (tetrahydro-2H-pyran- 4-yl)-2,3,4,5- tetrahydro-1H- pyrido[4,3-b]indole | MS (M + 1): 397.0. 1H NMR (600 MHz, CD3OD) δ ppm 1.88-2.01(m, 2H), 2.12- 2.27 (m, 2H), 2.97 (s, 3 H), 3.15-3.24 (m, 2 H), 3.27- 3.34 (m, 4 H), 3.39-3.67 (m, 7 H), 3.70-3.76 (m, 1 H), 3.77 (s, 3 H), 4.01-4.09 (m, 1 H), 4.11-4.18 (m, 2 H), 4.48- 4.57 (m, 1 H), 4.70-4.78 (m, 1 H), 7.38 (d, J = 8.45 Hz, 1 H), 7.54 (d, J = 8.45 Hz, 1 H), 7.72 (s, 1 H). |
| 158 | 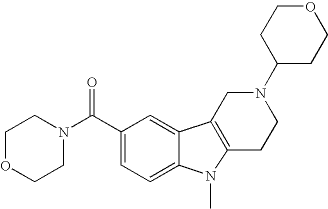 | 5-methyl-8- (morpholin-4- ylcarbonyl)-2- (tetrahydro-2H-pyran- 4-yl)-2,3,4,5- tetrahydro-1H- pyrido[4,3-b]indole | MS (M + 1): 384.0. 1H NMR (600 MHz, CD3OD) δ ppm 1.90-1.99 (m, 2 H), 2.17- 2.24 (m, 2 H), 3.26-3.31 (m, 2 H), 3.47-3.56 (m, 3 H), 3.56-3.64 (m, 2 H), 3.66- 3.76 (m, 7 H), 3.77 (s, 3 H), 4.02-4.09 (m, 1 H), 4.12- 4.18 (m, 2 H), 4.53 (d, J = 13.57 Hz, 1 H), 4.75 (d, J = 13.57 Hz, 1 H), 7.32 (d, J = 8.35 Hz, 1 H), 7.52 (d, J = 8.35 Hz, 1 H), 7.66 (s, 1 H). |
| 159 | 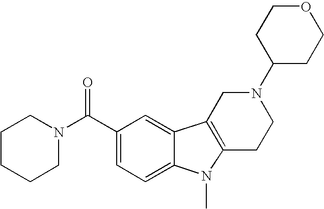 | 5-methyl-8-(piperidin- 1-ylcarbonyl)-2- (tetrahydro-2H-pyran- 4-yl)-2,3,4,5- tetrahydro-1H- pyrido[4,3-b]indole | MS (M + 1): 382.0. 1H NMR (600 MHz, CD3OD) δ ppm 1.51-1.66 (m, 2 H), 1.72- 1.76 (m, 4 H), 1.90-2.00 (m, 2 H), 2.15-2.25 (m, 2 H), 3.24-3.31 (m, 2 H), 3.40- 3.57 (m, 4 H), 3.56-3.64 (m, 1 H), 3.68-3.76 (m, 3 H), 3.75- 3.80 (m, 3 H), 4.00-4.09 (m, 1 H), 4.15 (d, J = 11.26 Hz, 2 H), 4.53 (d, J = 13.45 Hz, 1 H), 4.74 (d, J = 13.45 Hz; 1 H), 7.29 (d, J = 8.19 Hz, 1 H), 7.51 (d, J = 8.19 Hz, 1 H), 7.61 (s, 1 H). |
| 160 | 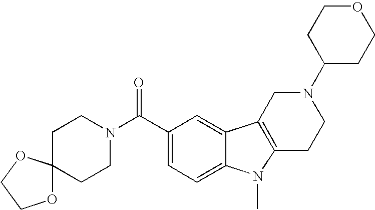 | 8-(1,4-dioxa-8- azaspiro[4.5]dec-8- ylcarbonyl)-5-methyl- 2-(tetrahydro-2H- pyran-4-yl)-2,3,4,5- tetrahydro-1H- pyrido[4,3-b]indole | MS (M + 1): 440.0: 1H NMR (600 MHz, CD3OD) δ ppm 1.63-1.76 (m, 2 H), 1.77- 1.87 (m, 2 H), 1.90-1.99 (m, 2 H), 2.15-2.24 (m, 2 H), 3.46-3.56 (m, 3 H), 3.57- 3.63 (m, 3 H), 3.69-3.77 (m, 2 H), 3.77 (s, 3 H), 3.80-3.92 (m, 2 H), 3.95-4.02 (m, 4 H), 4.03-4.10 (m, 1 H), 4.15 (d, J = 11.26 Hz, 2H), 4.53 (d, J = 13.65 Hz, 1 H), 4.75 (d, J = 13.65 Hz, 1 H), 7.32 (d, J = 8.20 Hz, 1 H), 7.52 (d, J = 8.20 Hz, 1 H), 7.65 (s, 1 H). |
| 161 | 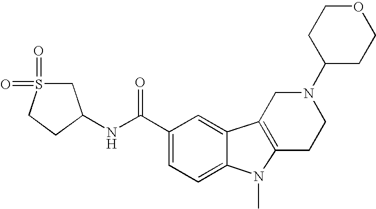 | N-(1,1- dioxidotetrahydro-3- thienyl)-5-methyl-2- (tetrahydro-2H-pyran- 4-yl)-2,3,4,5- tetrahydro-1H- pyrido[4,3-b]indole-8- carboxamide | MS (M + 1): 432.0. 1H NMR (400 MHz, CD3OD) δ ppm 1.82-1.99 (m, 2 H), 2.09- 2.22 (m, 2 H), 2.26-2.39 (m, 1 H), 2.54-2.66 (m, 1 H), 3.05-3.23 (m, 3 H), 3.31- 3.33 (m, 2 H), 3.43-3.61 (m, 4 H), 3.65-3.72 (m, 1 H), 3.74 (s, 3 H), 4.00-4.07 (m, 1 H), 4.10-4.15 (m, 2 H), 4.49- 4.57 (m, 1 H), 4.66-4.83 (m, 2 H), 7.48 (d, J = 8.75 Hz, 1 H), 7.71 (d, J = 8.75 Hz, 1 H), 8.05 (s, 1 H). |
| 162 | 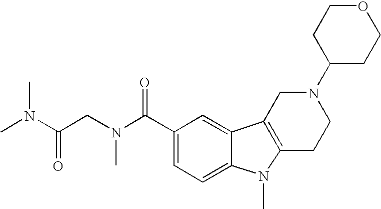 | N-[2-(dimethylamino)- 2-oxoethyl]-N,5- dimethyl-2-(tetrahydro- 2H-pyran-4-yl)-2,3,4,5- tetrahydro-1H- pyrido[4,3-b]indole-8- carboxamide | MS (M + 1): 413.0. 1H NMR (400 MHz, CD3OD) δ ppm 1.83-1.99 (m, 2 H), 2.10- 2.23 (m, 2 H), 2.99 (s, 3 H), 3.03 (s, 3 H), 3.09 (s, 3 H), 3.18-3.26 (m, 2 H), 3.42- 3.59 (m, 3 H), 3.72 (s, 3 H), 3.96-4.06 (m, 1 H), 4.07-4.15 (m, 2 H), 4.18-4.23 (m, 1H), 4.41 (s, 2 H), 4.45-4.53 (m, 1 H), 4.58-4.74 (m, 1 H), 7.35 (d, J = 8.40 Hz, 1 H), 7.46 (d, J = 8.40 Hz, 1 H), 7.65 (s, 1 H). |
| 163 | 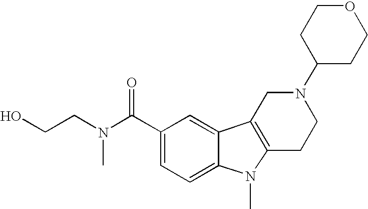 | N-(2-hydroxyethyl)- N,5-dimethyl-2- (tetrahydro-2H-pyran- 4-yl)-2,3,4,5- tetrahydro-1H- pyrido[4,3-b]indole-8- carboxamide | MS (M + 1): 372.0. 1H NMR (400 MHz, CD3OD) δ ppm 1.83-1.98 (m, 2 H), 2.09- 2.23 (m, 2 H), 3.03-3.14 (m, 3 H), 3.20-3.28 (m, 3 H), 3.41- 3.53 (m, 4 H), 3.61-3.70 (m, 2 H), 3.72 (s, 3 H), 3.78-3.85 (m, 1 H), 3.99-4.04 (m, 1 H), 4.07-4.16 (m, 2 H), 4.43- 4.55 (m, 1 H), 4.62-4.77 (m, 1 H), 7.30 (d, J = 8.40 Hz, 1 H), 7.46 (d, J = 8.40 Hz, 1 H), 7.61 (s, 1 H). |
| 164 | 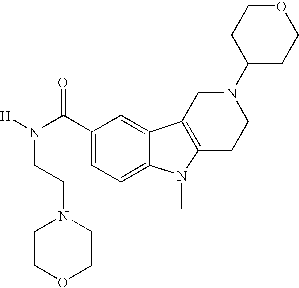 | 5-methyl-N-(2- morpholin-4-ylethyl)- 2-(tetrahydro-2H- pyran-4-yl)-2,3,4,5- tetrahydro-1H- pyrido[4,3-b]indole-8- carboxamide | MS (M + 1): 427.0. 1H NMR (400 MHz, CD3OD) δ ppm 1.82-1.98 (m, 2 H), 2.11- 2.22 (m, 2H), 3.19-3.27 (m, 4 H), 3.16-3.26 (m, 3 H), 3.41 (t, J = 5.66 Hz, 2H), 3.49 (t, J = 11.65 Hz, 2 H), 3.61-3.72 (m, 2 H), 3.74 (s, 3 H), 3.77- 3.85 (m, 4 H), 3.95-4.08 (m, 2 H), 4.07-4.15 (m, 2 H), 4.48- 4.61 (m, 1 H), 4.64-4.77 (m, 1 H), 7.48 (d, J = 8.73 Hz, 1 H), 7.75 (dd, J = 8.73 Hz, 1 H), 8.10 (s, 1 H). |
| 165 | 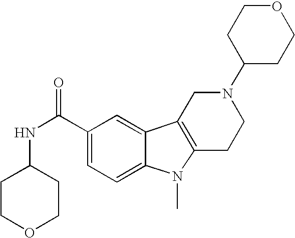 | 5-methyl-N,2- ditetrahydro-2H-pyran- 4-yl-2,3,4,5-tetrahydro- 1H-pyrido[4,3- b]indole-8- carboxamide | MS (M + 1): 398.0. 1H NMR (400 MHz, CD3OD) δ ppm 1.60-1.73 (m, 2 H), 1.83- 1.95 (m, 4 H), 2.09-2.22 (m, 2 H), 3.42-3.62 (m, 6 H), 3.73 (s, 3 H), 3.92-4.03 (m, 4 H), 4.05-4.15 (m, 4 H), 4.46- 4.57 (m, 1 H), 4.67-4.73 (m, 1 H), 7.45 (d, J = 8.75 Hz, 1 H), 7.69 (d, J = 8.75 Hz, 1 H), 8.02 (s, 1 H). |
| 166 | 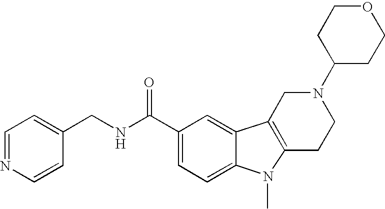 | 5-methyl-N-(pyridin-4- ylmethyl)-2- (tetrahydro-2H-pyran- 4-yl)-2,3,4,5- tetrahydro-1H- pyrido[4,3-b]indole-8- carboxamide | MS (M + 1): 405.0. 1H NMR (400 MHz, _{CD3OD}CD3OD) δ ppm 1.83-1.96 (m, 2 H), 2.12-2.19 (m, 2 H), 3.23-3.33 (m, 3H), 3.47 (t, J = 11.52 Hz, 2 H), 3.65-3.73 (m, 2 H), 3.74 (s, 3 H), 4.05-4.12 (m, 2 H), 4.50-4.74 (m, 2 H), 4.79 (s, 2 H), 7.50 (d, J = 8.65 Hz, 1 H), 7.78 (d, J = 8.65 Hz, 1 H), 7.87-8.02 (m, 2 H), 8.12 (s, 1 H), 8.62-8.95 (m, 2 H). |
| 167 | 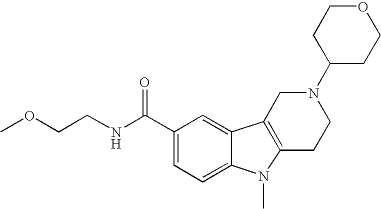 | N-(2-methoxyethyl)-5- methyl-2-(tetrahydro- 2H-pyran-4-yl)-2,3,4,5- tetrahydro-1H- pyrido[4,3-b]indole-8- carboxamide | MS (M + 1): 372.0. 1H NMR (400 MHz, CD3OD) δ ppm 1.86-1.96 (m, 2 H), 2.10- 2.21 (m, 2 H), 3.36 (s, 3 H), 3.45-3.54 (m, 3 H), 3.56 (s, 6 H), 3.66-3.71 (m, 1 H), 3.72- 3.75 (m, 3 H), 3.98-4.05 (m, 1 H), 4.09-4.16 (m, 2 H), 4.48- 4.58 (m, 1 H), 4.70-4.76 (m, 1 H), 7.46 (d, J = 8.59 Hz, 1 H), 7.69 (dd, J = 8.59 Hz, 1 H), 8.02 (s, 1 H). |
| 168 | 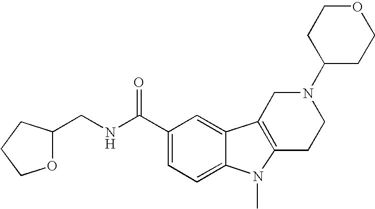 | 5-methyl-N- (tetrahydrofuran-2- ylmethyl)-2- (tetrahydro-2H-pyran- 4-yl)-2,3,4,5- tetrahydro-1H- pyrido[4,3-b]indole-8- carboxamide | MS (M + 1): 398.0. 1H NMR (400 MHz, CD3OD) δ ppm 1.60-1.73 (m, 1 H), 1.84- 1.97 (m, 4 H), 1.97-2.08 (m, 1 H), 2.11-2.23 (m, 2 H), 3.19-3.26 (m, 2 H), 3.35- 3.60 (m, 5 H), 3.67-3.78 (m, 2 H), 3.72 (s, 3 H), 3.85-3.93 (m, 1 H), 4.00-4.06 (m, 1 H), 4.08-4.15 (m, 3 H), 4.47- 4.56 (m, 1 H), 4.67-4.76 (m, 1 H), 7.45 (d, J = 8.79 Hz, 1 H), 7.69 (d, J = 8.79 Hz, 1 H), 8.02 (s, 1 H). |
| 169 | 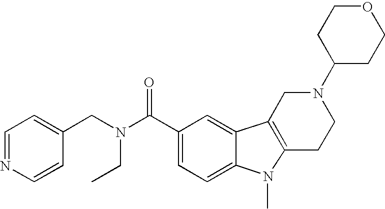 | N-ethyl-5-methyl-N- (pyridin-4-ylmethyl)-2- (tetrahydro-2H-pyran- 4-yl)-2,3,4,5- tetrahydro-1H- pyrido[4,3-b]indole-8- carboxamide | MS (M + 1): 433.0. 1H NMR (400 MHz, CD3OD) δ ppm 1.10-1.27 (m, 3 H), 1.85- 1.97 (m, 2 H), 2.11-2.24 (m, 2 H), 3.27-3.29 (m, 2 H), 3.42-3.60 (m, 5 H), 3.65- 3.73 (m, 1 H), 3.74 (s, 3 H), 3.89-4.06 (m, 1 H), 4.06- 4.17 (m, 2 H), 4.44-4.78 (m, 2 H), 4.97 (s, 2 H), 7.28-7.42 (m, 1 H), 7.48-7.54 (m, 1 H), 7.69-7.72 (m, 1 H), 7.93-8.05 (m, 2 H), 8.70-8.82 (m, 2 H). |
| 170 | 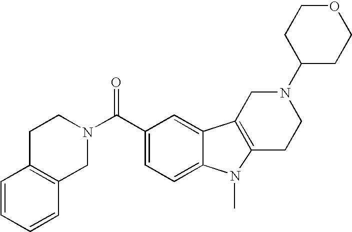 | 8-(3,4- dihydroisoquinolin- 2(1H)-ylcarbonyl)-5- methyl-2-(tetrahydro- 2H-pyran-4-yl)-2,3,4,5- tetrahydro-1H- pyrido[4,3-b]indole | MS (M + 1): 390.0. 1H NMR (400 MHz, CD3OD) δ ppm 1.81-1.96 (m, 2 H), 2.09- 2.22 (m, 2 H), 2.85-3.00 (m, 2 H), 3.21-3.30 (m, 4 H), 3.42- 3.52 (m, 2 H), 3.53-3.60 (m, 1 H), 3.63-3.73 (m, 2 H), 3.73- 3.78 (m, 3 H), 3.90-4.06 (m, 1 H), 4.04-4.13 (m, 2 H), 4.42-4.57 (m, 1 H), 4.60- 4.76 (m, 2H) , 7.07-7.25 (m, 4H), 7.32 (d, J = 8.40 Hz, 1 H), 7.50 (d, J = 8.40 Hz, 1 H), 7.65 (s, 1 H). |
| 171 | 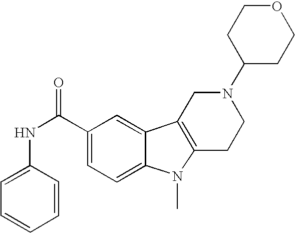 | 5-methyl-N-phenyl-2- (tetrahydro-2H-pyran- 4-yl)-2,3,4,5- tetrahydro-1H- pyrido[4,3-b]indole-8- carboxamide | MS (M + 1): 390.0. 1H NMR (400 MHz, CD3OD) δ ppm 1.84-1.98 (m, 2 H), 2.12- 2.21 (m, 2H), 3.28-3.29 (m, 2 H), 3.43-3.54 (m, 2 H), 3.56- 3.63 (m, 1 H), 3.67-3.74 (m, 1 H), 3.76 (s, 3 H), 3.97- 4.08 (m, 1 H), 4.109-4.15 (m, 2 H), 4.48-4.61 (m, 1 H), 4.70-4.82 (m, 1 H), 7.09- 7.16 (m, 1 H), 7.34 (t, J = 7.85 Hz, 2 H), 7.52 (d, J = 8.65 Hz, 1 H), 7.67 (d, J = 7.85 Hz, 2 H), 7.83 (d, J = 8.65 Hz, 1 H), 8.15 (s, 1 H). |
| 172 | 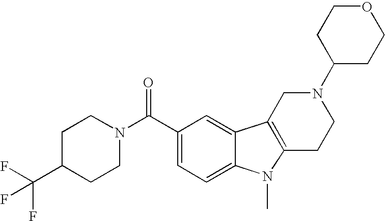 | 5-methyl-2-(tetrahydro- 2H-pyran-4-yl)-8-{[4- (trifluoromethyl) piperidin-1-yl] carbonyl}- 2,3,4,5-tetrahydro-1H- pyrido[4,3-b]indole | MS (M + 1): 450.0. 1H NMR (400 MHz, CD3OD) δ ppm 1.43-1.60 (m, 2 H), 1.83- 1.98 (m, 4 H), 2.08-2.21 (m, 2 H), 2.82-3.09 (m, 1 H), 3.21-3.27 (m, 2 H), 3.27- 3.31 (m, 3 H), 3.42-3.56 (m, 4 H), 3.64-3.70 (m, 1 H), 3.73 (s, 3 H), 3.97-4.06 (m, 1 H), 4.11 (dd, J = 11.72, 3.91 Hz, 2 H), 4.44-4.54 (m, 1 H), 4.66-4.76 (m, 1 H), 7.26 (dd, J = 8.55, 1.27 Hz, 1 H), 7.48 (d, J = 8.55 Hz, 1 H), 7.60 (s, 1 H). Anal. Calcd for C₂₄ H₃₀ F₃ N₃ O₂. 2C₂HF₃O₂: C, 49.63; H, 4.76; N, 6.20. Found: C, 49.89; H, 5.00; N, 6.49 |
| 173 | 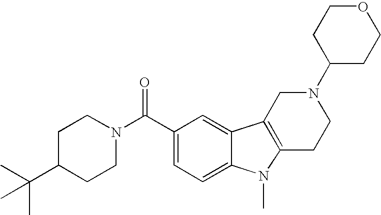 | 8-[(4-tert- butylpiperidin-1- yl)carbonyl]-5-methyl- 2-(tetrahydro-2H- pyran-4-yl)-2,3,4,5- tetrahydro-1H- pyrido[4,3-b]indole | MS (M + 1): 438.0. 1H NMR (400 MHz, CD3OD) δppm 0.87 (s, 9 H), 1.13-1.41 (m, 3 H), 1.57-1.76 (m, 1 H), 1.79 2.00 (m, 3 H), 2.08-2.22 (m, 2 H), 2.66-2.89 (m, 1 H), 2.95-3.12 (m, 1 H), 3.21- 3.26 (m, 2 H), 3.40-3.60 (m, 3 H), 3.63-3.71 (m, 1 H), 3.72 (s, 3 H), 3.80-3.95 (m, 1 H), 3.96-4.06 (m, 1 H), 4.10 (dd, J = 11.82, 4.00 Hz, 2 H), 4.43-4.55 (m, 1 H), 4.64- 4.77 (m, 2 H), 7.24 (dd, J = 8.40, 1.17 Hz, 1 H), 7.46 (d, J = 8.40 Hz, 1 H), 7.57 (s, 1 H). |
| 174 | 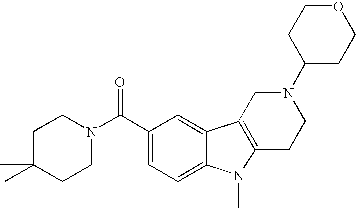 | 8-[(4,4- dimethylpiperidin-1- yl)carbonyl]-5-methyl- 2-(tetrahydro-2H- pyran-4-yl)-2,3,4,5- tetrahydro-1H- pyrido[4,3-b]indole | MS (M + 1): 410.0. 1H NMR (400 MHz, CD3OD) δ ppm 1.00 (s, 6 H), 1.26-1.54 (m, 4 H), 1.84-1.96 (m, 2 H), 2.11- 2.22 (m, 2 H), 3.21-3.26 (m, 2 H), 3.39-3.60 (m, 6 H), 3.62-3.70 (m, 2 H), 3.72 (s, 3 H), 3.97-4.05 (m, 1 H), 4.11 (dd, J = 11.80, 4.01 Hz, 2 H), 4.43-4.55 (m, 1 H), 4.65- 4.76 (m, 1 H), 7.24 (dd, J = 8.40, 1.37 Hz, 1 H), 7.46 (d, J = 8.40 Hz, 1 H), 7.57 (s, 1 H). |
| 175 | 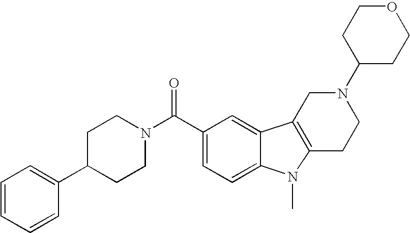 | 5-methyl-8-[(4- phenylpiperidin-1- yl)carbonyl]-2- (tetrahydro-2H-pyran- 4-yl)-2,3,4,5- tetrahydro-1H- pyrido[4,3-b]indole | MS (M + 1): 458.0. 1H NMR (400 MHz, CD3OD) δ ppm 1.61-1.82 (m, 3 H), 1.84- 1.99 (m, 3 H), 2.09-2.22 (m, 2 H), 2.77-3.00 (m, 1 H), 3.17-3.26 (m, 4 H), 3.40- 3.62 (m, 4 H), 3.64-3.72 (m, 1 H), 3.73 (s, 3 H), 3.86-4.06 (m, 2 H), 4.11 (dd, J = 11.72, 3.91 Hz, 2 H), 4.44 -4.55 (m, 1 H), 4.68-4.76 (m, 1 H), 7.13-7.20 (m, 1 H), 7.21- 7.28 (m, 4 H), 7.31 (dd, J = 8.59, 1.37 Hz, 1 H), 7.49 (d, J = 8.59 Hz, 1 H),7.63 (s, 1 H). Anal. Calcd for C₂₉ H₃₅ N₃ O₂. 1.75C₂HF₃O₂.0.5H₂O: C, 58.60; H, 5.63; N, 6.31. Found: C, 58.63; H, 5.71; N, 6.26 |

### Example 176

### N-(5-allyl-2-cyclopentyl-2,3,4,4a,5,9b-hexahydro-1H-pyrido[4,3-b]indol-8-yl)-N-methylbenzenesulfonamide

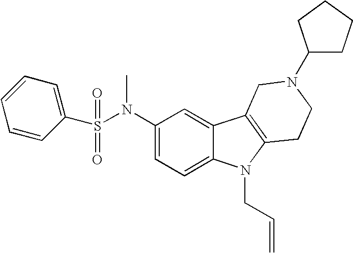

### Step A: The preparation of 8-allyl 2-tert-butyl 5-allyl-1,3,4,4a,5,9b-hexahydro-2H-pyrido[4,3-b]indole-2,8-dicarboxylate

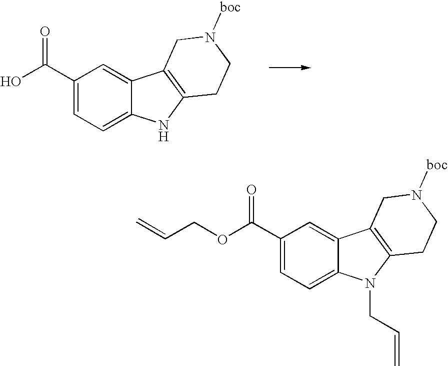

To a solution of 2-(tert-butoxycarbonyl)-2,3,4,4a,5,9b-hexahydro-1H-pyrido[4,3-b]indole-8-carboxylic acid (2.6 g, 8.2 mmol) in dry DMF (30 mL) was added 60% NaH (984 mg, 24.6 mmol) in one portion at 0° C. under nitrogen. The mixture was stirred at room temperature for 30 min, then allyl bromide (2.1 mL, 24.6 mmol) was added at room temperature, and the reaction mixture was stirred at room temperature for overnight. The solvent was removed in vacuo and the residue was dissolved in dichloromethane (200 mL), washed with water (50 mL), dried over sodium sulfate. Removal of solvent gave the crude product, which was purified on silica gel (5-60% EtOAc in dichloromethane) to give the desired product as yellow oil (1.58 g, 48%). MS (M+1): 397.3.

### Step B: The preparation of 5-allyl-2-(tert-butoxycarbonyl)-2,3,4,4a,5,9b-hexahydro-1H-pyrido[4,3-b]indole-8-carboxylic acid

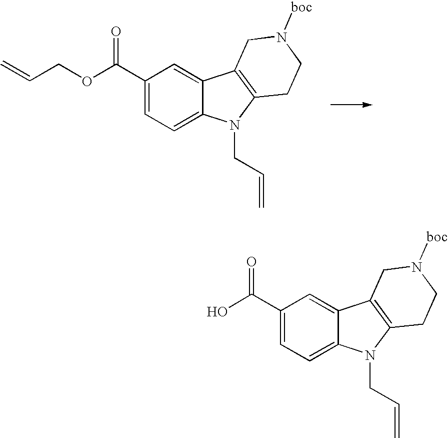

To a solution of 8-allyl 2-tert-butyl 5-allyl-1,3,4,4a,5,9b-hexahydro-2H-pyrido[4,3-b]indole-2,8-dicarboxylate (900 mg, 2.27 mmol) in dioxane/methanol/water (1:1:1 v/v, 15 mL) was added LiOH (136 mg, 5.68 mmol). The reaction mixture was stirred at 80° C. for 3 h. After cooled to room temperature, the reaction mixture was concentrated, then water (10 mL) was added. The solution was neutralized with HOAc to pH˜5.0, the precipitate was collected by filtration, yielded desired product as white solid (560 mg, 69%). MS (M+1): 357.0.

### Step C: The preparation of tert-butyl 5-allyl-8-({([2-(trimethylsilyl)ethoxy]carbonyl}amino)-1,3,4,4a,5,9b-hexahydro-2H-pyrido[4,3-b]indole-2-carboxylate

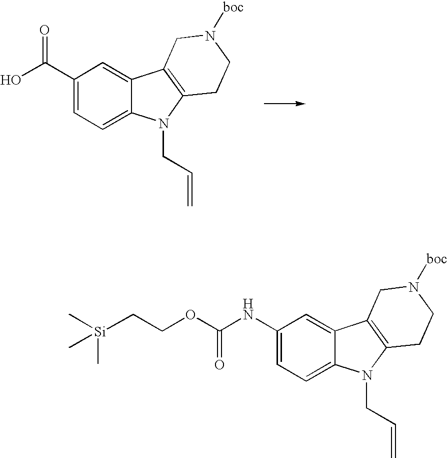

To the suspension of 5-allyl-2-(tert-butoxycarbonyl)-2,3,4,4a,5,9b-hexahydro-1H-pyrido[4,3-b]indole-8-carboxylic acid (190 mg, 0.53 mmol) in dry toluene (12 mL) was added diphenylphosphoryl azide (0.64 mmol) followed by triethyl amine (0.64 mmol), then trimethylsilyl ethanol (1.06 mmol) was added. The mixture was stirred at 100° C. for 6 h. After cooled to room temperature, the reaction mixture was concentrated, then dichloromethane (20 mL) was added, extracted with saturated sodium bicarbonate (10 mL), dried over sodium sulfate. Removal of solvent gave the crude product which was purified on silica gel (5% EtOAc in dichloromethane) to give the desired product as yellow oil (225 mg, 90%). MS (M+1): 472.3. 1H NMR (400 MHz, CDCl3) δ ppm 0.04 (s, 9H), 1.00-1.07 (m, 2H), 1.48 (s, 9H), 2.72 (s, 2H), 3.79 (s, 2H), 4.22-4.27 (m, 2H), 4.54-4.62 (m, 4H), 4.82 (d, J=17.18 Hz, 1H), 5.08 (d, J=10.15 Hz, 1H), 5.80-5.92 (m, 1H), 6.70 (s, 1H), 6.98 (s, 1H), 7.13 (d, J=8.59 Hz, 1H), 7.66 (s, 1H).

### Step D: The preparation of tert-butyl 5-allyl-8-(methyl{[2-(trimethylsilyl)ethoxy]carbonyl}amino)-1,3,4,4a,5,9b-hexahydro-2H-pyrido[4,3-b]indole-2-carboxylate

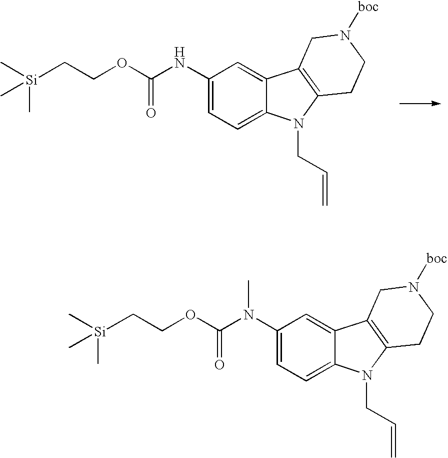

To a solution of tert-butyl 5-allyl-8-({[2-(trimethylsilyl)ethoxy]carbonyl}amino)-1,3,4,4a,5,9b-hexahydro-2H-pyrido[4,3-b]indole-2-carboxylate (225 mg, 0.48 mmol) in dry DMF (5 mL) was added 60% NaH (0.72 mmol) in one portion at 0° C. under nitrogen. The mixture was stirred at room temperature for 30 min, then methyl iodide (0.72 mmol) was added at room temperature, and the reaction mixture was stirred at room temperature for 3 h. The solvent was removed in vacuo and the residue was dissolved in dichloromethane (20 mL), washed with water (10 mL), dried over sodium sulfate. Removal of solvent gave the crude product, which was used without further purification. MS (M+1): 486.1.

### Step E: The preparation of tert-butyl 5-allyl-8-(methylamino)-1,3,4,4a,5,9b-hexahydro-2H-pyrido[4,3-b]indole-2-carboxylate

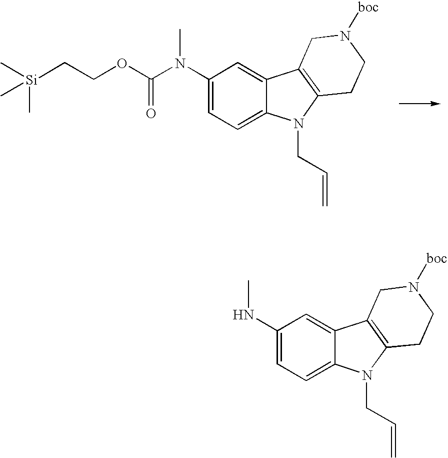

To the solution of crude tert-butyl 5-allyl-8-(methyl{[2-(trimethylsilyl)ethoxy]carbonyl}amino)-1,3,4,4a,5,9b-hexahydro-2H-pyrido[4,3-b]indole-2-carboxylate (0.48 mmol) in dry THF (6 mL) was added TBAF (1M in THF, 0.5 mL). The solution was stirred at room temperature for 3 h. The solvent was removed in vacuo and the residue was dissolved in dichloromethane (20 mL), washed with saturated sodium bicarbonate (10 mL), dried over sodium sulfate. Removal of solvent gave the crude product, which was used without further purification. MS (M+1): 342.1.

### Step F: The preparation of tert-butyl 5-allyl-8-[methyl(phenylsulfonyl)amino]-1,3,4,4a,5,9b-hexahydro-2H-pyrido[4,3-b]indole-2-carboxylate

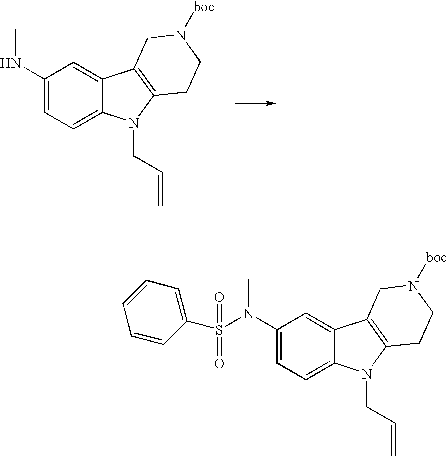

To a solution of crude tert-butyl 5-allyl-8-(methylamino)-1,3,4,4a,5,9b-hexahydro-2H-pyrido[4,3-b]indole-2-carboxylate (Example XL-4G, 0.48 mmol) in dry dichloromethane (5 mL) was added benzenesulfonyl chloride (0.96 mmol) followed by triethyl amine (0.96 mmol). The reaction mixture was stirred at room temperature for overnight. Dichloromethane (20 mL) was added and the solution was washed with water (10 mL), dried over sodium sulfate. Removal of solvent gave the crude product, which was purified by flash chromatography (20% ethyl acetate in hexanes) to give desired product as an oil (103 mg, 45% for 3 steps). MS (M+1): 482.3.

### Step G: The preparation of N-(5-allyl-2,3,4,4a,5,9b-hexahydro-1H-pyrido[4,3-b]indol-8-yl)-N-methylbenzenesulfonamide

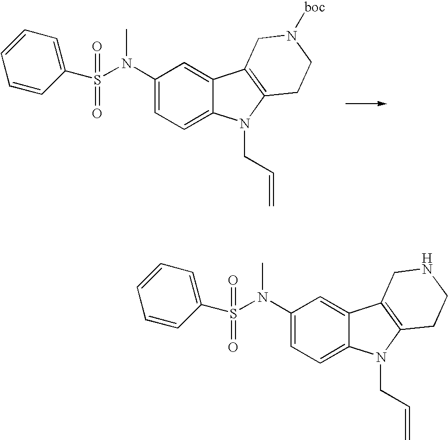

To a solution of tert-butyl 5-allyl-8-[methyl(phenylsulfonyl)amino]-1,3,4,4a,5,9b-hexahydro-2H-pyrido[4,3-b]indole-2-carboxylate (103 mg, 0.21 mmol) in dioxane (2 mL) was added 4N HCl in dioxane (1.5 mL). The solution was stirred at room temperature for 3 h. The solvent was removed in vacuo and the residue was dissolved in dichloromethane (30 mL), washed with saturated sodium bicarbonate (10 mL), dried over sodium sulfate. Removal of solvent gave the desired product, which was used without further purification. MS (M+1): 382.3.

### Step H: The preparation of N-(5-allyl-2-cyclopentyl-2,3,4,4a,5,9b-hexahydro-1H-pyrido[4,3-b]indol-8-yl)-N-methylbenzenesulfonamide

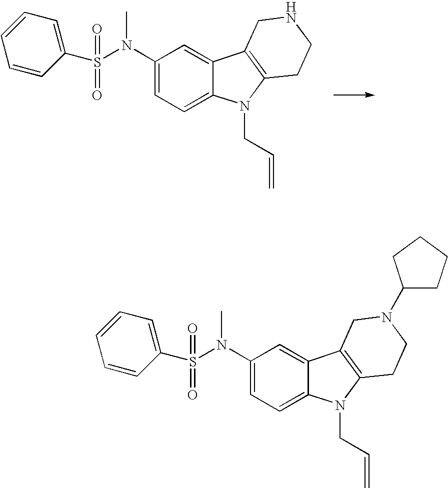

N-(5-allyl-2-cyclopentyl-2,3,4,4a,5,9b-hexahydro-1H-pyrido[4,3-b]indol-8-yl)-N-methylbenzenesulfonamide (0.1 mmol, 48 mg) was treated with cyclopentanone (0.2 mmol) and sodium triacetoxyborohydride (0.15 mmol) in dichloromethane. The mixture was stirred at room temperature overnight, diluted with dichloromethane (15 mL), washed with saturated NaHCO₃ solution (5 mL), dried over Na₂SO₄. The dichloromethane solution was concentrated in vacuo and the residue was purified with reverse phase HPLC to give the desired product as its TFA salt (16 mg, 28%). MS (M+1): 450.0. 1H NMR (400 MHz, CDCl3) δ ppm 1.24 (s, 1H), 1.58-1.73 (m, 2H), 1.82-1.96 (m, 2H), 1.97-2.21 (m, 4H), 2.89-3.01 (m, 1H), 3.21 (s, 3H), 3.30-3.44 (m, 2H), 3.59 (m, 1H), 3.91 (m, 1H), 4.12 (d, J=14.06 Hz, 1H), 4.62 (s, 2H), 4.68 (d, J=14.25 Hz, 1H), 4.80 (d, J=16.99 Hz, 1H), 5.14 (d, J=10.15 Hz, 1H), 5.79-5.96 (m, 1H), 6.84 (d, J=9.96 Hz, 1H), 7.09-7.19 (m, 2H), 7.43 (t, J=7.71 Hz, 2H), 7.57 (t, J=8.20 Hz, 3H).

### Example 177

### N-methyl-N-[5-propyl-2-(tetrahydro-2H-pyran-4-yl)-2,3,4,4a,5,9b-hexahydro-1H-pyrido[4,3-b]indol-8-yl]benzenesulfonamide

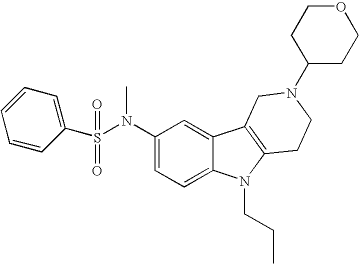

### Step A. The preparation of N-[5-allyl-2-(tetrahydro-2H-pyran-4-yl)-2,3,4,4a,5,9b-hexahydro-1H-pyrido[4,3-b]indol-8-yl]-N-methylbenzenesulfonamide

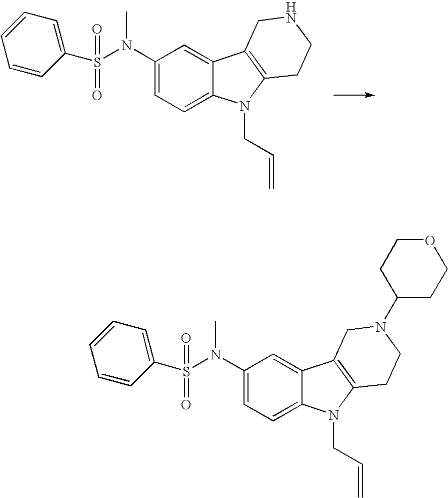

N-(5-allyl-2-cyclopentyl-2,3,4,4a,5,9b-hexahydro-1H-pyrido[4,3-b]indol-8-yl)-N-methylbenzenesulfonamide (0.05 mmol, 20 mg) was treated with tetrahydro-4H-pyran-4-one (0.1 mmol) and sodium triacetoxyborohydride (0.075 mmol) in dichloromethane (2 mL). The mixture was stirred at room temperature overnight, diluted with dichloromethane (15 mL), washed with saturated NaHCO₃ solution (5 mL), dried over Na₂SO₄. The dichloromethane solution was concentrated in vacuo and the residue was used without further purification. MS (M+1): 466.1.

### Step B: The preparation of N-methyl-N-[5-propyl-2-(tetrahydro-2H-pyran-4-yl)-2,3,4,4a,5,9b-hexahydro-1H-pyrido[4,3-b]indol-8-yl]benzenesulfonamide

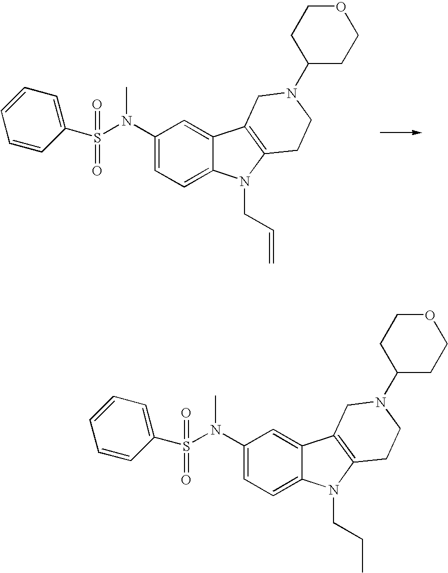

The solution of crude N-[5-allyl-2-(tetrahydro-2H-pyran-4-yl)-2,3,4,4a,5,9b-hexahydro-1H-pyrido[4,3-b]indol-8-yl]-N-methylbenzenesulfonamide (0.05 mmol) in methaol (5 mL) was hydrogenated in the presence of 5% Pd/C at 30 psi (room temperature) for 3 h. After filtration, the solution was concentrated and the residue was purified with reverse phase HPLC to give the desired product as a TFA salt (6 mg, 21% for 2 steps). MS (M+1): 468.1. 1 H NMR (600 MHz, CDCl3) δ ppm 0.99 (m, 3H), 1.34 (m, 2H), 2.14 (m, 3H), 2.25 (m, 1H), 3.06 (m, 1H), 3.32 (s, 3H), 3.45 (m, 1H), 3.57 (m, 2H), 3.64 (m, 1H), 3.80 (m, 1H), 4.06 (m, 3H), 4.27 (m, 3H), 4.63 (m, 1H), 6.92 (m, 1H), 7.27 (m, 2H), 7.56 (m, 2H), 7.68 (m, 3H).

### Example 178

### N-[5-allyl-2-(tetrahydro-2H-pyran-4-yl)-2,3,4,4a,5,9b-hexahydro-1H-pyrido[4,3-b]indol-8-yl]benzenesulfonamide

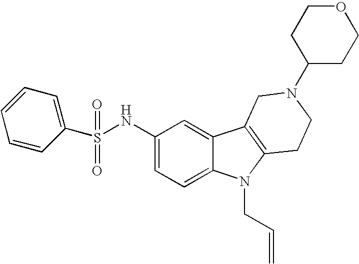

### Step A: The preparation of 2-(trimethylsilyl)ethyl (5-allyl-2,3,4,4a,5,9b-hexahydro-1H-pyrido[4,3-b]indol-8-yl)carbamate

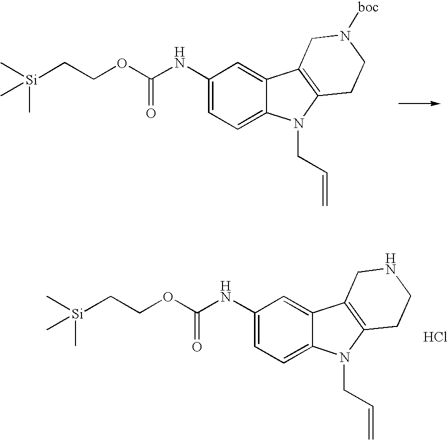

To a solution of crude tert-butyl 5-allyl-8-({[2-(trimethylsilyl)ethoxy]carbonyl}amino)-1,3,4,4a,5,9b-hexahydro-2H-pyrido[4,3-b]indole-2-carboxylate (930 mg, 1.97 mmol) in dioxane (10 mL) was added 4N HCl in dioxane (6 mL). The solution was stirred at room temperature for 3 h. The solvent was removed in vacuo and the residue was washed with diethyl ether, the solid was collected by filtration, dried under vacuum to give the desired product as its HCl salt (620 mg, 76%), which was used without further purification. MS (M+1): 372.1.

### Step B. The preparation of 2-(trimethylsilyl)ethyl[5-allyl-2-(tetrahydro-2H-pyran-4-yl)-2,3,4,4a,5,9b-hexahydro-1H-pyrido[4,3-b]indol-8-yl]carbamate

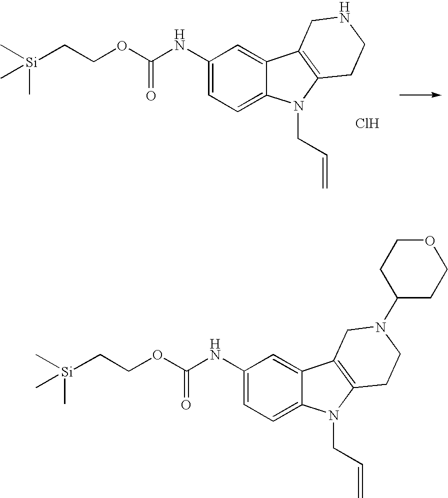

2-(trimethylsilyl)ethyl (5-allyl-2,3,4,4a,5,9b-hexahydro-1H-pyrido[4,3-b]indol-8-yl)carbamate (HCl salt, 310 mg, 0.76 mmol) was treated with triethyl amine (0.76 mmol) followed by tetrahydro-4H-pyran-4-one (1.52 mmol) and sodium triacetoxyborohydride (1.14 mmol) in dichloromethane (20 mL). The mixture was stirred at room temperature overnight, diluted with dichloromethane (50 mL), washed with saturated NaHCO₃ solution (20 mL), dried over Na₂SO₄. The dichloromethane solution was concentrated in vacuo, and the residue was purified by passing through a short pad of silica gel (30˜90% ethyl acetate in dichloromethane) to give the desired product (90 mg, 27%). MS (M+1): 456.4.

### Step C: The preparation of 5-allyl-2-(tetrahydro-2H-pyran-4-yl)-2,3,4,4a,5,9b-hexahydro-1H-pyrido[4,3-b]indol-8-amine

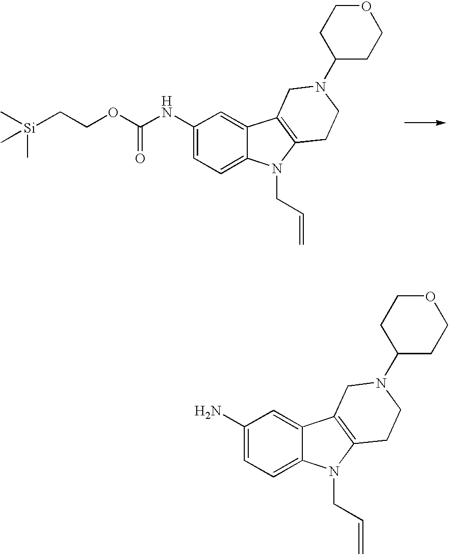

To the solution of 2-(trimethylsilyl)ethyl[5-allyl-2-(tetrahydro-2H-pyran-4-yl)-2,3,4,4a,5,9b-hexahydro-1H-pyrido[4,3-b]indol-8-yl]carbamate (90 mg, 0.2 mmol) in dry THF (3 mL) was added TBAF (1M in THF, 0.5 mL). The solution was stirred at room temperature for 3 h. The solvent was removed in vacuo and the residue was dissolved in dichloromethane (20 mL), washed with saturated sodium bicarbonate (10 mL), dried over sodium sulfate. Removal of solvent gave the crude product (62 mg, 99%), which was used without further purification. MS (M+1): 312.1.
Step D: The preparation of N-[5-allyl-2-(tetrahydro-2H-pyran-4-yl)-2,3,4,4a,5,9b-hexahydro-1H-pyrido[4,3-b]indol-8-yl]benzenesulfonamide

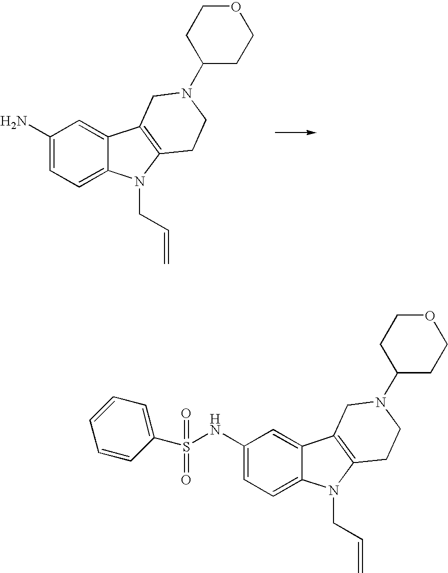

To a solution of crude 5-allyl-2-(tetrahydro-2H-pyran-4-yl)-2,3,4,4a,5,9b-hexahydro-1H-pyrido[4,3-b]indol-8-amine (32 mg, 0.1 mmol) in dry dichloromethane (2 mL) was added benzenesulfonyl chloride (0.11 mmol) followed by triethyl amine (0.2 mmol). The reaction mixture was stirred at room temperature for overnight. Dichloromethane (10 mL) was added and the solution was washed with water (10 mL), dried over sodium sulfate. Removal of solvent gave the crude product which was purified by reverse phase HPLC to give desired product as a TFA salt (34 mg, 60%). MS (M+1): 452.0. 1H NMR (600 MHz, CDCl3) δ ppm 1.42 (m, 2H), 1.60-1.73 (m, 2H), 3.03 (m, 2H), 3.21 (m, 2H), 3.32 (m, 1H), 3.50 (m, 2H), 3.80 (m, 1H), 3.91 (m, 1H), 4.16 (d, J=9.73 Hz, 2H), 4.24 (m, 1H), 4.62 (m, 1H), 4.74 (d, J=17.15 Hz, 1H), 5.11 (d, J=10.50 Hz, 1H), 5.84 (m, 1H), 6.76 (d, J=8.19 Hz, 1H), 7.02 (d, J=8.70 Hz, 1H), 7.23 (s, 1H), 7.39 (m, 3H), 7.50 (m, 1H), 7.70 (d, J=7.68 Hz, 2H).

### Example 179

### 5-allyl-2-cyclopentyl-8-nitro-2,3,4,4a,5,9b-hexahydro-1H-pyrido[4,3-b]indole

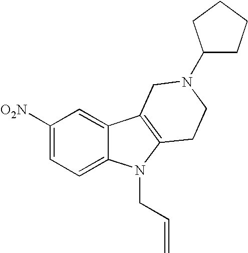

### Step A: The preparation of 8-nitro-2,3,4,4a,5,9b-hexahydro-1H-pyrido[4,3-b]indole

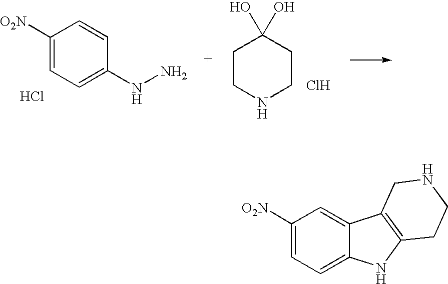

A mixture of 4-nitrophenylhydrazine hydrochloride (5 mmol) and 4-piperidone monohydrate hydrochloride (5 mmol) in a sealed tube was added formic acid (5 mL) and stirred at 120° C. for overnight. After cooling to room temperature, dioxane (5 mL) was added and the solid was collected by filtration to give desired product as a brown solid in its formic acid salt form (560 mg, 43%). MS (M+1): 217.9.

### Step B: The preparation of 2-cyclopentyl-8-nitro-2,3,4,4a,5,9b-hexahydro-1H-pyrido[4,3-b]indole

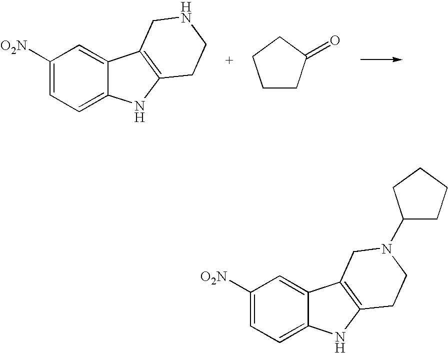

To a suspension of 8-nitro-2,3,4,4a,5,9b-hexahydro-1H-pyrido[4,3-b]indole (HCOOH salt, 1.5 g, 5.73 mmol) in methanol (80 mL) was added triethyl amine (8.60 mmol) followed by cyclopentanone (8.60 mmol) and sodium cyanoborohydride (11.5 mmol). The mixture was stirred at 50° C. for overnight. Another equivalent of sodium cyanoborohydride (5.73 mmol) was added and stirred at 50° C. for 6 h. The solvent was removed under reduced pressure, the residue was taken into dichloromethane (100 mL), washed with saturated sodium bicarbonate (20 mL), dried over sodium sulfate. Removal of solvent gave the crude product (903 mg, 55%), which was used without further purification. MS (M+1): 286.0. Remaining starting material precipitated during work up was recovered by filtration (˜810 mg).

### Step C: The preparation of 5-allyl-2-cyclopentyl-8-nitro-2,3,4,4a,5,9b-hexahydro-1H-pyrido[4,3-b]indole

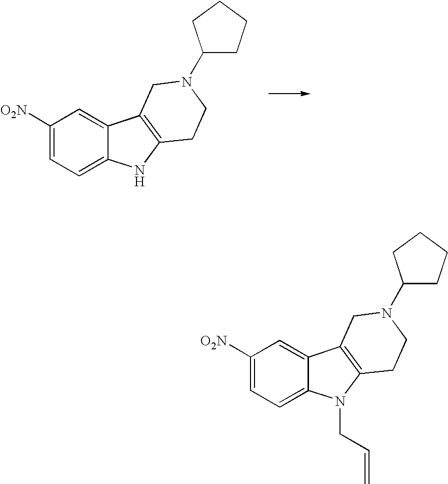

2-cyclopentyl-8-nitro-2,3,4,4a,5,9b-hexahydro-1H-pyrido[4,3-b]indole (4.2 mmol) was treated with 60% NaH (8.4 mmol) in DMF (25 mL) at 0° C. for 30 min and then added ally bromide (5.04 mmol). After being stirred at room temperature for overnight, the solvent was removed in vacuo and the residue was dissolved in dichloromethane (80 mL), washed with water (20 mL), dried over sodium sulfate. Removal of solvent gave the crude product, which was purified with flash chromatography (10-80% EtOAc in dichloromethane) to give the desired product as brown oils (1.3 g, 95%). MS (M+1): 326.29. The sample for biological test was further purified with reverse phase HPLC to give a TFA salt as light yellow powders. 1H NMR (600 MHz, CDCl3) δ ppm 1.70 (m, 4H), 1.95 (m, 2H), 2.17 (m, 2H), 2.97 (m, 1H), 3.34 (m, 1H), 3.60 (m, 2H), 4.03 (m, 1H), 4.19 (m, 1H), 4.73 (m, 2H), 4.81 (m, 1H), 4.87 (d, J=14.34 Hz, 1H), 5.22 (d, J=10.50 Hz, 1H), 5.94 (m, 1H), 7.33 (d, J=8.96 Hz, 1H), 8.16 (d, J=8.96 Hz, 1H), 8.42 (s, 1H).

### Example 180

### 5-allyl-8-nitro-2-(tetrahydro-2H-pyran-4-yl)-2,3,4,4a,5,9b-hexahydro-1H-pyrido[4,3-b]indole

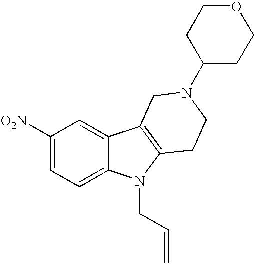

### Step A. The preparation of 8-nitro-2-(tetrahydro-2H-pyran-4-yl)-2,3,4,4a,5,9b-hexahydro-1H-pyrido[4,3-b]indole

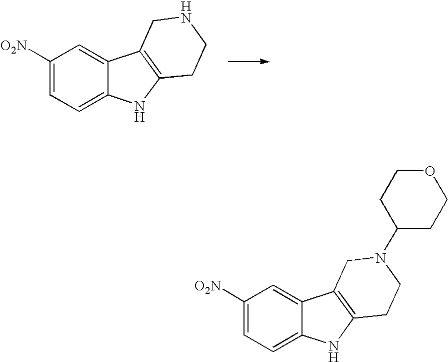

To a suspension of 8-nitro-2,3,4,4a,5,9b-hexahydro-1H-pyrido[4,3-b]indole (HCOOH salt, 1.7 g, 6.49 mmol) in methanol (100 mL) was added triethyl amine (9.74 mmol) followed by tetrahydro-4H-pyran-4-one (9.74 mmol) and sodium cyanoborohydride (13.0 mmol). The mixture was stirred at 50° C. for overnight. Another part of tetrahydro-4H-pyran-4-one (3.25 mmol) and sodium cyanoborohydride (6.49 mmol) were added and stirred at 50° C. for overnight. The solvent was removed under reduced pressure, the residue was taken into dichloromethane (100 mL), washed with saturated sodium bicarbonate (20 mL), dried over sodium sulfate. Removal of solvent gave the crude product (1.84 g, 94%), which was used without further purification. MS (M+1): 302.25.

### Step B: The preparation of 5-allyl-8-nitro-2-(tetrahydro-2H-pyran-4-yl)-2,3,4,4a,5,9b-hexahydro-1H-pyrido[4,3-b]indole

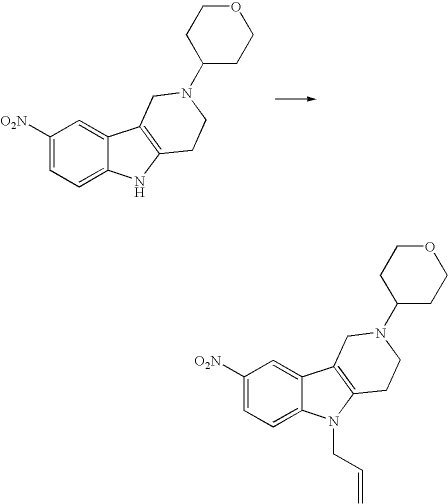

8-nitro-2-(tetrahydro-2H-pyran-4-yl)-2,3,4,4a,5,9b-hexahydro-1H-pyrido[4,3-b]indole (5.0 mmol) was treated with 60% NaH (10.0 mmol) in DMF (20 mL) at 0° C. for 30 min and then added allyl bromide (6.0 mmol). After being stirred at room temperature for overnight, the solvent was removed in vacuo and the residue was dissolved in dichloromethane (80 mL), washed with water (20 mL), dried over sodium sulfate. Removal of solvent gave the crude product, which was purified with flash chromatography (10-80% EtOAc in dichloromethane) to give the desired product as brown oils (1.62 g, 95%). MS (M+1): 342.04. The sample for biological test was further purified with reverse phase HPLC to give a TFA salt as light yellow powders. 1H NMR (600 MHz, CDCL3) δ ppm 1.86 (m, 2H), 2.07 (m, 2H), 2.98 (m, 1H), 3.35 (m, 1H), 3.54 (m, 4H), 3.76 (t, J=11.78 Hz, 1H), 3.95 (m, 1H), 4.20 (m, 2H), 4.29 (m, 1H), 4.73 (s, 2H), 4.80 (d, J=17.15 Hz, 1H), 5.22 (d, J=10.24 Hz, 1H), 5.93 (m, 1H), 7.33 (d, J=8.86 Hz, 1H), 8.16 (d, J=8.86 Hz, 1H), 8.41 (s, 1H).

### Example 181

### N-(2-cyclopentyl-5-propyl-2,3,4,4a,5,9b-hexahydro-1H-pyrido[4,3-b]indol-8-yl)benzenesulfonamide

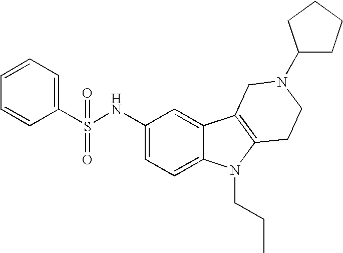

### Step A: The preparation of 2-cyclopentyl-5-propyl-2,3,4,4a,5,9b-hexahydro-1H-pyrido[4,3-b]indol-8-amine

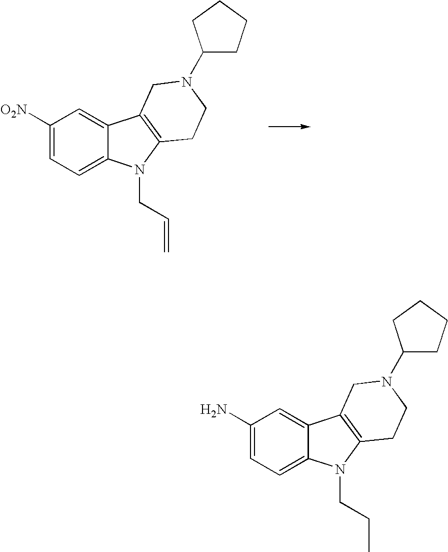

To a solution of 5-allyl-2-cyclopentyl-8-nitro-2,3,4,4a,5,9b-hexahydro-1H-pyrido[4,3-b]indole
(490 mg, 1.51 mmol) in ethyl acetate/methanol (1:1 v/v, 30 mL) was added 10% Pd/C (50 mg). The mixture was hydrogenated at room temperature (40 psi) for 5 h. After filtration and concentration, yielded crude product (403 mg, 90%) as an oil. MS (M+1): 298.33.

### Step B: The preparation of N-(2-cyclopentyl-5-propyl-2,3,4,4a,5,9b-hexahydro-1H-pyrido[4,3-b]indol-8-yl)benzenesulfonamide

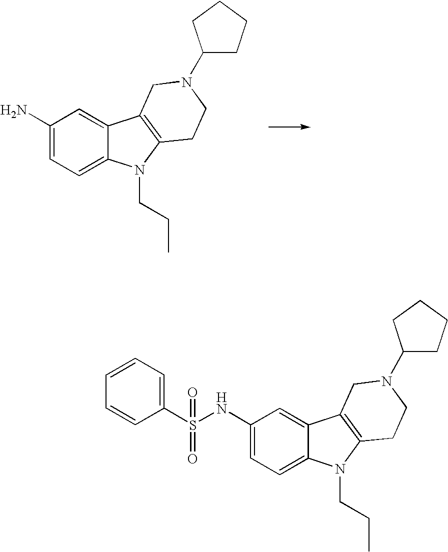

To a solution of crude 2-cyclopentyl-5-propyl-2,3,4,4a,5,9b-hexahydro-1H-pyrido[4,3-b]indol-8-amine (30 mg, 0.1 mmol) in dry dichloromethane (2 mL) was added benzenesulfonyl chloride (0.11 mmol) followed by triethyl amine (0.2 mmol). The reaction mixture was stirred at room temperature for overnight. Dichloromethane (10 mL) was added and the solution was washed with water (10 mL), dried over sodium sulfate. Removal of solvent gave the crude product, which was purified by reverse phase HPLC to give desired product as a TFA salt (36 mg, 65%). MS (M+1): 438.0. 1H NMR (600 MHz, CDCl3) δ ppm 0.88 (t, J=7.34 Hz, 3H), 1.72 (m, 4H), 1.91 (m, 2H), 2.13 (m, 4H), 2.95 (m, 1H), 3.34 (m, 1H), 3.46 (m, 1H), 3.61 (m, 1 H), 3.93 (m, 3H), 4.10 (m, 1H), 4.70 (m, 1H), 6.81 (d, J=8.80 Hz, 1H), 6.86 (s, 1H), 7.11 (d, J=8.80 Hz, 1H), 7.18 (s, 1H), 7.41 (t, J=7.63 Hz, 2H), 7.52 (m, 1H), 7.71 (d, J=7.63 Hz, 2H).

### Example 182

### N-(2-cyclopentyl-5-propyl-2,3,4,4a,5,9b-hexahydro-1H-pyrido[4,3-b]indol-8-yl)cyclopropanesulfonamide

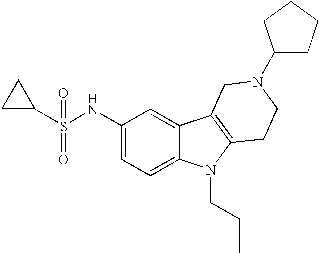

To a solution of crude 2-cyclopentyl-5-propyl-2,3,4,4a,5,9b-hexahydro-1H-pyrido[4,3-b]indol-8-amine (30 mg, 0.1 mmol) in dry dichloromethane (2 mL) was added cyclopropanesulfonyl chloride (0.11 mmol) followed by triethyl amine (0.2 mmol). The reaction mixture was stirred at room temperature for overnight. Dichloromethane (10 mL) was added and the solution was washed with water (10 mL), dried over sodium sulfate. Removal of solvent gave the crude product, which was purified by reverse phase HPLC to give desired product as a TFA salt (32 mg, 61%). MS (M+1): 402.0. 1H NMR (600 MHz, CDCl3) δ ppm 0.85-1.01 (m, 4H), 1.15 (m, 2H), 1.69 (m, 2H), 1.78 (m, 2H), 1.4-1.99 (m, 3H), 2.15 (m, 4H), 2.47 (m, 1H), 2.98 (m, 1H), 3.36 (m, 1H), 3.50 (m, 1H), 3.63 (m, 1H), 3.92-4.05 (m, 3H), 4.16 (d, J=13.75 Hz, 1H), 4.79 (d, J=13.75 Hz, 1H), 6.41 (s, 1H), 7.14 (d, J=8.45 Hz, 1H), 7.27 (d, J=8.45 Hz, 1H), 7.36 (s, 1H).

### Example 183

### N′-(2-cyclopentyl-5-propyl-2,3,4,4a,5,9b-hexahydro-1H-pyrido[4,3-b]indol-8-yl)-N,N-dimethylsulfamide

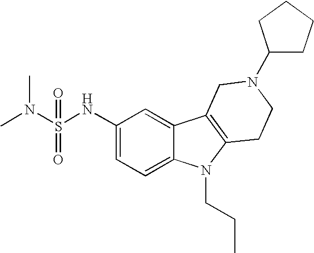

To a solution of crude 2-cyclopentyl-5-propyl-2,3,4,4a,5,9b-hexahydro-1H-pyrido[4,3-b]indol-8-amine (30 mg, 0.1 mmol) in dry acetonitrile (2 mL) was added dimethylsulfamoyl sulfonyl chloride (0.11 mmol) followed by triethyl amine (0.2 mmol). The reaction mixture was stirred at 50° C. for overnight. Dichloromethane (10 mL) was added and the solution was washed with water (10 mL), dried over sodium sulfate. Removal of solvent gave the crude product, which was purified by reverse phase HPLC to give desired product as a TFA salt (23 mg, 45%). MS (M+1): 405.0. 1H NMR (600 MHz, CDCl3) δ ppm 0.92 (t, J=7.04 Hz, 3H), 1.68 (m, 2H), 1.78 (m, 2H), 1.91 (m, 2H), 2.00-2.38 (m, 4H), 2.84 (s, 6H), 2.97 (m, 1H), 3.37 (m, 1H), 3.48 (m, 1H), 3.62 (m, 1H), 4.00 (m, 3H), 4.17 (m, 1H), 4.77 (m, 1H), 6.58 (s, 1H), 7.12 (d, J=8.22 Hz, 1H), 7.24 (d, J=8.22 Hz, 1H), 7.31 (s, 1H).

### Example 184

### N-(2-cyclopentyl-5-propyl-2,3,4,4a,5,9b-hexahydro-1H-pyrido[4,3-b]indol-8-yl)ethanesulfonamide

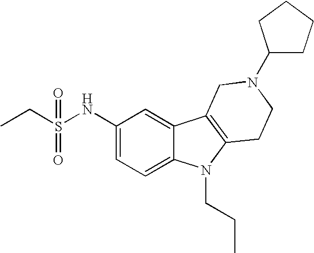

To a solution of crude 2-cyclopentyl-5-propyl-2,3,4,4a: 5,9b-hexahydro-1H-pyrido[4,3-b]indol-8-amine (30 mg, 0.1 mmol) in dry dichloromethane (2 mL) was added ethanesulfonyl chloride (0.11 mmol) followed by triethyl amine (0.2 mmol). The reaction mixture was stirred at room temperature for overnight. Dichloromethane (10 mL) was added and the solution was washed with water (10 mL), dried over sodium sulfate. Removal of solvent gave the crude product, which was purified by reverse phase HPLC to give desired product as a TFA salt (28 mg, 56%). MS (M+1): 390.0. 1H NMR (600 MHz, CDCl3) δ ppm 0.91 (t, J=7.21 Hz, 3H), 1.40 (t, J=7.04 Hz, 3H), 1.69 (m, 1H), 1.77 (m, 1H), 1.90 (m, 2H), 2.03 (m, 4H), 2.17 (m, 2H), 2.98 (m, 1H), 3.10 (q, J=7.04 Hz, 2H), 3.34 (m, 1H), 3.52 (m, 1H), 3.61 (m, 1H), 4.00 (m, 3H), 4.15 (m, 1H), 4.78 (m, 1H), 6.49 (s, 1H), 7.06 (d, J=8.80 Hz, 1H), 7.25 (d, J=8.80 Hz, 1H), 7.33 (s, 1H).

### Example 185

### N-(2-cyclopentyl-5-propyl-2,3,4,4a,5,9b-hexahydro-1H-pyrido[4,3-b]indol-8-yl)benzamide

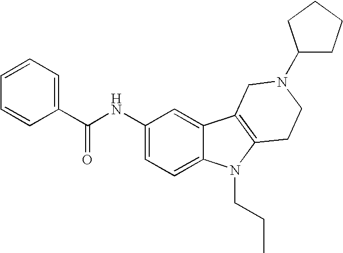

To a solution of crude 2-cyclopentyl-5-propyl-2,3,4,4a,5,9b-hexahydro-1H-pyrido[4,3-b]indol-8-amine (30 mg, 0.1 mmol) in dry dichloromethane (2 mL) was added benzoyl chloride (0.11 mmol) followed by triethyl amine (0.2 mmol). The reaction mixture was stirred at room temperature for overnight. Dichloromethane (10 mL) was added and the solution was washed with water (10 mL), dried over sodium sulfate. Removal of solvent gave the crude product, which was purified by reverse phase HPLC to give desired product as a TFA salt (33 mg, 63%). MS (M+1): 402.0. 1H NMR (600 MHz, CDCl3) δ ppm 0.92 (t, J=7.30 Hz, 3H), 1.67 (m, 2H), 1.79 (m, 2H), 1.89 (m, 2H), 2.07 (m, 1H), 2.16 (m, 3H), 2.99 (m, 1H), 3.42 (m, 2H), 3.60 (m, 1H), 4.01 (m, 3H), 4.19 (m, 1H), 4.76 (m, 1H), 7.28 (m, 1H), 7.29 (s, 1H), 7.52 (m, 2H), 7.58 (m, 1H), 7.92 (m, 3H), 8.01 (s, 1H).

### Example 186

### N-(2-cyclopentyl-5-propyl-2,3,4,4a,5,9b-hexahydro-1H-pyrido[4,3-b]indol-8-yl)propanamide

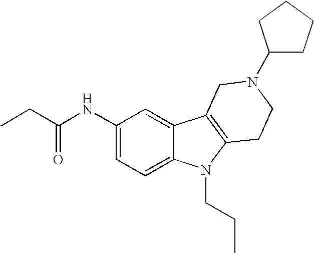

To a solution of crude 2-cyclopentyl-5-propyl-2,3,4,4a,5,9b-hexahydro-1H-pyrido[4,3-b]indol-8-amine (30 mg, 0.1 mmol) in dry dichloromethane (2 mL) was added propionyl chloride (0.11 mmol) followed by triethyl amine (0.2 mmol). The reaction mixture was stirred at room temperature for overnight. Dichloromethane (10 mL) was added and the solution was washed with water (10 mL), dried over sodium sulfate. Removal of solvent gave the crude product, which was purified by reverse phase HPLC to give desired product as a TFA salt (29 mg, 62%). MS (M+1): 354.0. 1H NMR (600 MHz, CDCl3) δ ppm 0.90 (t, J=7.14 Hz, 3H), 1.29 (t, J=7.41 Hz, 3H), 1.66 (m, 2H), 1.77 (m, 2H), 1.90 (s, 2H), 2.13 (m, 4H), 2.43 (m, 2H), 2.97 (m, 1H), 3.40 (m, 2H), 3.58 (m, 1H), 3.97 (m, 3H), 4.16 (m, 1H), 4.71 (m, 1H), 7.15 (d, J=8.51 Hz, 1H), 7.23 (d, J=8.51 Hz, 1H), 7.39 (s, 1H), 7.77 (s, 1H).

### Example 187

### N-(2-cyclopentyl-5-propyl-2,3,4,4a,5,9b-hexahydro-1H-pyrido[4,3-b]indol-8-yl)cyclopropanecarboxamide

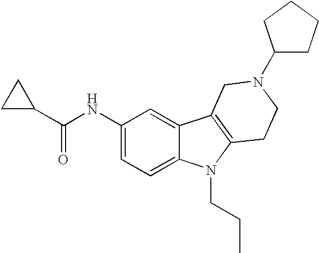

To a solution of crude 2-cyclopentyl-5-propyl-2,3,4,4a,5,9b-hexahydro-1H-pyrido[4,3-b]indol-8-amine (30 mg, 0.1 mmol) in dry dichloromethane (2 mL) was added cyclopropanecarbonyl chloride (0.11 mmol) followed by triethyl amine (0.2 mmol). The reaction mixture was stirred at room temperature for overnight. Dichloromethane (10 mL) was added and the solution was washed with water (10 mL), dried over sodium sulfate. Removal of solvent gave the crude product, which was purified by reverse phase HPLC to give desired product as a TFA salt (36 mg, 75%). MS (M+1): 366.0. 1H NMR (600 MHz, CDCl3) δ ppm 0.83-0.95 (m, 5H), 1.10 (m, 2H), 1.56 (m, 1H), 1.67 (m, 2H), 1.77 (m, 2H), 1.90 (m, 2H), 1.99-2.16 (m; 4H), 2.96 (m, 1H), 3.38 (m, 2H), 3.58 (m, 1H), 3.97 (m, 3H), 4.14 (m, 1H), 4.70 (m, 1H), 7.15 (d, J=8.68 Hz, 1H), 7.22 (d, J=8.68 Hz, 1H), 7.62 (s, 1H), 7.78 (s, 1H). Anal. Calcd for C23H31N3O.1.5C2HF3O2: C, 58.20; H, 6.10; N, 7.83. Found: C, 58.03; H, 5.89; N, 8.28.

### Example 188

### N-(2-cyclopentyl-5-propyl-2,3,4,4a,5,9b-hexahydro-1H-pyrido[4,3-]indol-8-yl)cyclopentanecarboxamide

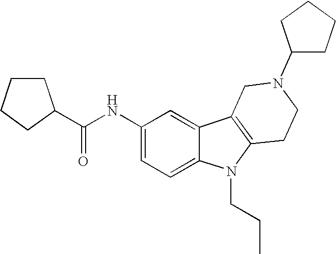

To a solution of crude 2-cyclopentyl-5-propyl-2,3,4,4a,5,9b-hexahydro-1H-pyrido[4,3-b]indol-8-amine (30 mg, 0.1 mmol) in dry dichloromethane (2 mL) was added cyclopentanecarbonyl chloride (0.11 mmol) followed by triethyl amine (0.2 mmol). The reaction mixture was stirred at room temperature for overnight. Dichloromethane (10 mL) was added and the solution was washed with water (10 mL), dried over sodium sulfate. Removal of solvent gave the crude product, which was purified by reverse phase HPLC to give desired product as a TFA salt (28 mg, 55%). MS (M+1): 394.0. 1H NMR (600 MHz, CDCL3) δppm 0.90 (t, J=7.27 Hz, 3H), 1.58-1.72 (m, 4H), 1.73-1.86 (m, 4H), 1.86-2.02 (m, 6H), 2.02-2.18 (m, 4H), 2.72 (m, 1H), 2.96 (m, 1H), 3.41 (m, 2H), 3.58 (m, 1H), 3.98 (m, 3H), 4.15 (m, 1H), 4.71 (m, 1H), 7.13 (d, J=8.65 Hz, 1H), 7.23 (d, J=8.65 Hz, 1H), 7.36 (s, 1H), 7.83 (s, 1H).

### Example 189

### N-(2-cyclopentyl-5-propyl-2,3,4,4a,5,9b-hexahydro-1H-pyrido[4,3-b]indol-8-yl)-N′-ethylurea

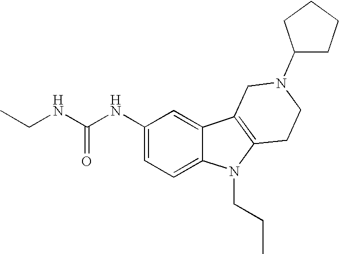

To a solution of crude 2-cyclopentyl-5-propyl-2,3,4,4a,5,9b-hexahydro-1H-pyrido[4,3-b]indol-8-amine (30 mg, 0.1 mmol) in dry DMF (2 mL) was added ethyl isocyanate (0.11 mmol) followed by triethyl amine (0.2 mmol). The reaction mixture was stirred at room temperature for overnight. Removal of solvent gave the crude product, which was purified by reverse phase HPLC to give desired product as a TFA salt (28 mg, 58%). MS (M+1): 369.0. 1H NMR (600 MHz, CD3OD) δppm 0.94 (t, J=7.34 Hz, 3H), 1.17 (t, J=7.34 Hz, 3H), 1.75-1.95 (m, 8H), 2.33 (m, 2H), 3.25 (m, 4H), 3.56 (m, 1H), 3.82 (m, 1H), 4.00 (m, 1H), 4.10 (m, 2H), 4.39 (d, J=13.50 Hz, 1H), 4.71 (d, J=13.50 Hz, 1H), 7.03 (d, J=8.55 Hz, 1H), 7.35 (d, J=8.55 Hz, 1H), 7.59 (s, 1H).

### Example 190

### N-cyclopentyl-Y-(2-cyclopentyl-5-propyl-2,3,4,4a,5,9b-hexahydro-1H-pyrido[4,3-b]indol-8-yl)urea

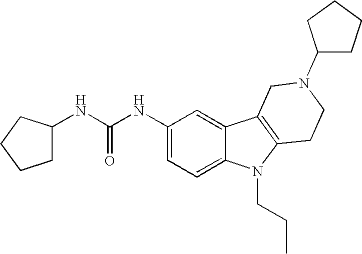

To a solution of crude 2-cyclopentyl-5-propyl-2,3,4,4a,5,9b-hexahydro-1H-pyrido[4,3-b]indol-8-amine (30 mg, 0.1 mmol) in dry DMF (2 mL) was added cyclopentyl isocyanate (0.11 mmol) followed by triethyl amine (0.2 mmol). The reaction mixture was stirred at room temperature for overnight. Removal of solvent gave the crude product, which was purified by reverse phase HPLC to give desired product as a TFA salt (26 mg, 69%). MS (M+1): 409.0. 1H NMR (600 MHz, CD3OD) δ ppm 0.94 (t, J=7.34 Hz, 3H), 1.25 (t, J=7.04 Hz, 2H), 1.48 (m, 1H), 1.65 (m, 1H), 1.70-1.84 (m, 7H), 1.85-2.00 (m, 5H), 2.33 (m, 2H), 3.23 (m, 2H), 3.56 (m, 1H), 3.82 (m, 1H), 4.00 (m, 1H), 4.10 (m, 3H), 4.39 (d, J=14.09 Hz, 1H), 4.70 (d, J=13.50 Hz, 1H), 7.01 (d, J=8.80 Hz, 1H), 7.34 (d, J=8.80 Hz, 1H), 7.60 (s, 1H).

### Example 191

### N-(2-cyclopentyl-5-propyl-2,3,4,4a,5,9b-hexahydro-1H-pyrido[4,3-b]indol-8-yl)-N′-phenylurea

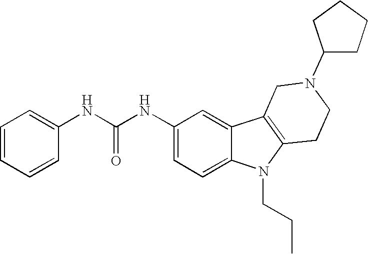

To a solution of crude 2-cyclopentyl-5-propyl-2,3,4,4a,5,9b-hexahydro-1H-pyrido[4,3-b]indol-8-amine (30 mg, 0.1 mmol) in dry DMF (2 mL) was added phenyl isocyanate (0.11 mmol) followed by triethyl amine (0.2 mmol). The reaction mixture was stirred at room temperature for overnight. Removal of solvent gave the crude product, which was purified by reverse phase HPLC to give desired product as a TFA salt (32 mg, 60%). MS (M+1): 417.0. 1H NMR (600 MHz, CD3OD) δppm 0.95 (t, J=7.34 Hz, 3H), 1.70-1.95 (m, 6H), 2.34 (m, 2H), 3.25 (m, 2H), 3.57 (m, 2H), 3.82 (m, 1H), 4.00 (m, 1H), 4.11 (m, 3H), 4.40 (d, J=13.50 Hz, 1H), 4.74 (d, J=14.09 Hz, 1H), 7.03 (m, 1H), 7.10 (d, J=8.56 Hz, 1H), 7.30 (t, J=7.63 Hz, 2H), 7.39 (d, J=8.56 Hz, 1H), 7.45 (d, J=7.63 Hz, 2H), 7.71 (s, 1H).

### Example 192

### 8-[(4-methylpiperidin-1-yl)carbonyl]-5-(phenylsulfonyl)-2-(tetrahydro-2H-pyran-4-yl)-2,3,4,5-tetrahydro-1-pyrido[4,3-b]indole

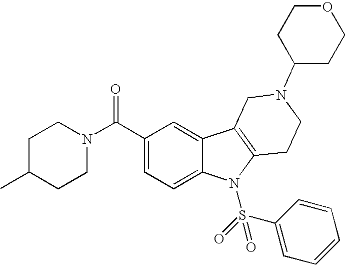

### Step A. The preparation of tert-butyl 8-[(4-methylpiperidin-1-yl)carbonyl]-5-(phenylsulfonyl)-1,3,4,5-tetrahydro-2H-pyrido[4,3-b]indole-2-carboxylate

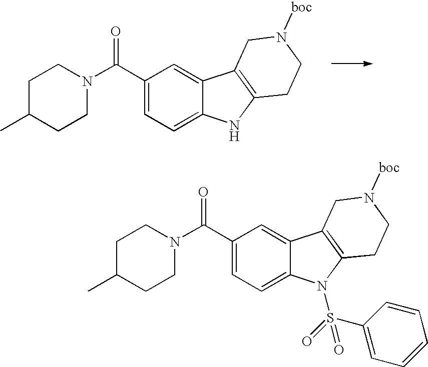

To a solution of 2-(tert-butoxycarbonyl)-2,3,4,5-tetrahydro-1H-pyrido[4,3-b]indole-8-carboxylic acid (196 mg, 0.493 mmol) and potassium tert-butoxide (123 mg, 1.10 mmol) in dry DMF (5 mL) was added benzenesulfonyl chloride (90 μL, 0.70 mmol) dropwise under nitrogen. The mixture was stirred at room temperature for 1 h, then H₂O (15 ml) was added and the solid was collected and dried to afford crude product (256 mg). MS (M+1): 538.21.

### Step B. 8-[(4-methylpiperidin-1-yl)carbonyl]-5-(phenylsulfonyl)-2,3,4,5-tetrahydro-1H-pyrido[4,3-b]indole TFA salt

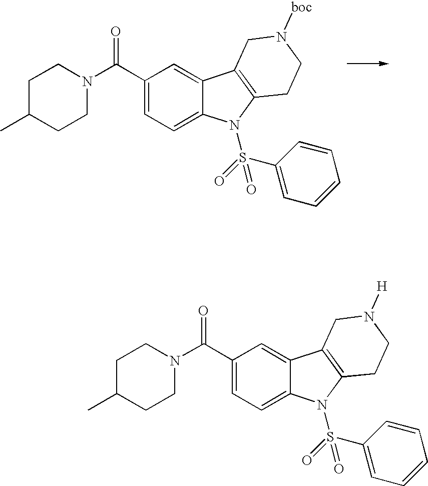

To a solution of tert-butyl 8-[(4-methylpiperidin-1-yl)carbonyl]-5-(phenylsulfonyl)-1,3,4,5-tetrahydro-2H-pyrido[4,3-b]indole-2-carboxylate (256 mg, 0.476 mmol) in dichloromethane (5 ml) was added trifluoroacetic acid (1 ml) at room temperature. The solution was stirred at room temperature for 30 min. Removal of solvent and excess trifluoroacetic acid _{in vacuo} afforded the desired product as its TFA salt (280 mg). MS (M+1): 437.96.

### Step C. The preparation of 8-[(4-methylpiperidin-1-yl)carbonyl]-5-(phenylsulfonyl)-2-(tetrahydro-2H-pyran-4-yl)-2,3,4,5-tetrahydro-1H-pyrido[4,3-b]indole

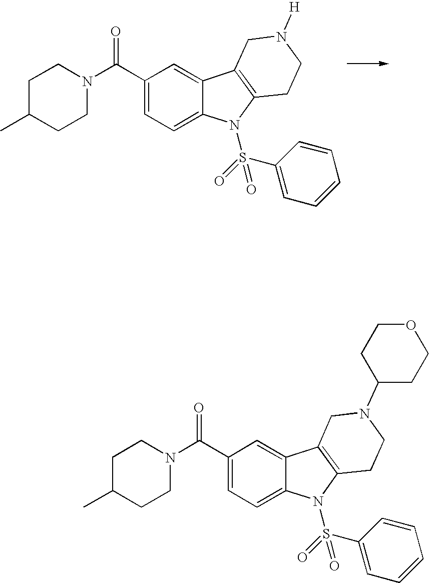

To a solution 8-[(4-methylpiperidin-1-yl)carbonyl]-5-(phenylsulfonyl)-2,3,4,5-tetrahydro-1H-pyrido[4,3-b]indole TFA salt (250 mg, 0.453 mmol), tetrahydro-4H-pyran-4-one (150 mg, 1.50 mmol) in dichloromethane (5 ml) was added sodium triacetoxyborohydride (215 mg, 1.01 mmol). The mixture was stirred overnight under nitrogen, and then 1 N NaOH (2 ml) was added and the mixture was stirred for 1 min. The reaction mixture was diluted with dichloromethane (20 ml), and the organic layer was passed through a pre-packed hydromatrix cartridge (10 g), and washed with dichloromethane (20 ml). Dichloromethane was concentrated _{in vacuo} and the residue was purified on preparative HPLC (10-70% CH₃CN, 30 min) to afford the title compound as its TFA salt (85 mg). MS (M+1): 521.92. 1H NMR (400 MHz, CD3OD) δ ppm 0.96 (d, J=6.45 Hz, 3H), 1.03-1.26 (m, 2H), 1.48-1.95 (m, 5H), 2.12 (d, J=10.16 Hz, 2H), 2.83 (t, J=12.30 Hz, 1H), 2.98-3.15 (m, 1H), 3.31-3.76 (m, 7H), 3.82-4.14 (m, 3H), 4.33-4.75 (m, 3H), 7.41 (d, J=8.59 Hz, 1H), 7.47-7.57 (m, 3H), 7.65 (t, J=7.32 Hz, 1H), 7.93 (d, J=8.01 Hz, 2H), 8.21 (d, J=8.79 Hz, 1H).

### Example 193

### Methyl 2-{[8-[(4-methylpiperidin-1-yl)carbonyl]-2-(tetrahydro-2H-pyran-4-yl)-1,2,3,4-tetrahydro-5H-pyrido[4,3-b]indol-5-yl]sulfonyl}benzoate

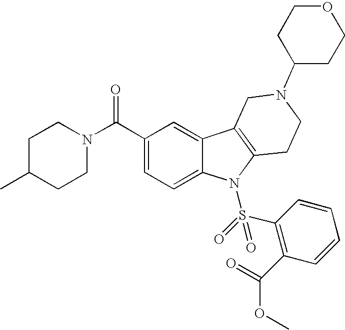

### Step A: The preparation of tert-butyl 5-{[2-(methoxycarbonyl)phenyl]sulfonyl}-8-[(4-methylpiperidin-1-yl)carbonyl]-1,3,4,5-tetrahydro-2H-pyrido[4,3-b]indole-2-carboxylate

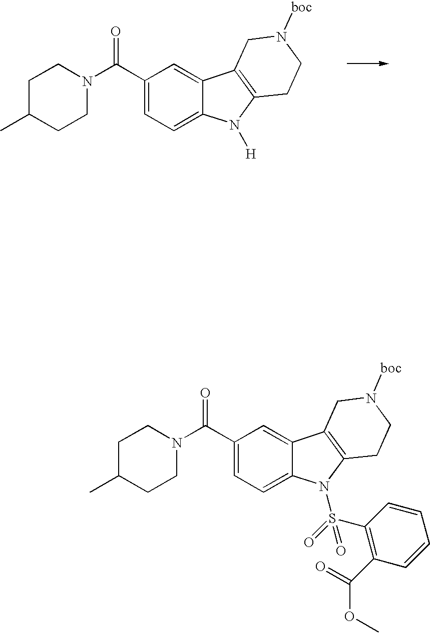

To a solution of 2-(tert-butoxycarbonyl)-2,3,4,5-tetrahydro-1H-pyrido[4,3-b]indole-8-carboxylic acid (202 mg, 0.508 mmol) and potassium tert-butoxide (112 mg, 1.00 mmol) in dry DMF (5 mL) was added 90% methyl (2-chlorosulfonyl)benzoate (258 mg, 0.989 mmol) portionwise under nitrogen. The mixture was stirred at room temperature for 1.5 h, then H₂O (15 ml) was added and the solid was collected and dried to afford crude product which was used without purification. MS (M+1): 595.93.

### Step B: The preparation of methyl 2-({8-[(4-methylpiperidin-1-yl)carbonyl]-1,2,3,4-tetrahydro-5H-pyrido[4,3-b]indol-5-yl}sulfonyl)benzoate TFA salt

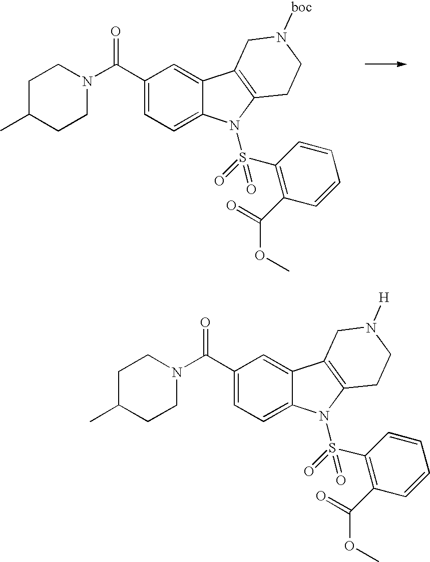

To a solution of crude tert-butyl 5-{[2-(methoxycarbonyl)phenyl]sulfonyl}-8-[(4-methylpiperidin-1-yl)carbonyl]-1,3,4,5-tetrahydro-2H-pyrido[4,3-b]indole-2-carboxylate from Step A in dichloromethane (5 ml) was added trifluoroacetic acid (2 ml) at room temperature. The solution was stirred at room temperature for overnight. The excess TFA and dichloromethane was removed in vacuo, and the residue was lipholized to afford crude product, which was used without purification. MS (M+1): 495.92.

### Step C: The preparation of methyl 2-{[8-[(4-methylpiperidin-1-yl)carbonyl]-2-(tetrahydro-2H-pyran-4-yl)-1,2,3,4-tetrahydro-5H-pyrido[4,3-b]indol-5-yl]sulfonyl}benzoate

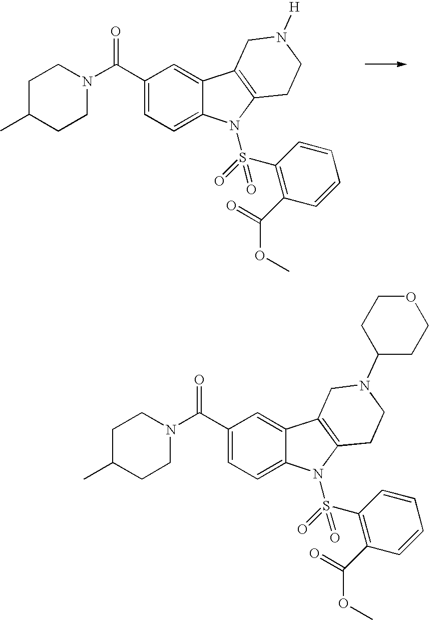

Following the same method as described in Example 192, Step C: The title compound as its TFA salt was prepared from methyl 2-({8-[(4-methylpiperidin-1-yl)carbonyl]-1,2,3,4-tetrahydro-5H-pyrido[4,3-b]indol-5-yl}sulfonyl)benzoate TFA salt and tetrahydro-4H-pyran-4-one. MS (M+1): 579.90. 1H NMR (400 MHz, CD3OD) δ ppm 0.94 (d, J=7.03 Hz, 3H), 1.00-1.26 (m, 2H), 1.38-1.96 (m, 4H), 2.14 (d, J=12.50 Hz, 2H), 2.70-3.14 (m, 2H), 3.31-3.77 (m, 7H), 3.77-4.03 (m, 1H), 3.89 (s, 3H), 4.09 (d, J=11.72 Hz, 3H), 4.53 (d, 3H), 7.33 (d, J=8.59 Hz, 1H), 7.43-7.59 (m, 2H), 7.61 (s, 1H), 7.67-7.77 (m, 2H), 7.92 (d, J=8.59 Hz, 1H).

### Example 194

### 2-{[8-[(4-methylpiperidin-1-yl)carbonyl]-2-(tetrahydro-2H-pyran-4-yl)-1,2,3,4-tetrahydro-5H-pyrido[4,3-b]indol-5-yl]sulfonyl}benzoic acid

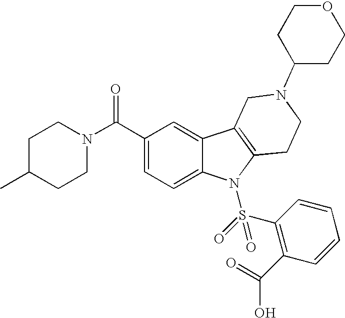

To the solution of methyl 2-{[8-[(4-methylpiperidin-1-yl)carbonyl]-2-(tetrahydro-2H-pyran-4-yl)-1,2,3,4-tetrahydro-5H-pyrido[4,3-b]indol-5-yl]sulfonyl}benzoate TFA salt (21 mg, 0.036 mmol) in methanol (1 mL) was added 1 N NaOH (0.5 mL), and the mixture was stirred for 2 h. The solvent was removed _{in vacuo} and the residue was redissolved in H2O (1 mL), and acetic acid (0.1 mL) was added. The mixture was purified by preparative HPLC (10-70%, 30 min) and lipholized to afford the title compound as its TFA salt (11 mg). MS (M+1): 566.0. 111 NMR (400 MHz, _{CD3OD}) δ ppm 0.95 (d, J=6.45 Hz, 3H), 0.99-1.30 (m, 2H), 1.40-2.00 (m, 5H), 2.04-2.21 (m, 2H), 2.64-3.16 (m, 2H), 3.33-3.82 (m, 8H), 4.10 (dd, J=11.62, 4.20 Hz, 2H), 4.41-4.72 (m, 3H), 7.32 (dd, J=8.59, 1.56 Hz, 1H), 7.54-7.63 (m, 2H), 7.65-7.76 (m, 3H), 7.92 (d, J=8.59 Hz, 1H).

### Example 195

### Methyl 2-{[8-[(4-methylpiperidin-1-yl)carbonyl]-2-(tetrahydro-2H-pyran-4-yl)-1,2,3,4-tetrahydro-5H-pyrido[4,3-b]indol-5-yl]sulfonyl}benzoate TFA salt

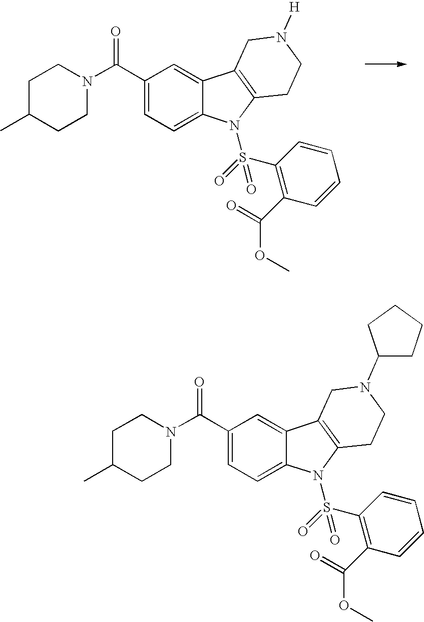

Following the same method as described in Example 192, Step C: The title compound as its TFA salt was prepared from methyl 2-({8-[(4-methylpiperidin-1-yl)carbonyl]-1,2,3,4-tetrahydro-5H-pyrido[4,3-b]indol-5-yl}sulfonyl)benzoate TFA salt and cyclopentanone. MS (M+1): 563.88. 1H NMR (400 MHz, CD3OD) δ ppm 0.93 (t, J=6.25 Hz, 3H), 0.99-1.27 (m, 2H), 1.40-1.97 (m, 8H), 2.18-2.38 (m, 2H), 2.70-3.13 (m, 2H), 3.28-3.67 (m, 4H), 3.73-4.00 (m, 3H), 3.82-3.90 (m, 3H), 4.20-4.79 (m, 3H), 7.22-7.39 (m, 1H), 7.40-7.58 (m, 2H), 7.58-7.65 (m 1H), 7.69 (d, J=5.47 Hz, 2H), 7.83-7.98 (m, 1H).

### Example 196

### 2-cyclobutyl-5-[(1-methyl-1H-imidazol-4-yl)sulfonyl]-8-[(4-methylpiperidin-1-yl)carbonyl]-2,3,4,5-tetrahydro-1H-pyrido[4,3-b]indole

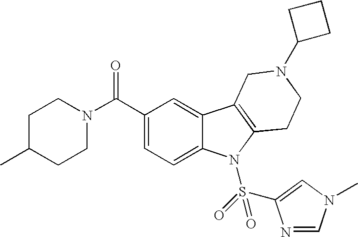

### Step A: The preparation of tert-butyl 5-[(1-methyl-1H-imidazol-4-yl)sulfonyl]-8-[(4-methylpiperidin-1-yl)carbonyl]-1,3,4,5-tetrahydro-2H-pyrido[4,3-b]indole-2-carboxylate

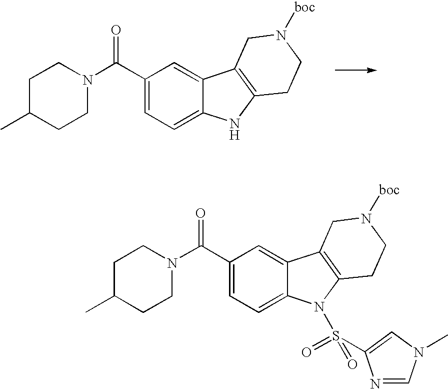

Potassium tert-butoxide (98 mg, 0.875 mmol) was added to a solution of 2-(tert-butoxycarbonyl)-2,3,4,5-tetrahydro-1H-pyrido[4,3-b]indole-8-carboxylic acid (202 mg, 0.508 mmol) in anhydrous DMF (3 mL) at room temperature. The mixture was stirred at room temperature for 5 min, then 1-methyl-1H-imidazole-4-sulfonyl chloride (142 mg, 0.788 mmol) was added portionwise. The reaction mixture was stirred at room temperature for 1 h, quenched with 1 N NaOH (1 mL) and H2O (10 mL) was added. The solid was collected and dried to afford the crude product, which was purified on silica gel to give the desired product (155 mg). MS (M+1): 541.93.

### Step B: The preparation of 5-[(1-methyl-1H-imidazol-4-yl)sulfonyl]-8-[(4-methylpiperidin-1-yl)carbonyl]-2,3,4,5-tetrahydro-1H-pyrido[4,3-b]indole TFA salt

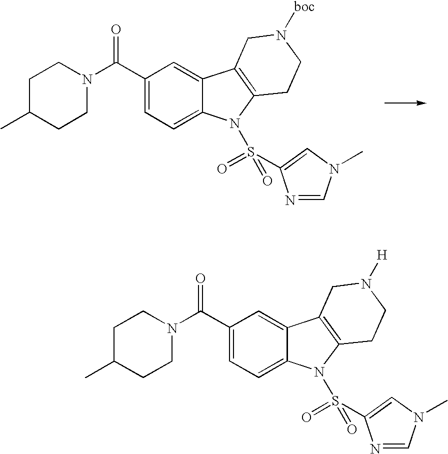

Following the same procedure described in Example 192, Step B: The desired product as its TFA salt was prepared from tert-butyl 5-[(1-methyl-1H-imidazol-4-yl)sulfonyl]-8-[4 (4-methylpiperidin-1-yl)carbonyl]-1,3,4,5-tetrahydro-2H-pyrido[4,3-b]indole-2-carboxylate. MS (M+1): 441.93.

### Step C: The preparation of 2-cyclobutyl-5-[(1-methyl-1H-imidazol-4-yl)sulfonyl]-8-[(4-methylpiperidin-1-yl)carbonyl]-2,3,4,5-tetrahydro-1H-pyrido[4,3-b]indole TFA salt

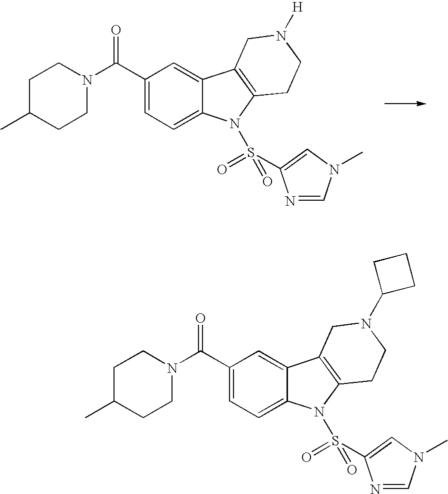

Following the similar procedure of Example 192, Step C: The title compound as its TFA salt was prepared from 5-[(1-methyl-1H-imidazol-4-yl)sulfonyl]-8-[(4-methylpiperidin-1-yl)carbonyl]-2,3,4,5-tetrahydro-1H-pyrido[4,3-b]indole TFA salt and cyclobutanone. MS (M+1): 495.98. 1HNMR (400 MHz, CD3OD) δ ppm 0.95 (d, J=6.44 Hz, 3H), 1.02-1.28 (m, 2H), 1.40-2.04 (m, 5H), 2.17-2.53 (m, 4H), 2.83 (t, J=11.23 Hz, 1H), 2.98-3.17 (m, 1H), 3.23-4.00 (m, 6H), 3.68 (s, 3H), 4.13 (d, J=13.09 Hz, 1H), 4.47-4.72 (m, 2H), 7.34 (dd, J=8.78, 1.56 Hz, 1H), 7.52 (d, J=0.98 Hz, 1H), 7.65 (s, 1H), 8.06 (d, J=8.79 Hz, 1H), 8.07 (s, 1H).

### Example 197

### 5-[(1-methyl-1H-imidazol-4-yl)sulfonyl]-8-[(4-methylpiperidin-1-yl)carbonyl]-2-(tetrahydro-2H-pyran-4-yl)-2,3,4,5-tetrahydro-1H-pyrido[4,3-b]indole

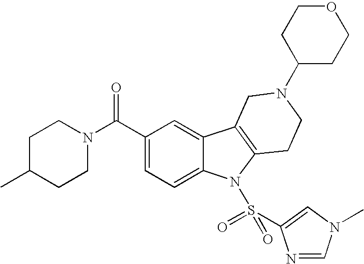

Following the similar procedure of Example 192, Step C: The title compound as its TFA salt was prepared from 5-[(1-methyl-1H-imidazol-4-yl)sulfonyl]-8-[(4-methylpiperidin-1-yl)carbonyl]-2,3,4,5-tetrahydro-1H-pyrido[4,3-b]indole TFA salt and tetrahydro-4H-pyran-4-one. MS (M+1): 526.96. 1HNMR (400 MHz, CD3OD) δ ppm 0.94 (d, J=6.25 Hz, 3H), 1.00-1.26 (m, 2H), 1.36-1.80 (m, 3H), 1.79-1.94 (m, 2H), 2.13 (d, J=10.94 Hz, 2H), 2.66-3.15 (m, 2H), 3.34-3.80 (m, 10H), 3.82-4.16 (m, 3H), 4.31-4.73 (m, 3H), 7.32 (d, J=8.59 Hz, 1H), 7.53 (s, 1H), 7.63 (s, 1H), 8.04 (d, J=8.59 Hz, 1H), 8.05 (s, 1H).

### Example 198

### 8-(3,4-dihydroisoquinolin-2(1H)-ylcarbonyl)-5-(ethylsulfonyl)-2-(tetrahydro-2H-pyran-4-yl)-2,3,4,5-tetrahydro-1H-pyrido[4,3-b]indole

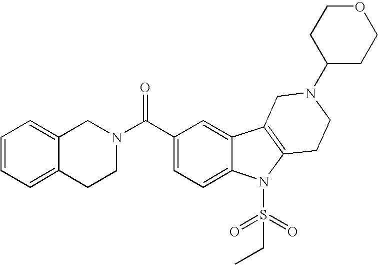

### Step A: The preparation of tert-butyl 8-(3,4-dihydroisoquinolin-2(1H)-ylcarbonyl)-1,3,4,5-tetrahydro-2H-pyrido[4,3-b]indole-2-carboxylate

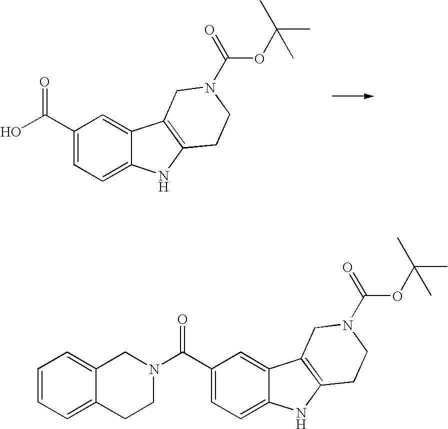

Following the same HATU coupling procedure described in Example 145, Step A: The title compound was prepared from 2-(tert-butoxycarbonyl)-2,3,4,5-tetrahydro-1H-pyrido[4,3-b]indole-8-carboxylic acid and 3,4-dihydroisoquino-2(1H)-line. MS (M+1):

### Step B: The preparation of tert-butyl 8-(3,4-dihydroisoquinolin-2(1H)-ylcarbonyl)-5-(ethylsulfonyl)-1,3,4,5-tetrahydro-2H-pyrido[4,3-b]indole-2-carboxylate

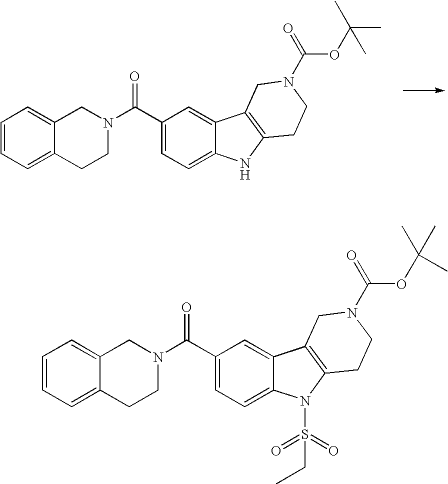

To a solution of tert-butyl 8-(3,4-dihydroisoquinolin-2(1H)-ylcarbonyl)-1,3,4,5-tetrahydro-2H-pyrido[4,3-b]indole-2-carboxylate (0.8 mmol) in dry DMF (5 mL) was added NaH (60%, 4.0 mmol) at 0° C. and stirred for 30 min at the same temperature. Then ethanesulfonyl chloride (2.4 mmol) was added stirred at room temperature for overnight. The solvent was removed and the residue was taken up into dichloromethane (30 mL), extracted with water and brine, dried over Na₂SO₄, concentrated. The crude product was purified by flash chromatography to yield the desired product (305 mg, 73%). MS (M+1): 524.0.

### Step C: The preparation of 8-(3,4-dihydroisoquinolin-2(1H)-ylcarbonyl)-5-(ethylsulfonyl)-2-(tetrahydro-2H-pyran-4-yl)-2,3,4,5-tetrahydro-1H-pyrido[4,3-b]indole

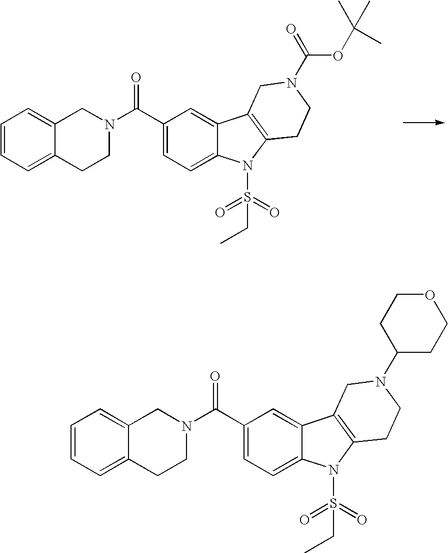

The intermediate tert-butyl 8-(3,4-dihydroisoquinolin-2(1H)-ylcarbonyl)-5-(ethylsulfonyl)-1,3,4,5-tetrahydro-2H-pyrido[4,3-b]indole-2-carboxylate from the previous step (0.1 mmol) was treated with 4N HCl in dioxane (2 mL) for 2 h at room, then evaporated to dryness. The crude HCl salt was treated with tetrahydro-4H-pyran-4-one (0.2 mmol), and sodium triacetoxyborohydride (0.2 mmol) in dichloromethane (3 mL). The mixture was stirred at room temperature overnight, diluted with dichloromethane (15 mL), washed with saturated NaHCO₃ solution (5 mL), dried over Na₂SO₄. The solution was concentrated in vacuo and the residue was purified with reverse phase HPLC to give the desired product as its TFA salt (14 mg, 22%). MS (M+1): 508.0. 1H NMR (400 MHz, CD3OD) δ ppm 1.23 (t, J=7.32 Hz, 3H), 1.78-2.00 (m, 2H), 2.08-2.22 (m, 2H), 2.83-3.04 (m, 2H), 3.40-3.58 (m, 10H), 3.61-3.79 (m, 2H), 3.91-4.05 (m, 1H), 4.11 (dd, J=11.43, 3.61 Hz, 2H), 4.44-4.76 (m, 2H), 7.05-7.27 (m, 4H), 7.49 (d, J=8.69 Hz, 1H), 7.71 (s, 1H), 8.07 (d, J=8.69 Hz, 1H).

### Example 199

### 8-(3,4-dihydroisoquinolin-2(1H)-ylcarbonyl)-5-(ethylsulfonyl)-2-(tetrahydrofuran-2-yl)-2,3,4,5-tetrahydro-1H-pyrido[4,3-b]indole

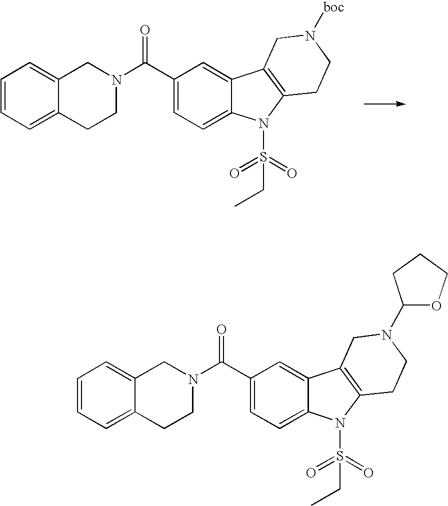

The intermediate tert-butyl 8-(3,4-dihydroisoquinolin-2(1H)-ylcarbonyl)-5-(ethylsulfonyl)-1,3,4,5-tetrahydro-2,1-pyrido[4,3-b]indole-2-carboxylate from Example 198, Step A (0.1 mmol) was treated with 4N HCl in dioxane (2 mL) for 2 h at room, then evaporated to dryness. The crude HCl salt was treated with dihydrofuran-3(2H)-one (0.2 mmol) and sodium triacetoxyborohydride (0.2 mmol) in dichloromethane (3 mL). The mixture was stirred at room temperature overnight, diluted with dichloromethane (15 mL), washed with saturated NaHCO₃ solution (5 mL), dried over Na₂SO₄. The solution was concentrated in vacuo and the residue was purified with reverse phase HPLC to give the desired product as its TFA salt (9 mg, 15%). MS (M+1): 494.0. 1H NMR (400 MHz, CD3OD) δ ppm 1.23 (t, J=7.32 Hz, 3H), 2.26-2.37 (m, 1H), 2.44-2.58 (m, 1H), 2.84-3.03 (m, 2H), 3.40-3.56 (m, 6H), 3.61-3.84 (m, 4H), 3.92 (m, 2H), 4.08-4.18 (m, 1H), 4.18-4.33 (m, 2H), 4.51-4.72 (m, 2H), 7.08-7.26 (m, 4H), 7.49 (d, J=8.61 Hz, 1H), 7.70 (s, 1H), 8.07 (d, J=8.61 Hz, 1H).

### Example 200

### 8-(3,4-dihydroisoquinolin-2(1H)-ylcarbonyl)-2-(1-methylpiperidin-4-yl)-5-(propylsulfonyl)-2,3,4,5-tetrahydro-1H-pyrido[4,3-b]indole

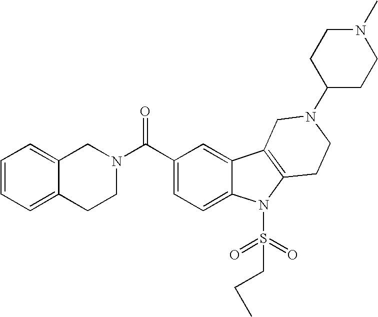

### Step A. The preparation of tert-butyl 8-(3,4-dihydroisoquinolin-2(1H)-ylcarbonyl)-5-(propylsulfonyl)-1,3,4,5-tetrahydro-2H-pyrido[4,3-b]indole-2-carboxylate

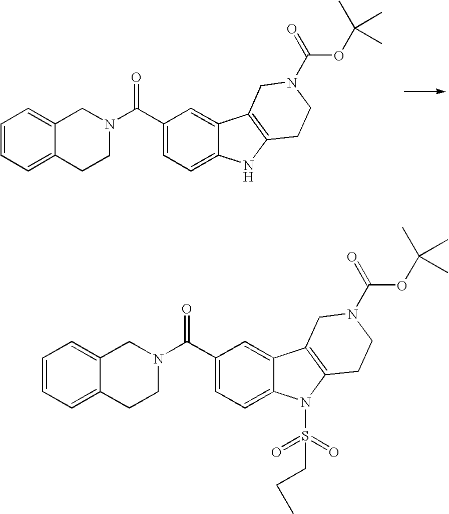

To a solution of tert-butyl 8-(3,4-dihydroisoquinolin-2(1H)-ylcarbonyl)-1,3,4,5-tetrahydro-2H-pyrido[4,3-b]indole-2-carboxylate (0.24 mmol) in dry DMF (2 mL) was added t-BuOK (0.48 mmol) at 0° C. and stirred for 10 min at the same temperature. Then propanesulfonyl chloride (0.6 mmol) was added stirred at room temperature for overnight. The solvent was removed and the residue was taken up into dichloromethane (30 mL), extracted with water and brine, dried over Na₂SO₄, concentrated. The crude product was used for the next step without further purification. MS (M+1): 538.0.

### Step B: The preparation of 8-(3,4-dihydroisoquinolin-2(1H)-ylcarbonyl)-2-(1-methylpiperidin-4-yl)-5-(propylsulfonyl)-2,3,4,5-tetrahydro-1H-pyrido[4,3-b]indole

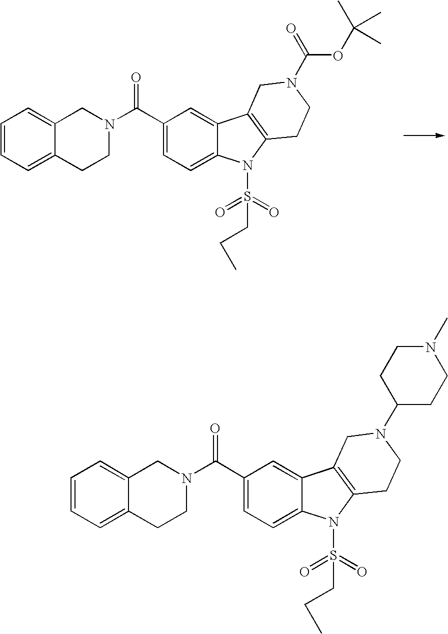

The crude tert-butyl 8-(3,4-dihydroisoquinolin-2(1H)-ylcarbonyl)-5-(propylsulfonyl)-1,3,4,5-tetrahydro-2H-pyrido[4,3-b]indole-2-carboxylate from previous step was dissolved in dichloromethane (5 mL) and treated with TFA (1 mL), stirred at room temperature for 2 h. After being evaporated in dryness, the crude TFA salt was treated with 1-methylpiperidin-4-one (0.4 mmol) and sodium triacetoxyborohydride (0.4 mmol) in dichloromethane (5 mL). The mixture was stirred at room temperature overnight, diluted with dichloromethane (20 mL), washed with saturated NaHCO₃ solution (10 mL), and dried over Na₂SO₄. The solution was concentrated in vacuo and the residue was purified with reverse phase HPLC to give the desired product as its TFA salt (29 mg, 16% for 3 steps). MS (M+1): 535.0. 1H NMR (400 MHz, CD3OD) δ ppm 0.95 (t, J=7.42 Hz, 3H), 1.60-1.73 (m, 2H), 2.11-2.27 (m, 2H), 2.45-2.59 (m, 2H), 2.82-3.01 (m, 3H), 2.90 (s, 3H), 3.08-3.21 (m, 2H), 3.38-3.50 (m, 4H), 3.63-3.81 (m, 7H), 4.54-4.67 (m, 3H), 7.0-7.26 (m, 4H), 7.48 (d, J=8.59 Hz, 1H), 7.68 (s, 1H), 8.06 (d, J=8.59 Hz, 1H).

### Example 201

### 5-(1H-imidazol-1-ylsulfonyl)-8-[(4-methylpiperidin-1-yl)carbonyl]-2-(tetrahydro-2H-pyran-4-yl)-2,3,4,5-tetrahydro-1H-pyrido[4,3-b]indole

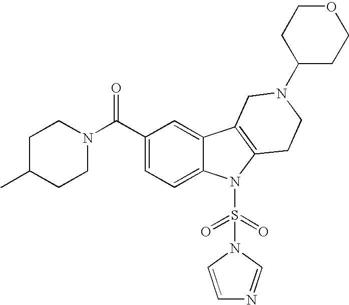

### Step A: The preparation of 8-[(4-methylpiperidin-1-yl)carbonyl]-2-(tetrahydro-2H-pyran-4-yl)-2,3,4,5-tetrahydro-1H-pyrido[4,3-b]indole

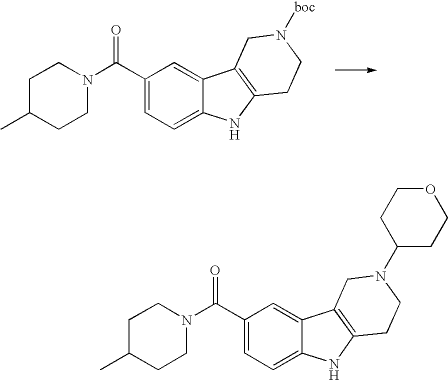

To a solution of tert-butyl 8-[(4-methylpiperidin-1-yl)carbonyl]-1,3,4,5-tetrahydro-2H-pyrido[4,3-b]indole-2-carboxylate (796 mg, 2.0 mmol) in dichloromethane (20 mL) was added TFA (3 mL), the reaction mixture was stirred at room temperature for 3 h. After concentrated, the residue was treated with tetrahydro-4H-pyran-4-one (6.0 mmol) and sodium triacetoxyborohydride (6.0 mmol) in dichloromethane (30 mL). The mixture was stirred at room temperature overnight, diluted with dichloromethane (50 mL), washed with saturated NaHCO₃ solution (20 mL), and dried over Na₂SO₄. The solution was concentrated in vacuo and the residue was purified with flash chromatography to give the desired product as a brown oil (650 mg, 86%). MS (M+1): 382.0.

### Step B: The preparation of 5-(1H-imidazol-1-ylsulfonyl)-8-[(4-methylpiperidin-1-yl)carbonyl]-2-(tetrahydro-2H-pyran-4-yl)-2,3,4,5-tetrahydro-1H-pyrido[4,3-b]indole

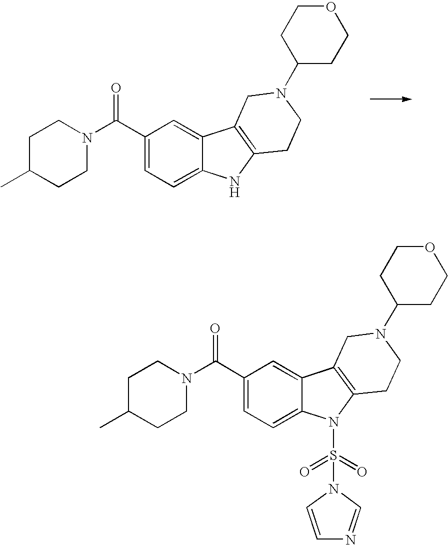

To a solution of 8-[(4-methylpiperidin-1-yl)carbonyl]-2-(tetrahydro-2H-pyran-4-yl)-2,3,4,5-tetrahydro-1H-1-pyrido[4,3-b]indole (80 mg, 0.21 mmol) in dry DMF (5 mL) was added NaH (60%, 0.42 mmol) at 0° C. and stirred for 30 min at the same temperature. Then 3-(imidazole-1-sulfonyl)-1-methyl-31−/−imidazol-1-ium triflate (0.42 mmol) was added in one portion and stirred at 80° C. for overnight. The solvent was removed and the residue was taken up into dichloromethane (20 mL), extracted with water and brine, dried over Na₂SO₄, concentrated. The crude product was purified by reverse phase HPLC to yield product 63 mg (41%). MS (M+1): 512.0. 1H NMR (400 MHz, CD3OD) δppm 0.96 (d, J=6.45 Hz, 3H), 1.04-1.30 (m, 2H), 1.48-1.82 (m, 3H), 1.83-1.97 (m, 2H), 2.08-2.23 (m, 2H), 2.77-2.94 (m, 1H), 3.01-3.16 (m, 1H), 3.39-3.57 (m, 5H), 3.55-3.84 (m, 3H), 3.86-4.01 (m, 1H), 4.11 (dd, J=11.43, 3.22 Hz, 2H), 4.46-4.73 (m, 2H), 7.00-7.67 (m, 4H), 8.07-8.91 (m, 2H).

### Example 202

### 8-[(4-methylpiperidin-1-yl)carbonyl]-5-(morpholin-4-ylcarbonyl)-2-(tetrahydro-2H-pyran-4-yl)-2,3,4,5-tetrahydro-1H-pyrido[4,3-b]indole

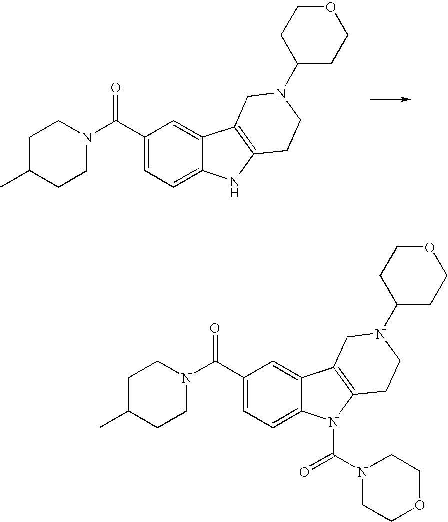

To a solution of 8-[(4-methylpiperidin-1-yl)carbonyl]-2-(tetrahydro-2H-pyran-4-yl)-2,3,4,5-tetrahydro-1H-pyrido[4,3-b]indole (0.2 mmol) in dry DMF (3 mL) was added NaH (60%, 0.4 mmol) at 0° C. and stirred for 30 min at the same temperature. Then morpholine-4-carbonyl chloride (0.4 mmol) was added stirred at room temperature for overnight. The solvent was removed and the residue was taken up into dichloromethane (20 mL), extracted with water and brine; dried over Na₂SO₄, concentrated. The crude product was purified by reverse phase HPLC to yield TFA salt 39 mg (32%). MS (M+1): 495.0. 1H NMR (400 MHz, CD3OD) δ ppm 0.96 (d, J=6.45 Hz, 3H), 1.04-1.29 (m, 2H), 1.51-1.82 (m, 3H), 1.84-1.98 (m, 2H), 2.11-2.22 (m, 2H); 2.76-2.95 (m, 1H), 3.02-3.17 (m, 1H), 3.42-3.61 (m, 7H), 3.63-3.75 (m, 5H), 3.75-3.86 (m, 3H), 3.93-4.04 (m, 1H), 4.11 (dd, J=11.43, 3.61 Hz, 2H), 4.43-4.55 (m, 1H), 4.54-4.64 (m, 1H), 4.66-4.78 (m, 1H), 7.36 (d, J=8.00 Hz, 1H), 7.54 (d, J=8.00 Hz, 1H), 7.62 (s, 1H).

### Example 203

### 5-[(5-methylisoxazol-4-yl)sulfonyl]-8-[(4-methylpiperidin-1-yl)carbonyl]-2-(tetrahydro-2H-pyran-4-yl)-2,3,4,5-tetrahydro-1H-pyrido[4,3-b]indole

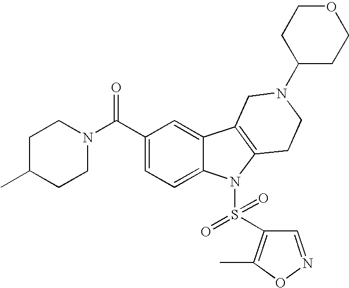

60% NaH (16 mg, 0.4 mmol) was added in one portion to a suspension of 8-[(4-methylpiperidin-1-yl)carbonyl]-2-(tetrahydro-2H-pyran-4-yl)-2,3,4,5-tetrahydro-1H-pyrido[4,3-b]indole (75 mg, 0.2 mmol) in DMF (2 mL) at room temperature under nitrogen. The mixture was stirred for 30 min, 5-methylisoxazole-4-sulfonyl chloride (54 mg, 0.3 mmols) was added at room temperature and the reaction mixture was stirred at room temperature for 2 hours. The solvent was removed in vacuo and the residue was dissolved in dichloromethane and washed with water dried over sodium sulfate. Removal of solvent gave the crude product, which was purified over reverse phase HPLC (10-75% CH₃CN) followed by recristallisation with MeOH to provide the title compound (4.5 mg). MS (M+1): 527.0. 1H NMR (400 MHz, DMSO-D6) δ ppm 0.92 (d, J=6.25 Hz, 3H), 0.99-1.16 (m, 2H), 1.49-1.85 (m, 7H), 2.08-2.14 (m, 2H), 2.69-3.08 (m, 2H), 3.27-3.45 (m, 8H), 3.56-3.79 (m, 2H), 3.88-3.98 (m, 1H), 4.01 (dd, J=11.33, 3.71 Hz, 2H), 4.34-4.52 (m, 1H), 4.56-4.67 (m, 1H), 7.26 (d, J=8.59, 1H), 7.58 (s, 1H), 7.87 (d, J=8.40 Hz, 1H), 9.85 (bs, 1H).

### Example 204

### 2-cyclopentyl-5-(1H-imidazol-1-ylsulfonyl)-8-[(4-methylpiperidin-1-yl)carbonyl]-2,3,4,5-tetrahydro-1H-pyrido[4,3-b]indole

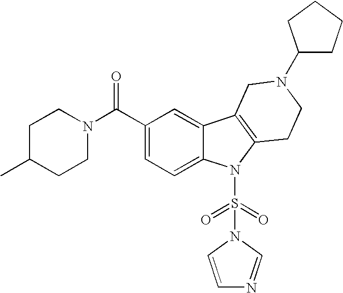

### Step A: The preparation of tert-butyl 5-(1H-imidazol-1-ylsulfonyl)-8-[(4-methylpiperidin-1-yl)carbonyl]-1,3,4,5-tetrahydro-2H-pyrido[4,3-b]indole-2-carboxylate

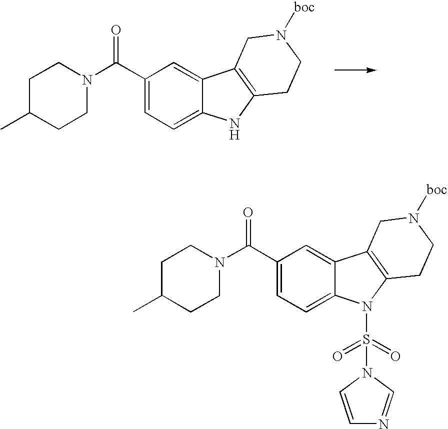

To a solution of tert-butyl 8-[(4-methylpiperidin-1-yl)carbonyl]-1,3,4,5-tetrahydro-2H-pyrido[4,3-b]indole-2-carboxylate (2.0 mmol) in dry DMF (15 mL) was added NaH (60%, 10.0 mmol) at 0° C. and stirred for 30 min at the same temperature. Then 3-(imidazole-1-sulfonyl)-1-methyl-3H-imidazol-1-ium triflate (6.0 mmol, for preparation see: Serge Beaudoin, Kenneth E. Kinsey, and James F. Burns, J. Org. Chem., 2003, 68(1), 115) was added in one portion and stirred at 80° C. for 6 h. The solvent was removed and the residue was taken up into dichloromethane (50 mL), extracted with water and brine, dried over Na₂SO₄, concentrated. The crude product was purified by reverse phase HPLC to yield product (203 mg, 19%). MS (M+1): 528.39.

### Step B: The preparation of 2-cyclopentyl-5-(1H-imidazol-1-ylsulfonyl)-8-[(4-methylpiperidin-1-yl)carbonyl]-2,3,4,5-tetrahydro-1H-pyrido[4,3-b]indole

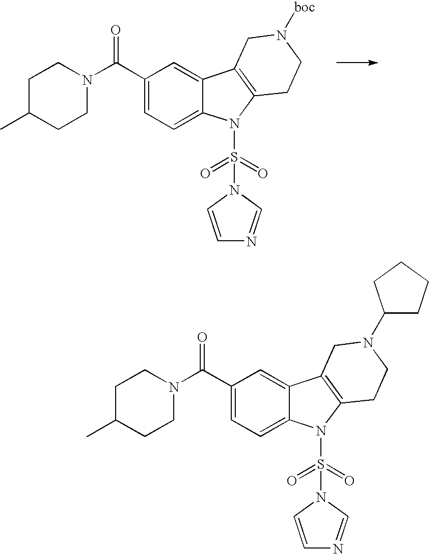

The solution of tert-butyl 5-(1H-imidazol-1-ylsulfonyl)-8-[(4-methylpiperidin-1-yl)carbonyl]-1,3,4,5-tetrahydro-2H-pyrido[4,3-b]indole-2-carboxylate (60 mg, 0.114 mmol) in 1M HCl (in diethyl ether, 3 mL) was stirred at room temperature for overnight, white precipitate was formed. After removal of solvents, the solid was taken up into dichloromethane (3 mL) and was treated with cyclopentanone (0.23 mmol) and sodium triacetoxyborohydride (0.23 mmol). The mixture was stirred at room temperature overnight, diluted with dichloromethane (15 mL), washed with saturated NaHCO₃ solution (5 mL), and dried over Na₂SO₄. The dichloromethane solution was concentrated in vacuo and the residue was purified with reverse phase HPLC to give the desired product as its TFA salt (11 mg). MS (M+1): 496.0. 1H NMR (400 MHz, CD3OD) δ ppm 0.96 (d, J=6.45 Hz, 3H), 1.05-1.34 (m, 2H), 1.51-1.96 (m, 9H), 2.21-2.36 (m, 2H), 2.74-2.93 (m, 1H), 3.01-3.19 (m, 1H), 3.40-3.66 (m, 3H), 3.70-3.85 (m, 2H), 3.88-4.01 (m, 1H), 4.28-4.42 (m, 1H), 4.53-4.65 (m, 1H), 4.65-4.77 (m, 1H), 6.99-7.28 (m, 1H), 7.46-7.66 (m, 3H), 8.07-8.44 (m, 2H).

### Example 205

### N-ethyl-8-[(4-methylpiperidin-1-yl)carbonyl]-2-(tetrahydro-2H-pyran-4-yl)-1,2,3,4-tetrahydro-5H-pyrido[4,3-b]indole-5-sulfonamide

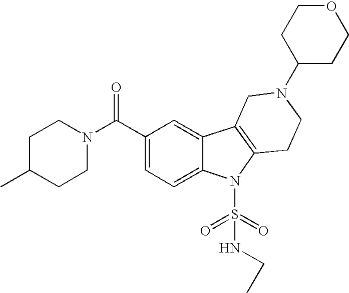

### Step A: The preparation of 1-({2-(tert-butoxycarbonyl)-8-[(4-methylpiperidin-1-yl)carbonyl]-1,2,3,4-tetrahydro-5H-pyrido[4,3-b]indol-5-yl}sulfonyl)-3-methyl-1H-imidazol-3-ium triflate

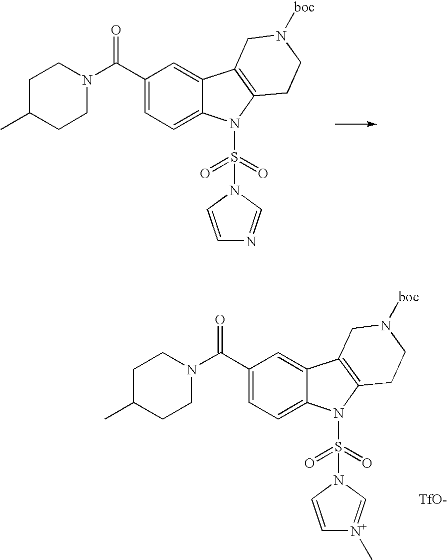

The solution of tert-butyl 5-(1H-imidazol-1-ylsulfonyl)-8-[(4-methylpiperidin-1-yl)carbonyl]-1,3,4,5-tetrahydro-2H-pyrido[4,3-b]indole-2-carboxylate (60 mg, 0.114 mmol) in dry acetonitrile (2 mL) was added a solution of methyl trifluoromethanesulfonate in dry acetonitrile (0.1 M, 1.25 mL) at 0° C., then stirred at the same temperature for 1 h. After evaporated, the crude was used for the next step without further purification. MS (M+1): 542.00.

### Step B: tert-butyl 5-[(ethylamino)sulfonyl]-8-[(4-methylpiperidin-1-yl)carbonyl]-1,3,4,5-tetrahydro-2H-pyrido[4,3-b]indole-2-carboxylate

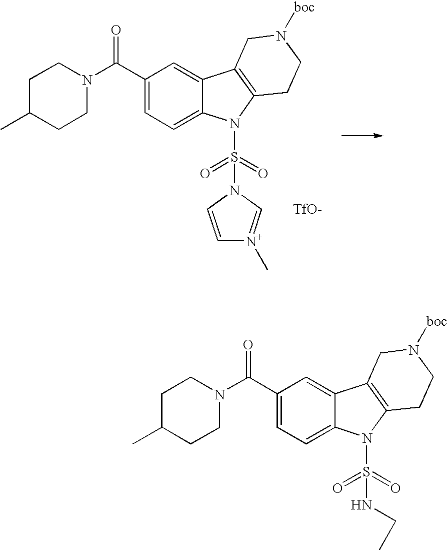

To a solution of crude 1-({2-(tert-butoxycarbonyl)-8-[(4-methylpiperidin-1-yl)carbonyl]-1,2,3,4-tetrahydro-5H-pyrido[4,3-b]indol-5-yl}sulfonyl)-3-methyl-1H-imidazol-3-ium triflate (0.1 mmol) in dry acetonitrile (3 mL) was added 2M ethylamine in THF (0.5 mmol), then stirred at room temperature for overnight. After evaporated, the crude was used for the next step without further purification. MS (M+1): 505.25.
Step C. N-ethyl-8-[(4-methylpiperidin-1-yl)carbonyl]-2-(tetrahydro-2H-pyran-4-yl)-1,2,3,4-tetrahydro-5H-pyrido[4,3-b]indole-5-sulfonamide.

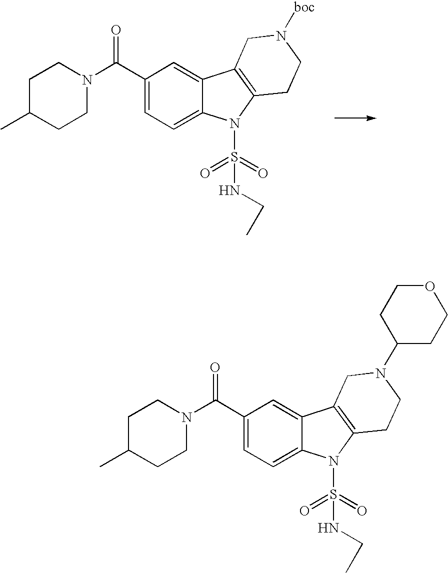

The crude tert-butyl 5-[(ethylamino)sulfonyl]-8-[(4-methylpiperidin-1-yl)carbonyl]-1,3,4,5-tetrahydro-2H-pyrido[4,3-b]indole-2-carboxylate from the previous step was treated with 4N HCl in dioxane (2 mL) for 2 h at room, then evaporated to dryness. The crude HCl salt was treated with tetrahydro-4H-pyran-4-one (0.2 mmol) and sodium triacetoxyborohydride (0.2 mmol) in dichloromethane (3 mL). The mixture was stirred at room temperature overnight, diluted with dichloromethane (15 mL), washed with saturated NaHCO₃ solution (5 mL), and dried over Na₂SO₄. The dichloromethane solution was concentrated in vacuo and the residue was purified with reverse phase HPLC to give the desired product as its TFA salt (3.0 mg, 4.4% for 4 steps). MS (M+1): 489.0.

### Example 206

### 5-(ethylsulfonyl)-8-[(4-methoxypiperidin-1-yl)carbonyl]-2-(tetrahydro-2H-pyran-4-yl)-2,3,4,5-tetrahydro-1H-pyrido[4,3-b]indole

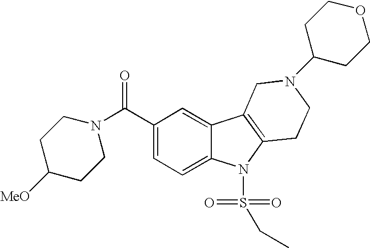

### Step A: The preparation of 8-benzyl 2-tert-butyl 1,3,4,5-tetrahydro-2H-pyrido[4,3-b]indole-2,8-dicarboxylate

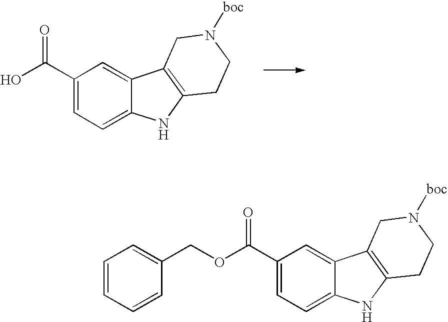

2-(tert-butoxycarbonyl)-2,3,4,5-tetrahydro-1H-pyrido[4,3-b]indole-8-carboxylic acid (2 g, 6.33 mmols) was dissolved in DMF (40 mL) and DBU (0.95 mL, 6.35 mmol) was added at 0° C. Benzyl bromide (0.75 mL, 6.30 mmols) was then added drop. The reaction mixture was stirred until no more starting material was observed then condensed under reduced pressure. The mixture was then diluted in dichloromethane and washed with water and brine then dried and condensed under reduced pressure. The residue was then recristallized in MeOH to provide a white solid (1.72 g, 67%). MS (M+1): 407.1.

### Step B. The preparation of 8-benzyl 2-tert-butyl 5-(ethylsulfonyl)-1,3,4,5-tetrahydro-2H-pyrido[4,3-b]indole-2,8-dicarboxylate

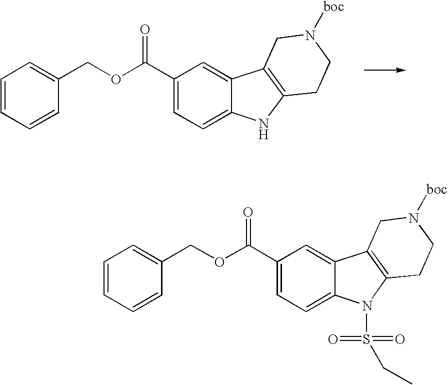

60% NaH (350 mg, 8.75 mmol) was added in one portion to a suspension of 8-benzyl 2-tert-butyl 1,3,4,5-tetrahydro-2H-pyrido[4,3-b]indole-2,8-dicarboxylate (1.12 g, 2.76 mmol) in DMF (17 mL) at room temperature under nitrogen. The mixture was stirred for 30 min, ethylsulfonyl chloride (500 μL, 5.28 mmols) was added at room temperature and the mixture was stirred for 2 hours at room temperature. The solvent was removed in vacuo and the residue was dissolved in dichloromethane and washed with water and dried over sodium sulfate. Removal of solvent gave the crude product. MS (M+1): 490.9.

### Step C. The preparation of 2-(tert-butoxycarbonyl)-5-(ethylsulfonyl)-2,3,4,5-tetrahydro-1H-pyrido[4,3-b]indole-8-carboxylic acid

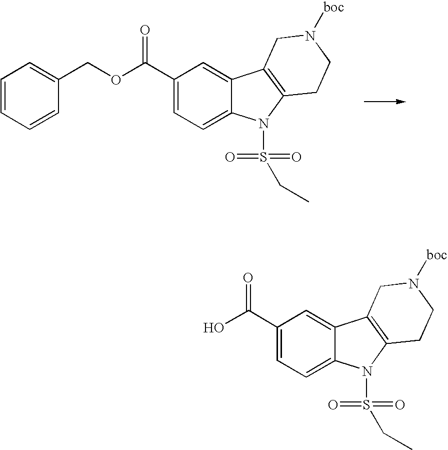

crude 8-benzyl 2-tert-butyl 5-(ethylsulfonyl)-1,3,4,5-tetrahydro-2H-pyrido[4,3-b]indole-2,8-dicarboxylate from last step was dissolved in MeOH and catalytic amount of 10% Pd/C was added. The mixture was hydrogenated overnight. After filtration, the solvent was removed under reduced pressure. MS: 408.8.

### Step D: The preparation of tert-butyl 5-(ethylsulfonyl)-8-[(4-methoxypiperidin-1-yl)carbonyl]-1,3,4,5-tetrahydro-2H-pyrido[4,3-b]indole-2-carboxylate

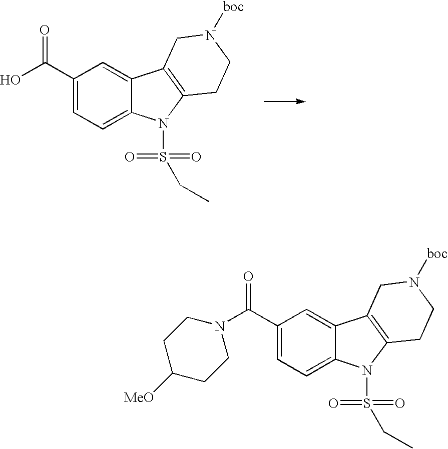

To a solution of 2-(tert-butoxycarbonyl)-5-(ethylsulfonyl)-2,3,4,5-tetrahydro-1H-pyrido[4,3-b]indole-8-carboxylic acid (0.88 mmol), diisopropylethylamine (1.06 mmol) and 4-methoxypieperidine (1.06 mmol) in DMF (4 mL) was added HATU (1.06 mmol) at room temperature. The mixture was stirred at room temperature for several hours, and then condensed under reduced pressure, diluted in dichloromethane and washed successively with a saturated aqueous NaHCO₃, water and brine. Removal of solvent under reduced pressure afforded the crude amide intermediate, which was used without purification. MS (M+1): 506.0.

### Step E: The preparation of 5-(ethylsulfonyl)-8-[(4-methoxypiperidin-1-yl)carbonyl]-2,3,4,5-tetrahydro-1H-pyrido[4,3-b]indole

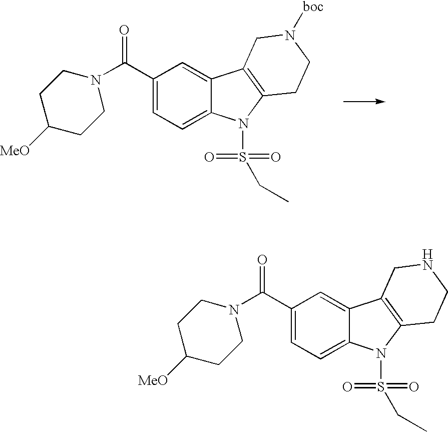

Following the same procedure described in Example 192, Step B: The desired product as its TFA salt was prepared from crude tert-butyl 5-(ethylsulfonyl)-8-[(4-methoxypiperidin-1-yl)carbonyl]-1,3,4,5-tetrahydro-2H-pyrido[4,3-b]indole-2-carboxylate. MS (M+1): 406.9.
Step F: The preparation of 5-(ethylsulfonyl)-8-[(4-methoxypiperidin-1-yl)carbonyl]-2-(tetrahydro-2H-pyran-4-yl)-2,3,4,5-tetrahydro-1H-pyrido[4,3-b]indole

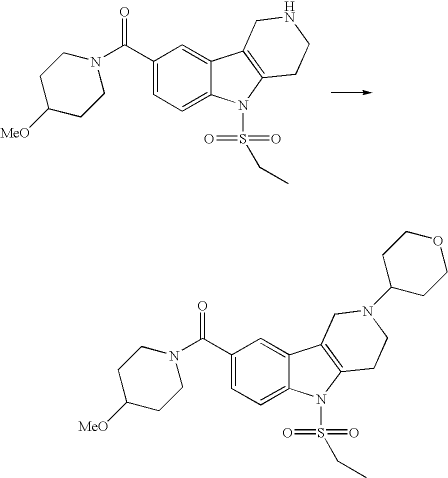

Sodium triacetoxyborohydride (60 mg, 0.57 mmol) was added to 5-(ethylsulfonyl)-8-[(4-methoxypiperidin-1-yl)carbonyl]-2,3,4,5-tetrahydro-1H-pyrido[4,3-b]indole.TFA (100 mg, 0.19 mmol) and tetrahydro-4H-pyran-4-one (70 μL, 0.76 mmol) in dichloromethane (3 mL). The mixture was stirred at room temperature. More sodium triacetoxyborohydride, (60 mg) and tetrahydro-4H-pyran-4-one (35 μL) was then added to complete the reaction. The mixture was then diluted with dichloromethane and washed with saturated NaHCO₃ solution, dried over Na₂SO₄ and condensed under reduced pressure. Purification of the crude over reverse phase HPLC (5-75% CH₃CN) provided the desired product as its di-TFA salt as a white solid. (36 mg, 30%) MS (M+1): 490.0. 1H NMR (400 MHz, CD3OD) δ ppm 1.06 (t, J=7.32 Hz, 3H), 1.30-1.60 (m, 3H), 1.62-1.91 (m, 4H), 2.04 (d, J=10.35 Hz, 2H), 3.22 (s, 3H), 3.25-3.54 (m, 11H), 3.54-3.65 (m, 1H), 3.79-4.00 (m, 1H), 3.98 (dd, J=11.72, 4.10 Hz, 2H), 4.13-4.69 (m, 2H), 7.28 (d, J=8.69, 1H), 7.52 (s, 1H), 7.89 (d, J=8.59 Hz, 1H).

### Example 207

### (1-{[2-cyclobutyl-5-(ethylsulfonyl)-2,3,4,5-tetrahydro-1H-pyrido[4,3-b]indol-8-yl]carbonyl}piperidin-4-yl)methanol

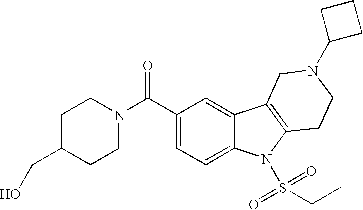

### Step A. The preparation of tert-butyl 5-(ethylsulfonyl)-8-{[4-(hydroxymethyl)piperidin-1-yl]carbonyl}-1,3,4,5-tetrahydro-2H-pyrido[4,3-b]indole-2-carboxylate

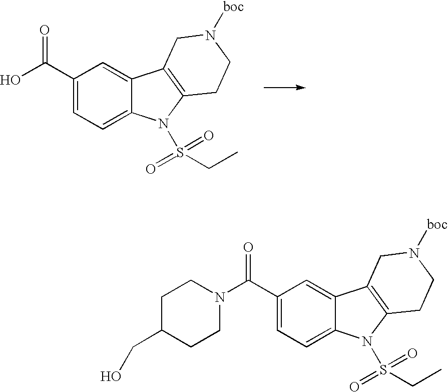

Following the similar procedure in Example 206, Step D: The title compound was prepared from 2-(tert-butoxycarbonyl)-5-(ethylsulfonyl)-2,3,4,5-tetrahydro-1H-pyrido[4,3-b]indole-8-carboxylic acid and piperidin-4-methanol. MS (M+1): 506.0.

### Step B: The preparation of 1-{[5-(ethylsulfonyl)-2,3,4,5-tetrahydro-1H-pyrido[4,3-b]indol-8-yl]carbonyl}piperidin-4-yl)methyl trifluoroacetate

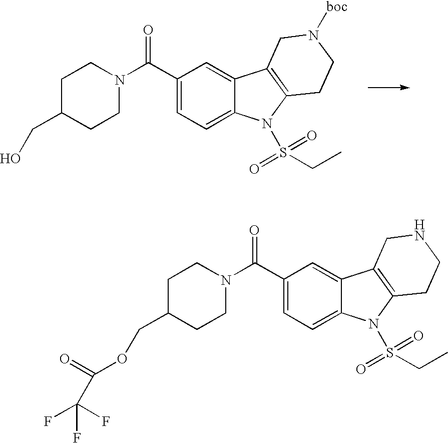

Following the same procedure described in Example 192, Step B: The title compound as its TFA salt was prepared from tert-butyl 5-(ethylsulfonyl)-8-{[4-(hydroxymethyl)piperidin-1-yl]carbonyl}-1,3,4,5-tetrahydro-2H-pyrido[4,3-b]indole-2-carboxylate. MS (M+1): 502.2.

### Step C: The preparation of (1-{[2-cyclobutyl-5-(ethylsulfonyl)-2,3,4,5-tetrahydro-1H-pyrido[4,3-b]indol-8-yl]carbonyl}piperidin-4-yl)methanol

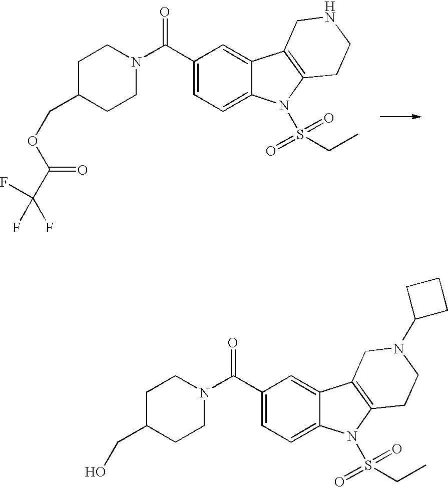

Sodium triacetoxyborohydride (130 mg, 0.61 mmol) was added to (1-{[5-(ethylsulfonyl)-2,3,4,5-tetrahydro-1H-pyrido[4,3-b]indol-8-yl]carbonyl}piperidin-4-yl)methyl trifluoroacetate TFA (160 mg, 0.32 mmol) and cyclobutanone (48 μL, 0.64 mmol) in dichloromethane (3 mL). The mixture was stirred at room temperature for a couple hours then additional sodium triacetoxyborohydride (76 mg) was added to complete the reaction. The mixture was then diluted with dichloromethane and washed with saturated NaHCO₃ solution, dried over Na₂SO₄ and condensed under reduced pressure. Purification of the crude over reverse phase HPLC (5-75% CH₃CN) provided the desired product as its di-TFA salt as a white solid. (36 mg, 24%). MS (M+1): 460.0. 1H NMR (400 MHz, CD3OD) δ ppm 0.99-1.18 (m, 2H), 1.03 (t, J=7.32 Hz, 3H), 1.45-1.86 (m, 5H), 2.32-2.40 (m, 4H), 2.63-2.82 (m, 1H), 2.86-3.04 (m, 1H), 3.11-3.40 (m, 8H), 3.50-3.73 (m, 2H), 3.74-3.88 (m, 1H), 3.90-4.12 (m, 1H), 4.41-4.66 (m, 2H), 7.24 (d, J=8.79, 1H), 7.46 (s, 1H), 7.86 (d, J=8.79 Hz, 1H).

### Example 208

### 1-{[2-cyclobutyl-5-(ethylsulfonyl)-2,3,4,5-tetrahydro-1,7-pyrido[4,3-b]indol-8-yl]carbonyl}piperidin-4-ol

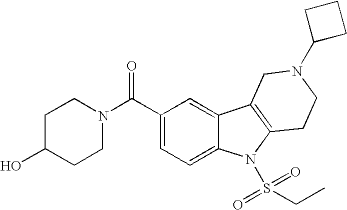

### Step A: The preparation of tert-butyl 5-(ethylsulfonyl)-8-[(4-hydroxypiperidin-1-yl)carbonyl]-1,3,4,5-tetrahydro-2H-pyrido[4,3-b]indole-2-carboxylate

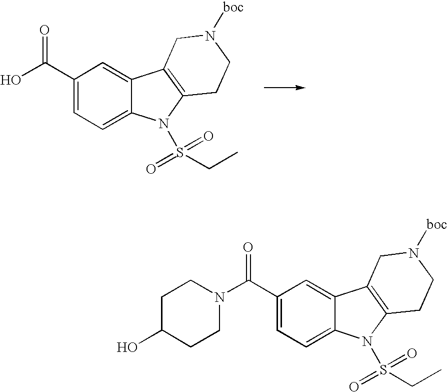

Following the similar procedure in Example 206, Step D: The title compound was prepared from 2-(tert-butoxycarbonyl)-5-(ethylsulfonyl)-2,3,4,5-tetrahydro-1H-pyrido[4,3-b]indole-8-carboxylic acid and piperidin-4-ol. MS (M+1): 492.0.

### Step B: The preparation of trifluoromethyl 1-{[5-(ethylsulfonyl)-2,3,4,5-tetrahydro-1H-pyrido[4,3-b]indol-8-yl]carbonyl}piperidine-4-carboxylate

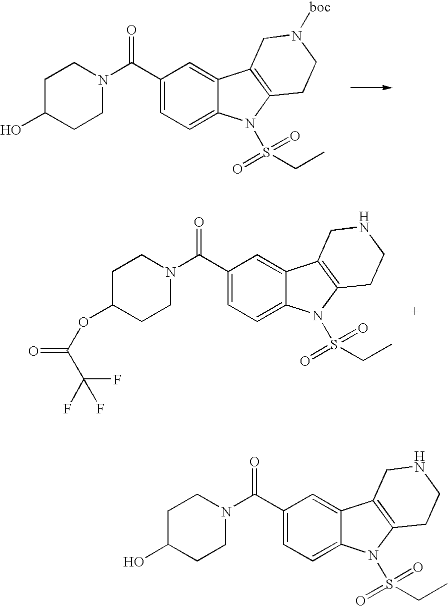

Following the same procedure described in Example 192, Step B: The title compound as its TFA salt, together with its corresponding alcohol was prepared from tert-butyl 5-(ethylsulfonyl)-8-[(4-hydroxypiperidin-1-yl)carbonyl]-1,3,4,5-tetrahydro-2H-pyrido[4,3-b]indole-2-carboxylate. MS (M+1): 488.2 and 392.2.

### Step C: (1-{[2-cyclobutyl-5-(ethylsulfonyl)-2,3,4,5-tetrahydro-1H-pyrido[4,3-b]indol-8-yl]carbonyl}piperidin-4-yl)methanol

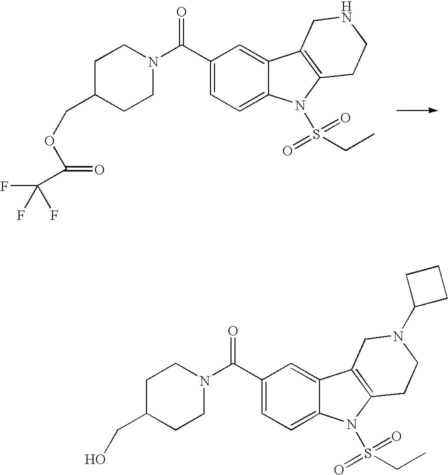

Following the similar procedure of Example 207, Step C: The title compound as its TFA salt was prepared from crude product from last step (the mixture of trifluoromethyl 1-{[5-(ethylsulfonyl)-2,3,4,5-tetrahydro-1H-pyrido[4,3-b]indol-8-yl]carbonyl}piperidine-4-carboxylate.TFA and its corresponding alcohol) and cyclobutanone. MS (M+1): 460.0. 1H NMR (400 MHz, CD3OD) δ ppm 1.16 (t, J=7.42 Hz, 3H), 1.34-1.61 (m, 2H), 1.69-1.83 (m, 1H), 1.84-1.99 (m, 3H), 2.27-2.49 (m, 4H), 3.17-3.51 (m, 6H), 3.44 (q, J=7.42 Hz, 2H), 3.54-3.69 (m, 1H), 3.70-3.82 (m, 1H), 3.82-3.98 (m, 2H), 4.06-4.22 (m, 2H), 4.60-4.73 (m, 1H), 7.37 (dd, J=8.59, 1.56 Hz, 1H), 7.60 (s, 1H), 7.99 (d, J=8.59 Hz, 1H).

### Example 209

### N-[5-(methylsulfonyl)-2-(tetrahydro-2,7-pyran-4-yl)-2,3,4,5-tetrahydro-1H-pyrido[4,3-b]indol-8-yl]cyclopentanecarboxamide

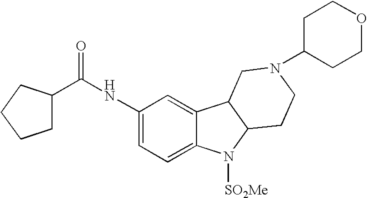

### Step A: The preparation of tert-butyl 5-(methylsulfonyl)-8-nitro-1,3,4,4a,5,9b-hexahydro-2H-pyrido[4,3-b]indole-2-carboxylate

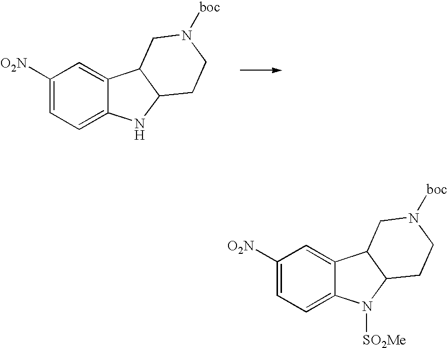

60% NaH (110 mg, 2.75 mmol) was added in one portion to a suspension of tert-butyl 8-nitro-1,3,4,4a,5,9b-hexahydro-2H-pyrido[4,3-b]indole-2-carboxylate (600 mg, 1.89 mmol) in DMF (10 mL) at room temperature under nitrogen. The mixture was stirred for 30 min, methylsulfonylchloride (170 μL, 2.19 mmol) was added at 0° C., and the reaction mixture was allowed to rise to room temperature. The solvent was removed in vacuo and the residue was dissolved in dichloromethane and washed with water dried over sodium sulfate. Removal of solvent gave the crude product, which was used for next step without purification. MS (M+1): 396.9.

### Step B: The preparation of tert-butyl 8-amino-5-(methylsulfonyl)-1,3,4,4a,5,9b-hexahydro-2H-pyrido[4,3-b]indole-2-carboxylate

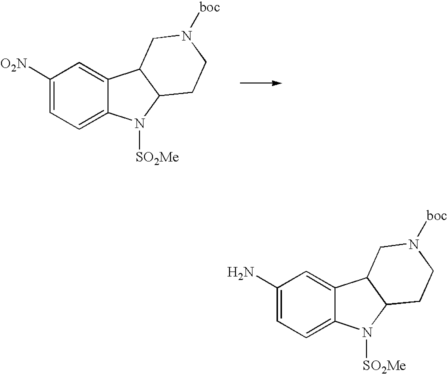

Crude tert-butyl 5-(methylsulfonyl)-8-nitro-1,3,4,4a,5,9b-hexahydro-2H-pyrido[4,3-b]indole-2-carboxylate was dissolved in THF (15 mL) and 10% Pd/C (60 mg) was added. The reaction mixture was hydrogenated overnight. The mixture was then filtered and condensed under reduced pressure. MS (M+1): 366.9.

### Step C: tert-butyl 8-[(cyclopentylcarbonyl)amino]-5-(methylsulfonyl)-1,3,4,4a,5,9b-hexahydro-2H-pyrido[4,3-b]indole-2-carboxylate

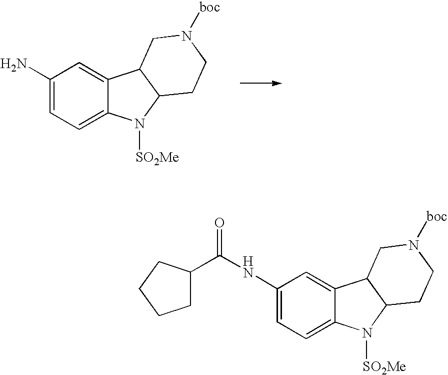

To a solution of tert-butyl-amino-5-(methylsulfonyl)-1,3,4,4a,5,9b-hexahydro-2H-pyrido[4,3-b]indole-2-carboxylate (0.8 mmol) in dichloromethane (10 mL) was added triethylamine (1.3 mmol). Cyclopentanecarbonyl chloride (0.92 mmol) was then added dropwise at 0° C. The reaction was stirred until no more starting material was observed. The mixture was diluted in dichloromethane and washed with water and brine, dried and condensed under reduced pressure.

### Step D: N-[5-(methylsulfonyl)-2,3,4,4a,5,9b-hexahydro-1H-pyrido[4,3-b]indol-8-yl]cyclopentanecarboxamide

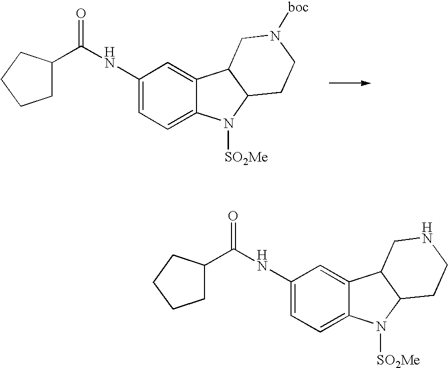

Following the same procedure described in Example 192, Step B: The desired product as its TFA salt was prepared from tert-butyl 8-[(cyclopentylcarbonyl)amino]-5-(methylsulfonyl)-1,3,4,4a,5,9b-hexahydro-2H-pyrido[4,3-b]indole-2-carboxylate. MS (M+1): 362.0.

### Step E: The preparation of N-[5-(methylsulfonyl)-2-(tetrahydro-2H-pyran-4-yl)-2,3,4,5-tetrahydro-1H-pyrido[4,3-b]indol-8-yl]cyclopentanecarboxamide

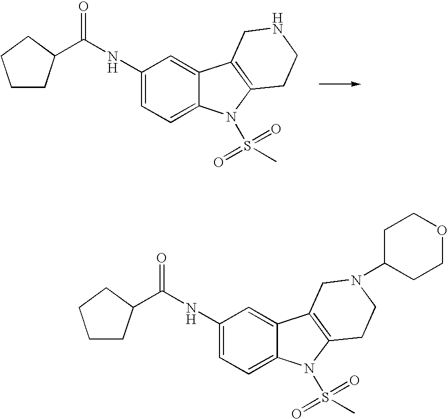

Following the similar procedure of Example 208, Step C: The title compound as its TFA salt was prepared N-[5-(methylsulfonyl)-2,3,4,4a,5,9b-hexahydro-1H-pyrido[4,3-b]indol-8-yl]cyclopentanecarboxaraide and tetrahydro-4H-pyran-4-one. MS (M+1): 446.0. 1H NMR (400 MHz, CD3OD) δ ppm 1.51-1.92 (m, 11H), 2.08 (d, J=10.74 Hz, 2H), 2.73 (q, J=7.88 Hz, 1H), 3.12 (s, 3H), 3.25-3.47 (m, 1H), 3.41 (t, J=11.23 Hz, 3H), 3.57-3.70 (m, 1H), 4.03 (dd, J=11.62, 4.20 Hz, 2H), 4.35-4.58 (m, 2H), 7.23 (dd, J=8.98, 1H), 7.81 (d, J=8.98 Hz, 1H), 7.90 (s, 1H).

### Example 210

### N-[5-(ethylsulfonyl)-2-(tetrahydro-2H-pyran-4-yl)-2,3,4,5-tetrahydro-1H-pyrido[4,3-b]indol-8-yl]pyrrolidine-1-carboxamide

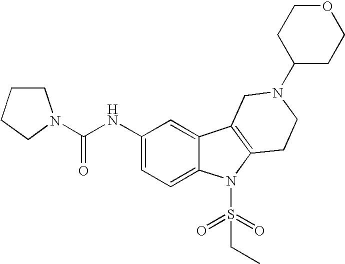

### Step A: The preparation of tert-butyl 5-(ethylsulfonyl)-8-nitro-1,3,4,5-tetrahydro-2H-pyrido[4,3-b]indole-2-carboxylate

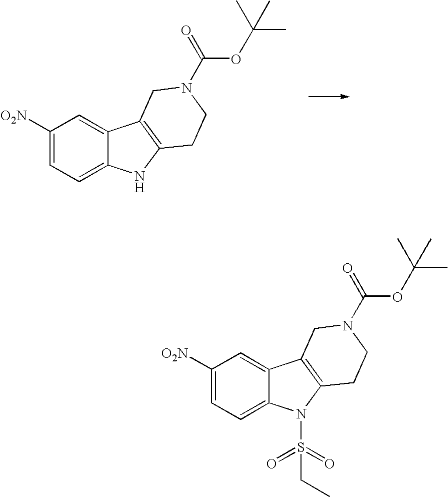

To a solution of tert-butyl 8-nitro-1,3,4,5-tetrahydro-2H-pyrido[4,3-b]indole-2-carboxylate (3.0 mmol) in dry DMF (10 mL) was added t-BuOK (6.0 mmol) at 0° C. and stirred for 10 min at the same temperature. Then ethanesulfonyl chloride (6.0 mmol) was added stirred at room temperature for 3 h. The solvent was removed and the residue was taken up into dichloromethane (40 mL), extracted with water and brine, dried over Na₂SO₄, concentrated to yield crude product as yellow solids (1.15 g, 94%). MS (M+1): 410.0.

### Step B: The preparation of tert-butyl 8-amino-5-(ethylsulfonyl)-1,3,4,5-tetrahydro-2H-pyrido[4,3-b]indole-2-carboxylate

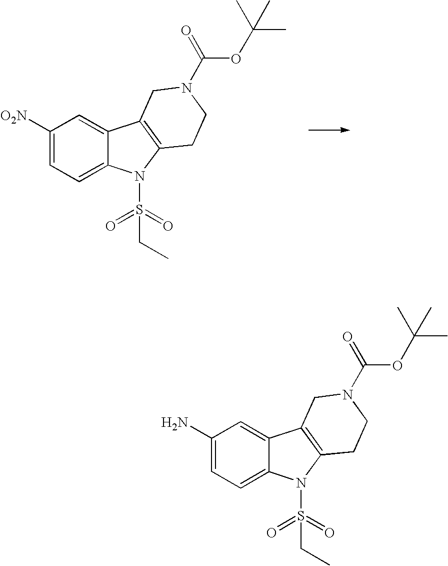

The solution of tert-butyl 5-(ethylsulfonyl)-8-nitro-1,3,4,5-tetrahydro-2H-pyrido[4,3-b]indole-2-carboxylate (1.0 mmol) in methanol (30 mL) was hydrogenated at 40 psi in the presence of 10% Pd/C at room temperature for 2 h. After filtration, the crude aniline was obtained as off-white solids (368 mg, 97%), which used for the next step without further purification. MS (M+1): 380.0.

### Step C: The preparation of tert-butyl 5-(ethylsulfonyl)-8-[(pyrrolidin-1-ylcarbonyl)amino]-1,3,4,5-tetrahydro-2H-pyrido[4,3-b]indole-2-carboxylate

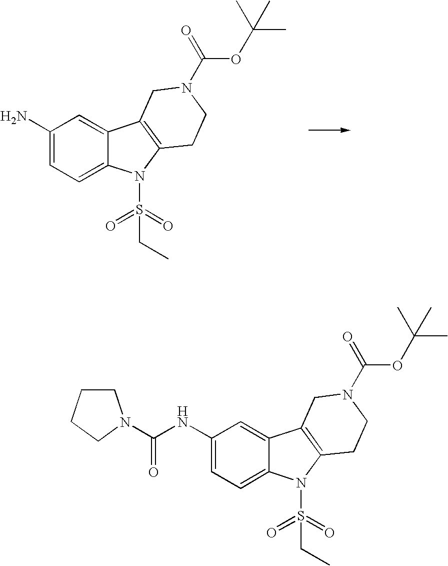

To a solution of tert-butyl 8-amino-5-(ethylsulfonyl)-1,3,4,5-tetrahydro-2H-pyrido[4,3-b]indole-2-carboxylate (0.3 mmol) in dry acetonitrile (5 mL) was added pyrrolidine-1-carbonyl chloride (0.45 mmol) followed by triethyl amine (0.45 mmol), the reaction mixture was stirred at 80° C. for overnight. After being concentrated, the residue was taken up into dichloromethane (30 mL), washed with saturated NaHCO₃ solution (10 mL), dried over Na₂SO₄. The solution was concentrated in vacuo and the residue was purified with flash chromatography to give the desired product as an oil (132 mg, 92%). MS (M+1): 477.0.

### Step D: The preparation of N-[5-(ethylsulfonyl)-2-(tetrahydro-2H-pyran-4-yl)-2,3,4,5-tetrahydro-1H-pyrido[4,3-b]indol-8-yl]pyrrolidine-1-carboxamide

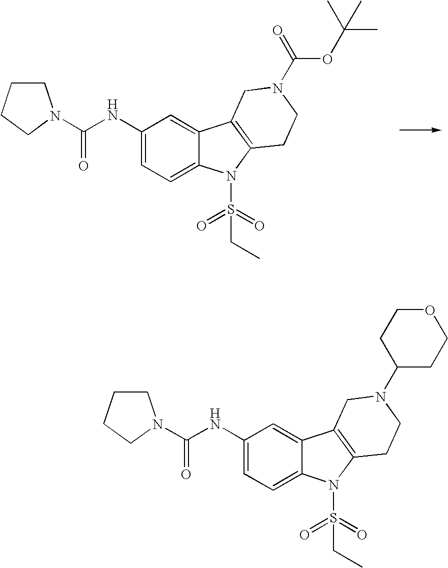

To a solution of tert-butyl 5-(ethylsulfonyl)-8-[(pyrrolidin-1-ylcarbonyl)amino]-1,3,4,5-tetrahydro-2H-pyrido[4,3-b]indole-2-carboxylate (0.28 mmol) in dichloromethane (5 mL) was added TFA (1 mL) and the reaction mixture was stirred at room temperature for 3 h. After being concentrated, the crude TFA salt was treated with tetrahydro-4H-pyran-4-one (0.56 mmol), and sodium triacetoxyborohydride (0.56 mmol) in dichloromethane (5 mL). The mixture was stirred at room temperature overnight, diluted with dichloromethane (20 mL), washed with saturated NaHCO₃ solution (10 mL), dried over Na₂SO₄. The solution was concentrated in vacuo and the residue was purified with reverse phase HPLC to give the desired product as its TFA salt (84 mg, 52%). MS (M+1): 461.0. 1H NMR (400 MHz, CD3OD) δ ppm 1.17 (t, J=7.42 Hz, 3H), 1.81-1.93 (m, 2H), 1.96 (t, J=6.44 Hz, 4H), 2.09-2.20 (m, 2H), 3.36-3.54 (m, 11H), 3.64-3.76 (m, 1H), 3.92-4.05 (m, 1H), 4.11 (dd, J=11.62, 4.00 Hz, 2H), 4.42-4.54 (m, 1H), 4.55-4.67 (m, 1H), 7.27 (dd, J=8.98, 1.95 Hz, 1H), 7.69 (d, J=1.95 Hz, 1H), 7.83 (d, J=8.98 Hz, 1H).

### Example 211

### N-[5-(ethylsulfonyl)-2-(tetrahydro-2H-pyran-4-yl)-2,3,4,5-tetrahydro-1H-pyrido[4,3-b]indol-8-yl]-1-methyl-1H-imidazole-4-sulfonamide

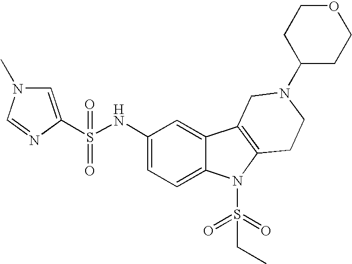

### Step A: The preparation of tert-butyl 5-(ethylsulfonyl)-8-{[(1-methyl-1H-imidazol-4-yl)sulfonyl]amino}-1,3,4,5-tetrahydro-2H-pyrido[4,3-b]indole-2-carboxylate

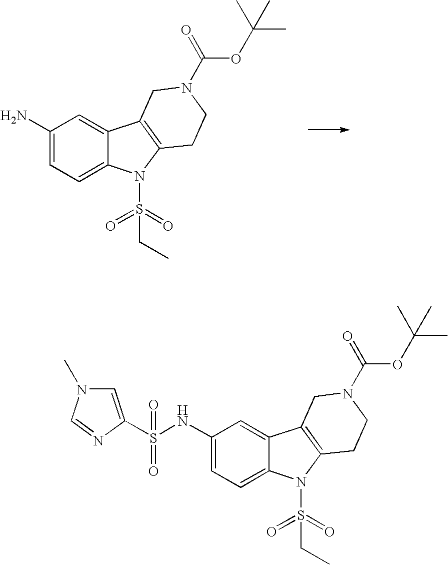

To a solution of tert-butyl 8-amino-5-(ethylsulfonyl)-1,3,4,5-tetrahydro-2H-pyrido[4,3-b]indole-2-carboxylate (0.26 mmol) in dry dichloromethane (8 mL) was added 1-methyl-1H-imidazole-4-sulfonyl chloride (0.32 mmol) followed by triethyl amine (0.52 mmol), the reaction mixture was stirred at room temperature for overnight and diluted with dichloromethane (20 mL), washed with saturated NaHCO₃ solution (10 mL), dried over Na₂SO₄. The solution was concentrated in vacuo and the crude product was used for the next step without further purification. MS (M+1): 524.0.

### Step B: The preparation of N-[5-(ethylsulfonyl)-2-(tetrahydro-2H-pyran-4-yl)-2,3,4,5-tetrahydro-1H-pyrido[4,3-b]indol-8-yl]-1-methyl-1H-imidazole-4-sulfonamide

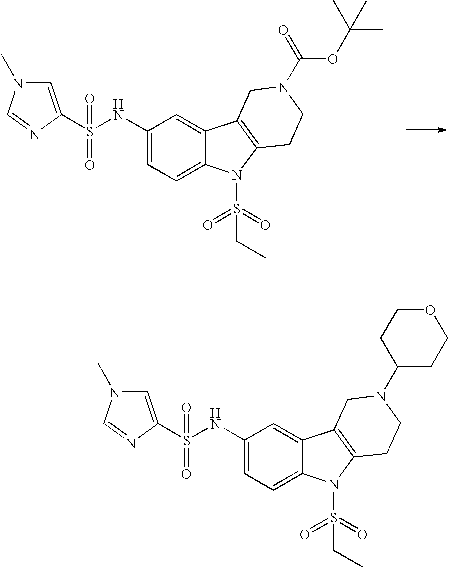

To a solution of crude tert-butyl 5-(ethylsulfonyl)-8-{[(1-methyl-1H-imidazol-4-yl)sulfonyl]amino}-1,3,4,5-tetrahydro-2H-pyrido[4,3-b]indole-2-carboxylate (0.26 mmol) in dichloromethane (3 mL) was added TFA (0.5 mL) and the reaction mixture was stirred at room temperature for 3 h. After being concentrated, the crude TFA salt was treated with tetrahydro-4H-pyran-4-one (0.52 mmol), and sodium triacetoxyborohydride (0.52 mmol) in dichloromethane (5 mL). The mixture was stirred at room temperature overnight, diluted with dichloromethane (20 mL), washed with saturated NaHCO₃ solution (10 mL), and dried over Na₂SO₄. The solution was concentrated in vacuo and the residue was purified with reverse phase HPLC to give the desired product as its TFA salt (38 mg, 21% for 3 steps). MS (M+1): 508.0. 1H NMR (400 MHz, CD3OD) δ ppm 1.16 (t, J=7.32 Hz, 3H), 1.80-2.00 (m, 2H), 2.09-2.21 (m, 2H), 3.40 (q, J=7.29 Hz, 4H), 3.44-3.54 (m, 3H), 3.66 (s, 3H), 3.68-3.76 (m, 1H), 3.87-4.05 (m, 1H), 4.08-4.16 (m, 2H), 4.39-4.54 (m, 1H), 4.56-4.72 (m, 1H), 7.06 (dd, J=8.95, 2.00 Hz, 1H), 7.46 (d, J=2.00 Hz, 1H), 7.60 (s, 1H) 7.64 (s, 1H), 7.77 (d, J=8.95 Hz, 1H).

### Example 212

### tert-butyl(trans-4-{[(2-cyclopentyl-5-propyl-2,3,4,5-tetrahydro-1H-pyrido[4,3-b]indol-8-yl)amino]carbonyl}cyclohexyl)carbamate

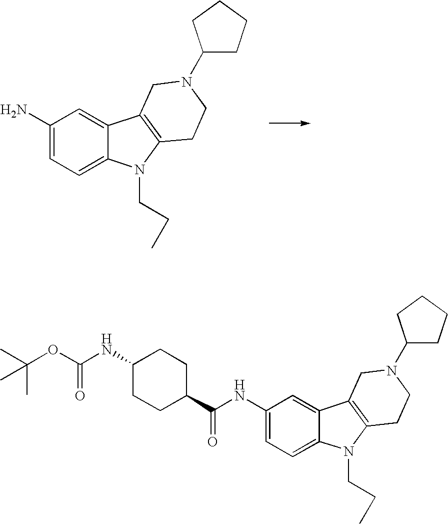

To s solution of 2-cyclopentyl-5-propyl-2,3,4,5-tetrahydro-1H-pyrido[4,3-b]indol-8-amine (0.74 mmol) in DMF (8 mL) was added tert-butyl[trans-4-(aminocarbonyl)cyclohexyl]carbamate (0.89 mmol) followed by HATU (0.89 mmol) and diisopropylethylamine (0.89 mmol), the reaction mixture was stirred at room temperature for 3 h. After being concentrated, the residue was taken up into dichloromethane (50 mL), extracted with saturated NaHCO₃ solution (10 mL) and brine (10 mL), dried over Na₂SO₄, concentrated. The crude product (353 mg, 91%) can be used for the next step without further purification. A part of crude product was purified by reverse phase HPLC to yield TFA salt. MS (M+1): 523.0. 1H NMR (400 MHz, CD3OD) δ ppm 0.90 (t, J=7.42 Hz, 3H), 1.18-1.33 (m, 2H), 1.41 (s, 9H), 1.52-1.69 (m, 2H), 1.71-1.83 (m, 5H), 1.83-2.04 (m, 8H), 2.22-2.36 (m, 2H), 3.17-3.24 (m, 3H), 3.46-3.59 (m, 1H), 3.79 (t, J=7.71 Hz, 1H), 3.96 (d, J=12.11 Hz, 1H), 4.02-4.13 (m, 2H), 4.35 (d, J=14.06 Hz, 1H), 4.67 (d, J=14.06 Hz, 1H), 7.09-7.14 (m, 1H), 7.34 (d, J=8.79 Hz, 1H), 7.80 (d, J=1.76 Hz, 1H).

### Example 213

### trans-N-(2-cyclopentyl-5-propyl-2,3,4,5-tetrahydro-1H-pyrido[4,3-b]indol-8-yl)-4-[(2,2-dimethylpropanoyl)amino]cyclohexanecarboxamide

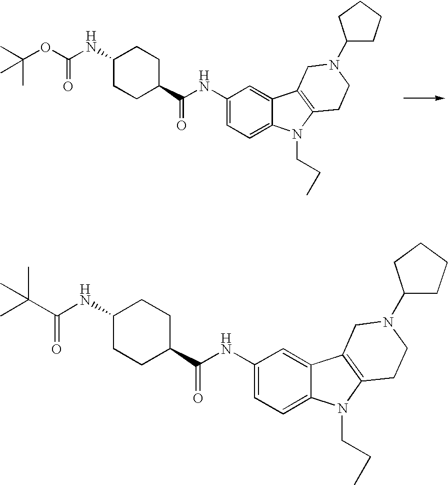

The crude tert-butyl(trans-4-{[(2-cyclopentyl-5-propyl-2,3,4,5-tetrahydro-1H-pyrido[4,3-b]indol-8-yl)amino]carbonyl}cyclohexyl)carbamate (0.15 mmol) in EtOAc (5 mL) was treated with 4N HCl in dioxane (1 mL) for 5 h at room, then evaporated to dryness. The crude HCl salt was treated with cyclopentanone (0.3 mmol), and sodium triacetoxyborohydride (0.3 mmol) in dichloromethane (3 mL). The mixture was stirred at room temperature overnight, diluted with dichloromethane (15 mL), washed with saturated NaHCO₃ solution (5 mL), and dried over Na₂SO₄. The solution was concentrated in vacuo and the residue was purified with reverse phase HPLC to give the desired product as its TFA salt (28 mg, 30%). MS (M+1): 507.0. 1H NMR (400 MHz, CD3OD) δ ppm 0.90 (t, J=7.42 Hz, 3H), 1.15 (s, 9H), 1.26-1.49 (m, 2H), 1.61-1.85 (m, 8H), 1.85-2.00 (m, 7H), 2.25-2.38 (m, 2H), 3.15-3.23 (m, 2H), 3.45-3.59 (m, 1H), 3.61-3.73 (m, 1H), 3.79 (t, J=7.81 Hz, 1H), 3.92-4.01 (m, 1H), 4.03-4.13 (m, 2H), 4.35 (d, J=14.06 Hz, 1H), 4.67 (d, J=14.06 Hz, 1H), 7.11 (dd, J=8.75, 1.80 Hz, 1H), 7.34 (d, J=8.75 Hz, 1H), 7.80 (d, J=1.80 Hz, 1H).

### Example 214

### N-[5-propyl-2-(tetrahydro-2H-pyran-4-yl)-2,3,4,5-tetrahydro-1H-pyrido[4,3-b]indol-8-yl]cyclohexanecarboxamide

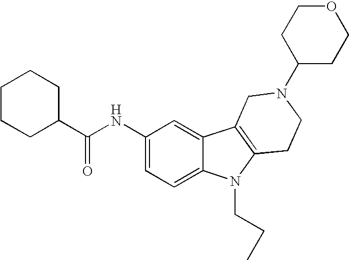

### Step A: The preparation of 5-propyl-2-(tetrahydro-2H-pyran-4-yl)-2,3,4,5-tetrahydro-1H-pyrido[4,3-b]indol-8-amine

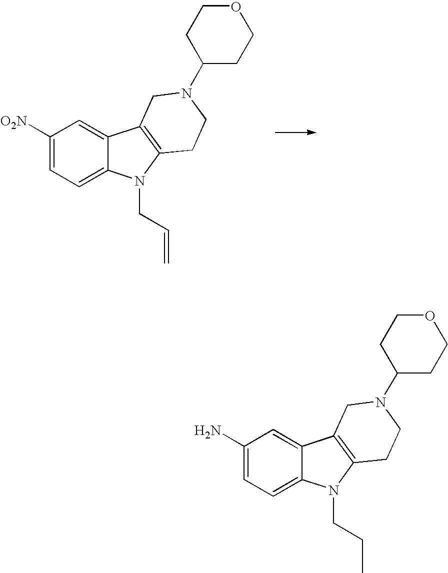

To a solution of 5-allyl-8-nitro-2-(tetrahydro-2H-pyran-4-yl)-2,3,4,4a,5,9b-hexahydro-1H-pyrido[4,3-b]indole (1.6 g, 4.69 mmol) in ethyl acetate/methanol (1:1 v/v, 50 mL) was added 10% Pd/C (200 mg). The mixture was hydrogenated at room temperature (40 psi) for overnight. After filtration and concentration, yielded crude product (1.28 g, 87%) as an oil. MS (M+1): 314.07.

### Step B: The preparation of N-[5-propyl-2-(tetrahydro-2H-pyran-4-yl)-2,3,4,5-tetrahydro-1H-pyrido[4,3-b]indol-8-yl]cyclohexanecarboxamide

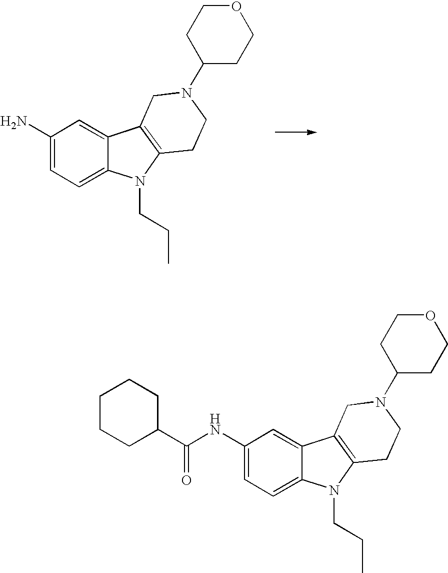

To a solution of 5-propyl-2-(tetrahydro-2H-pyran-4-yl)-2,3,4,5-tetrahydro-1H-pyrido[4,3-b]indol-8-amine (0.25 mmol) in dry DMF (3 mL) was added cyclohexanecarboxylic acid (0.3 mmol) followed by disiopropylethylamine (0.3 mmol) and HATU (0.3 mmol). The mixture was stirred at room temperature for 3 h. The solvent was removed in vacuo and the residue was treated with water (10 mL) and extracted with dichloromethane (2×20 mL), dried over Na₂SO₄, evaporated. The residue was purified with reverse phase HPLC to give the desired product as its TFA salt (54 mg, 40%). MS (M+1): 426.0. 1H NMR (400 MHz, CD3OD) δ ppm 0.86 (t, J=7.03 Hz, 3H), 1.17-1.40 (m, 3H), 1.42-1.57 (m, 2H), 1.61-1.95 (m, 9H), 2.05-2.19 (m, 2H), 2.25-2.42 (m, 1H), 3.12-3.22 (m, 3H), 3.33-3.57 (m, 3H), 3.58-3.73 (m, 1H), 3.89-4.01 (m, 1H), 4.02-4.17 (m, 3H), 4.34-4.50 (m, 1H), 4.52-4.66 (m, 1H), 7.07-7.14 (m, 1H), 7.27-7.35 (m, 1H), 7.73-7.80 (m, 1H).

### Example 215-218

The following examples were prepared by the same method described in Example 214, Step B.

| Exam- | | | |
|---|---|---|---|
| ples | AZ No. | Structures | Data |
| 215 | 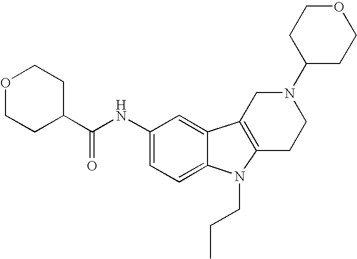 | N-[5-propyl-2- (tetrahydro-2H-pyran-4- yl)-2,3,4,5-tetrahydro- 1H-pyrido[4,3-b]indol-8- yl]tetrahydro-2H-pyran- 4-carboxamide | MS (M + 1): 426.0. 1H NMR (400 MHz, CD3OD) δ ppm 0.85-0.90 (m, 3 H), 1.67-1.80 (m, 4 H), 1.80-1.97 (m, 3 H), 2.06-2.22 (m, 2 H), 2.55- 2.67 (m, 1 H), 3.15- 3.23 (m, 2 H), 3.46 (m, 5 H), 3.59-3.73 (m, 1 H), 3.90-4.01 (m, 4 H), 4.02- 4.14 (m, 4 H), 4.39-4.51 (m, 1 H), 4.56-4.67 (m, 1H), 7.08-7.15 (m, 1 H), 7.33 (d, J = 8.59 Hz, 1 H), 7.79 (s, 1 H). |
| 216 | 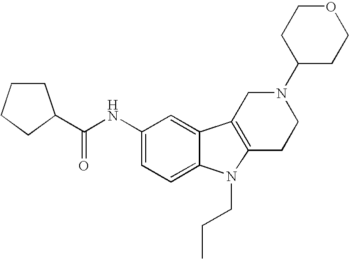 | N-[5-propyl-2- (tetrahydro-2H-pyran-4- yl)-2,3,4,5-tetrahydro- 1H-pyrido[4,3-b]indol-8- yl] cyclopentanecarboxamide | MS (M + 1): 410.0. 1H NMR (400 MHz, CD3OD) δ ppm 0.84- 0.93 (m, 3 H), 1.56-1.68 (m, 2H), 1.68-1.84 (m, 6 H), 1.84-1.97 (m, 4 H), 2.06-2.19 (m, 2 H), 2.71- 2.85 (m, 1 H), 3.16- 3.23 (m, 2 H), 3.40-3.55 (m, 3 H), 3.60-3.73 (m, 1H), 3.90-4.01 (m, 1 H), 4.02-4.14 (m, 4H), 4.39- 4.50 (m, 1 H), 4.55- 4.66 (m, 1 H), 7.11 (d, J = 8.59 Hz, 1 H), 7.32 (d, J = 8.59 Hz, 1 H), 7.78 (s, 1 H). |
| 217 | 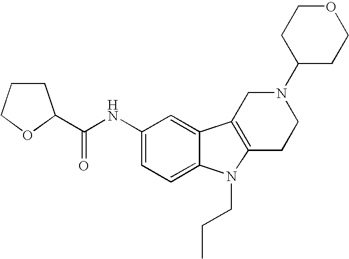 | N-[5-propyl-2- (tetrahydro-2H-pyran-4- yl)-2,3,4,5-tetrahydro- 1H-pyrido[4,3-b]indol-8- yl]tetrahydrofuran-2- carboxamide | MS (M + 1): 412.0. 1H NMR (400 MHz, CD3OD) δ ppm 0.82- 0.91 (m, 3 H), 1.70-1.80 (m, 2 H), 1.82-1.99 (m, 4 H), 1.98-2.08 (m, 1 H), 2.08-2.19 (m, 2 H), 2.23- 2.38 (m, 1 H), 3.15-3.23 (m, 2 H), 3.41-3.56 (m, 3 H), 3.60-3.71 (m, 1 H), 3.85-3.94 (m, 1 H), 3.94- 4.02 (m, 1 H), 4.02- 4.14 (m, 5 H), 4.36-4.50 (m, 2 H), 4.57-4.68 (m, 1 H), 7.21 (d, J = 8.59 Hz, 1 H), 7.34 (d, J = 8.59 Hz, 1 H), 7.75 (s, 1 H). |
| 218 | 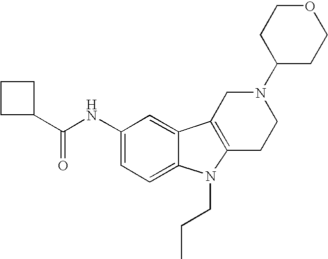 | N-[5-propyl-2- (tetrahydro-2H-pyran-4- yl)-2,3,4,5-tetrahydro- 1H-pyrido[4,3-b]indol-8- yl] cyclobutanecarboxamide | MS (M + 1): 396.0. 1H NMR (400 MHz, CD3OD) δ ppm 0.87 (t, J = 6.54 Hz, 3 H), 1.69- 1.80 (m, 2 H), 1.81-1.95 (m, 4 H), 1.95-2.06 (m, 1 H), 2.08-2.22 (m, 4 H), 2.25-2.39 (m, 2 H), 3.16- 3.24 (m, 2 H),3.38- 3.56 (m, 3 H), 3.60-3.73 (m, 1 H), 3.93-4.02 (m, 1 H), 4.01-4.15 (m, 4 H), 4.39-4.51 (m, 1 H), 4.55- 4.66 (m, 1 H), 7.08- 7.15 (m, 1 H),7.29-7.36 (m, 1 H), 7.78 (s, 1 H). |

### Example 219

### 8-[(4-methylpiperidin-1-yl)carbonyl]-5-(pyridin-2-ylmethyl)-2-(tetrahydro-2H-pyran-4-yl)-2,3,4,5-tetrahydro-1H-pyrido[4,3-b]indole

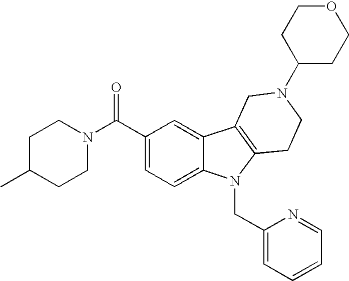

### Step A: The preparation of tert-butyl 8-[(4-methylpiperidin-1-yl)carbonyl]-5-(pyridin-2-ylmethyl)-1,3,4,5-tetrahydro-2H-pyrido[4,3-b]indole-2-carboxylate

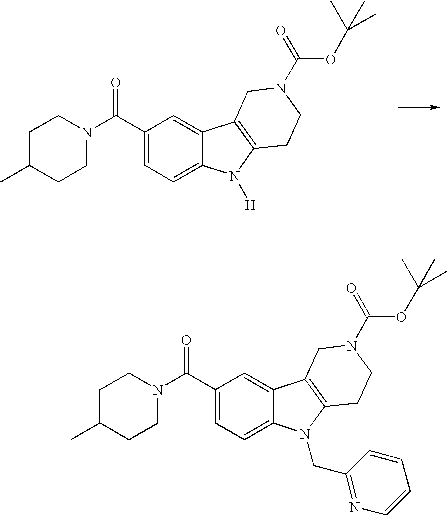

Following the similar procedure of Example 135, Step A: The title compound was prepared from tert-butyl 8-[(4-methylpiperidin-1-yl)carbonyl]-1,3,4,5-tetrahydro-2H-pyrido[4,3-b]indole-2-carboxylate and 2-(chloromethyl)pyridine. MS (M+1): 489.14.

### Step B. The preparation of 8-[(4-methylpiperidin-1-yl)carbonyl]-5-(pyridin-2-ylmethyl)-2,3,4,5-tetrahydro-1H-pyrido[4,3-b]indole

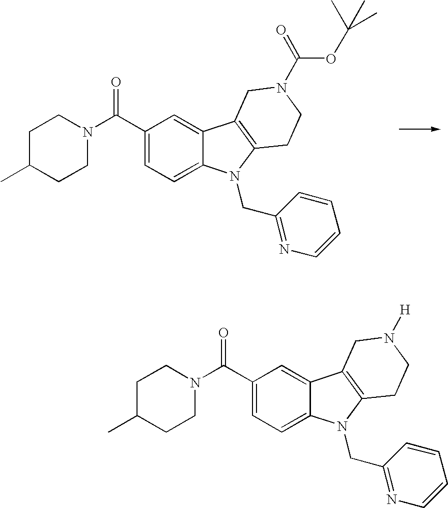

Following the similar procedure of Example 135, Step B: The title compound was prepared from tert-butyl 8-[(4-methylpiperidin-1-yl)carbonyl]-5-(pyridin-2-ylmethyl)-1,3,4,5-tetrahydro-2H-pyrido[4,3-b]indole-2-carboxylate. MS (M+1): 389.06.

### Step C: The preparation of 8-[(4-methylpiperidin-1-yl)carbonyl]-5-(pyridin-2-ylmethyl)-2-(tetrahydro-2H-pyran-4-yl)-2,3,4,5-tetrahydro-1H-pyrido[4,3-b]indole

Following the similar procedure of Example 135, Step B: The title compound as its TFA salt was prepared from 8-[(4-methylpiperidin-1-yl)carbonyl]-5-(pyridin-2-ylmethyl)-2,3,4,5-tetrahydro-1H-pyrido[4,3-b]indole and tetrahydro-4H-pyran-4-one. MS (MS+1): 473.0. 1H NMR (600 MHz, CDCL3) δ ppm 0.87-1.30 (m, 5H), 1.41-1.84 (m, 3H), 1.92-2.05 (m, 2H), 2.06-2.19 (m, 2H), 2.73-3.20 (m, 4H), 3.26-3.39 (m, 1H), 3.46 (t, J=11.25 Hz, 2H), 3.54-3.72 (m, 2H), 3.73-3.87 (m, 1H), 4.13 (d, J=8.78 Hz, 2H), 4.21-4.36 (m, 1H), 4.49-4.66 (m, 2H), 5.47-5.63 (m, 2H), 6.93-7.11 (m, 3H), 7.35-7.44 (m, 1H), 7.54-7.64 (m, 1H), 7.99-8.10 (m, 1H), 8.55-8.67 (m, 1H).

### Example 220

### 5-(ethylsulfonyl)-2-(1-methylpiperidin-4-yl)-8-[(4-methylpiperidin-1-yl)carbonyl]-2,3,4,5-tetrahydro-1H-pyrido[4,3-b]indole

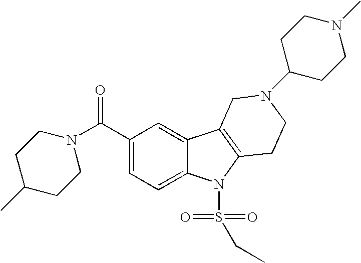

Following the similar procedure of Example 99, Step C: The title compound was prepared from 5-(ethylsulfonyl)-8-[(4-methylpiperidin-1-yl)carbonyl]-2,3,4,5-tetrahydro-1H-pyrido[4,3-b]indole TFA salt and 1-methylpiperidin-4-one. MS (MS+1): 487.0. 1H NMR (400 MHz, CD3OD) δ ppm 0.98 (d, J=6.45 Hz, 3H), 1.07-1.28 (m, 2H), 1.18 (t, J=7.32 Hz, 3H), 1.54-1.86 (m, 3H), 2.21-2.39 (m, 2H), 2.56 (d, J=12.69 Hz, 2H), 2.80-2.98 (m, 1H), 2.92 (s, 3H), 3.04-3.27 (m, 1H), 3.19 (t, J=12.40 Hz, 2H), 3.41-3.52 (m, 4H), 3.63-3.90 (m, 6H), 4.57-4.69 (m, 3H), 7.40 (d, J=8.59 Hz, 1H), 7.62 (d, J=1.17 Hz, 1H), 8.02 (d, J=8.59 Hz, 1H).

### Example 221

### 5-allyl-8-[(4-methylpiperidin-1-yl)carbonyl]-2-(tetrahydrofuran-3-yl)-2,3,4,5-tetrahydro-1H-pyrido[4,3-b]indole

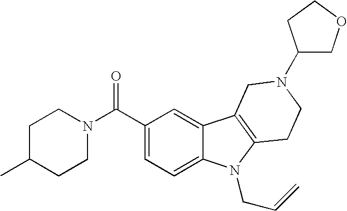

Following the similar procedure described in Example 89, Step C: The title compound was prepared from dihydrofuran-3(2H)-one from Example 91 step A and 5-allyl-8-[(4-methylpiperidin-1-yl)carbonyl]-2,3,4,5-tetrahydro-1H-pyrido[4,3-b]indole TFA salt from Example 1 Step D. MS (M+1): 408.0. 1H NMR (400 MHz, METHANOL-D4) δ ppm 0.99 (d, J=6.64 Hz, 3H), 1.06-1.31 (m, 2H), 1.48-1.89 (m, 3H), 2.25-2.42 (m, 1H), 2.47-2.60 (m, 1H), 2.76-2.98 (m, 1H), 2.99-3.17 (m, 1H), 3.19-3.26 (m, 2H), 3.64-3.89 (m, 1H), 3.79 (q, J=8.01 Hz, 3H), 3.91-4.03 (m, 1H), 4.15 (dt, J=8.69, 4.10 Hz, 1H), 4.21-4.39 (m, 3H), 4.34-4.77 (m, 2H), 4.34-4.77 (m, 3 H), 5.15 (d, J=10.55 Hz, 1H), 5.90-6.06 (m, 1H), 7.25 (d, J=8.59 Hz, 1H), 7.46 (d, J=8.40 Hz, 1H), 7.59 (d, J=0.98 Hz, 1H).

1. A compound of formula I, a pharmaceutically acceptable salt thereof, diastereomers, enantiomers, or mixtures thereof: 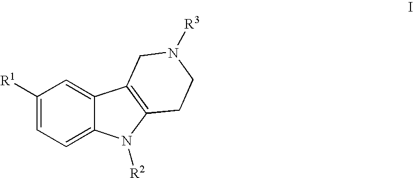
wherein
R¹ is selected from —C(═O)—R⁴, —NH—C(═O)—R⁵, —NH—C(═O)—NH—R⁶, —NR⁷—S(═O)₂—R⁸, —NR⁷—S(═O)₂—NR⁹R¹⁰;
R² is selected from —H, C₁₋₆alkyl, C₂₋₆alkenyl, —C(═O)—NR⁹R¹⁰, —S(═O)₂—NR⁹R¹⁰, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl used in defining R² is optionally substituted with one or more group selected from —OR, R, —CO₂H, —CO₂—R; —SO₂—R; halogen, —NO₂, —OH, —NH₂, —NHR, —C(═O)—NH₂, and —C(═O)—NHR;
R³ is selected from C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₁₋₆alkenyl, C₆₋₁₀aryl-C₁₋₄alkyl, C₃₋₆heteroaryl-C₁₋₄alkyl, —C(═O)—C₁₋₆alkyl, —C(═O)—C₃₋₆cycloalkyl and —C(═NH)—C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkenyl, C₃₋₆heteroaryl-C₁₋₄alkyl, —C(═O)—C₁₋₆alkyl, —C(═O)—C₃₋₆cycloalkyl and —C(═NH)—C₁₋₆alkyl used in defining R³ is optionally substituted with one or more groups selected from —OR, R, NO₂, —CO₂H, —CO₂—R; —SO₂—R; halogen; —OH; —NH₂; —NHR, —C(═O)—NH₂, and —C(═O)—NHR;
R is C₁₋₆alkyl;
R⁴ is selected from a nitrogen containing C₃₋₉heterocyclyl and —NR⁹R¹⁰, wherein said nitrogen containing C₃₋₉heterocyclyl may be optionally substituted with one or more groups selected from C₁₋₆alkyl, phenyl, C₁₋₆alkoxy, —NH₂, —OH, halogenated C₁₋₆alkyl, and halogen; and
R⁵, R⁶, R⁷, R⁸, R⁹ and R¹⁰ are independently selected from —H, C₁₋₆alkyl, C₆₋₁₀aryl, C₆₋₁₀aryl-C₁₋₄alkyl, C₃₋₆heterocyclyl, C₃₋₆heterocyclyl-C₁₋₄alkyl, C₂₋₆alkenyl, C₃₋₆cycloalkyl, and C₃₋₆cycloalkyl-C₁₋₄alkyl; N,N-di(C₁₋₄alkyl)amido-C₁₋₆alkyl, hydroxy-C₁₋₆alkyl and C₁₋₆alkoxy-C₁₋₆alkyl.

2. A compound as claimed in claim 1, wherein
R¹ is selected from —C(═O)—R⁴, —NH—C(═O)—R⁵, —NH—C(═O)—NH—R⁶, —NR⁷—S(═O)₂—R⁸, —NR⁷—S(═O)₂—NR⁹R¹⁰;
R² is selected from C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—NR⁹R¹⁰, —S(═O)₂—NR⁹R¹⁰, —C(═O)—C₁₋₆alkyl; C₆₋₁₀aryl-C₁₋₄alkyl; and C₃₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl used in defining R² is optionally substituted with one or more group selected from methoxy, ethoxy, —S(═O)₂CH₃, methyl, ethyl, —NO₂, —CO₂H, —CO₂CH₃, —CO₂CH₂CH₃ and halogen;
R³ is selected from C₃₋₆heterocycloalkyl, C₃₋₆heterocycloalkyl-C₁₋₄alkyl, C₃₋₆cycloalkyl, C₃₋₆cycloalkyl-C₁₋₄alkyl and C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl and C₁₋₆alkyl used in defining R³ is optionally substituted with one or more groups selected from methoxy, ethoxy, —S(═O)₂CH₃, methyl, ethyl, —NO₂, —CO₂H, —CO₂CH₃, —CO₂CH₂CH₃ and halogen;
R⁴ is selected from a nitrogen containing C₃₋₉heterocycloalkyl and —NR⁹R¹⁰, wherein said nitrogen containing C₃₋₉heterocycloalkyl may be optionally substituted with one or more groups selected from methyl, ethyl, phenyl, methoxy, ethoxy, —OH, trifluoromethyl and halogen; and
R⁵, R⁶, R⁷, R⁸, R⁹ and R¹⁰ are independently selected from —H, C₁₋₆alkyl, phenyl, benzyl, C₃₋₆heterocyclyl, C₃₋₆heterocyclyl-methyl, C₂₋₆alkenyl, C₃₋₆cycloalkyl, and C₃₋₆cycloalkyl-methyl; hydroxy-C₁₋₆alkyl, methoxy-C₁₋₆alkyl and ethoxy-C₁₋₆alkyl.

3. A compound as claimed in claim 1,
wherein
R¹ is selected from —C(═O)—R⁴ and —NH—C(═O)—R⁵;
R² is selected from —H, C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—NR⁹R¹⁰, —S(═O)₂—NR⁹R¹⁰, —C(═O)—C₁₋₆alkyl, benzyl and C₃₋₅heteroaryl-methyl, wherein said C₁₋₆alkyl, C₂₋₆alkenyl, —S(═O)₂—C₁₋₆alkyl, —C(═O)—NR⁹R¹⁰, —S(═O)₂—NR⁹R¹⁰, —C(═O)—C₁₋₆alkyl, benzyl and C₃₋₅heteroaryl-methyl used in defining R² is optionally substituted with one or more group selected from methoxy, ethoxy, —NO₂, —S(═O)₂CH₃, methyl, ethyl, —CO₂H, —CO₂CH₃, —CO₂CH₂CH₃ and halogen;
R³ is selected from C₃₋₆heterocycloalkyl, C₃₋₆heterocycloalkyl-methyl, C₃₋₆cycloalkyl, C₃₋₆cycloalkyl-methyl and C₁₋₆alkyl, wherein said C₃₋₆heterocycloalkyl, C₃₋₆heterocycloalkyl-methyl, C₃₋₆cycloalkyl, C₃₋₆cycloalkyl-methyl and C₁₋₆alkyl used in defining R³ is optionally substituted with one or more groups selected from methoxy, ethoxy, methyl, ethyl and halogen;
R⁴ is selected from piperidinyl, piperazinyl and morpholinyl, wherein said piperidinyl, piperazinyl and morpholinyl are optionally substituted with one or more groups selected from methyl, ethyl, methoxy, ethoxy, —OH, hydroxylmethyl, trifluoromethyl and halogen; and
R⁹ and R¹⁰ are independently selected from —H, C₁₋₆alkyl and C₂₋₆alkenyl.

4. A compound as claimed in claim 1, wherein
R¹ is selected from 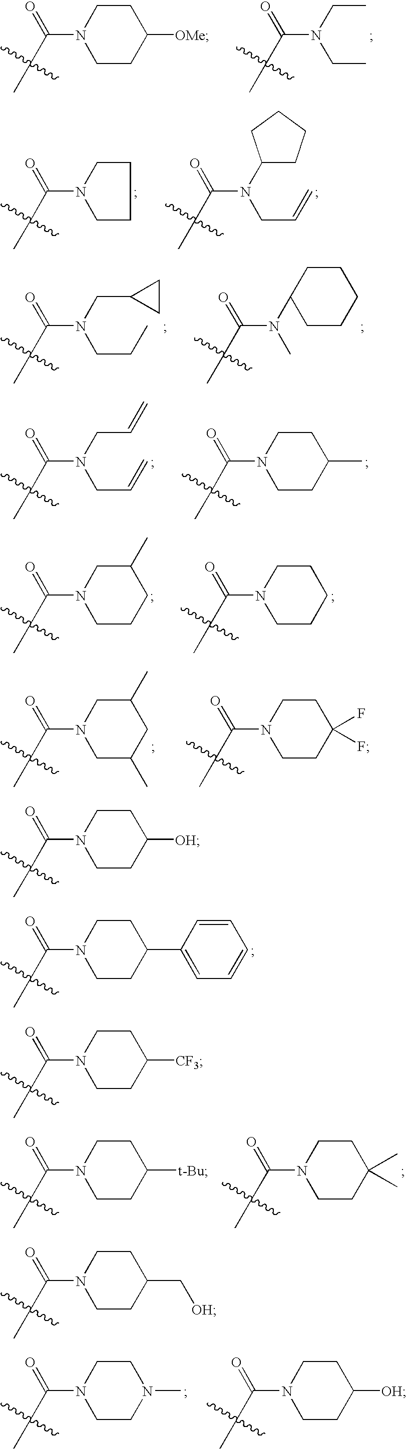 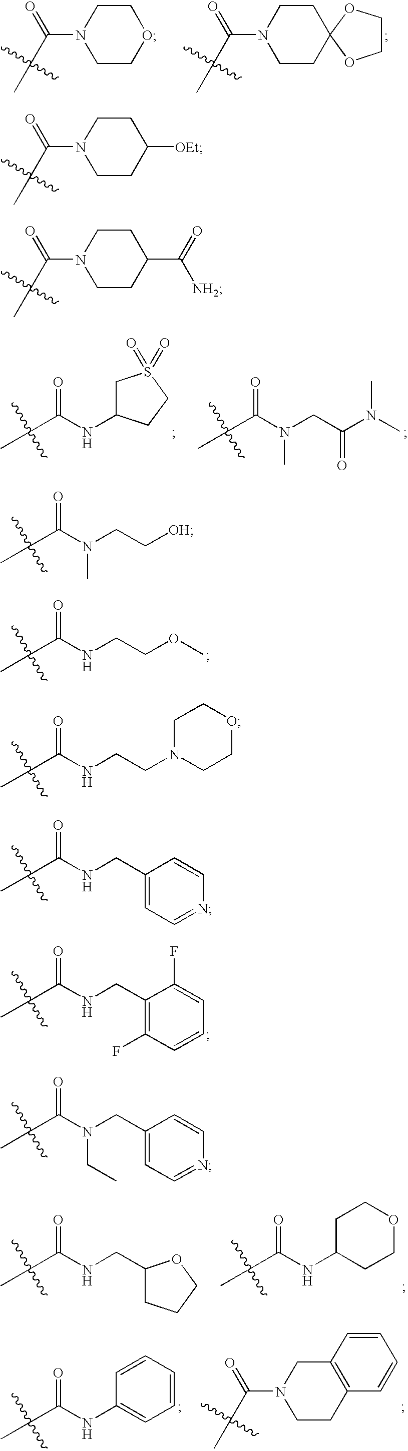 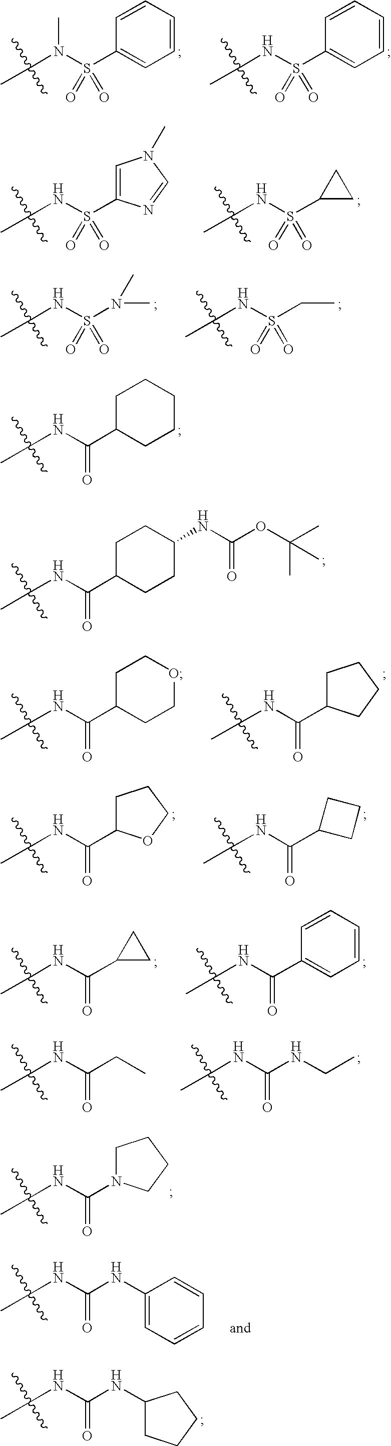
R² is selected from methyl, ethyl, 1-propyl, 2-propyl, 1-butyl, 2-butyl, t-butyl, allyl, —S(═O)₂—CH₃, —S(═O)₂—CH₂CH₃, 2-methoxyethyl, tetrahydropyran-4-yl-methyl, 1-propylsulfonyl, cyclopropylsulfonyl, phenyl, phenylsulfonyl, 2-(methoxycarbonyl)-phenylsulfonyl; 2-(hydroxycarbonyl)-phenylsulfonyl, 1-methyl-1H-imidazol-4-yl-sulfonyl, 1H-imidazol-1-yl-sulfonyl, (5-methylisoxazol-4-yl)sulfonyl, morpholin-4-ylcarbonyl, 4-amino-phenyl, —CH₂—C(═O)—N(CH₃)₂, —C(═O)—N(CH₃)₂, —S(═O)₂—N(CH₃)₂, —S(═O)₂—NHCH₂CH₃, —C(═O)—CH₂CH₂CH₃, —CH₂—C(═O)—OCH₃, —CH₂—C(═O)—OCH₂CH₃, —CH₂—CO₂H, benzyl, 4-aminobenzyl, 4-nitrobenzyl, 4-methylsulfonyl-benzyl, 4-methylthio-benzyl, 4-acetylamino-benzyl, 4-methoxy-benzyl, 4-ethoxy-benzyl, 2,6-difluorobenzyl, (6-chloro-1,3-benzodioxol-5-yl)methyl, (5-ethoxycarbonyl)-fur-2-yl-methyl, (2-methyl-1,3-thiazol-4-yl)methyl, (5-methyl-isoxazol-4-yl)-methyl, pyridin-2-ylmethyl, cyclobutylmethyl, and cyclopropylmethyl;
R³ is selected from ethyl, isopropyl, propyl, 2-methyl-propyl, 1-butyl, 1-pentyl, 1-acetyl-piperidin-4-yl, tetrahydrothien-3-yl, cyclopropylmethyl, cyclobutylmethyl, cyclopentylmethyl, cyclobutyl, cyclopentyl, cyclohexyl, 4-tetrahydro-2H-pyranyl, tetrahydro-thiopyran-4-yl, 2-pyrimidinyl, 1-iminoethyl, 2-pyridinyl, 3,4,5,6-tetrahydropyrdin-2-yl, 3,4-dihydro-2H-pyrrol-5-yl, 2-pyridinyl-methyl, 3-pyridinylmethyl, 4-pyridinylmethyl, 1-methyl-4-piperidinyl, 4-piperidinyl, (6-methyl-pyridin-2-yl)methyl, (2-ethyl-4-methyl-1H-imidazol-5-yl)methyl, tetrahydrofuran-2-yl, tetrahydrofuran-3-yl, tetrahydrofuran-3-ylmethyl, 1-ethyl-1H-pyrazol-4-yl, 1,3-dimethyl-1H-pyrazol-5-yl, (3-methylpyridin-4-yl)methyl, 1,3-oxazol-2-ylmethyl, 1,3-oxazol-5-ylmethyl, 2-(tetrahydro-2H-pyran-4-yl)ethyl, tetrahydro-2H-pyran-4-ylmethyl, 2-phenylethyl, 2-methoxybenzyl, 3,3,3-trifluoropropyl, 2,2-difluoroethyl, 2-hydroxycyclopentyl, (1-ethyl-3-methyl-1H-pyrazol-5-yl)methyl, 2,1,3-benzoxadiazol-5-ylmethyl, 3-thienylmethyl, 2-trifluoromethyl-benzyl, 3-methylbutyl, cyclohex-3-en-1-ylmethyl, 2-fluoro-6-methoxybenzyl, 2-phenyl-propyl, 2-ethyl-butyl, cyclobutylcarbonyl, 2,2-difluoropropanoyl, cyclopentylcarbonyl, tetrahydro-2H-pyran-4-ylcarbonyl, cyclopropylcarbonyl, propylcarbonyl, N-ethylaminocarbonyl, N-isopropylaminocarbonyl, cyclopropylsulfonyl, and ethylsulfonyl.

5. A compound as claimed in claim 1, wherein
R¹ is selected from 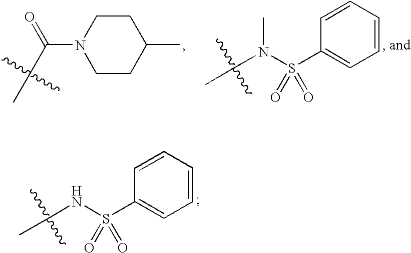
R² is selected from —H, methyl, ethyl, 1-propyl, 2-propyl, 1-butyl, 2-butyl, t-butyl, allyl, —S(═O)₂—CH₃, —S(═O)₂—CH₂CH₃, 2-methoxyethyl, tetrahydropyran-4-yl-methyl, 1-propylsulfonyl, cyclopropylsulfonyl, phenyl, phenylsulfonyl, 2-(methoxycarbonyl)-phenylsulfonyl; 2-(hydroxycarbonyl)-phenylsulfonyl, 1-methyl-1H-imidazol-4-yl-sulfonyl, 1H-imidazol-1-yl-sulfonyl, (5-methylisoxazol-4-yl)sulfonyl, morpholin-4-ylcarbonyl, 4-amino-phenyl, —CH₂—C(═O)—N(CH₃)₂, —C(═O)—N(CH₃)₂, —S(═O)₂—N(CH₃)₂, —S(═O)₂—NHCH₂CH₃, —C(═O)—CH₂CH₂CH₃, —CH₂—C(═O)—OCH₃, —CH₂—C(═O)—OCH₂CH₃, —CH₂—CO₂H, benzyl, 4-aminobenzyl, 4-nitrobenzyl, 4-methylsulfonyl-benzyl, 4-methylthio-benzyl, 4-acetylamino-benzyl, 4-methoxy-benzyl, 4-ethoxy-benzyl, 2,6-difluorobenzyl, (6-chloro-1,3-benzodioxol-5-yl)methyl, (5-ethoxycarbonyl)-fur-2-yl-methyl, (2-methyl-1,3-thiazol-4-yl)-methyl, (5-methyl-isoxazol-4-yl)-methyl, pyridin-2-ylmethyl, cyclobutylmethyl, and cyclopropylmethyl; and
R³ is selected from ethyl, isopropyl, propyl, 2-methyl-propyl, 1-butyl, 1-pentyl, 1-acetyl-piperidin-4-yl, tetrahydrothien-3-yl, cyclopropylmethyl, cyclobutylmethyl, cyclopentylmethyl, cyclobutyl, cyclopentyl, cyclohexyl, 4-tetrahydro-2H-pyranyl, tetrahydro-thiopyran-4-yl, 1-iminoethyl, 3,4,5,6-tetrahydropyrdin-2-yl, 3,4-dihydro-2H-pyrrol-5-yl, tetrahydrofuran-3-ylmethyl, tetrahydrofuran-2-yl, tetrahydrofuran-3-yl, 1-methyl-4-piperidinyl, 2-(tetrahydro-2H-pyran-4-yl)ethyl, tetrahydro-2H-pyran-4-ylmethyl, 3,3,3-trifluoropropyl, 2,2-difluoroethyl, 2-hydroxycyclopentyl, 3-methylbutyl, cyclohex-3-en-1-ylmethyl, and 2-ethyl-butyl.

6. A compound selected from
5-allyl-2-(cyclopropylmethyl)-8-[(4-methylpiperidin-1-yl)carbonyl]-2,3,4,5-tetrahydro-1H-pyrido[4,3-b]indole;
5-allyl-8-[(4-methylpiperidin-1-yl)carbonyl]-2-(tetrahydro-2H-pyran-4-yl)-2,3,4,5-tetrahydro-1H-pyrido[4,3-b]indole;
5-allyl-2-isopropyl-8-[(4-methylpiperidin-1-yl)carbonyl]-2,3,4,5-tetrahydro-1H-pyrido[4,3-b]indole;
2-(1-acetylpiperidin-4-yl)-5-allyl-8-[(4-methylpiperidin-1-yl)carbonyl]-2,3,4,5-tetrahydro-1H-pyrido[4,3-b]indole;
5-allyl-8-[(4-methylpiperidin-1-yl)carbonyl]-2-(tetrahydro-3-thienyl)-2,3,4,5-tetrahydro-1H-pyrido[4,3-b]indole;
5-allyl-2-(cyclopentylmethyl)-8-[(4-methylpiperidin-1-yl)carbonyl]-2,3,4,5-tetrahydro-1H-pyrido[4,3-b]indole;
5-allyl-2-cyclopentyl-8-[(4-methylpiperidin-1-yl)carbonyl]-2,3,4,5-tetrahydro-1H-pyrido[4,3-b]indole;
5-allyl-2-cyclobutyl-8-[(4-methylpiperidin-1-yl)carbonyl]-2,3,4,5-tetrahydro-1H-pyrido[4,3-b]indole;
5-allyl-2-cyclohexyl-8-[(4-methylpiperidin-1-yl)carbonyl]-2,3,4,5-tetrahydro-1H-pyrido[4,3-b]indole;
5-allyl-8-[(4-methylpiperidin-1-yl)carbonyl]-2-(tetrahydro-2H-thiopyran-4-yl)-2,3,4,5-tetrahydro-1H-pyrido[4,3-b]indole;
5-allyl-8-[(4-methylpiperidin-1-yl)carbonyl]-2-pentyl-2,3,4,5-tetrahydro-1H-pyrido[4,3-b]indole;
5-allyl-8-[(4-methylpiperidin-1-yl)carbonyl]-2-(pyridin-2-ylmethyl)-2,3,4,5-tetrahydro-1H-pyrido[4,3-b]indole;
5-allyl-8-[(4-methylpiperidin-1-yl)carbonyl]-2-[(6-methylpyridin-2-yl)methyl]-2,3,4,5-tetrahydro-1H-pyrido[4,3-b]indole;
5-allyl-8-[(4-methylpiperidin-1-yl)carbonyl]-2-(pyridin-3-ylmethyl)-2,3,4,5-tetrahydro-1H-pyrido[4,3-b]indole;
5-allyl-8-[(4-methylpiperidin-1-yl)carbonyl]-2-(pyridin-4-ylmethyl)-2,3,4,5-tetrahydro-1H-pyrido[4,3-b]indole;
5-allyl-2-[(2-ethyl-4-methyl-1H-imidazol-5-yl)methyl]-8-[(4-methylpiperidin-1-yl)carbonyl]-2,3,4,5-tetrahydro-1H-pyrido[4,3-b]indole;
5-allyl-2-(3-methylbutyl)-8-[(4-methylpiperidin-1-yl)carbonyl]-2,3,4,5-tetrahydro-1H-pyrido[4,3-b]indole;
5-allyl-2-(cyclohex-3-en-1-ylmethyl)-8-[(4-methylpiperidin-1-yl)carbonyl]-2,3,4,5-tetrahydro-1H-pyrido[4,3-b]indole;
5-allyl-2-(2-fluoro-6-methoxybenzyl)-8-[(4-methylpiperidin-1-yl)carbonyl]-2,3,4,5-tetrahydro-1H-pyrido[4,3-b]indole;
5-allyl-8-[(4-methylpiperidin-1-yl)carbonyl]-2-(2-phenylpropyl)-2,3,4,5-tetrahydro-1H-pyrido[4,3-b]indole;
5-allyl-2-(2-ethylbutyl)-8-[(4-methylpiperidin-1-yl)carbonyl]-2,3,4,5-tetrahydro-1H-pyrido[4,3-b]indole;
5-allyl-2-butyl-8-[(4-methylpiperidin-1-yl)carbonyl]-2,3,4,5-tetrahydro-1H-pyrido[4,3-b]indole;
5-allyl-8-[(4-methylpiperidin-1-yl)carbonyl]-2-(tetrahydrofuran-3-ylmethyl)-2,3,4,5-tetrahydro-1H-pyrido[4,3-b]indole;
5-allyl-2-[(1-ethyl-1H-pyrazol-4-yl)methyl]-8-[(4-methylpiperidin-1-yl)carbonyl]-2,3,4,5-tetrahydro-1H-pyrido[4,3-b]indole;
5-allyl-2-[(1,3-dimethyl-1H-pyrazol-5-yl)methyl]-8-[(4-methylpiperidin-1-yl)carbonyl]-2,3,4,5-tetrahydro-1H-pyrido[4,3-b]indole;
5-allyl-8-[(4-methylpiperidin-1-yl)carbonyl]-2-[(3-methylpyridin-4-yl)methyl]-2,3,4,5-tetrahydro-1H-pyrido[4,3-b]indole;
5-allyl-8-[(4-methylpiperidin-1-yl)carbonyl]-2-(1,3-oxazol-2-ylmethyl)-2,3,4,5-tetrahydro-1H-pyrido[4,3-b]indole;
5-allyl-8-[(4-methylpiperidin-1-yl)carbonyl]-2-[2-(tetrahydro-2H-pyran-4-yl)ethyl]-2,3,4,5-tetrahydro-1H-pyrido[4,3-b]indole;
5-allyl-8-[(4-methylpiperidin-1-yl)carbonyl]-2-(2-phenylethyl)-2,3,4,5-tetrahydro-1H-pyrido[4,3-b]indole;
5-allyl-2-(2-methoxybenzyl)-8-[(4-methylpiperidin-1-yl)carbonyl]-2,3,4,5-tetrahydro-1H-pyrido[4,3-b]indole;
5-allyl-8-[(4-methylpiperidin-1-yl)carbonyl]-2-(3,3,3-trifluoropropyl)-2,3,4,5-tetrahydro-1H-pyrido[4,3-b]indole;
5-allyl-2-[(1-ethyl-3-methyl-1H-pyrazol-5-yl)methyl]-8-[(4-methylpiperidin-1-yl)carbonyl]-2,3,4,5-tetrahydro-1H-pyrido[4,3-b]indole;
5-allyl-2-(2,1,3-benzoxadiazol-5-ylmethyl)-8-[(4-methylpiperidin-1-yl)carbonyl]-2,3,4,5-tetrahydro-1H-pyrido[4,3-b]indole;
5-allyl-2-[(1-ethyl-3-methyl-1H-pyrazol-4-yl)methyl]-8-[(4-methylpiperidin-1-yl)carbonyl]-2,3,4,5-tetrahydro-1H-pyrido[4,3-b]indole;
5-allyl-8-[(4-methylpiperidin-1-yl)carbonyl]-2-(3-thienylmethyl)-2,3,4,5-tetrahydro-1H-pyrido[4,3-b]indole;
5-allyl-8-[(4-methylpiperidin-1-yl)carbonyl]-2-[2-(trifluoromethyl)benzyl]-2,3,4,5-tetrahydro-1H-pyrido[4,3-b]indole;
5-allyl-8-[(4-methylpiperidin-1-yl)carbonyl]-2-piperidin-4-yl-2,3,4,5-tetrahydro-1H-pyrido[4,3-b]indole;
5-allyl-8-[(4-methylpiperidin-1-yl)carbonyl]-2-(tetrahydro-2H-pyran-4-ylmethyl)-2,3,4,5-tetrahydro-1H-pyrido[4,3-b]indole;
5-allyl-2-(2,2-difluoropropanoyl)-8-[(4-methylpiperidin-1-yl)carbonyl]-2,3,4,5-tetrahydro-1H-pyrido[4,3-b]indole;
5-allyl-2-(cyclopentylcarbonyl)-8-[(4-methylpiperidin-1-yl)carbonyl]-2,3,4,5-tetrahydro-1H-pyrido[4,3-b]indole;
5-allyl-8-[(4-methylpiperidin-1-yl)carbonyl]-2-(tetrahydro-2H-pyran-4-ylcarbonyl)-2,3,4,5-tetrahydro-1H-pyrido[4,3-b]indole;
5-allyl-2-(cyclopropylcarbonyl)-8-[(4-methylpiperidin-1-yl)carbonyl]-2,3,4,5-tetrahydro-1H-pyrido[4,3-b]indole;
5-allyl-2-butyryl-8-[(4-methylpiperidin-1-yl)carbonyl]-2,3,4,5-tetrahydro-1H-pyrido[4,3-b]indole;
5-allyl-N-ethyl-8-[(4-methylpiperidin-1-yl)carbonyl]-1,3,4,4a,5,9b-hexahydro-2H-pyrido[4,3-b]indole-2-carboxamide;
5-allyl-N-isopropyl-8-[(4-methylpiperidin-1-yl)carbonyl]-1,3,4,4a,5,9b-hexahydro-2H-pyrido[4,3-b]indole-2-carboxamide;
5-allyl-2-(cyclopropylsulfonyl)-8-[(4-methylpiperidin-1-yl)carbonyl]-2,3,4,5-tetrahydro-1H-pyrido[4,3-b]indole;
5-allyl-2-(ethylsulfonyl)-8-[(4-methylpiperidin-1-yl)carbonyl]-2,3,4,5-tetrahydro-1H-pyrido[4,3-b]indole;
5-(2-methoxyethyl)-8-[(4-methylpiperidin-1-yl)carbonyl]-2-(tetrahydro-2H-pyran-4-yl)-2,3,4,5-tetrahydro-1H-pyrido[4,3-b]indole;
5-(2-methoxyethyl)-8-[(4-methylpiperidin-1-yl)carbonyl]-2-(pyridin-2-ylmethyl)-2,3,4,5-tetrahydro-1H-pyrido[4,3-b]indole;
5-(2-methoxyethyl)-8-[(4-methylpiperidin-1-yl)carbonyl]-2-[(6-methylpyridin-2-yl)methyl]-2,3,4,5-tetrahydro-1H-pyrido[4,3-b]indole;
5-(2-methoxyethyl)-8-[(4-methylpiperidin-1-yl)carbonyl]-2-(1,3-oxazol-5-ylmethyl)-2,3,4,5-tetrahydro-1H-pyrido[4,3-b]indole;
5-(2-methoxyethyl)-8-[(4-methylpiperidin-1-yl)carbonyl]-2-(1,3-oxazol-2-ylmethyl)-2,3,4,5-tetrahydro-1H-pyrido[4,3-b]indole;
2-ethyl-5-(2-methoxyethyl)-8-[(4-methylpiperidin-1-yl)carbonyl]-2,3,4,5-tetrahydro-1H-pyrido[4,3-b]indole;
5-(2-methoxyethyl)-8-[(4-methylpiperidin-1-yl)carbonyl]-2-[2-(tetrahydro-2H-pyran-4-yl)ethyl]-2,3,4,5-tetrahydro-1H-pyrido[4,3-b]indole;
2-(cyclopentylmethyl)-5-(2-methoxyethyl)-8-[(4-methylpiperidin-1-yl)carbonyl]-2,3,4,5-tetrahydro-1H-pyrido[4,3-b]indole;
5-(2-methoxyethyl)-8-[(4-methylpiperidin-1-yl)carbonyl]-2-(pyridin-3-ylmethyl)-2,3,4,5-tetrahydro-1H-pyrido[4,3-b]indole;
5-(2-methoxyethyl)-2-(3-methylbutyl)-8-[(4-methylpiperidin-1-yl)carbonyl]-2,3,4,5-tetrahydro-1H-pyrido[4,3-b]indole;
2-(cyclohex-3-en-1-ylmethyl)-5-(2-methoxyethyl)-8-[(4-methylpiperidin-1-yl)carbonyl]-2,3,4,5-tetrahydro-1H-pyrido[4,3-b]indole;
2-(cyclopropylmethyl)-5-(2-methoxyethyl)-8-[(4-methylpiperidin-1-yl)carbonyl]-2,3,4,5-tetrahydro-1H-pyrido[4,3-b]indole;
2-butyl-5-(2-methoxyethyl)-8-[(4-methylpiperidin-1-yl)carbonyl]-2,3,4,5-tetrahydro-1H-pyrido[4,3-b]indole;
5-(2-methoxyethyl)-8-[(4-methylpiperidin-1-yl)carbonyl]-2-(tetrahydrofuran-3-ylmethyl)-2,3,4,5-tetrahydro-1H-pyrido[4,3-b]indole;
5-(2-methoxyethyl)-8-[(4-methylpiperidin-1-yl)carbonyl]-2-(pyridin-4-ylmethyl)-2,3,4,5-tetrahydro-1H-pyrido[4,3-b]indole;
5-(2-methoxyethyl)-8-[(4-methylpiperidin-1-yl)carbonyl]-2-pentyl-2,3,4,5-tetrahydro-1H-pyrido[4,3-b]indole;
5-(2-methoxyethyl)-8-[(4-methylpiperidin-1-yl)carbonyl]-2-(2-phenylethyl)-2,3,4,5-tetrahydro-1H-pyrido[4,3-b]indole;
2-cyclopentyl-8-[(4-methylpiperidin-1-yl)carbonyl]-5-propyl-2,3,4,5-tetrahydro-1H-pyrido[4,3-b]indole;
8-[(4-methylpiperidin-1-yl)carbonyl]-5-propyl-2-(3,3,3-trifluoropropyl)-2,3,4,5-tetrahydro-1H-pyrido[4,3-b]indole;
8-[(4-methylpiperidin-1-yl)carbonyl]-5-propyl-2-(tetrahydro-2H-pyran-4-yl)-2,3,4,5-tetrahydro-1H-pyrido[4,3-b]indole;
8-[(4-methylpiperidin-1-yl)carbonyl]-5-propyl-2-(tetrahydrofuran-3-ylmethyl)-2,3,4,5-tetrahydro-1H-pyrido[4,3-b]indole;
2-(2,2-difluoroethyl)-8-[(4-methylpiperidin-1-yl)carbonyl]-5-propyl-2,3,4,5-tetrahydro-1H-pyrido[4,3-b]indole;
(1R*,2R*)-2-{8-[(4-methylpiperidin-1-yl)carbonyl]-5-propyl-1,3,4,5-tetrahydro-2H-pyrido[4,3-b]indol-2-yl}cyclopentanol;
8-[(4-methylpiperidin-1-yl)carbonyl]-5-propyl-2-pyridin-2-yl-2,3,4,5-tetrahydro-1H-pyrido[4,3-b]indole;
8-[(4-methylpiperidin-1-yl)carbonyl]-5-propyl-2-(3,4,5,6-tetrahydropyridin-2-yl)-2,3,4,5-tetrahydro-1H-pyrido[4,3-b]indole;
2-(3,4-dihydro-2H-pyrrol-5-yl)-8-[(4-methylpiperidin-1-yl)carbonyl]-5-propyl-2,3,4,5-tetrahydro-1H-pyrido[4,3-b]indole;
2-(cyclobutylcarbonyl)-8-[(4-methylpiperidin-1-yl)carbonyl]-5-propyl-2,3,4,5-tetrahydro-1H-pyrido[4,3-b]indole;
2-(cyclopentylcarbonyl)-8-[(4-methylpiperidin-1-yl)carbonyl]-5-propyl-2,3,4,5-tetrahydro-1H-pyrido[4,3-b]indole;
2-(2,2-difluoropropanoyl)-8-[(4-methylpiperidin-1-yl)carbonyl]-5-propyl-2,3,4,5-tetrahydro-1H-pyrido[4,3-b]indole;
2-(cyclopropylmethyl)-5-(4-ethoxybenzyl)-8-[(4-methylpiperidin-1-yl)carbonyl]-2,3,4,5-tetrahydro-1H-pyrido[4,3-b]indole;
5-(4-ethoxybenzyl)-2-isobutyl-8-[(4-methylpiperidin-1-yl)carbonyl]-2,3,4,5-tetrahydro-1H-pyrido[4,3-b]indole;
5-(4-ethoxybenzyl)-8-[(4-methylpiperidin-1-yl)carbonyl]-2-propyl-2,3,4,5-tetrahydro-1H-pyrido[4,3-b]indole;
2-cyclobutyl-5-(4-ethoxybenzyl)-8-[(4-methylpiperidin-1-yl)carbonyl]-2,3,4,5-tetrahydro-1H-pyrido[4,3-b]indole;
5-(4-ethoxybenzyl)-8-[(4-methylpiperidin-1-yl)carbonyl]-2-(tetrahydro-2H-pyran-4-yl)-2,3,4,5-tetrahydro-1H-pyrido[4,3-b]indole;
2-(cyclobutylcarbonyl)-5-(4-ethoxybenzyl)-8-[(4-methylpiperidin-1-yl)carbonyl]-2,3,4,5-tetrahydro-1H-pyrido[4,3-b]indole;
5-(4-ethoxybenzyl)-8-[(4-methylpiperidin-1-yl)carbonyl]-2-pyrimidin-2-yl-2,3,4,5-tetrahydro-1H-pyrido[4,3-b]indole;
5-(4-ethoxybenzyl)-8-[(4-methylpiperidin-1-yl)carbonyl]-2-(3,4,5,6-tetrahydropyridin-2-yl)-2,3,4,5-tetrahydro-1H-pyrido[4,3-b]indole;
1-{5-(4-ethoxybenzyl)-8-[(4-methylpiperidin-1-yl)carbonyl]-1,3,4,5-tetrahydro-2H-pyrido[4,3-b]indol-2-yl}ethanimine;
2-(3,4-dihydro-2H-pyrrol-5-yl)-5-(4-ethoxybenzyl)-8-[(4-methylpiperidin-1-yl)carbonyl]-2,3,4,5-tetrahydro-1H-pyrido[4,3-b]indole;
2-(cyclopropylmethyl)-8-[(4-methylpiperidin-1-yl)carbonyl]-5-(tetrahydro-2H-pyran-4-ylmethyl)-2,3,4,5-tetrahydro-1H-pyrido[4,3-b]indole;
8-[(4-methylpiperidin-1-yl)carbonyl]-5-(propylsulfonyl)-2-(tetrahydro-2H-pyran-4-yl)-2,3,4,5-tetrahydro-1H-pyrido[4,3-b]indole;
8-[(4-methylpiperidin-1-yl)carbonyl]-5-(propylsulfonyl)-2-(3,3,3-trifluoropropyl)-2,3,4,5-tetrahydro-1H-pyrido[4,3-b]indole;
2-cyclopentyl-8-[(4-methylpiperidin-1-yl)carbonyl]-5-(propylsulfonyl)-2,3,4,5-tetrahydro-1H-pyrido[4,3-b]indole TFA salt;
8-[(4-methylpiperidin-1-yl)carbonyl]-5-(propylsulfonyl)-2-(tetrahydrofuran-3-yl)-2,3,4,5-tetrahydro-1H-pyrido[4,3-b]indole;
2-(3,4-dihydro-2H-pyrrol-5-yl)-8-[(4-methylpiperidin-1-yl)carbonyl]-5-(propylsulfonyl)-2,3,4,5-tetrahydro-1H-pyrido[4,3-b]indole;
2-cyclopentyl-8-[(4-methylpiperidin-1-yl)carbonyl]-5-(methylsulfonyl)-2,3,4,5-tetrahydro-1H-pyrido[4,3-b]indole;
8-[(4-methylpiperidin-1-yl)carbonyl]-5-(methylsulfonyl)-2-(tetrahydro-2H-pyran-4-yl)-2,3,4,5-tetrahydro-1H-pyrido[4,3-b]indole;
2-cyclobutyl-8-[(4-methylpiperidin-1-yl)carbonyl]-5-(methylsulfonyl)-2,3,4,5-tetrahydro-1H-pyrido[4,3-b]indole;
2-(cyclopropylmethyl)-8-[(4-methylpiperidin-1-yl)carbonyl]-5-(methylsulfonyl)-2,3,4,5-tetrahydro-1H-pyrido[4,3-b]indole;
8-[(4-methylpiperidin-1-yl)carbonyl]-5-(methylsulfonyl)-2-(3,3,3-trifluoropropyl)-2,3,4,5-tetrahydro-1H-pyrido[4,3-b]indole;
5-(cyclopropylsulfonyl)-8-[(4-methylpiperidin-1-yl)carbonyl]-2-(tetrahydro-2H-pyran-4-yl)-2,3,4,5-tetrahydro-1H-pyrido[4,3-b]indole;
2-cyclopentyl-5-(ethylsulfonyl)-8-[(4-methylpiperidin-1-yl)carbonyl]-2,3,4,5-tetrahydro-1H-pyrido[4,3-b]indole;
5-(ethylsulfonyl)-8-[(4-methylpiperidin-1-yl)carbonyl]-2-(tetrahydro-2H-pyran-4-yl)-2,3,4,5-tetrahydro-1H-pyrido[4,3-b]indole;
2-cyclobutyl-5-(ethylsulfonyl)-8-[(4-methylpiperidin-1-yl)carbonyl]-2,3,4,5-tetrahydro-1H-pyrido[4,3-b]indole;
2-(cyclopropylmethyl)-5-(ethylsulfonyl)-8-[(4-methylpiperidin-1-yl)carbonyl]-2,3,4,5-tetrahydro-1H-pyrido[4,3-b]indole;
5-(ethylsulfonyl)-8-[(4-methylpiperidin-1-yl)carbonyl]-2-(tetrahydrofuran-3-yl)-2,3,4,5-tetrahydro-1H-pyrido[4,3-b]indole;
2-(2,2-difluoroethyl)-5-(ethylsulfonyl)-8-[(4-methylpiperidin-1-yl)carbonyl]-2,3,4,5-tetrahydro-1H-pyrido[4,3-b]indole;
5-(isopropylsulfonyl)-8-[(4-methylpiperidin-1-yl)carbonyl]-2-(tetrahydro-2H-pyran-4-yl)-2,3,4,5-tetrahydro-1H-pyrido[4,3-b]indole;
N,N-dimethyl-8-[(4-methylpiperidin-1-yl)carbonyl]-2-(tetrahydro-2H-pyran-4-yl)-1,2,3,4-tetrahydro-5H-pyrido[4,3-b]indole-5-sulfonamide;
N,N-dimethyl-8-[(4-methylpiperidin-1-yl)carbonyl]-2-(3,3,3-trifluoropropyl)-1,2,3,4-tetrahydro-5H-pyrido[4,3-b]indole-5-sulfonamide;
2-cyclopentyl-N,N-dimethyl-8-[(4-methylpiperidin-1-yl)carbonyl]-1,2,3,4-tetrahydro-5H-pyrido[4,3-b]indole-5-sulfonamide;
N,N-dimethyl-8-[(4-methylpiperidin-1-yl)carbonyl]-2-(tetrahydrofuran-3-yl)-1,2,3,4-tetrahydro-5H-pyrido[4,3-b]indole-5-sulfonamide;
2-cyclobutyl-N,N-dimethyl-8-[(4-methylpiperidin-1-yl)carbonyl]-1,2,3,4-tetrahydro-5H-pyrido[4,3-b]indole-5-sulfonamide;
N,N-dimethyl-8-[(4-methylpiperidin-1-yl)carbonyl]-2-(tetrahydro-2H-pyran-4-yl)-1,2,3,4-tetrahydro-5H-pyrido[4,3-b]indole-5-carboxamide;
2-cyclobutyl-N,N-dimethyl-8-[(4-methylpiperidin-1-yl)carbonyl]-1,2,3,4-tetrahydro-5H-pyrido[4,3-b]indole-5-carboxamide;
5-butyryl-2-cyclobutyl-8-[(4-methylpiperidin-1-yl)carbonyl]-2,3,4,5-tetrahydro-1H-pyrido[4,3-b]indole;
5-butyryl-8-[(4-methylpiperidin-1-yl)carbonyl]-2-(tetrahydro-2H-pyran-4-yl)-2,3,4,5-tetrahydro-1H-pyrido[4,3-b]indole;
2-cyclopentyl-8-[(4-methylpiperidin-1-yl)carbonyl]-2,3,4,5-tetrahydro-1H-pyrido[4,3-b]indole;
2-cyclopentyl-5-methyl-8-[(4-methylpiperidin-1-yl)carbonyl]-2,3,4,5-tetrahydro-1H-pyrido[4,3-b]indole;
5-methyl-8-[(4-methylpiperidin-1-yl)carbonyl]-2-(tetrahydro-2H-pyran-4-yl)-2,3,4,5-tetrahydro-1H-pyrido[4,3-b]indole;
2-cyclopentyl-5-ethyl-8-[(4-methylpiperidin-1-yl)carbonyl]-2,3,4,5-tetrahydro-1H-pyrido[4,3-b]indole;
5-ethyl-8-[(4-methylpiperidin-1-yl)carbonyl]-2-(tetrahydro-2H-pyran-4-yl)-2,3,4,5-tetrahydro-1H-pyrido[4,3-b]indole;
2-cyclopentyl-5-(cyclopropylmethyl)-8-[(4-methylpiperidin-1-yl)carbonyl]-2,3,4,5-tetrahydro-1H-pyrido[4,3-b]indole;
5-(cyclobutylmethyl)-2-cyclopentyl-8-[(4-methylpiperidin-1-yl)carbonyl]-2,3,4,5-tetrahydro-1H-pyrido[4,3-b]indole;
2-cyclopentyl-5-[(5-methylisoxazol-4-yl)methyl]-8-[(4-methylpiperidin-1-yl)carbonyl]-2,3,4,5-tetrahydro-1H-pyrido[4,3-b]indole;
2-cyclopentyl-8-[(4-methylpiperidin-1-yl)carbonyl]-5-[(2-methyl-1,3-thiazol-4-yl)methyl]-2,3,4,5-tetrahydro-1H-pyrido[4,3-b]indole;
2-{2-cyclobutyl-8-[(4-methylpiperidin-1-yl)carbonyl]-1,2,3,4-tetrahydro-5H-pyrido[4,3-b]indol-5-yl}-N,N-dimethylacetamide;
2-{2-cyclopentyl-8-[(4-methylpiperidin-1-yl)carbonyl]-1,2,3,4-tetrahydro-5H-pyrido[4,3-b]indol-5-yl}-N,N-dimethylacetamide;
2-{2-cyclohexyl-8-[(4-methylpiperidin-1-yl)carbonyl]-1,2,3,4-tetrahydro-5H-pyrido[4,3-b]indol-5-yl}-N,N-dimethylacetamide;
methyl {2-cyclobutyl-8-[(4-methylpiperidin-1-yl)carbonyl]-1,2,3,4-tetrahydro-5H-pyrido[4,3-b]indol-5-yl}acetate;
methyl {2-cyclopentyl-8-[(4-methylpiperidin-1-yl)carbonyl]-1,2,3,4-tetrahydro-5H-pyrido[4,3-b]indol-5-yl}acetate;
{2-cyclopentyl-8-[(4-methylpiperidin-1-yl)carbonyl]-1,2,3,4-tetrahydro-5H-pyrido[4,3-b]indol-5-yl}acetic acid;
Methyl {2-cyclohexyl-8-[(4-methylpiperidin-1-yl)carbonyl]-1,2,3,4-tetrahydro-5H-pyrido[4,3-b]indol-5-yl}acetate;
methyl 5-({2-cyclopentyl-8-[(4-methylpiperidin-1-yl)carbonyl]-1,2,3,4-tetrahydro-5H-pyrido[4,3-b]indol-5-yl}methyl)-2-furoate;
5-({2-cyclopentyl-8-[(4-methylpiperidin-1-yl)carbonyl]-1,2,3,4-tetrahydro-5H-pyrido[4,3-b]indol-5-yl}methyl)-2-furoic acid;
2-cyclopentyl-8-[(4-methylpiperidin-1-yl)carbonyl]-5-[4-(methylthio)benzyl]-2,3,4,5-tetrahydro-1H-pyrido[4,3-b]indole;
2-cyclopentyl-8-[(4-methylpiperidin-1-yl)carbonyl]-5-[4-(methylthio)benzyl]-2,3,4,5-tetrahydro-1H-pyrido[4,3-b]indole 2-oxide;
2-cyclopentyl-8-[(4-methylpiperidin-1-yl)carbonyl]-5-[4-(methylsulfonyl)benzyl]-2,3,4,5-tetrahydro-1H-pyrido[4,3-b]indole;
8-[(4-methylpiperidin-1-yl)carbonyl]-5-(4-nitrophenyl)-2-(tetrahydro-2H-pyran-4-yl)-2,3,4,5-tetrahydro-1H-pyrido[4,3-b]indole;
{-4-[8-[(4-methylpiperidin-1-yl)carbonyl]-2-(tetrahydro-2H-pyran-4-yl)-1,2,3,4-tetrahydro-5H-pyrido[4,3-b]indol-5-yl]phenyl}amine;
N-{4-[8-[(4-methylpiperidin-1-yl)carbonyl]-2-(tetrahydro-2H-pyran-4-yl)-1,2,3,4-tetrahydro-5H-pyrido[4,3-b]indol-5-yl]phenyl}acetamide;
4-{2-cyclopentyl-8-[(4-methylpiperidin-1-yl)carbonyl]-1,2,3,4-tetrahydro-5H-pyrido[4,3-b]indol-5-yl}phenyl)amine;
2-isopropyl-8-[(4-methylpiperidin-1-yl)carbonyl]-2,3,4,5-tetrahydro-1H-pyrido[4,3-b]indole;
5-benzyl-2-isopropyl-8-[(4-methylpiperidin-1-yl)carbonyl]-2,3,4,5-tetrahydro-1H-pyrido[4,3-b]indole;
2-isopropyl-5-(4-methoxybenzyl)-8-[(4-methylpiperidin-1-yl)carbonyl]-2,3,4,5-tetrahydro-1H-pyrido[4,3-b]indole;
2-isopropyl-8-[(4-methylpiperidin-1-yl)carbonyl]-5-pentyl-2,3,4,5-tetrahydro-1H-pyrido[4,3-b]indole;
2-isopropyl-8-[(4-methylpiperidin-1-yl)carbonyl]-5-propyl-2,3,4,5-tetrahydro-1H-pyrido[4,3-b]indole;
5-[(6-chloro-1,3-benzodioxol-5-yl)methyl]-2-isopropyl-8-[(4-methoxypiperidin-1-yl)carbonyl]-2,3,4,5-tetrahydro-1H-pyrido[4,3-b]indole;
5-(4-ethoxybenzyl)-2-isopropyl-8-[(4-methoxypiperidin-1-yl)carbonyl]-2,3,4,5-tetrahydro-1H-pyrido[4,3-b]indole;
5-(2,6-difluorobenzyl)-2-isopropyl-8-[(4-methoxypiperidin-1-yl)carbonyl]-2,3,4,5-tetrahydro-1H-pyrido[4,3-b]indole;
5-(4-ethoxybenzyl)-8-[(4-ethoxypiperidin-1-yl)carbonyl]-2-isopropyl-2,3,4,5-tetrahydro-1H-pyrido[4,3-b]indole;
2-cyclopentyl-8-[(4,4-difluoropiperidin-1-yl)carbonyl]-5-methyl-2,3,4,5-tetrahydro-1H-pyrido[4,3-b]indole;
8-[(4,4-difluoropiperidin-1-yl)carbonyl]-5-methyl-2-(tetrahydro-2H-pyran-4-yl)-2,3,4,5-tetrahydro-1H-pyrido[4,3-b]indole;
8-[(4,4-difluoropiperidin-1-yl)carbonyl]-5-methyl-2-(tetrahydrofuran-3-yl)-2,3,4,5-tetrahydro-1H-pyrido[4,3-b]indole;
5-allyl-2-(cyclobutyl)-8-[(3-methylpiperidin-1-yl)carbonyl]-2,3,4,5-tetrahydro-1H-pyrido[4,3-b]indole; 1-{[5-methyl-2-(tetrahydro-2H-pyran-4-yl)-2,3,4,5-tetrahydro-1H-pyrido[4,3-b]indol-8-yl]carbonyl}piperidine-4-carboxamide;
1-{[5-methyl-2-(tetrahydro-2H-pyran-4-yl)-2,3,4,5-tetrahydro-1H-pyrido[4,3-b]indol-8-yl]carbonyl}piperidin-4-ol;
(1-{[5-methyl-2-(tetrahydro-2H-pyran-4-yl)-2,3,4,5-tetrahydro-1H-pyrido[4,3-b]indol-8-yl]carbonyl}piperidin-4-yl)methanol;
5-methyl-8-[(4-methylpiperazin-1-yl)carbonyl]-2-(tetrahydro-2H-pyran-4-yl)-2,3,4,5-tetrahydro-1H-pyrido[4,3-b]indole;
5-methyl-8-(morpholin-4-ylcarbonyl)-2-(tetrahydro-2H-pyran-4-yl)-2,3,4,5-tetrahydro-1H-pyrido[4,3-b]indole;
5-methyl-8-(piperidin-1-ylcarbonyl)-2-(tetrahydro-2H-pyran-4-yl)-2,3,4,5-tetrahydro-1H-pyrido[4,3-b]indole;
8-(1,4-dioxa-8-azaspiro[4.5]dec-8-ylcarbonyl)-5-methyl-2-(tetrahydro-2H-pyran-4-yl)-2,3,4,5-tetrahydro-1H-pyrido[4,3-b]indole;
N-(1,1-dioxidotetrahydro-3-thienyl)-5-methyl-2-(tetrahydro-2H-pyran-4-yl)-2,3,4,5-tetrahydro-1H-pyrido[4,3-b]indole-8-carboxamide;
N-[b 2-(dimethylamino)-2-oxoethyl]-N,5-dimethyl-2-(tetrahydro-2H-pyran-4-yl)-2,3,4,5-tetrahydro-1H-pyrido[4,3-b]indole-8-carboxamide;
N-(2-hydroxyethyl)-N,5-dimethyl-2-(tetrahydro-2H-pyran-4-yl)-2,3,4,5-tetrahydro-1H-pyrido[4,3-b]indole-8-carboxamide;
5-methyl-N-(2-morpholin-4-ylethyl)-2-(tetrahydro-2H-pyran-4-yl)-2,3,4,5-tetrahydro-1H-pyrido[4,3-b]indole-8-carboxamide;
5-methyl-N,2-ditetrahydro-2H-pyran-4-yl-2,3,4,5-tetrahydro-1H-pyrido[4,3-b]indole-8-carboxamide;
5-methyl-N-(pyridin-4-ylmethyl)-2-(tetrahydro-2H-pyran-4-yl)-2,3,4,5-tetrahydro-1H-pyrido[4,3-b]indole-8-carboxamide;
N-(2-methoxyethyl)-5-methyl-2-(tetrahydro-2H-pyran-4-yl)-2,3,4,5-tetrahydro-1H-pyrido[4,3-b]indole-8-carboxamide;
5-methyl-N-(tetrahydrofuran-2-ylmethyl)-2-(tetrahydro-2H-pyran-4-yl)-2,3,4,5-tetrahydro-1H-pyrido[4,3-b]indole-8-carboxamide;
N-ethyl-5-methyl-N-(pyridin-4-ylmethyl)-2-(tetrahydro-2H-pyran-4-yl)-2,3,4,5-tetrahydro-1H-pyrido[4,3-b]indole-8-carboxamide;
8-(3,4-dihydroisoquinolin-2(1H)-ylcarbonyl)-5-methyl-2-(tetrahydro-2H-pyran-4-yl)-2,3,4,5-tetrahydro-1H-pyrido[4,3-b]indole;
5-methyl-N-phenyl-2-(tetrahydro-2H-pyran-4-yl)-2,3,4,5-tetrahydro-1H-pyrido[4,3-b]indole-8-carboxamide;
5-methyl-2-(tetrahydro-2H-pyran-4-yl)-8-{[4-(trifluoromethyl)piperidin-1-yl]carbonyl}-2,3,4,5-tetrahydro-1H-pyrido[4,3-b]indole;
8-[(4-tert-butylpiperidin-1-yl)carbonyl]-5-methyl-2-(tetrahydro-2H-pyran-4-yl)-2,3,4,5-tetrahydro-1H-pyrido[4,3-b]indole;
8-[(4,4-dimethylpiperidin-1-yl)carbonyl]-5-methyl-2-(tetrahydro-2H-pyran-4-yl)-2,3,4,5-tetrahydro-1H-pyrido[4,3-b]indole;
5-methyl-8-[(4-phenylpiperidin-1-yl)carbonyl]-2-(tetrahydro-2H-pyran-4-yl)-2,3,4,5-tetrahydro-1H-pyrido[4,3-b]indole;
N-(5-allyl-2-cyclopentyl-2,3,4,4a,5,9b-hexahydro-1H-pyrido[4,3-b]indol-8-yl)-N-methylbenzenesulfonamide;
N-methyl-N-[5-propyl-2-(tetrahydro-2H-pyran-4-yl)-2,3,4,4a,5,9b-hexahydro-1H-pyrido[4,3-b]indol-8-yl]benzenesulfonamide;
N-[5-allyl-2-(tetrahydro-2H-pyran-4-yl)-2,3,4,4a,5,9b-hexahydro-1H-pyrido[4,3-b]indol-8-yl]benzenesulfonamide;
N-(2-cyclopentyl-5-propyl-2,3,4,4a,5,9b-hexahydro-1H-pyrido[4,3-b]indol-8-yl)benzenesulfonamide;
N-(2-cyclopentyl-5-propyl-2,3,4,4a,5,9b-hexahydro-1H-pyrido[4,3-b]indol-8-yl)cyclopropanesulfonamide;
N′-(2-cyclopentyl-5-propyl-2,3,4,4a,5,9b-hexahydro-1H-pyrido[4,3-b]indol-8-yl)-N,N-dimethylsulfamide;
N-(2-cyclopentyl-5-propyl-2,3,4,4a,5,9b-hexahydro-1H-pyrido[4,3-b]indol-8-yl)ethanesulfonamide;
N-(2-cyclopentyl-5-propyl-2,3,4,4a,5,9b-hexahydro-1H-pyrido[4,3-b]indol-8-yl)benzamide;
N-(2-cyclopentyl-5-propyl-2,3,4,4a,5,9b-hexahydro-1H-pyrido[4,3-b]indol-8-yl)propanamide;
N-(2-cyclopentyl-5-propyl-2,3,4,4a,5,9b-hexahydro-1H-pyrido[4,3-b]indol-8-yl)cyclopropanecarboxamide;
N-(2-cyclopentyl-5-propyl-2,3,4,4a,5,9b-hexahydro-1H-pyrido[4,3-b]indol-8-yl)cyclopentanecarboxamide;
N-(2-cyclopentyl-5-propyl-2,3,4,4a,5,9b-hexahydro-1H-pyrido[4,3-b]indol-8-yl)-N'-ethylurea;
N-cyclopentyl-N'-(2-cyclopentyl-5-propyl-2,3,4,4a,5,9b-hexahydro-1H-pyrido[4,3-b]indol-8-yl)urea;
N-(2-cyclopentyl-5-propyl-2,3,4,4a,5,9b-hexahydro-1H-pyrido[4,3-b]indol-8-yl)-N'-phenylurea;
8-[(4-methylpiperidin-1-yl)carbonyl]-5-(phenylsulfonyl)-2-(tetrahydro-2H-pyran-4-yl)-2,3,4,5-tetrahydro-1H-pyrido[4,3-b]indole;
Methyl 2-{[8-[(4-methylpiperidin-1-yl)carbonyl]-2-(tetrahydro-2H-pyran-4-yl)-1,2,3,4-tetrahydro-5H-pyrido[4,3-b]indol-5-yl]sulfonyl}benzoate;
2-{[8-[(4-methylpiperidin-1-yl)carbonyl]-2-(tetrahydro-2H-pyran-4-yl)-1,2,3,4-tetrahydro-5H-pyrido[4,3-b]indol-5-yl]sulfonyl}benzoic acid;
Methyl 2-{[8-[(4-methylpiperidin-1-yl)carbonyl]-2-(tetrahydro-2H-pyran-4-yl)-1,2,3,4-tetrahydro-5H-pyrido[4,3-b]indol-5-yl]sulfonyl}benzoate TFA salt;
2-cyclobutyl-5-[(1-methyl-1H-imidazol-4-yl)sulfonyl]-8-[(4-methylpiperidin-1-yl)carbonyl]-2,3,4,5-tetrahydro-1H-pyrido[4,3-b]indole;
5-[(1-methyl-1H-imidazol-4-yl)sulfonyl]-8-[(4-methylpiperidin-1-yl)carbonyl]-2-(tetrahydro-2H-pyran-4-yl)-2,3,4,5-tetrahydro-1H-pyrido[4,3-b]indole;
8-(3,4-dihydroisoquinolin-2(1H)-ylcarbonyl)-5-(ethylsulfonyl)-2-(tetrahydro-2H-pyran-4-yl)-2,3,4,5-tetrahydro-1H-pyrido[4,3-b]indole;
8-(3,4-dihydroisoquinolin-2(1H)-ylcarbonyl)-5-(ethylsulfonyl)-2-(tetrahydrofuran-2-yl)-2,3,4,5-tetrahydro-1H-pyrido[4,3-b]indole;
8-(3,4-dihydroisoquinolin-2(1H)-ylcarbonyl)-2-(1-methylpiperidin-4-yl)-5-(propylsulfonyl)-2,3,4,5-tetrahydro-1H-pyrido[4,3-b]indole;
5-(1H-imidazol-1-ylsulfonyl)-8-[(4-methylpiperidin-1-yl)carbonyl]-2-(tetrahydro-2H-pyran-4-yl)-2,3,4,5-tetrahydro-1H-pyrido[4,3-b]indole;
8-[(4-methylpiperidin-1-yl)carbonyl]-5-(morpholin-4-ylcarbonyl)-2-(tetrahydro-2H-pyran-4-yl)-2,3,4,5-tetrahydro-1H-pyrido[4,3-b]indole;
5-[(5-methylisoxazol-4-yl)sulfonyl]-8-[(4-methylpiperidin-1-yl)carbonyl]-2-(tetrahydro-2H-pyran-4-yl)-2,3,4,5-tetrahydro-1H-pyrido[4,3-b]indole;
2-cyclopentyl-5-(1H-imidazol-1-ylsulfonyl)-8-[(4-methylpiperidin-1-yl)carbonyl]-2,3,4,5-tetrahydro-1H-pyrido[4,3-b]indole;
N-ethyl-8-[(4-methylpiperidin-1-yl)carbonyl]-2-(tetrahydro-2H-pyran-4-yl)-1,2,3,4-tetrahydro-5H-pyrido[4,3-b]indole-5-sulfonamide;
5-(ethylsulfonyl)-8-[(4-methoxypiperidin-1-yl)carbonyl]-2-(tetrahydro-2H-pyran-4-yl)-2,3,4,5-tetrahydro-1H-pyrido[4,3-b]indole;
(1-{[2-cyclobutyl-5-(ethylsulfonyl)-2,3,4,5-tetrahydro-1H-pyrido[4,3-b]indol-8-yl]carbonyl}piperidin-4-yl)methanol;
1-{[2-cyclobutyl-5-(ethylsulfonyl)-2,3,4,5-tetrahydro-1H-pyrido[4,3-b]indol-8-yl]carbonyl}piperidin-4-ol;
N-[5-(methylsulfonyl)-2-(tetrahydro-2H-pyran-4-yl)-2,3,4,5-tetrahydro-1H-pyrido[4,3-b]indol-8-yl]cyclopentanecarboxamide;
N-[5-(ethylsulfonyl)-2-(tetrahydro-2H-pyran-4-yl)-2,3,4,5-tetrahydro-1H-pyrido[4,3-b]indol-8-yl]pyrrolidine-1-carboxamide;
N-[5-(ethylsulfonyl)-2-(tetrahydro-2H-pyran-4-yl)-2,3,4,5-tetrahydro-1H-pyrido[4,3-b]indol-8-yl]-1-methyl-1H-imidazole-4-sulfonamide;
tert-butyl(trans-4-{[(2-cyclopentyl-5-propyl-2,3,4,5-tetrahydro-1H-pyrido[4,3-b]indol-8-yl)amino]carbonyl}cyclohexyl)carbamate;
trans-N-(2-cyclopentyl-5-propyl-2,3,4,5-tetrahydro-1H-pyrido[4,3-b]indol-8-yl)-4-[(2,2-dimethylpropanoyl)amino]cyclohexanecarboxamide;
N-[5-propyl-2-(tetrahydro-2H-pyran-4-yl)-2,3,4,5-tetrahydro-1H-pyrido[4,3-b]indol-8-yl]cyclohexanecarboxamide;
N-[5-propyl-2-(tetrahydro-2H-pyran-4-yl)-2,3,4,5-tetrahydro-1H-pyrido[4,3-b]indol-8-yl]tetrahydro-2H-pyran-4-carboxamide;
N-[5-propyl-2-(tetrahydro-2H-pyran-4-yl)-2,3,4,5-tetrahydro-1H-pyrido[4,3-b]indol-8-yl]cyclopentanecarboxamide;
N-[5-propyl-2-(tetrahydro-2H-pyran-4-yl)-2,3,4,5-tetrahydro-1H-pyrido[4,3-b]indol-8-yl]tetrahydrofuran-2-carboxamide;
N-[5-propyl-2-(tetrahydro-2H-pyran-4-yl)-2,3,4,5-tetrahydro-1H-pyrido[4,3-b]indol-8-yl]cyclobutanecarboxamide;
8-[(4-methylpiperidin-1-yl)carbonyl]-5-(pyridin-2-ylmethyl)-2-(tetrahydro-2H-pyran-4-yl)-2,3,4,5-tetrahydro-1H-pyrido[4,3-b]indole;
5-(ethylsulfonyl)-2-(1-methylpiperidin-4-yl)-8-[(4-methylpiperidin-1-yl)carbonyl]-2,3,4,5-tetrahydro-1H-pyrido[4,3-b]indole;
5-allyl-8-[(4-methylpiperidin-1-yl)carbonyl]-2-(tetrahydrofuran-3-yl)-2,3,4,5-tetrahydro-1H-pyrido[4,3-b]indole;
and pharmaceutically acceptable salts thereof.

7. (canceled)

8. (canceled)

9. A method for the treatment of anxiety disorders in a warm-blooded animal, comprising the step of administering to said animal in need of such therapy a therapeutically effective amount of a compound according to claim 1.

10. A method for the treatment of cancer, multiple sclerosis, Parkinson's disease, Huntington's chorea, Alzheimer's disease, gastrointestinal disorders or cardiovascular disorders in a warm-blooded animal, comprising the step of administering to said animal in need of such therapy a therapeutically effective amount of a compound according to claim 1.

11. A pharmaceutical composition comprising a compound according to claim 1 and a pharmaceutically acceptable carrier.

12. A method for the therapy of pain in a warm-blooded animal, comprising the step of administering to said animal in need of such therapy a therapeutically effective amount of a compound according to claim 1.

13. A process for preparing a compound of Formula II, comprising: 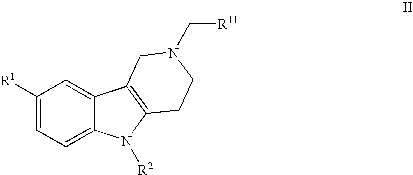
reacting a compound of Formula III with a compound of R¹¹—CHO, 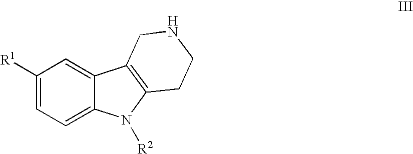
wherein
R¹ is selected from —C(═O)—R⁴, —NH—C(═O)—R⁵, —NH—C(═O)—NH—R⁶, —NR⁷—S(═O)₂—R⁸, —NR⁷—S(═O)₂—NR⁹R¹⁰;
R² is selected from —H, C₁₋₆alkyl, C₂₋₆alkenyl, —C(═O)—NR⁹R¹⁰, —S(═O)₂—NR⁹R¹⁰, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl used in defining R² is optionally substituted with one or more group selected from —OR, R, —CO₂H, —CO₂—R; —SO₂—R; halogen, —NO₂, —OH, —NH₂, —NHR, —C(═O)—NH₂, and —C(═O)—NHR;
R is C₁₋₆alkyl;
R⁴ is selected from a nitrogen containing C₃₋₉heterocyclyl and —NR⁹R¹⁰, wherein said nitrogen containing C₃₋₉heterocyclyl may be optionally substituted with one or more groups selected from C₁₋₆alkyl, phenyl, C₁₋₆alkoxy, —NH₂, —OH, halogenated C₁₋₆alkyl, and halogen;
R⁵, R⁶, R⁷, R⁸, R⁹ and R¹⁰ are independently selected from —H, C₁₋₆alkyl, C₆₋₁₀aryl, C₆₋₁₀aryl-C₁₋₄alkyl, C₃₋₆heterocyclyl, C₃₋₆heterocyclyl-C₁₋₄alkyl, C₂₋₆alkenyl, C₃₋₆cycloalkyl, and C₃₋₆cycloalkyl-C₁₋₄alkyl; N,N-di(C₁₋₄alkyl)amido-C₁₋₆alkyl, hydroxy-C₁₋₆alkyl and C₁₋₆alkoxy-C₁₋₆alkyl; and
R¹¹ is selected from C₃₋₆heterocycloalkyl, C₃₋₆cycloalkyl and C₁₋₆alkyl, wherein said C₃₋₆heterocycloalkyl, C₃₋₆cycloalkyl and C₁₋₆alkyl used in defining R¹¹ is optionally substituted with one or more groups selected from methoxy, ethoxy, methyl, ethyl and halogen.

14. A process for preparing a compound of Formula IV, comprising: 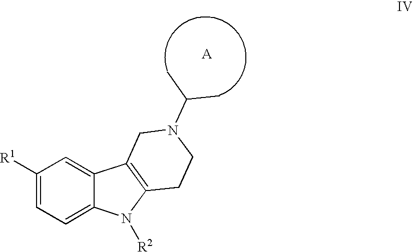
reacting a compound of Formula III with a compound of 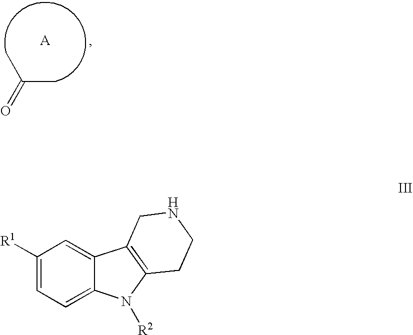
wherein
R¹ is selected from C(═O)—R⁴, —NH—C(═O)—R⁵, —NH—C(═O)—NH—R⁶, —NR⁷—S(═O)₂—R⁸, —NR⁷—S(═O)₂—NR⁹R¹⁰;
R² is selected from —H, C₁₋₆alkyl, C₂₋₆alkenyl, —C(═O)—NR⁹R¹⁰, —S(═O)₂—NR⁹R¹⁰, S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl used in defining R² is optionally substituted with one or more group selected from —OR, R, —CO₂H, —CO₂—R; —SO₂—R; halogen, —NO₂, —OH, —NH₂, —NHR, —C(═O)—NH₂, and —C(═O)—NHR;
R is C₁₋₆alkyl;
R⁴ is selected from a nitrogen containing C₃₋₉heterocyclyl and —NR⁹R¹⁰, wherein said nitrogen containing C₃₋₉heterocyclyl may be optionally substituted with one or more groups selected from C₁₋₆alkyl, phenyl, C₁₋₆alkoxy, —NH₂, —OH, halogenated C₁₋₆alkyl, and halogen;
R⁵, R⁶, R⁷, R⁸, R⁹ and R¹⁰ are independently selected from —H, C₁₋₆alkyl, C₆₋₁₀aryl, C₆₋₁₀aryl-C₁₋₄alkyl, C₃₋₆heterocyclyl, C₃₋₆heterocyclyl-C₁₋₄alkyl, C₂₋₆alkenyl, C₃₋₆cycloalkyl, and C₃₋₆cycloalkyl-C₁₋₄alkyl; N,N-di(C₁₋₄alkyl)amido-C₁₋₆alkyl, hydroxy-C₁₋₆alkyl and C₁₋₆alkoxy-C₁₋₆alkyl; and 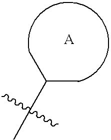
is selected from C₃₋₆heterocycloalkyl and C₃₋₆cycloalkyl, wherein said C₃₋₆heterocycloalkyl and C₃₋₆cycloalkyl are optionally substituted with one or more groups selected from methoxy, ethoxy, methyl, ethyl and halogen.

15. A process for preparing a compound of Formula V, comprising 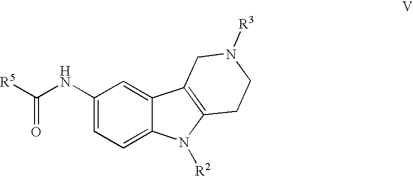
reacting a compound of Formula VI with R⁵—C(═O)—Cl, 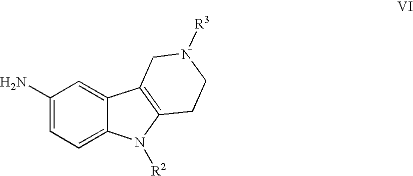
wherein
R² is selected from —H, C₁₋₆alkyl, C₂₋₆alkenyl, —C(═O)—NR⁹R¹⁰, —S(═O)₂—NR⁹R¹⁰, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl used in defining R² is optionally substituted with one or more group selected from —OR, R, —CO₂H, —CO₂—R; —SO₂—R; halogen, —NO₂, —OH, —NH₂, —NHR, —C(═O)—NH₂, and —C(═O)—NHR;
R³ is selected from C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₁₋₆alkenyl, C₆₋₁₀aryl-C₁₋₄alkyl, C₃₋₆heteroaryl-C₁₋₄alkyl, —C(═O)—C₁₋₆alkyl, —C(═O)—C₃₋₆cycloalkyl and —C(═NH)—C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₁₋₆alkenyl, C₆₋₁₀aryl-C₁₋₄alkyl, C₃₋₆heteroaryl-C₁₋₄alkyl, —C(═O)—C₁₋₆alkyl, —C(═O)—C₃₋₆cycloalkyl and —C(═NH)—C₁₋₆alkyl used in defining R³ is optionally substituted with one or more groups selected from —OR, R, NO₂, —CO₂H, —CO₂—R; —SO₂—R; halogen; —OH; —NH₂; —NHR, —C(═O)—NH₂, and —C(═O)—NHR;
R is C₁₋₆alkyl; and
R⁵, R⁹ and R¹⁰ are independently selected from —H, C₁₋₆alkyl, C₆₋₁₀aryl, C₆₋₁₀aryl-4alkyl, C₃₋₆heterocyclyl, C₃₋₆heterocyclyl-C₁₋₄alkyl, C₂₋₆alkenyl, C₃₋₆cycloalkyl, and C₃₋₆cycloalkyl-C₁₋₄alkyl; N,N-di(C₁₋₄alkyl)amido-C₁₋₆alkyl, hydroxy-C₁₋₆alkyl and C₁₋₆alkoxy-C₁₋₆alkyl.

16. A process for preparing a compound of Formula VII, comprising 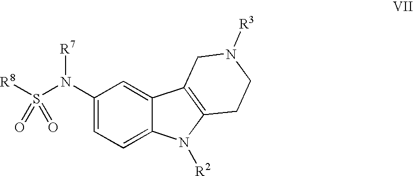
reacting a compound of Formula VIII with R⁹—SO₂—Cl, 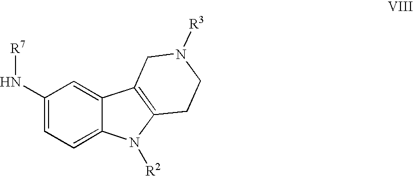
wherein
R² is selected from —H, C₁₋₆alkyl, C₂₋₆alkenyl, —C(═O)—NR⁹R¹⁰, —S(═O)₂—NR⁹R¹⁰, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl used in defining R² is optionally substituted with one or more group selected from —OR, R, —CO₂H, —CO₂—R; —SO₂—R; halogen, —NO₂, —OH, —NH₂, —NHR, —C(═O)—NH₂, and —C(═O)—NHR;
R³ is selected from C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₁₋₆alkenyl, C₆₋₁₀aryl-C₁₋₄alkyl, C₃₋₆heteroaryl-C₁₋₄alkyl, —C(═O)—C₁₋₆alkyl, —C(═O)—C₃₋₆cycloalkyl and —C(═NH)—C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₃₋₆alkyl, C₁₋₆alkenyl, C₃₋₆heteroaryl-C₁₋₄alkyl, —C(═O)—C₁₋₆alkyl, —C(═O)—C₃₋₆cycloalkyl and —C(═NH)—C₁₋₆alkyl used in defining R³ is optionally substituted with one or more groups selected from —OR, R, NO₂, —CO₂H, —CO₂—R; —SO₂—R; halogen; —OH; —NH₂; —NHR, —C(═O)—NH₂, and —C(═O)—NHR;
R is C₁₋₆alkyl; and
R⁷, R⁸, R⁹ and R¹⁰ are independently selected from —H, C₁₋₆alkyl, C₆₋₁₀aryl, C₆₋₁₀aryl-C₁₋₄alkyl, C₃₋₆heterocyclyl, C₃₋₆heterocyclyl-C₁₋₄alkyl, C₂₋₆alkenyl, C₃₋₆cycloalkyl, and C₃₋₆cycloalkyl-C₁₋₄alkyl; N,N-di(C₁₋₄alkyl)amido-C₁₋₆alkyl, hydroxy-C₁₋₆alkyl and C₁₋₆alkoxy-C₁₋₆alkyl.

17. A process for preparing a compound of Formula IX, comprising 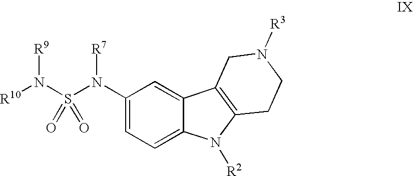
reacting a compound of Formula VIII with R¹⁰R⁹N—SO₂—Cl, 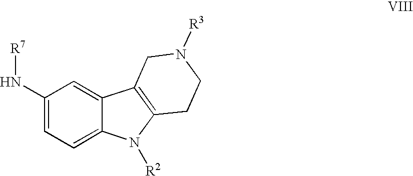
wherein
R² is selected from —H, C₁₋₆alkyl, C₂₋₆alkenyl, —C(═O)—NR⁹R¹⁰, —S(═O)₂—NR⁹R¹⁰, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl used in defining R² is optionally substituted with one or more group selected from —OR, R, —CO₂H, —CO₂—R; —SO₂—R; halogen, —NO₂, —OH, —NH₂, —NHR, —C(═O)—NH₂, and —C(═O)—NHR;
R³ is selected from C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₁₋₆alkenyl, C₆₋₁₀aryl-C₁₋₄alkyl, C₃₋₆heteroaryl-C₁₋₄alkyl, —C(═O)—C₁₋₆alkyl, —C(═O)—C₃₋₆cycloalkyl and —C(═NH)—C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₁₋₆alkenyl, C₃₋₆heteroaryl-C₁₋₄alkyl, —C(═O)—C₁₋₆alkyl, —C(═O)—C₃₋₆cycloalkyl and —C(═NH)—C₁₋₆alkyl used in defining R³ is optionally substituted with one or more groups selected from —OR, R, NO₂, —CO₂H, —CO₂—R; —SO₂—R; halogen; —OH; —NH₂; —NHR, —C(═O)—NH₂, and —C(═O)—NHR;
R is C₁₋₆alkyl; and
R⁷, R⁹ and R¹⁰ are independently selected from —H, C₁₋₆alkyl, C₆₋₁₀aryl-C₁₋₄alkyl, C₃₋₆heterocyclyl, C₃₋₆heterocyclyl-C₁₋₄alkyl, C₂₋₆alkenyl, C₃₋₆cycloalkyl, and C₃₋₆cycloalkyl-C₁₋₄alkyl; N,N-di(C₁₋₄alkyl)amido-C₁₋₆alkyl, hydroxy-C₁₋₆alkyl and C₁₋₆alkoxy-C₁₋₆alkyl.

18. A process for preparing a compound of Formula X, comprising 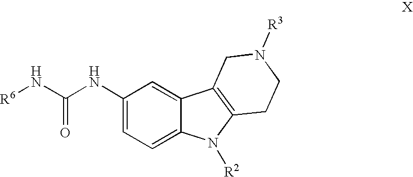
reacting a compound of Formula VI with R⁶NCO, 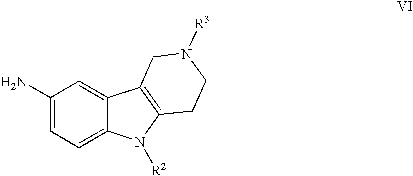
wherein
R² is selected from —H, C₁₋₆alkyl, C₂₋₆alkenyl, —C(═O)—NR⁹R¹⁰, —S(═O)₂—NR⁹R¹⁰, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl, wherein said C₁₋₆alkyl, C₂₋₆alkenyl, —S(═O)₂—C₁₋₆alkyl, —S(═O)₂—C₆₋₁₀aryl, —S(═O)₂—C₃₋₅heteroaryl, —C(═O)—C₁₋₆alkyl; C₆₋₁₀aryl-C₁₋₄alkyl; and C₃₋₅heteroaryl-C₁₋₄alkyl used in defining R² is optionally substituted with one or more group selected from —OR, R, —CO₂H, —CO₂—R; —SO₂—R; halogen, —NO₂, —OH, —NH₂, —NHR, —C(═O)—NH₂, and —C(═O)—NHR;
R³ is selected from C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₁₋₆alkenyl, C₆₋₁₀aryl-C₁₋₄alkyl, C₃₋₆heteroaryl-C₁₋₄alkyl, —C(═O)—C₁₋₆alkyl, —C(═O)—C₃₋₆cycloalkyl and —C(═NH)—C₁₋₆alkyl, wherein said C₃₋₆heterocycloalkyl, C₃₋₆heterocycloalkyl-C₁₋₄alkyl, C₃₋₆cycloalkyl, C₃₋₆cycloalkyl-C₁₋₄alkyl, C₁₋₆alkyl, C₁₋₆alkenyl, C₃₋₆heteroaryl-C₁₋₄alkyl, —C(═O)—C₁₋₆alkyl, —C(═O)—C₃₋₆cycloalkyl and —C(═NH)—C₁₋₆alkyl used in defining R³ is optionally substituted with one or more groups selected from —OR, R, NO₂, —CO₂H, —CO₂—R; —SO₂—R; halogen; —OH; —NH₂; —NHR; —C(═O)—NH₂, and —C(═O)—NHR;
R is C₁₋₆alkyl; and
R⁶, R⁹ and R¹⁰ are independently selected from —H, C₁₋₆alkyl, C₆₋₁₀aryl, C₆₋₁₀aryl-C₁₋₄alkyl, C₃₋₆heterocyclyl, C₃₋₆heterocyclyl-C₁₋₄alkyl, C₂₋₆alkenyl, C₃₋₆cycloalkyl, and C₃₋₆cycloalkyl-C₁₋₄alkyl; N,N-di(C₁₋₄alkyl)amido-C₁₋₆alkyl, hydroxy-C₁₋₆alkyl and C₁₋₆alkoxy-C₁₋₆alkyl.
